SEQUENCE LISTING (35 sequences)

SEQ ID NO: 1 - DNA, 4746 bp, mus musculus
gaggtgaaaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggaggtaagtgaagggacttggctgggctggcagaggcagcagtgaagggaattgggaacatgtagggtagccaccctgcctgccaaaggtggtgatggctgccgggcctcctgagaagcacgacgcagtgtggactagaacccagaattgcaagaatcagaaaccggcctggattgtttcggcctggcccttgtcatgtaggtcacctaggcctggcctgtgtcccgacacttgcttcatgccatcactgtctgtacaccagtgatgcgtgaaaatcagcccccccccaaaaaaaaaaacatatcagcccctctggggacttggatcacagtcggacccaggaacttggccttaaggttaggcatggctgggggggtaaaaaatggtgcttatcctggagttattgttactgaagaggttgggtgtgactggctgctgataggagctcttgtttgggccatgtgtggagtagggctcaccttcagtcaagtttacggcctgtctactttagcctcagactccatgagtcacctttacacgagcagacccttgtagtgcctgaggtgcagatctgatcgatttcagcctttctacctttccttgtaaacaagaaagggacacccttgggtaggggagttttatctccaggccatcttaagatcattctgtgagtgcacgggccttgcttagtgtctgatggcctacagccagcactctggagcaagtgtaagcaattagccttaagaacaaggtgcgagtggataccgatgcccgccgggagttccgacagcttagcgattgttgtagcaggagtcccctccctaagtgccagtttctgtgttatctcaggtcctgtatgccgccgggagtcccctaggaaggcattaatagtttatctcacatcttaaatggcccttaatgaagcaagagatttgaaccttagttaagctaatcccaaatcctcaaaataggatttagaaaagccaaagacactgctgagggcgattacaagttttggtcttttgaggagcagttggagatgaaagtctgtctgaagccgagagaatccttttccattgaaatggcattgaggtgtgcctcactggctgctgcttctgtctgtgccctgggttggccagcctttgtggagcacctcagccctccatcctggacctttgctccaacaacctgctcctcttccgccctcaaggctgacttgcatctccccagatgactgcctccatttctgtcttctgttagagacagaaaagcctgagaaaccgacagccattttggggggggggggtccggttcacacgctgcaacttagaaagcacactcaactggccatctgttataccctccccacctggtcccaaccatcactgtgtactactgagaagaaggcagccttagccacaccctcgagtgcccctgccgttctattgctcatacatcgattgatatccctgtttcaactttgaaaaaaaaaaatttttttttttttgtggtgtgtgcatgcctgctactgtacacctgtgggcgtcagaggtggtcctctgcaccctccggccagtaccgcatccagggtgagtcagatgatttcctgtggtttgggcctcaaggcttctcacctccagaggcttctagcctgctgccttgctttctctgtcgcactctagtacagcaggagttttcttcgcactccggagtgttgtcagctcctggggcatggacatttggctacttagagtgtgctgtgtaggttttcatttagagctgaacagagggatggatcttattaccccagcccttgagacactgaggcaggagagcttcctagtgagtccctgtttcaatatcttcactaatactgtgtcatactttgggactttctttcttcctttctttcttttgattttttttttttttatatgagtacagtgtacctgtcttcagacacacaccagaagagggcatcagatcctactacagaaggttgtgagccaccttgtggttgctgggaattgaactccggatctccggaagagaagtccgtataccaacttctgtattagtcagggttctctagagtcacagaacttatggacagtctctagatagtaaaggaatttattgatgacttacagtcggcagcccaattcccaacaatggttcagtcgcagctgtgaatggaagtccaaggatctagcagttacttagtctcacgcagcaagcaggcgaaggagcaagagctagagcttaactgctgagccatgtgtttcttgagtaaagggattacatgctcgttcgtctggtcaattctgcagccttaaaacttcttcagaatagggtgacattttgtcctcagtggggcggttttgagtaatctgtgagcagataggaacttgctggggtactgcacagaactctgggtagtgtggtactgtagatggctaggttctggggggggaaagagccatctatgtcacctaggaatagagtgaataacatttatataatcagaccagcccttgaggaggctgagatcttttcatggggcaccctagggtcacagtcccagctggtgtgactctgacaagtctgcctttctcactacagtcccaggacatgaaagccctgcagaaggagctagaacagtttgccaagctgctgaagcagaagaggatcaccttggggtacacccaggccgacgtggggctcaccctgggcgttctctttggtgggtctcccccagcatgttctgatctcacggctcttaatgtaggcgcaagggggtggggcatcttaggagctgcttctccacaggtaagggaggattagacgcttgtagcttgaactgtcagaggtgggggcttgggctcccttcttgctgcctcactcactctgtttgatcggcctttcaggaaaggtgttcagccagaccaccatctgtcgcttcgaggccttgcagctcagccttaagaacatgtgtaagctgcggcccctgctggagaagtgggtggaggaagccgacaacaatgagaaccttcaggaggtgaggagtggcaggatgtgtgcaatgtctgccaggcacagtcccttctgctgcttccattcctggcttgaaactcctccctctccaaccggagctcgcaggagaagttctgtgtccttattctgctgctatgaattggaatccagagccttaacatttgctaatcaatcaggctctctccttctgagtcaccctctgcccccaccagcctgacaatggtccctccccagaaccccgtctagtgctggtgaaggctcagacctaggtctaccagccccttccagagcccctttcagtaacccctggctctggggccacatccagtcaatgctcccttagcacaatcccttagcggtttgttcttcagtcccatctcaaggtggggctgttgccaagtcaaatactaaagttgctcttgtcgcccccatcttcccctgcccagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtgagtgccaagatcctgccctgtggtacctggatgtttccctgttcccattccccaccccccccacccccccacccccaccgccgccaccgctgactgcagcatcccagagcttatgatctgatgtccatctctgtgcccatcctaggtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagct

SEQ ID NO: 2 - protein, 352 aa, mus musculus
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 3 - DNA, 2457 bp, mus musculus
ctattaacttgttcaaaaaagtatcaggagttgtcaaggcagagaagagagtgtttgcaaaaagggaaaagtactttgctgcctctttaagactagggctgggagaaagaagaggagagagaaagaaaggagagaagtttggagcccgaggcttaagcctttccaaaaactaatcacaacaatcgcggcggcccgaggaggagagcgcctgttttttcatcccaattgcacttcgcccgtctcgagctccgcttccccccaactattctccgccagatctccgcgcagggccgtgcacgccgaggcccccgcccgcggcccctgcatcccggcccccgagcgcggcccccacagtcccggccgggccgagggttggcggccgccggcgggccgcgcccgcccagcgcccgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggactgcgaactggagaaggggagagattttcaaagagatacaagggaattgggaggggtgcaaaaagaggagagtaggaaaaatctgataatgctcaaaaggaaaaaaaatctccgcagcgaaacgacagctgcggaaaaaaaccaccaatcccatccaaattaacgcaaaaaccgtgatgccgactagaaaacttttatgagagatcttgggacttctttttgggggactatttttgtacagagaaaacctgagggcggcggggagggcgggggaatcggaccatgtatagatctggaggaaaaaaactacgcaaaacttttttttaaagttctagtggtacgttaggcgcttcgcagggagttcgcaaaagtctttaccagtaatatttagagctagactccgggcgatgaaaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaatttaggaccgttacaaacaaggaaggagtttattcggatttgaacattttagttttaaaattgtacaaaaggaaaacatgagagcaagtactggcaagaccgttttcgtggtcttgtttaaggcaaacgttctagattgtactaaatttttaacttactgttaaaggcaaaaaaaaaatgtccatgcaggttgatatcgttggtaatttataatagcttttgttcaatcctaccctttcattttgttcacataaaaaatatggaattactgtgtttgaaatattttcttatggtttgtaatatttctgtaaattgtgatattttaaggtttttccccccttttattttccgtagttgtattttaaaagattcggctctgttattggaatcaggctgccgagaatccatgtatatatttgaactaataccatccttataacagctacattttcaacttaagtttttactccattatgcacagtttgagataaataaatttttgaaatatggacactgaaa

SEQ ID NO: 4 - protein, 319 aa, mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 5 - DNA, 5339 bp, mus musculus
agttccccggccaagagagcgagcgcggctccgggcgcgcggggagcagaggcggtggcgggcggcggcggcacccggagccgccgagtgcccctccccgcccctccagccccccacccagcaacccgcccgtgacccgcgcccatggccgcgcgcacccggcacagtccccaggactccgcaccccgcgccaccgcccagctcgcagttccgcgccaccgcggccattctcacctggcggcgccgcccgcccaccgcccggaccacagcccccgcgccgccgacagccacagtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaagaaggatctcgggcaatctgggggttttggtttgaggttttgtttctaaagtttttaatcttcgttgactttggggctcaggtacccctctctcttcttcggactccggaggaccttctgggcccccacattaatgaggtaggtgagatgttggacttgggaacagaaaggggtgaggattggagggaagcttcggagcagtggccagggtgtcccctgactcctggtccctgtgcgccccgcccggcccgcaggcagccacctggcgagtctgacatggctgtcagcgacgctctgctcccgtccttctccacgttcgcgtccggcccggcgggaagggagaagacactgcgtccagcaggtgccccgactaacgtgagtgtcgcgctagtgccctgtagatttctgcagatggggtggggtgtatgggcggaatccgggggtcaccaagagtccttggttaggacaaggattgccctccattggaactctacagccttccgaggtggctcctcggcccttgagtcggccttaagtgcgtgtggtcccggcactgcggtaccgccaggggcgcacagcggttccccactagtcttcgggcgtccggaccgtggggtgggcgcactcagtccactatattttaggtggacaagtctcgagaccctttgggccggagtgatcgtagcgcgtgggcgacccggtggctatggtgcatggggagaaagccaccgtgtcccagtgtcccaattccgtgggcggctggagcgtcccgcggatctggcggggaagaggactgggctggtggagcgacggcactagcttgcaggagaacgaacgcgccacctcttaccttggtgttagctgagattgcagtgtggcccctggagtccccgaggctgccttcctgttctgtccagttcctatagcagggtgttggtgtgcttcacccaacaagtcaatattaggggtggcaacagggtgctacaattaacagtttctctctccctccctgttcttgcagcgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaaggttagtggggggaagggactggctggtagagatggcgctgggagctttgtttctggggatcttgagactatccagattgaagttgaccccactttctttacccccccccctcccagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggtaagggaaccagcactgtggaggagggttggtgcctgtctgctccgtaggctggctgtgggctccttagggctttcttgggtgctggggtgtatctcttctccatctcctggatcacagaggagggcagcaaacaggcgcctatggttttgcccagaagccttttgcttgatggatgctatctgctgcctgaatgtgcagtttctcattgggcctttaaaagatagggagtttcctgtgtgtgcctgcccctcctagcctggtaatcaagaagttatactttgcaatgcaaaactgtggcggtatctttaaaagaaaagttttaaattttctagattattttcattgttgtctcctcctcctcttagatctacctacccttgactctattctggtctttagaataattagatcgtatttacgtttgaagaatttaatggttcccattgcagggattctggtgtaaaactgtccctgcaagctgggtagggtgacactcaactttggagttacaaataccaaagttgaaagaatcctgttgattcaagaccgatctggtcaagaacccgagtctcgaaataacaaagaacgcacaggtagtcttttagtgggtagagtcttccagatgcatttatgaaaacagccagaacggctctgctggagaaattagccacgggtggcattctggacccagcgtaatggcatgggatcctttgggtttcaacagagccaacttcttttttttttttttccattttttattaagtatttagctcatttacatttccaatgctataccaaaagtcccccatacccacccacccccactcccctacccgcccactccccctttttggccctggcgttcccctgtactggggcatataaagtttgcctgtccaatgggcctctctttccagtgatggccgactaggccatcttttgatatatatgcaactagagacaagagctccagggtactggttagttcataatgttgttccacctataggattgcagatccctttagcagagccaacttctaatccagtccatttccagtgctggttaggctcgagagtgaaaactccgtactgtgggggtggacgctttgagagggtatctgctttttttttttttaagttactaactactgtttctccatgagtttaaactcataacacatgtcattcctcttcatctgaaattcagtatggccctaaagtgtcataaagacattcctgaaggttttttttattattattattatatttttactttcttgaaaggagagagggtggggcatgcagtagaatttgctctctggcctctcccttttttttaagaaaaaaaaggggggtggctaattcttccttctagcaatggcttcagcagcccgttctctttccctttacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagttcaagagacaaaacagaaatactaaaaacaaacaaacaaaaaaacaaacaaaaaaaacaagaaaaaaaaatcacagaacagatggggtctgatactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttgtatgctctttgccttcttaaaaaaaaaaaaagccattattgtgtcggaggaagaggaagcgattcaggtacagaacatgttctaacagcctaaatgatggtgcttggtgagtcgtggttctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaacgacaa

SEQ ID NO: 6 - protein, 483 aa, mus musculus
MRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - DNA, 5021 bp, mus musculus
cccgcccacccgccctttatattccgggggtctgcgcggccgaggacccctgggctgcgctgctctcagctgccgggtccgactcgcctcactcagctcccctcctgcctcctgaagggcagggcttcgccgacgcttggcgggaaaaagaagggaggggagggatcctgagtcgcagtataaaagaagcttttcgggcgtttttttctgactcgctgtagtaattccagcgagagacagagggagtgagcggacggttggaagagccgtgtgtgcagagccgcgctccggggcgacctaagaaggcagctctggagtgagaggggctttgcctccgagcctgccgcccactctccccaaccctgcgactgacccaacatcagcggccgcaaccctcgccgccgctgggaaactttgcccattgcagcgggcagacacttctcactggaacttacaatctgcgagccaggacaggactccccaggctccggggagggaatttttgtctatttggggacagtgttctctgcctctgcccgcgatcagctctcctgaaaagagctcctcgagctgtttgaaggctggatttcctttgggcgttggaaaccccggtaagcacagatctggtggtctttccctgtgttctttctgcgtcttgaatgtagcggccggttaggacagtctttcttccattcctgtgcttttgacacttttctcaagagtagttggggtaggctggggtagatctgagtcggggtagagcgacttgtcaagatgacagaggaaaggggaagggaaaaaccgggatgcattttgaagcggggttcccgaggttactatgggctgacgctgacccggccggttggacattcttgctttgctacattaattgatatgtgtcctttgaggggtcaaaccgggaggtcgcttcgtggtggccaaagaaagcccttggaatcctgaggtctttggagaagggattaccttttgcgtttgggagcgagaaggctccgtagcttctgacttaccagtctctgagagggcatttaaatttcagcttggtgcatttctgacagcctgggaccgacacggaggtgcgtcccgcccgccaatccccggcggcgatcgcaacccgtccctgagccttttaagaagttgctattttggctttaaaaatagtgatcgtagtaaaatttaagcctgacccccgcggcactaggacttgatgttgggctagcgcagtgaggagaagcaaaattgggacagggatgtgaccgattcgttgacttgggggaaaccagagggaatcctcacattcctacttgggatccgcgggtatccctcgcgcccctgaattgctaggaagactgcggtgagtcgtgatctgagcggttccgtaacagctgctaccctcggcggggagagggaagacgccctgcacccagtgctgaatcgctgcagggtctctggtgcagtggcgtcgcggtttagagtgtagaagggaggtgtctcttattatttgacaccccctccccttttatttcgagaggcttgtgatagccggagactgagctctctcctccaagtcagcaatcggaaagaaaagccggcaaaggaaggaagggggcgcgctgggggtggagaaagaggagggcggagaggggcggcggcgccggctgggtaggagcgcggcgacggcgcgaatagggactcggacccggtcggcggcgcagagagccggcacacgggagggggccgagcgacgcggcgcctctcgcctttctccttcaggtggcgcaaaactttgcgcctcggctcttagcagactgtattccctacagtcgcctccctcagcctctgaagccaaggccgatggcgattcctgggcgtctgcagggctaagtccctgctcgaaggaggcggggactcggagcagctgctagtccgacgagcgtcactgatagtagggagtaaaagagtgcatgcctcccccccaaccacacacacacacacacacacacacacacacacacacacacacacacttggaagtacagcacgctgaaaggggagtggttcaggattggggtacgcgctgcgccaggtttccgcaccaaccagagctggataactctagacttgcttcccttgctgtgccccctccagcagacagccacgacgatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctggtaagctaccccattcacagcagggtaggaagcgagaggttggatggacctccttctccaccactcattggcattaattcaattggcctccggggctccccctttctttcccttctgtctaagagctcttcatccctggattcccgtgcttcagctatccctccctcccctcccccccccccccccagaccgccctggttgtcacccccaccccccacccttcccacccctcaggaatttcatttgggtttttaaatcttctggcttatctttcagctccatccactccctttacccctcctaagcattttaattaccctgggaagggtgtgaatgaggataaaggaactgatctggaggggggtgaattacctgcttcttttcttgactgccagaagaatatttgaatttaatggatacgtttgtctaagacccaagagaagcaatgacagaagctgggacagcctttatagccttagagccaggcactagtgaaagttcctaaagaattgaagagctgggctcttttgggtgttttgttttgctttgttttttcgtcctcttttgaacacttcaaagcaaattctgttcaatttggacttctccccccgtcccaacactcccccaacaccaggacgtttggcaaagctgcaagactttttttttttttttttttttttaattgtgcttccagtaaaatagggagttgctaaagtcatagcaagagatttgcagctatccctcacgggacctgaaaggttctcggtaaagtcccttaaaaataggaggtgcttgggaaatgtgctttgctttgggtgtgtctgaagcctcattaaatcttaggtaagaattggcaaggataccatatcctggtacatggtaattttctcacctgtgccctaaccctgttctgcctttctgggagaagggaagatggtgtctggatctgattcttactttcttccctttccaacttggtatttggatagcatcggtcaaatcctatgtatagcgtccgggattcaggaggcgtggctaactgtgatcttccacttcctcccttacagaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataaactgacctaactcgaggaggagctggaatctctcgtgagagtaaggagaacggttccttctgacagaactgatgcgctggaattaaaatgcatgctcaaagcctaacctcacaaccttggctggggctttgggactgtaagcttcagccataattttaactgcctcaaacttaaatagtataaaagaacttttttttatgcttcccatcttttttctttttccttttaacagatttgtatttaattgtttttttaaaaaaatcttaaaatctatccaattttcccatgtaaatagggccttgaaatgtaaataactttaataaaacgtttataacagttacaaaagattttaagacatgtaccataattttttttatttaaagacattttcatttttaaagttgatttttttctattgtttttagaaaaaaataaaataattggaaaaaatac

SEQ ID NO: 8 - protein, 438 aa, mus musculus
MELATRSQIPKEVADIFSAPSDDEEFVGFQDDVPMQNLSESCGSLDSRELEKQQNVCFRSKYFTEELRRIFKEDTDSEMEDFEGFTESELNMSSNPELMESELSDSDKAYPVMNDAEEDDEEEAAPRRGRSTRRSSFGLRVAFQFPTKKLARTPDKDSSHLLDSKTDLRRKKSSRQPKGKEDSASDAEDESRAESQENSDALLKRAMNIKENKAMLAQLLAELNSVPDFFPVRTPPSASRRRTPRRAFSEGQITRRMNPTRSARPPEKFALENFTFSATKLTEELYSFRRRKTISGGKCQTYRRHRISSFRSVKDITEEDLENIAITVRDKVYDKVLGNTCHQCRQKTIDTKTVCRNQSCGGVRGQFCGPCLRNRYGEDVRTALLDPKWTCPPCRGICNCSYCRRRDGRCATGILIHLAKFYGYDNVKEYLESLQKQL

SEQ ID NO: 9 - DNA, 6211 bp, mus musculus
tctatcgccttgagccgttggccttcagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctggtaaggaattcagtccccgaagaacccagtaaattagggtaagagtagaaaaaacgggctgaagggttattgcttctttatgaaccgtagtagtcattaacataagcgggtgtccttatcactcttctggaaacccctctgagtttgaccggtgatacgttggccttctagtctgaaatagagatccgggacacaggacggagcaatataaggctcttgtgtgtatttgagaacctaaatatttgggctctttggaatatgttcgggggcagtgagtggtgtcttcagtagcagggagacctaacatcctagcttggaatgagcaccccatttttttggtgaggttatacagttagtttgcaatttgcccaaatggccgttttctccagcagaagcagttagcatttctctcaacctttgggaatatggatgtcacttagcagttctgggtttgtgagctcagtgctccttccaaaccccaaacaagtgtacacagtttaagtcattcattcttatagctagcatctggagccaaggtctgaagctcattgtctgtacagagtcagcccagtactcaggcttgtaaatggtagaaccaagaggctgctaggctgtgtgtgccacggtgctcaggtgcgtgttgtgatgctcgggtgtgtgcagcgatgctcgggtgtgtgcagtgatgctcgggtgtgtgcagcggtgcttgggtgtgtggagcgctaggaagtgtgtgtccccttttttctgagcttttaagttaatgtctgtcatcttgatttttctgatttatgggttcaatttttgattttttttttgtttgtttttttttgttttttcgagacagggtttctctgtgtagccctggctgtcctggaactcactttgtagaccaggctggcctcaaactcagaaatccgcctgcctctgcctctgcctccctcaatttttgattttggaggtgggaaaagtgctgggttaggaatgaacgggtggggatagcctttgtaagtgatgtgtgtttttagagcatgtgtgtagagcatgtgtgtttgtagtgcgtgcccctagtgccagcagatggcataattttccatttagaagctgctttcagggctgtctacttatttgctagactgcaggctaacctcagcattgccatcagcgtggagcacagatgtcttcagtgtaggaggtgtcttctacctgctgctggatctaggaaatgttggggcaaagcttggtggaggagcacttgtctcttgctgaaggccctggaaaaaaacaaaaaagctaaccgaagagctagagggtcaaggataagagaaacgaatttggttaaccgaaattttactctagtaattaataattaactggaccctctgactggctgctcttgtactagcagaaaagatggtgggatatttttatttatttatttttttaaagatttatttattatatgtaagtacactgtagttgtcttcagacactccagaagagggcgtcagatcttgttacagatggttgtgagccaccatatggttgctgggatttgaactccagaccttcggaagagcagttgggtgctcttacccactgagccgttggatgctcttacccactgagccatctcaccagccccagtgggatcattttaaaatatacctttaaaaaaaaattgggggtggggggtgtctaaactcaggactgtgcattcctaacaacaagtgatttaaccctggaaatttccttagataacattcagtataatggatttcgtcatgacattttttgttcataaatattagttcatccctaaatgttaatgtgtgtgtgtgtgtgtgtgtgtgtatgtatgtatgtatgtatgtatgtatatatgtatgtatgtatagatatagctgactcagtggtagtagagtatgaaaggtcccaacaggattctattcacccagcaccatgcaaaaatctaggcgagataaaagtccccccaaacctgagtctttggataaacctccactcagaaggtagagaggtaggacccagagagcaggccagccttggctacctgagaccctatcccttagaaagaaaagaaaaccacaaacatagactagatcctctgctgcctaagctcttgtgctgtcttgtctgtctgtctctatttgttgagacgggctgatctcaaattctcccatctcagctactggagcaggggggctttagatttgtgctacccactgtcaggatcagaggtttacagtgtagatgcggtcacggttctaatgaagagggagcagggagaagggattggaaagagcagactacaaactcaaacctgtcttcatcctgccattgaactgagtattgtttggttagttgtctttttttttttttaaataggattcccagggtctaattgtataatttaaataactaatgaaacttaaaaacactgtatgtagggctggtgagatggctcaacaggtaaaagcactgactgcccttcccaaagtcctgagttcaaatcccagcaaccacatggtggctcacaaccatccgtaatgagatctgatgccctcttctggtgtgtctgaagacagctacagtgtacttacatataataataaataaatatttagcagagacattcttttttttttttaattaattaatcccatgtggttgctgggaattgaactcaggaccttcggaagagcagtctgcactcttaaccttaaccgctgagccatctctccagccccctaagtagaaacattcttaaaaaaaaaaaaaaaaaaaaaaaaaacactataaaagggagatctatgcaaaatggtgttacccaggggtgacaaagtattccaagaatactttccaaaaattaggtttttcttgttacttagatctatgaagataattgctttgaaagtaaatatagccgggcgtttaatcccagcacttgggaggcagaggcaggcgcatttctgagttcgaggccagcctggtctacaaagtgagctccaggacagccagggctatacagagaaaccctgtctcgaaaaaaccaaaaaaaaagaaaaaaaaaaaaaaaaaagaaagaaagaaagaaagaaagaaagaaagaaagaaagaaagaaagtaagtaaatatattaaatgtaacatttaattatctgggaaaatgatttatttgtttggttggttttgtttttgtttttttggtcagggtttctctgtgtagccctggccatcctggaatttactttgtaaaactgactggcctttttaaaatttatttattatatgtaagtacactgtagctgtcttcagacacaccagaagagggcatcagatctcattacggatggttgtgagccaccatgtggttgctgggatttgaactcaggaccttcggaagagcagtcagtgctcttaaccgctgagccataacccccaccccaccccgtgaactgactggacttaaacccataatctgcctgcttgagtgtcccaagtgctgggactaaaggtatgtaccaccatgccaggctgagaatgtcttatttattaacaagttatctatttatttctaacagcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggtggggtccttagaagttgctgtaatgtaacacaatgagactgacttgctaatgaatcgaactaacgtctggacgtctctctaactatcctatcctcctatcctatgcgcctgttaaaatcataattatgtgattgattgattgattgattatgtataagtacactatagctgtcttcagatacaccagaagagggcatcagatctcattacggatggttgtgagccaccatgtggttgctgggatttgaactcaggaccttcggaagagcagtcagtgctcttaaccgctgagccatctctccagccccaattatgtgatttatatatttcccagtttgtttcattggttactttatcttctacttcacctcccacacccatccccaaatcagggtctcactgtagccctgactggtctctgtagaccaggctgggagaggttcacctgcctctgtctcctgggtgctggaaataaaggtgtgcctggcttttttcctttcttttcttttttaagattgtttatttttaaattcctttagttcattggtgttttgcctgcatgtgtttgtctttgtgagagtatcagatctcctggactagagttacagacagtcgtgagctgccatgtggccgctgagaattgaacccaggtcctctggaagagcagccagtgctcttaacagaagagccatttcccccagcacctgtctttttcaagaaagagtttcactttacaggcctggctgtcatgtttaaggtcgggctgtagacctgtctgcattctgtatgtcttagcttgttcgaatgtcctgtcactctgcaggttaagacctggtttcaaaaccaaaggatgaagtgcaagcggtggcagaaaaaccagtggttgaagactagcaatggtctgattcaggtaacaagatgttttctctgctttcctttttccccctccatcttaccatcttattttatgtgtctgtgccgtgtgtgtgtgtgtgtgtgtgtgtgtggctgacctatggctgttgggtatggatcccagatttcctgtaagagcagtcagtggctctgcctttgcttcccagtgtcatcctgatccatctatgtccattagtcctgtccctttcccatccctcttcctccacgcactctttcttgtgatctttggttctttgaggcaggctctctctatgtagccctggcagtcagagatctgcctgcctagtctcaggagtgctggggttaacggcctgtgcggccatgctaatcttttcagtatttctgcagtttacatctttagagtccccttttcactcattgcattaatcacctctttcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttccttccttcttccaaatattttcatattttttttaaagatttatttattcattatatgtaagtacactgtagctgtcttcagacactccagaagagggcgtcagatcttgttacgtatggttgtgagccaccatgtggttgctgggatttgaactcctgaccttcggaagagcagtcgg

SEQ ID NO: 10 - protein, 305 aa, mus musculus
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 11 - DNA, 15487 bp, mus musculus
cctttgcctccggacttctctggggccagcagccgcccgacctggggcccggggccacgggctcagcagacgaccatgggctcggtgtccaaccagcagtttgcaggttcgagcttgcgattcagcgggcacaccttagggtgcagggagcccaatgtctaaatagaagggaggtgggaaagtgtcccacggacttgtccgcgggacccccaagtccctatggcctcctgcttctgggggccatctctggactagggaaccctggtgttccagtggcttgtacaccgagcccgtgggggcggagggagagaagaggaggggcctgtccctccgcgtgtgcacgggcaacttgttgtgggaagacaagcaaaacttttggggcgtcagcatcctctgtgtaagcttggggaggcagccaggacaggtttcttcttaccagctgcttggtagctgcctcctcctgggtgtctatccgcgggctgaccaccgagatctcccagacgggtccctgagagggcctgcgatcccttggcccccagctgatcacctggccctccctatctccaggtggctgcgccaaggcagcggagaaggcgccagaggaggcgccgcctgacgcggcccgagcggcagacgagccgcagctgctgcacggggccggcatctgtaagtggttcaacgtgcgcatggggttcggcttcctgtctatgaccgcccgcgctggggtcgcgctcgaccccccggtggacgtctttgtgcaccaggtgagactcattctggaactttgctaggggaaaaaaaagaaaaaggctgggagttccgaggtacggccttggctggtggcccctcctgtcggattggaggagataaatacccctcattgtgcgtccccatatttttcgggacaccccagttttgagtttgtagtaactgcagttgtgcgaggaggcactgctgactaccagcaccccctccccgcccccttccgggaacccctccggcttaccacgtgtctattacctctttcccctgggaagggagcagggggcagggaggtgaccccatgtggcagaaactaaggttgtattgtgcttgccggtgggggagggccagcaggccgaccagggaagcacatgcagggcctgagccgggcgttagcgccctgagttctaaaggtctgagtctaattcatagggaggagagagtgtgagccatttgctacccaacctacgggcgtgaacctgcgacgtcaggttcttcccgtttcaatgcagacctcaagtcaggtggccacccaaagccaggtcctgtagccagtgtaggtttggcaggtcccggaaggatcaacgtgtgagctgaaaagctcccctagaggtctggctacagatcagggctggcaggcacctctagctgtccacccggttctgccaggaaggaagaccttaagatcccaggccttcattccttggccgtggcaatcccaagttcgcagttaaagaagggctacacagtagggagtgaaattcagtgtcgagggggcaggcggtagaggaagactggcctctggccctattctcctcagcttggcatttatggccccagacagccattgattgctgtccctaacattcctaaccctaaacaaggaagagagtagagccttcaggtttccctcttcccggccattccctttcttaatatcctaccgggggcggggtgtgtgtgtgggggagtggtaccattccacgctggaaaggaagttactgctcctgcccttttccacatttggtgggttaaggctgggccaggaaagaagaaaattgctaatccctggcctgcgagcttgacacccgggccgggcagggaggggttacttcagggaggttagctttaggagctgtgggggaggggaaaggaggatgtggggattaagggttggagcctgagggatggcgttcttgagggggaggggtggggaaggagatgctgggtggactggttgggggctgctgggaaggctgaaccctgccctccagtcagaggttggggagtggaaactaagtagatttgggttgatggaatttgaggaccagctctttgctgttggtgtcctagggtcactaggcttctggaatatgtgttttgtttcttttttaagggacctggaacagtacctttaacttttctgcaccgcccccccccatcaccatcaccagactgctggtttctggatggtggctttgtgccccccatctcatttgcatatgtgaatgataacccacaggaagtaattgtgaggcagaagggaattggcctctttagatctattaaatcactttattgcaggaacccgcactgaatggccctcctcagcggggggcaagcctccccgattcggccatttatctgatcccagagatgaaaatttcctgggctcgagatggtttggggaacttcttcaatttttggaggctggggtgtaaaggtggcttgagcactttcctgactccttgcaaaggctaagtagggaggtgcccgggagaataggtaccttctgatctgaggttcattgctgttgggacaaatggagacgctgtgcctcctcacttaggagccataccaactgggaagctataggacatctcaggaggcagccttcagtacagactggcttggagacctgcctgcgttcgttcgtgggctttgactttggtttctttggggtttttttgtttttgttttgttttgtttttgagacgaggctggtggtggtggaggtgtctcactgtgtaactgtggctggcttggaattgccctgtcacccaggctggccttttaactcaagagagctgcctacctctgccttctggattttgggattaaaggcccatgcgaaggttcctggctggcgcccatatttgaaaacctctccacacacttttaactgctgttttaccaccacctgcttggtccacactcccaacagtaagtttggtcgttgagctgaggtatctcttcctgaggaatgtcgaagatgcaggttaatctctagaacttgtttactttggaaggggagtgtagggagggtaaagaaatctttaggggaacttcctcagctgccagaccctggggatggggtaggagtgcatggtgggggaggggaagattgtctcaagatgatatttgccttgccagggaaaggtgggggaatcaaaagaagacctttccagattgaggtaatacggttcccaacatcctgttaaccactgtccttgtgctcaaattttttttttttttttttttgagacagcaccttacactgtagtgtagtcaggcctgcaagtaattgtatatataggcaagttgaccggtagttgggtgacaccccttttctttggtgtcaccagagcctgacttgtatatactttattctgtgcccagtaaggtgtaggctgagggtaggggggctatgacacaggtttctcttaacacccattttatttggccaaaggtctttagatcttgaaatctggagttaattagttgtgagaaccaaaatgtctccctatcttgccaaatgtcccttgggaggcagaatcacttctagttgggaagatatttgtcaaaattgttagaagtccctgaccacttataagtttgggtttaattctggtttcctgtggttcaaaatctaaaccaccaagatttgtagaaattaggaaataaagttgtagtggttttcagcagctcctcagaacatcaagtttttgttttttaaggcaggctctttgataccacatgttgggcggtgatgctgtacctgcatagacccagtgctcagatcacaggtggtatcaggacctcccaaacctatgacattacataggtgacagcccaacctgcagactggttgccaagacaacaggcaccatattcccaacatccacttggctcaccttccacctttacctgccttactgtcattccccatataaataaggagaacacccctccctcactctcttcccatcttctctttctgccactaccttgtctctcccttccaataaaacctacctcttccacgtggagctttcttggccagcactttttagtgtgctgttgagacttccccctgccatcctaacactttatgtacaaccaccttctgccaaataagtttttttttggcatatatatatatatatatatatatatatatacatatacatacatacacacacacacacatgaatacatgataatgatatggcaaagggtttggaaacaacaaaaaatggatgttgatctagacattaaactaaaccaacttcgtgagccccagtggtttggaccttcagaaatgcctgagggatgacaaatggcctctgagggcctgggtagggcagctgggttgaggggcaggtgtgggatcttgccttcctggcttacaacactgcacctagtttcgatgtcttctgctgtctccaccaatgtggatgtcctttcaagagaagaacttgactgtcttagtagttttatagttcgcacttgatctaaatgaatgtggaaaaggaaaatgacttgaggaactgggaggatcagtagatgatgccgtcaccagaggaaacaaagaagggctgtatgtcaatgaggcactcgcttctgagctggctacgatatggcctggcacatgtagagtgctctgagcatttgtgaggtactctgaaccccagaactttggcttcatcttggaatcaaacccaggacactacctatgtcagtgggaagaggtggcttatggcttaggggtgtttggatttttatatatatatatattcattcacatgttattttgtcattaggaaatatttggcgccctctttctgaggagacatgcttttcctgggtaaaggctttgccaccatttcaagcaaagctttcaatatggtaaacttctgccttgtggggtgacattgctgcttgtcacgaactatcagtggttttcagtgacttggttttgagtataggacataactgttgctcaaacattcctaatcagaagttaaacttcatctatcaaatctttaattaattaattaattaattttgagatgctgtcactgtcacctgcgtgaagaacaggtctgtgtagaggtccaaggctggggtggaggttggagaagtatgtgtcattgtgactaccttttgagcacgatttcacggttgccctgttagatacgtggaaaccaatggggtagctttctctatggttctggagtaaactctgggaggtttttccctcccatttgcaccctgcagtccttcctaggggcccagggtgagatgggaagaaagcacccaaaccttgcaactccagcataaggggaaggatccagaaaaacaccaggagaagctgaggtgtccatgcacacgccaagtgctgcccttgcttcagagcctcgagtgggctgatgcaccacctggtggagagctgcgggggagcgtgtggcctgggccactgagcagaggtgaatggcagttaaccttagcttggggacatttatgataccaaccgccaggcctctagcaggggagctccggcgacagcatctatcactgcactatgatgccagtgttgaatctttgaaattaaaaagtattatttaggaattttaagaatatatggagcaaaaaatgatatccgacttgccctcctggtacatcttgctggctaagcaaaaccaggctcatggcaataggcccaacacttgtccgttactgcgatcagggcgcttatttttatctccagctcctgtccgggacactccctggctctcccaaatatatacttggctggtctcacttcattagggtgtctgatcagatttctctccttaagcagaggcatttatgaaccttgttggggtgctggggggggttgactgtttagctccttgctgcgtctaaagtaaacatttaatatgcattttggtttcaaaacccagaattgggcagagacatgtatgggattatgggatctgtcagaattggaatggctctgcgtgctagagaagaggagagagcgagtcttggtcttggtcttaataaactggttgggatacaagggaaattctcccataaagaaagaagcagatgacgtgaaaggtgtgtgcgaactggaatagacctgatagttgtaacgataagctttgacaaatgagtccaagaagcagaacttttgcttattcttctgtcctcattgatgttgggctggggtattcaatgtggggccggtgtggattggtcaaaagaaagaagcagaagccaggaagtgcttgtaactgaagagggcggtggggcaccggggaggaaatgctgtaactgtcctaagtgtcatcttcctgtggcccaagcaggctgcacacctgacttaattcaacaggttggaaaaagaaaatgggattcccgccccttctcctgttgaaacatttatctgctgtgggaaggacttggctgaaggcgccataggggaggcatcttctctagtccatcctggaggtatggggaggagggtggccgggaaggaagatggctgctagttgactagcaaacttccccttcccggagtctgactggggaaggggttcagagtccgtccgtccgttcctctcatcccaaccatagtggaaaggaaggaaggaactggtccggtttgaatgtggggggagggtgggggtgagccatcctagggggatctgtgccctggaactccaggaatctgaccaccagaaccaaccagtgtcaactctttgttccccagagctgggctctattgggggagctcagaccatgcccctccccccccccagagccttttctttttggaggggatgggaagaggggcggggcccggctttgtgtgtggttaggggtggactgggggtggtagaagaatttcaggctccttttggattctggccctgggagtctggcttggtggggctgcagcacactgattacctatctgataagagaagaattcgaactatgttttagaaaggagtgggggaggggacgggcagccacccctggagaccaccagtctaaatcctgcctggcctttctgctgaggtgctggctgaggcgggtcttctgcagatcttcagtggtgggggaagtaaggggggctagtcagtccccatcgccttaccacttattctgcccctttgggctcaccggtcttaggagtgggagtggagtagggaaggcagttgctctcagtaaaccggacctcatagagggctgtttttggagttcctaagaatggttagtaataaaaggagagttctctgagcctctcagcacttgactcatactgttaacctgtctccatctagcttgaaaggtaggtaattggtactccctaaggatttatacatggctggtgaagtgttaactactactctccatgcataaaaccccacagcacaaccataaaatcagtctatactccattttacaaatgagtgaggagggaaccggagctccgagacccggtccttcctaatcgcttatcttctgccagccccactggacctggggcgggaccctgctcaggcaggaattgcctcggagagtctcctgctccagcgcctccctagctatagcgcctgttgactttttttttttttttcccctcgagacagggtttctctgtgtagccttggctgtcctggaactcactttgtagaccaggctggcctcgaactcagaaatccgcctgcctctgcctcccgagtgctgggattaaaggcgtgtgccatggttgacttttaggagtggtttttgggtgggttctgtgtggtggggccgagaggtgggtccttttgagctctgtattactcgtatttcccggtttaaggttttatcccccttgctcacatcaaattgaggcaggaggtcaccgggccttggatattcctgtgtgtgtggcaatctggttagtttgcttggcaaagtgtggccctgggttcgatcctaagcacccttcataaagagtgagagagcatatctctgcactatctctatgacagtatttcttggacagttatacatcagcataaagtgtttaatttgggtgtcgtgagaccgttcccatcaggatctggacctaacacactgtttaggtcaaaaggtacttcctgcgctaagatgcctactaggggcctacatctgtcattccagcattcaggaggctaaaggtgggagaggtccagttcaaagccaccctggtttatataagcaaggtaaagagtctagtgggaattattctgccaagtagaatatactgagtgacacatactgatgctctcagacagtcctggctaggcacacaggggtctattttgtttctttgtttgtctctcaaccatgtagaccaggctagcctccgatgcagtggctcagctgggatcaaaggcgtgcaccaccccgcccagagccttttctttttcttggttgggaagtgagaaagaaccattgtatttaggcaggtacccggccattgagtcccatgtcttacggtgactctttgttgacatagttagacattcggaatcccttactctaagggctaggaggtgctgctgcctgcactaatgagcaatggatctggtctgtgctgaggtgacagctgtgagctaaggcatttttaaagggggctagcctcttgtcaaacggccttaggttttttcaagtttgggtacaacctgtggtgtttccaggaaactagtcttaatagaaatggctcaaaatccttatttacattccgaagcaaataatttattttctgtttccttgttgggcgtgttcacctggtcacagctgccctaggagcggagcagcttggggaacagagctgtcaattaaacaaacagaaagacagcagagtcaacaaacctacctccttgccctggaatacctgtgtaattgcaataactaggaggctgaggcaggagaatcatgaatttgaggtcaggcagtggcatagcctgtctcaaccgtgggccccccccccgccccctcccccaaacccacagggcagtggtgtggaaaacttttaatctcagcacttgggagtcagaaacaggtggatctcctgtcatgagtttgaaagtttgaagtcagcctggtctgcagagtgagttttcaggacagctaaggttacagagaaaccctgtctctaaaaaacaaaaacaaaaacacaaagcaaaagaagaattagaaactgtacttcataaattcatccaaaaaataggccaaaatgtggcatgaacaacaaattttgttgattttttttttctcatgctttttaaatgttgggggtggggaggcacacaggccacagtgtgcatgtggaaatcagaaaccaactatggggagtctgttcttctgtcacgtgcactccagaggctaaactcagttatcaggcttggtagccactgactttacccattgctccatcctgctagccccttaataataccttttaaaggagatctgtcattctaagtgttctggccttgaggtaactgtagcccaggctggcctccagctgcagctggtgctttctgtccaggttccctgagtgctggagttgtggggtgggcagaagtggatgccaggtctggctcattttttgtgcagtttccctctaagtgttttgatagatttgactgattgtgtcttttttttttttttttagatttatttattattatatctaagtacactgtagctgtcttcagacacaccagaagagggcgtcagatcccattacagatggttgtgagccaccatgtggttgctgggatttgaactcaggaccttcggaagagcagtcagtgctcttacccactgagccatctcacctgccccccccctactttttttttttcttttaaagaattgtttattggggctggagagattttaaaaagatttatttattttatgtatgtgggtactctgtacactgtcttcagacaccccagaggactcctggaatctctcttcatgacattccatttttaatcgttatcctgaaactggcttcagttttcagaaatactatctttctgatttcaaacttacaagattataggaatagatttgtcattaattgtgattcccagattcgtagattaacattttaccctctttgttgtatttttggggggggtaggggaaagatcaggaaggctctcactatattggcctgaaatgcatctgtgtatatataggtcagactggccccaacctagagatccatctgcctttgtctcacaagtacagggattaaaggcttgtactacccaacatggcccagttatattatcttagtaaccataaatacagcattcagtatatttctgtctacacactttcatgtgtatttgctaagaatagagagttcgcttgcttatggcggagtgggggttatgttttgtcttttgagacaggtcccctagtatagttcacactggcctcaaactggggtgacaggtatgtgctgccatcctctgctctgagtccattttcccatctgtgagttctgtcacttttcttcctgtcttccggggggccataggtttgcggttttgatttatggcacagttatgggggggcaagtagtaaaatctcgcctttccacttgacagagcaagctgcacatggaagggttccgaagcctcaaggagggtgaggcggtggagttcacctttaagaagtctgccaagggtctggaatccatccgtgtcactggccctggtggtgtgttctgtattgggagtgagcggcggccaaaagggaagaacatgcagaagcgaagatccaaaggagacaggtaggggcctggaagggcagcttgtagcttgtgtaagttgccgaggagatggggaacttcacacgggttgccacttggatcccattgccaaagataatttacgcgtgagactagcctctatccactatccacaaggactttagaaggaaaggttagaatgtggatctgtgtgtgatggggtgagatatgggatatttagtatctacagttcttgaccggagcttcaactggttacctagcgtctgcatttttgctgtggtgcttaaaacctcttagcctctgaagaggtaatataatttgcctttaaaaattctttcttagccagacatgcttttaatcccagcactcaggaggcagaggcaggattctttagtttgaggtctacagagggagtaccgagacagccaaggctcaaaccctggctcaaaaaacaaaaagcaaaacaaaaacacttcactctttagccagatcaggtggtgcatggatgtaaagccaggacttgggagacagaccaagaatggaccagttcaaggccaggcaggagcgtgtgtgtgtggatagatagatagatagatagatagatagatagatagatagatagatagatagatagatagatagatagatagatagatatagtgtatatagtgacaccttgtcttaaaaatgaatgaataaacaaataaataaggctgaagagatggctcaggagttaagagcactactctctggcagaggcagtctcctctctatccctcatgcagttcctcacagctgggatctctttgttctctttatcttttcaagttgattatctacaatgttgcagggtatttgattccactgggaaccctgagcttgtgaactggacaaacctgtttcctgttgtgttgtggctcagccctagcaagcacctttaactaaccctgagagctttctgtttactgtgaacaagtagatgtggtgggcctcagccctgacacacacccttaaccaagaggtctctgtaaacaagaactagggggctggcgagatggctcagtggttaagagcgtcgcctgctcttccgaagatcccgagttcaaatcccagcaaccaccatccgtagcaaaatctgatgccctcttctggagtgtctgaagacagctacagtgtacttacatataataaattaataaatctttaaaaaaaaaaaaaacaagaactaaataaagtcaagaatgggtcatgaggcagagctgttggggagtggacctgagagaacccagggaagagagaggaattggaaaagctttgttcttgtgcgtgtttggaggaggaggaaggtccgctgggtgctttctctgcctctgagctccaggcttttcccagtgtctgactccagagtcttcatttggtaaatcaaacatttgggattttgttttgaagatcaggaaagggaaaagattgatattcaaaagcaggaaataggtcaccagggaggcgggtgattatagtgataacgtggatggaagtgccagcctatcaggaggcttcaaacagtttcccctgtatctctctcatgttattataattttgtcccctaccaggtgctacaactgcggtgggctagaccatcatgccaaggaatgcaagctgccaccccagcccaagaagtgccacttttgccaaagcatcaaccatatggtggcctcgtgtccactgaaggcccagcagggccccagttctcagggaaagcctgcctacttccgggaggaagaggaagagatccacagccctgccctgctcccagaagcccagaattgaggcccaggagtcagggttattctttggctaatggggagtttaaggaaagaggcatcaatctgcagagtggagaaagtgggggtaagggtgggttgcgtgggtagcttgcactgccgtgtctcaggccggggttcccagtgtcaccctgtctttccttggagggaaggaaaggatgagacaaaggaactcctaccacactctatctgaaagcaagtgaaggcttttgtggggaggaaccaccctagaacccgaggctttgccaagtggctgggctagggaagttcttttgtagaaggctgtgtgatatttcccttgccagacgggaagcgaaacaagtgtcaaaccaagattactgaacctacccctccagctactatgttctggggaagggactcccaggagcagggcgaggttattttcacaccgtgcttattcataaccctgtcctttggtgctgtgctgggaatggtctctagcaacgggttgtgatgacaggcaaagagggtggttggggagacaactgctgacctgctgcccacacctcactcccagccctttctgggccaatgggattttaatttatttgctcccttaggtaactgcaccttgggtcccactttctccaggatgccaactgcactatctacgtgcgaatgacgtatcttgtgcgttttttttttttttaatttttaaaattttttttcatcttcttaatataaataatgggtttgtatttttgtatattttaatcttaaggccctcattcctgcactgtgttctcaggtacatgagcaatctcagggataataagtccgtagcagctccaggtctgctcagcaggaatactttgttttgttttgttttgatcaccatggagaccaaccatttggagtgcacagcctgttgaactacctcatttttgccgattacagctggcttttctgccatagcgtccttgaaaaatgtgtctcacgggtttcgattgagctgccccaagacttgatctggatttggcaaaacataggacatcactctaaacaggaaagggtggtacagagacattaaaaggctgggccaggtgaaaggcacaagaggaactttccataccagatccatccttttgccagattagtggaagcctgccatgcacagcagggtgtgagagagagagtgtgtatgtatgtgtgtgtggattttttttaatgcaaatttatgaagacgaggtgggttttgtttatttgattgctttttgtgctggggatggaatcttgggcttcatttgtgctaggaagtacactgccactgagttatcccagtaagaatgcaacttaagaccagtacccttattcccacactgtgctgtccaggcatgggaacatgaggcagggactcaactccttagcctttcacaatcttggctttctgagagactcatgagtatgggcctcagtggcaagtgtcctgccctgctgtagcgtgatggttgatagctaaaggaaagagggggtggggagtttcgtttacatgctttgagatcgccacaaacctacctcactgtgttgaaacgggacaaatgcaatagaacacattgggtggtgtgtgtgtgtgtctgatcttggtttcttgtctccctctccccccaaatgctgccctcacccctagttaattgtattcgtctggcctttgtaggacttttactgtctctgagttggtgattgctaggtggcctagttgtgtaaatataaatgtgttggtcttcatgttcttttggggttttattgtttacaaaacttttgttgtattgagagaaaaatagccaaagcatctttgacagaaagctctgcaccagacaacaccatctgaaacttaaatgtgcggtcctcttctcaaagtgaacctctgggaccatggcttatccttacctgttcctcctgtgtctcccattctggaccacagtgaccttcagacagcccctcttctccctcgtaagaaaacttaggctcatttacttctttgagcatctctgtaactcttgaaggacccatgtgaaaattctgaagaagccaggaacctcattctttccttgtccctaactcagtgaagagttttggttggtggttttgagacagggcctcactctgtagctggagatagagagcctcgggttcctggctctcctcctgccttctgcacagagtcccctgtgcagggattgcaggtgccgcttctccctggcaagaccatttatttcatggtgtgattcgcctttggatggatcaaaccaatgtaatctgtcacccttaggtcgagagaagcaattgtggggccttccatgtagaaagttggaatctggacaccagaaaagggactatgaatgtacagtgagtcactcaggaacttaatgccggtgcaagaaacttatgtcaaagaggccacaagattgttactaggagacggacgaatgtatctccatgtttactgctagaaaccaaagctttgtgagaaatcttgaatttatggggagggtgggaaagggtgtacttgtctgtcctttccccatctctttcctgaactgcaggagactaaggccccccaccccccggggcttggatgacccccacccctgcctggggtgttttatttcctagttgatttttactgtacccgggcccttgtattcctatcgtataatcatcctgtgacacatgctgacttttccttcccttctcttccctgggaaaataaagacttattggtactccagagttggtactg

SEQ ID NO: 12 - protein, 209 aa, mus musculus
MGSVSNQQFAGGCAKAAEKAPEEAPPDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKNMQKRRSKGDRCYNCGGLDHHAKECKLPPQPKKCHFCQSINHMVASCPLKAQQGPSSQGKPAYFREEEEEIHSPALLPEAQN

SEQ ID NO: 13 - DNA, 200341 bp, mus musculus
ggggacccagtggcgtccgaatccgggagctctggggtggcgcggggctcgccgaggggcgaggcgaatttgggggccctgaggcctcgctctcgcgggaatgatgctggaaatgatgctgaggctccggcgtgagacttgcggctgccggcggagcggagtgtgagccggtgaatggggtgtgtggcatggctggtctcggagggcgcatcgacgggtcgcgagtgtgcgtgcgagccggcgtgcgtgggcgactctggctgtgcgggctcccgggcgccggggctcctggatttacggctctcgagtgcgctttgtgggctcgcgcagtgggcgtgcgcgctcatcctcagcccggtgtctggctagtggcggggctcgcggaaattctgcgtctgcacccggcggcggtgcgtgttcagtgcctgggctctgctcagcccgccccggggcacctagaggtgcgcagccagggacccatctgagtctttccctgctcccctgtgaccctgctctgggagtcaggctgctgccctctccctgtccctttagggaattccggacaggaactcccatcccatcctagtttactgccctctccctgtccctttagggaactcccatcctatccttggtcttagttttgccattcgtctgaagagggctgaccctgggtggaagcggattagggtgggagggcgcaggggattctccggaacgtgcgggcctagggcgacggcagcgaaagcgggacgaccacggtgcgcggggtcaggtggctcagggaatggggtcggtgtggtgacgctaactttttttttttttttgagatcaggactaaatagaacgtgactgcccaaactgtgatctagggatggagtttcccgctttctaaagctcggtgctttcttccctccgcaggagcctggcgcgacccccagccgtgcgccccgccccggcgccatgcattgcgaggtggccgaggcactttcggacaagaggccaaaggaggcccctggtgctcctggccagggccgcgggcctgtcagcctgggagcgcacatggccttcaggattgctgtgagtggtggcggctgcggggacgggaacccgctagacctgctgcctcggctaccggtgccaccaccacgtgcccacgatctccttcggccccggagccctcgagactatggtgtgtccaagaccggcagcgggaagggtgagttccaccgcaccagtgtgctgaccgcgggcatgctgaggtcaggtctgtggtaccggtgtctctgccaccggctctgtagggattctagaacttcatggaacttccttaagagttccttaatgtctcagtatctctgtagttgattgggactcgcctgtcagtctgaactgatgcctggaagcatccaatttaaaccaaataggcatgtttgagaaggagatgtgtatgtagaaagcttacattacataattttgcttctatatgtttctgatgaaagtcgtgtgaaatgtcaaaattttattgtacattagtatctagtaattttttctaagcaacggaaggcatattttgaactatcacagtctctctaagaaaggaaggaaggaagaaagaaagaaaagacaaaaccaaatggaggattttagtaggacctcttccttaggaacctcagttttctcatcggtcaaattaggaacttattatgccaaatgatttttctgactcctgacttaagttcttttgagcctgtaatctgggttgggtcaagtgttgacttagaagtgacttttctgtgcttggtgaagccaaggcaggcccagaacccgctatgggcaaattcagggacaggtgctggtagactcctgaagatattcacccaatcatagttaattattaccgacatgttatttataggtttcatgttaacagataattaaccagacattagagattgttaagggatgtgccgatctgcccgacctgccctttcgttccccattagccgaaccagcaactttggggtttgatctcatcagctgcagtacctttttggaatgaaagccagtgttttgtagttgtagacaccgccagctcagcaccttaatccagcttgcttgcagttgccagcctggttggaagacataactgtagagaggtttctcttcggctttgtaggagagaggcgcttcttgtctgcagtctcttcttgaatgtattgagtatttttgctgggatgcatatttgagggagagagagagcgagcgagcgagcaggaggttagtaatgttggctgcagagagctcctccgctcctgcgttccttaaacagtcctttctcacagtcatacccatctgcagatcaaggctgcaccatcctagtgatggttgtccctcacataagtctgtctccttatcattgggagcatttcatttgatactttgtatattccctgtctctgtccttctcagcctcagtatgttttgtccgcccattgaccctatataaaagatcttcacgtcaggatgtcagggctccctttggaaaggacagggaaggtaatcgatggaagaaggagatcctttcttggtacctgaagctggtctttctggtgacggcctcccagtggcttctgagttggttttcttcacagctgtgaggctgttgggggtggagtagatggcgcccatttgcaaggtgagaaaactgagaggtcgcatctgagatctggagctctggcacaactcgggttagagagagagagggaaatcagtcagagtcaggcaaggacgtctgcgggaagagcagttttctcccagtcttctcaggctgccttcttagaaacgtcatgggtgagaggacaccttctccgaggcctcctcccacccagagagtccccggcttcccgggcctttaagagtgtgtgcagtccagcccatccactcaggcttattagaaacagaccaggagagatgccgactgaagcatatttattgttggagctggaaagataagacatctaactgcactgggtgggacagttatttctcccctctgagctcaggcctcctccttcagactgacttagactgagatagccatgtgtttgatgacctcagcctctggattctcatttgcatggagacggtgatgatgcccatcacagggcccataaaatgaggtggtgaggatgctatataaacggagaaggtcgcttttgttaatacttgcagagcaccgtgatagcaacaagcgggatcgaagcataagaaagatttaaaaatattaaatgtgaaggactctaattctggtgcactggctaggagaggccaagaggtgctgtgccttgggtcttcggtggcctctgttagccagtgaatggagagaagacacccatgcctggtgtggagtgagggtttaaatggtacatgccttctgccttgaattgaccttaagcacaggaagggactgaactaatgaatatttccaggtctgcactgggagggactaggcatggggtgacatcatggactaacgtcgtcatcagtcgtgataagaatcactatctgtttatgaccaagtcccatttatccagattccaaacagctgacagggccaaattgccaaaccctgcatttttaaaattattattaaacacctgacagtcttttgttagtccagacaaggaaagaaggaaatagcttggctttcctgggcggacagctttatggtcatggctggtggtgcttaccctgcccaggccttcacagtgttcagaatggatgtgtgtacatttccttcatctgtctttgcctacctcgtgggctcttcccaggatgaggtggaatgtgtgtattggccgggcagacactggctctcaggaaactgactcctttttatagattagtggtcggaggttatagggtttgttcaaagctatgggcagtgactggaagttcccgaggcctctagcctccagatctttatggttccgaaacttgtaccatctccatgtgttgcttgagtatgagacctggcattcataagttgggcccttcgaacatccccagagaactgaactgcatttgtttctggtatgtctcttgtcttaaatgtttggaaactggtaatcgtctctctgccatgtctagaaggctttgtagtttaccagatgattccatctgtccaaaccagttcatgatgttactgtttaactcacagcaagtcagtgggcctgggccctttctaccacgcaccgtacacgacaagccgaaaggggaggttgacaatcgtcgaggatgtgaactctgccattagcaaaaaacagtccccataacatggctgtaaatttccaaaagtagtcagtcttgtagcttaaaaagaaaacaaaagaaaagagaagaaaaagaaaaagagaagccagccagctagcaagcaagcagttgtaggctgtgtgcctgatgtcgggctgctggtgtgggcaagacctgatggtttcagtgtgctatctgggtgaggttgtttcatcacagcaggaagctactgccatgtcctgagggggtcaggagaggctggcacatctgtaatagaaaggtagaagtgatttctaaagcgtttcccccctctgggtctggtgggcaatttgcttgcatttcatccattttaaagtgctggtcctgggatccggaaataatcagatccaaattttttcagtgcttgaggtttcaaataagggggagaaaaacctgctggccaaatgtcaggctccccgagacaaagagaaacaagtaccgtggggcacacaagcgcgataggaacgcatctgcagtcacaagagttacggacccagcagatctcctatagcaggagaggtagcggaaatgttattcccaaagattcccttcaaatttcgtgcagctagaatttctttcccctctctgccttctctggaagtcaacactcggtcggcagtctggaaagggagccatcgcttaggactgtgcctagctggcccgggggtctcaggatccccttggctgcgtgacagtgtgacagggtgtgttgctcatctcctgtgtttctcgtttgcgtgagaagtaaagtgagccctcacgcttatttttaatgtgtgttaaaatcccattatggacataagtgcgtgaatcatttgaaagctatattcttatgttctttcctctgaaggttgcataattatcattttcttcggttttatttcatggagtttggctagcatcctagaggagaagagaaactcaagtttggaataggggtttcagtccttttttggttgggattagaaagaaaaaacaacaaacaagtaaactttatttggaagtcgatcatcagtaagagaattttctcagaaaatttgaagctgatacaggggtaccctcccccttgccccccccccacatacacacaccaaataaatgattacctgtggtttgctaatatttttatcccccgagactagattttaattgcaattaatttgggcctaaagaataggtaggggctagtttatttcagtgtgtaaaagcacaaactgtccagtttaagaagaaatcaagcaaactgcagtggggctttgagaagtttgagtcgcacaaaaagcacaaactgtaattctaggccaagaaacaaccccctccccgccccaggctcgtgaggtatctttactgtccccccagtgctcgtgatcccagccgttggaaacggaggacttgtttatcaacagccgctgcccaaattaatagtttacaatgaaaaaaaaaaaaaaagccttcgtctgtccggcattggaagcaaagtccgggctcggacagctgagttactgagtaaggagatcaggagaggggctgtgctgtggctgctttggtctcccaaagccttatttacaagaaaccgaaagaaaacagaggctgctgctgtgctgggtgggggtggaaagagcgcagccctaacccaggggtctcacggtttccagccactgcatgcatctgttttcttctttcctgtcttcccccttaaactcaaaagcattgtcctttggaaggttccggcagagtgagaggattgtgttagtgttacaagctgtgcttttaaagctagcgttttttgagtcagatgtgtggagaggagtcctcagcttgtgaactgaaggacgcgtttcctgtagggcctgagtttgattggcgactatggttgaactgcctagagcatacccgtgcaccatagtacagctttgatgcacaggggttgggctttttgccttgagcctcagtttcccctctgtagaatgggagcagtttgattggatgatccatatcccttttctgctctggatccacattacctaagctgagagagttgggagggtcccaagcgtccagcaagtctgtatttatctttgtgccaggctggggctctgactgttccttcgaagtcctgactgttggaggtgaatacggaaggaaaagcgaaagggagacccactaaccgttattgagcacctactgtgtgtgccaggtcagacatggcatagctgtgtgagtatatttaatcacacctggcatccctgtgtggagagtaaagaagcggtctctaagttctgcagaatggaggggtgcgctaggaagtaggacagtgacttgtccaaggttgggcaggcagtgagcagtgagcctggcatttgtgccatgttgtctgaaagcaaggccagtatttacccataaagtgggtcagcttttataacacttgtgtctatggttggctgaggaccccagtgtcccccttactgagtctcagctatcatgccaaagtgggagaccgcttggttacacattactccccattccctgccccccactgcaacactgagaggagatgggttccctgtgtgatttgtgtcctcaggaagctggggtcttgtgagggctcttaggctgaccatggaggcccccttctctgaagcaggagcaaaaccccaccctcccgccccacccagagttattgcaggttacacccttggctgaggccatctcctggtatggaagacttcctcacagaggccagtggctcagggatctatgaaatagtgattttgtttttagcacctggtaagataaaggtaggcttgtcactgtgggcacaccggggacctattgtcactgtgggcacaccggggacctaactgatctgagaatttgttttagaagtggatccatcctttatgtcaagagatgagctggtcataagaagcccagaggtctccttccaggctgatctggccttgtcccatgtacccagaagactcttaacgtttgtacattctgaaggagtcacacttaattctgggaaccggactgcaggagccttcctggcccccgtgttgacactggttttgaaagggaccgtgtgaagcagcatcagagatgtggcctgggttcttcccatttgggtctttctagccaccacgttacgtttttcttagactcagttgcttttgtttttaaataacaggcttgcaggaaaatagagctttagaaagaaaccaaggaccatgaggctggcttgaaaaagacagatcccatttgctgttttaagctctcgggagggctgggattgggtttactttggaccgctgctggaccatatgaattactgctctgagcactgggtatttaatacctagtagaagtttgtataagagctgatgatgtattttgccgacttttccaaccctgtaaataaattacacgtgccggcagaggctgccaggatccactgtgtcaattgaagcttaatttttatcccaaaccacttaatacgtctgtcctagtgagcggagtaagctacagcagcggtggctcgtccccagatgaagggaccagcgtggagctggcgtctgtgggttggagattttgccatagttgttggtgattaggtgttgtccctcacccccacccccaccctgctctcctcaaggcagacaatgataatatgaatttaaaagtattataaaacgaaactgggtggcttccactccggaagcctggctcgcctccctcttctagaggaaagggctgaaatcgtgtctttctatcttagccccgctgagcgttttatatctcatttgagcgcaaagaccaggtaataccactggcatctccactgcgaaacaaaataggggttctaatttggacggactattaaatatttcatgcttcaaccaaactcaattctttttttcctataataagtttaaatattaattctctggaatgtcagatgcctaaagcgaggcacttactgatccgcagtttcaagtgtctcgaggaagggttatgaaacaagatatttcactgggagggatgagcggcgtgtttcagagatatctgagttcaaggcagcagtcttctgatttactttttagagggaaaaggtcataaaatgagttgctttatgtgctgacggattaagtggcttaggctgaaataacagcagggactttactggcttcccctgttatattttaacaatgctttttgggggagggggcgggtcatttccccgcctgcttgtctcctcctctccgggccccccctcagccagatggctttaattccaaccaaatcccatgctgaggccaaatgattgatcaaacagtcatttgtacttacgtagcgctttgtgagaatatgtccgcgctgcagcttctcctggggcaggctcttcgtggggccttcagaggggtgcttggaggggtgggggttcaggggtgaatggaaagccgacatcctggaccgtgaatcctcagaaccgctttttaaaaacttttctaatagtgccaggcctaggcagcaggggtatcctaagtttcctaggcgcaggaccgtgcttgcagttagccagcgagtgcctgctgatggaagggctgcccgatgcgctctaggtgtctcctaattttaagcactcttattaagttggttggtgttttccaggaacagccacataggcacacgtctgggtaggcacagactgtcttgtggtgctttagcgatcttgacccggcagctagtgggagccttgctaaccagctcaggcaccgtctcgagctagtttcttgtctgtacagacgttcttatgtgctcctaggcagctttcctctgctcttcttttcttataaactacaaactctgttttaaacttgtttgtatttatttcacatgcattggtatttttgcctgcatgtgtacctatctgtgtgagggtgttagaacccttagagctggagctacaaatggctgtgatctgctatgtaggcactgggtattgaatccaggtcccctggaaaaggagccagtgttcttagccactgagctaactcctgccaagcggtacaccaaaaggctttccacactgagccatctctccagccccccacaaacttctttttatttatcattgtatatccttaaatcatttgggggaacaggggagaaaacagagttttaagatactcacttgtgtccagccctaagtctgacctgaagtgttgtgaagtgtgtgaggtatcggttgaggatgtgggtgtggagcatggtatggaacaggctaccaggttcacactgtggcttggtggcctcaacacccttagccataaactgggggtaatgatagttaaactttcatcaggctgtgatgaggggtgagggctaacataagtgcatgaaaccagactggctcccagtgagtgctcattaatagccctggttgttgtcactgcagcttctgtgctgctgtaaatggcatggggacctctgggaagagctttactccctttttgtctaccatgctgacccgacctaatctttcctaccagcttaagagccacctcactctagaagtctctctctcccgtcttctttctgcctagaactgttcatactctgtggcagttactacgtaggtgaatttgaggacagggatggagtgtcattgtctcttttgtcacagaagcccaagcacagacatgattcaggatgggtgaccaagaaattgggaaatggtgacccacttgctgccctcaaggagccttgagctgtgagggagaatgtttgatgctcctgttttctgtctccaaggatggacacacaaaggctctctgtaagaaagatttattttatcattgattcatgaatatgtgtatgtgagtgtcgtgccttaggaggtcagaagagggcaccagatcccctgaagcaggagttagaggaggctgtgacctgctatgtaggtgctgggaactgaactctagtcctctggaagaacagccagggatcttaactaaccactgaaccattttcccaatccccagcaaatgtgtagtgctcccgtgaggtgctccacactgctgtgctcaaccatttattccactctgtctttattggcgcaactaagaggctaactgggtttgaattccagctacagcagagtctggtttgctggtatcttgattttacagaggggtgaggaatggtcaggaaaagctgtcttttgttgagttgggtgtgtgtggcatcaactcaacttgtcacaagtcctggttgtgagtgggtccagaggtgccaacccccacccctcccatcccccggtctaattggctgggacttgctgtctggctccatcatcctgctgaacttcttgcttcttcctttcaagtttaaggcactcaatgctcagccagcccctcacaggcctgtgcagcctgcctctgtggtcctgtcaggctggcatttgttcattcctgctgagagcaagcatccttgaagccatcggttttctccatttgcctctcggagagtcgggtgccatcatccccattctacagagtgggcagcaggcctcaaggggcaagtgacctgaagcggtggagcggtggctgccatctaactttcctttcttctactccttcagccacagcctgattcattcatcatctttgagtaacttcctcctttcagctggggttaacctggacctccttcatgtcctaatcggtctttggttcttccctcccaggaagaggacatcttgtagagctcctggctttctttcccagggtgctcctgactcgatggtggggttggttcagaggatggtggtcatttcctctccagtgaaggggtgtttcctggggagttcctacgcagattttcagggccacatctagagtggttttgagcttctaagctttactcgttcattgaatatccactgttcagatatcagatctagccctggacacagtggtggactaacagagctcatagcctggcaggcaggtgaacggtggatgaggagctcacacgggatcccaggcctcctgtgtgcactctcaccagggtctctctcctaagcagactctcccgttctgcagctgctatcagctgatgacctcagccttgacttattcagtatgaagtggacatatgtgctggctttcctaggtgcagtgggatcagggttcaagagatcagtagggaaacggggcttagaccttagagggagttttcctgaagacaacttggaacttgaatctgcagaatagctctctgcaggatgctggaggtggagcccaccttggacagtctatcctggatagggactgtccttgcctttctcctgtaacttctcactagttcctgctgtgtgagctgctttcagaggctcttgttgccatgcaacaaggatacagaagagctgtttgccagggtgctagccactgtttgattttggccaagtgtgcgctgtgacctctggagaccctcctcctcacacccagcattttcagcactccagagccatcgttactgtgtgggcactgccagccactcccgtcactcccttaaaaaaatggattctctaattgaatgtcatgagtatcccgtgaagtacatttattcctccttaagaatagttacggccatgaattcattacacagagttaacaactcaatctttgaattgttgtaactgcattgcatgtactcagtaggcactgggcgagtggctacaccatggattgccgagtgagcatgctctcatggttgcagacagttctgctgggcagcaccggggttgggttcttgagttttctatctcttgtctgcttcctgatgccctctcctacttatggtttgcttagtgcccttggcctctactttggggaacatttcggggacatgccaacttaagggtgagcagcagactggttgccaggccaggctcctgttggggtcacagacgtgagccctagggtccgcctgctctgctctcagagtccacggaactaaagtgaggggagatctgacctggcttcctggagaggtgatgctgagtgggcaggtcccggtgggtgggaagcagcccagaggatatgctcatctctgcaagcctgtccccaggtcaggaacatgcacagggtgtgctggtctccacagcctgtccccaagccgagaacgtgcacagggagacctgtgcagcacctagcggcgatgggatctggttttatccttattgtttcctgacttctacaagttacttggccttactcctaatccttatttgggaggtgactggccctttgaagataatttaattcattaatctttatttttctgctcgtttaagtaatgcagacagtttggaagctgtagaaagagattaaatcattcttggcttataatcacactccctgcaaagcctattgcttacggtttggtatatttcttttcagttttgttcatatctaatacatattaatatgtattatatttgagttatatatcctgaatatagccttaagcacttatttgtaaagttaacattctaacacaatcatttccgtgagattaaaattcttggtaaacacccaaaaacgtattaaatttcattggctaggttattttcaataggtcacacattaaaatggtttcaaattcagaagacgccttttagtccttaggggtcccaggtctccttttttcttcacctgtgtctgataccttgtcgtcaaactagagatgcttgagtttgcctagcctctcagagatactttctgtgtatgaaaacacacatgcatacgtgagttacgtatgcacagctgttcacatcgcatgctagtagagaggacgaagctggagtgcatgtcttcctgcacagtcttcacctgagtcgctggtatcttctctgggtggggtctgcagggaggctgctaggctactgggtagtaaccctgagtagatgggacccctggacgttgcatcagttttcacaggcagacagtgccttctcagagaacacattgcctgcatcctcgcctgggcatccagcccccttccctgatttgaaggtgaacccgacagctggctatctctctgatttcgagtgtggtggagcctgcattgagtgttggctcatagttcccgtctctcctgccgaatatttctctctctaattgcgcttgcccaacacacctggggctctgagttcgagccccagtaccaccaaaataaatcaataaataagcctgctgacattaagcctggtctttaggttgctcacgtctttgtgagcctgctgtgtaaaggagttctttgtgatgctttgaaatgcattaaaaaaatcctttttatagttggattgcccagattttccattttcatcggtctgttctttgccactcaccgggtccctttgtttgctgttattgctatttgcggtaacttgttgtattaatgttttaaaataaagattttatttttagaaaaggaatgccaaaacatagcctttaaaataagccaaagtgataaagagaagctaggtctgccagccaccccccagagaggcagcctccagccagggtgtcagtgaggtctacctccagggggcaccagttctgcaagcttgtgtgacatggtggctccgctttggatccacttagttattttctaacattttctttcatacctatcagctatagtagtaaaactatcaacagcgagccctcccgtagtgattcagggtatcccacacaccattcctttaatcccgccagcagtccagggaggtgtgtctctttttagtctgtgtatttcactaacctgcaggtgaagtaacagaccagagagggaaaggcatcttgtccagggttacagggctggcaacgggtgcagggatggtctctctaacacagagttgcctggaatgttgtcctcactgctcaagtcctgccacagcagatggccgtttagttctcttaaacccagtcgtccagctgcacggtctttacctcctccccgttcttgaggccctgtggttgaaccagtgtgcaagtgtgacgagaggttgtctctttgggtctgtgatgaccctaagacagcaggcatgatgggggtgctggctgctatgtcctgaagcaggactctgctttgagagtcttgctcagcatgcaccccagaggggaggaagagagagatggaagagcccaaggagggggttttctctgaagagccggggtgtgtctttctactcatgccaagtggcagacgggattacctcttcacgaagcttaagagaccacacagggaacatggcacggccagaggaaccgagtgacaggtggtggattcatgagtgacagcaatgagaaggcaaggctctggttggggccactgcctgctaccaaagtgaggagatgacctctagaggtccttgtcaagcccaccttgccccaagtgtttcttgccaggcagaagaggcggaagtgtctcctaggacaccgaagggtagtagctgtctgctgaccccggaacccacagaagtgccttagcggcagagtggatgccgtctcctgtctcggagacgactctgtctgctgcccagtggacagtcagggacctggaacagtccgttccctgcccggcctcatgggaggaagatggctgtaagtggagtttggagttgagattattaatctggacgggacatcttaattgctaaaccggaggccgttcttaggactgaaagtccttgggactgaaagatgttctgagatctgagagtgacctggagtgtccagggaactcccaaactctcgcagctacagggtagggaacagggtgagtcttcagatggaatgtagagaaggagtggtggggaggggtgggtgggacagtgacagtgcctcttgagccttccttctcattgggacatggtgaccaagcagtcaggtcctgcacctcaggagccttttctttctgatgcgacctttcattctcttccctgccccctttttctgttttattttgtttcttcattttgatttccttggttttctctccttccctaattatttcatcagcctgtcagatgcctgtctgtgaggtgccagctagctctctctctctctcctgtatcccaacctggtttggaggacctctctgagaattttttcccagtcttccctaagcaggatctttacacgttggctcctcatctgtcatgtaccctgggagatgaagtggaaaagagactttagtgtaaggatgccgagtgggtctcagaacttccttcgaagactgaagaaggaggcttggtacctcgagcccactgcaggctgtccagtggtgcacactgctgtgggggaggggccagcagctgatgctcatcttggcctgggatctaagtgtggtttggagctgtgctcctacagctgtgttctaatcaaagtgtgcgtggtgtgtgtgtgtgtgtgtgtgtgtgtgtgtgtgtgtgtgtgtgtgtgtgtgtgccatcagagaccagcttgcaaggagccagttctcttccatcatgtgggtttctgggaccgacttcagatcatcagacttggtgggaagcacccactgaaccatcatcttacctgctcctaaccatcgtgctcgatcagcgtgaggtctgtgctgtgggaggtgggaaagcaagggttgtttgtcatttgtgttttgtagttttagacaaggtttcactgtgtaggtcgggccagctttgagcttgcaatcctgccttcgggtgctgccgttacaggcgtgtgttatcgtgtctggcacagtattgttaaatttttttttaaacttaacaaattttcattgagcacctgctgtgaggtacaaatacctcacgctgcaaagacaacgaagaacaggccatggccctgtgcaaggtttcagagctttacattctacaggggaacatgtgctcgtcaaataagcaggttaaataggaagttttccaagtggtccacagtaaagagaaagcaagggataggcttgggggccgttctgtagtgcggtcaggatagggaagctgacgttgttagcttctctgggtcttcagcctgcagaagtgtgcctgtgtgtgcatgcgggtgctccttggtgtgggctggtaggtgaagaaaggtcttcagattctagacagacagacagacagacagacagacacacacacacaggccattttcattgtgtttttcttggtttaactccaaagtttgctaggccatattctcctcatatgtgtcttaaaaggcctcacatttggtagtgttcgtgagagctaacacggacagcagtgaatttgcccgaattaattcatcctcacaaaccccagagggaggtggttctagttgttgtgatggccatttgacagatgaggaaacggagacctatgatgttaagtaacgtggccatgaaacctgggttgtttggctgcagggctgatcccgtggatttcaaggatgctctgctgtatcttcagtgcctcatgctgctgtgatgcctggctctgatctgatgcccctcctctataagtccatccagtctggtttttacgtttctaagtactctgtgtctttggtttgggatctcccaatggtgtgctttggtgtgggcttttctttaattgggctgggtacttagtaggttctttatttctggattgtcatgtctttcaattttagttgtcttgtccttttaaattttttttccccaatacttttactctgttgtcatagtctggaactctaactaattgtatattttgctttttgtattacttttaaaattctcctccctagcacctacctctttgtcttttaaatccgttttctgagaatttcctagaccttgtctcccattttaatttttcacttatttcaatttgcaagatcactcggtttgtctcatgtggctccctttttatgtattccatttgttaatatatacaatattttggggttttattaaactaccttttaccctattctctgccttcctgtttccgtttgtgtagtttatgttcactgatcatcgtctggtctccttccaggctgagggctttcctcatctgctcacgctgccttctgccggcgtgccgatacaggctatcctgaccctccactttgcagcaatcatggctctcttgagtctctgcgtcctcctttctcagtacccatcctcgtaatcccgtttcttagacaccctcctcacccttagtgcactgccacattcatggtcaggcccctgccctagttagaaccgactgagtgtgtcacacagtgccaaggaagcgaccccaaatgacccagtctcagtgagtgagctcagggctgcaggcatcgtcaaggcctggcttttctgcctttgtgcggtcggtcactccttatccccagccccagcatcctgtcctcacagacacctctcttctctcacaggctcgctgaaggtcccagtcagcacgcagacatgttaggaactggctgctccttgtcgaggtccacttactgcccaccgccccctctccccaccctggcctgtctatgcggctgaacctagctgcactaatctcattagacctaatgtctaggaaaggcccacttagggtcaccaggcagcttcccaaccctttctcaccgctcaggaccagcagctcttctgagttcagttctgaccctaggcacacacagttctgtgtctccaccttctggcttgtgcggccctgacacctctgtcctgtccttacaggatctccctgctgtggctgcccgggcttctctgtgctccgttctgtttctagcttcagggataggttgctcttacattgtgatagcacagcatctgtctgtttggattcccgtggctgtgttctcccctgctctgctggcctgcaaggcagacctgaagtccatctctgttgtctgtcttactcttcctctcttgccgcccctgtgctgacatggccctgcagttccatgagtcagttggagagtctggcctcttggcaggacctttatgcggcctcctctgctgcgtgcgccttcagctccccttcccacggctgactcaggcttgtgtgtgtcacttactggtgagccccccgcccccgccccccgccacacagtcctttcctatttgcttcattggctttccaccacagcccttgtcatgactcagtagcttggtagcttgctcatcctttactctcagcgacgagaacgccacaagcagttagccctcgcagggcccagggcagagtctgcaggagttggctatggtcatcaggtgggtggagcccaatgacagccctctgttcctcttggctgtgacggtgttgtatgtccatctctctccatctctactgtctcaggccaaggccatcatgtgtgaggctcggccactgctgctaggaatagatgcatttctgattaatcttgtgtgtgccttagttagcgtggtgtaggggaaggctctggggatagcagagagggcagagccagaccttcatgaccctggattttgattcttttgaggactggggtgctgtgtaaatactattgtgctaatctgtgtctgtactccagtgaggcctgggctactacatactgggtaaggaaggacttttagagacacagctgtgagcataacccgtgcttccctttgtgtgcagcgcccctggtggagatcctaggtgttgcacccaacggtgctctcctgttcatagaattagtgaacctgcttccacccaacccccaccccatcttgctgctgaagaagctggcttcctgaatgtcttgctggcagtgtttggctgttgtttttaggcacagctcagtctcctaccagcttcagaccttgtttgtcttcagagtgacaacaataaaaaatccaaattgctgtttccagtctatattttctcactaagtaaattttttttttttttgcatctctgagaattttatgcaatacattggagtcaaacctcctctcaatctctacatattcagctttgtgtcctaaaacaaaacaaagcacctatccagtttagtatactcttggatgtctggtcacccactggagggagggtgacctaccagggggcacagtattaaggaagtagctaggcatccttggctaggcatggggcttcctgcctaccttccttcttcaggctggatttttgtctggcttcagattgtacaggccttgtacatacagctgttgaggtcacatgtgtggctgtccaactgtgcctggaaacactgcagtcgttcacctcccatcccacggtgacccctgagcctttggaggacgggtgtgatgtggctgtcctgttgaagtctggatattctgtcgtctcattctctccgtatctcatttgtcctgagtcgctgtgttaaccactgtctatggaatggagacagttctctgagggggactggaggatgcactagtctactacaggcataaggataagtcgttggaagtcactgtgacactctggctgtctagtaggatgatagttgtaggttctgctctggaacctaggagctgttttgtcacaggcccttggtcccaataatgttaccagacattggtttctctccttgtggggggagctgtaagtccatccagagagtggttggttactcccttggtgtccttgccactattgctaggctggcggtcattgtagctctcaggcttcacagctgggtaactgtgactttgatggtgacttttctccccagtggcgtgcacagcaccacccagcatctccctgcaccatgagaccagcttgggttctccatgtttcatgatttgggtgtgtagtgtcacagccgtggggttttacccctcagtaatttttaccttgattagatcttaaactgtgggctcacattactaaatgtggggggttacaaaagacggcagtagtaagagtttcctgaacatacaaatgaccaaaaattagtgcagaatctaacatgtaatgagtctgaggtgtttgggtggcacacacctgtgctgccttgctgctaagctcatagcacatgtattggcaccgctgcaacaccagcggatgctgcccgagacacctcaactcagatttgagacggttgtatgttataaaatgcggagttttgctatgcgtgtagttttgtttttgttttttgcttttatagaggcaggcatagtggtttaattatagaaaacaaagactttaatactttcctacggtcattccttagcctgtacccagttatacatctctcgagtatactgtgttagaatgcttttcagaattgctgtgtaagttactgacatgagttttgattaaaaaaaaaatcagtagatgaattttcgcttgggattaggacagaatttccaaccatttccgaaatggccctaaacatacttctgcctttttgtactatagatttgtgcagcgtggccttttcagcatggcctgttataaaatcaaagtataaatcaattctgggacacgttgaagatggttatgtagaaactccttatagaaagacaggcatatgcatctcatgagtatacagacacactttcatctttgataaatagtaagcttatgtgtaaaccagagaattgttttaaaatgaatttctttatgatttattaacattatatgtttggtttgttttttaatttgttttttcctttatttttatgtgcctgcatgtatgtctgtgcaccacatgtgtgcaacgctcatggaagcatcagatcccctgggtggggctggtgttatcgatgactgtgaaccatcctgtgggtgccgggaattgaacctgggtcctctgtaagagcagcaagtgctcttaactactgagccatctctccagcccatttattagtaaacgttttggtttgtgtcactgttttatatctctttgtgtctgtgagtgtgtaaaaatttctgaagcaaaaccaagtggcagataggtgaagtgtgagaattcctgccgtaggcagtgagctggggaggagggaggagggaccttaattcctcctggtcgtgttctctaaggctcaggtggctcaggtggcaggcgtgtcatgcagaggcttcagggcttccagaccaggtatccttttgtgggaaaatcttctttcacgtgtccactgacgagtcaagagcttactactgctgaggccctgctttgtggcaggacttataaagaccctgcatctccagccttcataactggcaatcacctgtcacagtattggtgacagtgagcgatgtctaagcccatgttctaaccaagaccttcaagagagcagggcccagcctaggacccagcacgcagggcagagggtcggggtcagtgagcagaccctaaacgagtgcctctcagccttcctaacgctgcgatcctttaacacagttcctcatgtcatagtgacccctcaaccataaaattacttccattgctactccataactataattttgttattgttatgaatagtaatgtaaatatctggtattcaggatatctgatacgtaacttctgtggggatgggagccattaccccacgtgactcaatcactgggatgtatctcaaacctgtgcacaggactaggcagtcagatacaggcctgcctcctcccctgcccagggcttggaccctgctgcattgggagtggcttcataaattgtgtgtgtgtgtgtgtgtgtatgtgtgtgtatgtgtgtgtatgtgtgtgtatgtgtgtgtatgtgtatgtgtatgtgtgtccaccattacagcagagcttccagacacaccactggagggaaggacagattcatggcctgggaggatgggctgctgtgctggacactggaaaaaattaggtgttgccagcagaggtatggctgactgtgactggggcaggctgggaaggaaaattgtactttgtgctttggccttgaatgacccactggggatgagaaatggagtgggacccatcactcaaggccacactttttcattcaatgaggggcacactgaagcacagagaccccaagaaactaccttaaggccacacagcaaatcagaggggagcctgcactgagaaagcattttaaatgtcaggtctagaaccaagtgcctccctcaagccagtgaagtcttcatttcccagtggggtttccattaactaggaaacaaagcctctaagaggtaagaagacgcctagctgggtcctgtccttctgcgtgcttgaccacaagttcagaactccagctgtgctccctacctggagggtaggcagggccgtgtgagcctctgcccggacctggagggatgctcccagaggagagtgagctagagaatggcctggttaggctggcttcaggcagagtccactggtgctcactaagcccttccatggacttggatgggaagtccttcattccttccctgatgagtttacatgaagttctagtcgatagcctggaaggtccgtgcagagtctgactttgtgtgtgtcatgctggattgcagaggtgctggtgggtggctgagccctgaaaccacaagtgttttccttttggcctgtctgtaatcgggtgtgggtagtttccatgctggggtgggtgcagcccgagaaacccagcataaggaagcagagggtggcggttgtgttggggcggggactgaccagcctctccagcctggccaccgaggctggctccgccccacatgtgattctgattaggggcctgtgtaaaagttacctaaacaccactctaattactctgtttaggaacattcgcatgtgtgggagggtgcctggcactggcttcccacatgtgctgggggtctgcgggaggcaccgtgtttgcacgagcgtgacaggcacgggcagtggggagcccgtgggacaagtcagggagttcttccttgggttctagagttctggcaagccctgagtaggactggctgctctgctcgtccttgttagcttgttacctcttgcagaagcgaatgaacgtatgaacgaacgaatgaacgaatgaacgttcatgtcctctgggattagaccctcacctttccaatgggttctgcatctgcagcagcccgtctgtttcacgggcttgctgcgaaggcgagtgagctggagctcttcagagtccgtgagtgtgctgtgaggaccagggtatcctacagttaccagtggcgcatactcagctcacactggatgcagagtaagggcagtgggctgcctcagttatattgcaggggatgcctgcctgccaccctaggaaaagagctggtgtctgggaaggggcagcagagtgccccctgccaggtggattgcagctttagggaaatgtaccgtccggcctgatctgcagtcattggagagcacctcccacttttcttttgtccagggactcaaggcttttctcatctgtacaccggtgggtcctgtgtttctttgtgtctaccaccagccccgcaccagagagcctgccgtccgtccagccgcctcctcttttaaccttgtggcccacacatctgtgtccctacatgcttgatacccactgtattcacccgcctacccagccgttcttccacagacttttttttctaagtttaagatttatatattttatttcatgtataagattgttttgtttgcatgagtctctgtgtaccttgtgcatgacccgccttgtgctctggacagctgtgggcctgtgggtgccagagatcagacctgggtcctctgcaagagcaagtgctctcggccactgaaccatctctctagctcctgatccacagactcgagtcagcatgttacaggcgcagctctgatttcatcttagagcacacattcgtggcaggagttgcccctatgttgcggctcgaaggtgacgctctctgtctgatgtgaaggaggacctgccagcgcctgggccgtcctttaatgatgagtcaggagtgtagttccaatgactcaggcctctgtacgcttgcttatcattcattcgctcagttatctgtgctccagctgcctgacctgcctactcccgtgttaagtaccgatcttgccacctggctactgccgatagagctcccaccagctcgctcaccctcacatcattatcgtggggttcaggtgtgggacacacgtcggttcttgaacttgatctcagagacccttacccttccattctctgttaggcagaagcgtaaggtggggccccagttcttagtcgttcagccaatccaccatgttccacggtcactgagtgccactgtatgtttgatgacccttctctggcaaggggggtgctgttattcgacagttttcaacatgggctgtatgttctggtagagacagaataaacaactaaaaaatgaccatcagtgtagtgaatgcctctgtgtgctgtatgtggaacaggctgcctcaggggagacccaggctagacttgggcagaggcagggtgcctggactctgagctgggttctactctggaatgcaccttagtgccaagtagtataggcagccagagggacagctgaggaggccttgctggggaaggaaagccagcaattgcctcagagaatgggccgggaagaaagtcgggtcatagtactggggtcctgttaaaacatacatcttgccttttgtcccttgttacactctgatggctgggcactgagtggtggccctacttccaacccactgggcagtgcctgtgctgagctttacccctggtaccacatcagaaacagttgtgttacacaagatacgatgtttcctgaaccacgattctgaggtctgggagtttaattaaaacaggagccatacctctaggtatgtttgatgatagcattctagaatacaccacatgagaaatgatccctgtaacagctctcctccccactgcaccacactgcctccagcctttgctgtctacccgaaggtacgggaagagcttccatggggcgggtggatttgagcattgaaaagcaccagctcggtgcatggaccctatctttgtagtcctgagtggcagagctgggacgtgactcttagactccccgagtccccagacttatcccctttaccctgtctgcactcctggactcttggggggctacaaaggaagggaaataacacccaaggatctactcctgtaccctgtaccccccacccccattcttaagaaagatgcctccagagcacgccggccttctgcagtgggggcagctggggaccggtgagctaggtgtgctgtgatctgacaggcacagccaggtaggaagtcttctgttgaagggctccatgctggcactctgcagacaggccggcctccctgttaattttgaagccttagaatgttcccaaggatcccgcggattcctgctcatgaaactgcgcaggtcagaggccacacgtgttttgcttttcttacttgaggtattatgtgttcaaggtagatttatgctacaacctttgcttattgacttgttctttcccccgtgaacctgatggaacatgacagacgatatttacaaggcacgtgtttccgattaaatcatgtgagacattaaagcccgataaatgaaaacccctttgaaaggccaagttcagaggaggaggccatccctggcggggccttgaaggtggaatcgtcccttcagctggctgcagaaagtgctgtggagggctggcaggccctttgcctgcatggggccatttgtcactttttcccagccagtgactatggctgcttaaaactccgaggagccctgctccctctcacctgcctagggcggcaggagggacaagatgctccttgagtacctactgtgtgctgggctctgttgcccgagtgagggctttcctgcagtctagcaggacagcccagaaactctgaggctgctgctatctagtagatggaataagagtggaaccagccaaatgtgaccacagtccccaggatgctctgctttcactgcctgtgaccctggtcaagtttcttaacctcaccatcttggtcccttcccccgtgaaatggggacagtaatattgtctctttaggtatgctccatggtacccaggcccatgtagtgttcattattaagattatactttctgcagacactaaaggcaggcagttatagctgcaatctaggcctttggaccacaaagtttttctcagtattacctaggtggttctgccgggtgatacaggccagtcagtgactaccaagtgtggcttggctgcctgctgctccaggctgtggggcttccctcgtccttgtcagactgccctggatgatgggacatcttggtaggcagggttgcctactggagtctttccatggaggtcaattaagggatttgaacaaatctgcaaagtggaaacagcttgggattgaggctgcactccctccttctctttctcccctctgcctgactccctgtctgtcactgtgccttcaggagaagtctgctctcaggagggcactgaccacagggctgctatgggcctcataggccgtaggcctcaccttggcttcccctggatggctggttgcagaggcctacgagctactggagtgttttcattcttacagttcccgttttggttattggcagcttgggggctgctgtgtgggagcagagggtgatggagaagggtaggagtcctcctgcctcagcctcgcagaggaagaccttgctttggaaactgtggaagggccttcatcttctctcacctgccattttcctggaacctgggcattcagtttctaaagaaaccagcacagattgagtgaggtgtctgtgaggtggtaggcagtggctgtggagtcagcctgcctcgtgcgggctttaccacttgtggctgggttccctaggcaggtggcacctctccccctgggatttgtgtcctgttgcgataaacagctgcgatggaaataagacgcagttggatggaaggttctggattgacacagacaccatgttaggatctgctcatctgccccggggtgagcacagcctcggaacagacccatgtagcatgtctccttcctgtccccgtgcacccggtgcccctttgggcaaattctgggggctgtgtgagtttgaattccaggcggtcacctcccttctatagcctcccttactttatctacaacattgaaaaagttgccctctctgcttggcatgagttgtcatgcaaggaagcccccggaaactgcagatgtcattgatgggcatgcttgccgtgggaggtcacagcaggtcacaacatgcccttgacccccttcattctgcgccactcctggagtaccatgggtctgctcctgtattctcttaggggttcagtgcttctgggctccacagagcttgggcctcaccctatccttggagagaccttgtctcttggacatggaatgatccatgaaggcccctcaagttccttctaacctagagtcagttctcctcctccaacaccatccattgtgatccacatatagtggggtgtggctgtgtggaaggagaaggggagtcccttgagcaaaggcaagagactggcttgactttcagtgaacaagtgaacaagccctggtgctacatcctgaaatggaaacttcagggttctttggagaggcagttgtcggatggtgttttgctggggcatacatctgaagcagagttttcctgaagtggacacagatgaaagaataaggcagactggtgaaggaacatttagcagaagcagacacaggaggaaggatgttctgctaaagcaagcctgtgaaaggacatgtaacaacacgcatgtatcggtctgccttacattgagtagttgagctgcatttgtcaggactctgcagacttgggctaacctgatgcacgtgctgaggcaagacctgtgttgatgtttagaggtatacataggactctacagagtgatggaggctgagctaggcttgctgggacactagttgtgcaaggcttgtgggtcccgagtctttggtgatcttcgctttcctgagagagggacagctgagaacttctggtgttcccaccagtccctcctggctgtctctgctaggtcctgtcactgctgttgctaatctgactctaccatactggactgctggtgtatctgtgaagtgtttgcaagtggatggagctgccactgcctcctaacctatgaactgaactgctgatttccagacaacacagtcggaagttgttccaaagaacctttctaaacaggtccactccccctgtatcctttctttcccattacctctggtgggtggtaggctacaagggaggttaaagcatttaagaaccatggttaaaaataaagtttgaaaaaattaaagttataacatctcttggtgaagttactcttcttctctgtgccttagtgtctgcaactgtagaatgagattgataacacctgccctgctctggctacatagttcctgcaggggctaaaggctgtgtttgctgagagagggcaagaactaattataagtgctagttattggacaggttctgtggtgagcatggcgggcctgggggattgcagtgccctgcagggagcagacagttcccccagctgaggagggggtttgtgaaggccacgtgttgtcctcagtgcatagcgtaccgaggaccaagggggaagaacttatttctgtataaagtaatgctgatactcgggacaggctaaacaaatcccccttattgaagaatgtcgcttggggctgggcctgattaaaattggaaaatctaagcggcttaactgtgtggttttggtcatgggcggatcaggagctctgccttgctctgtagctccctggctctggtcatggactctgaggccacggagggtcagagacagtaataggcccattcagcaccctgtttagttgggagggggcagggctggaaggcccctctggccaagtaaaattcagagctccttgttcagagctccggtgactttgagctgggaagtaagtgttgggggattgtaaaagaatgtgttctgtggacccaatttttttcttagggtctccaggtcctgcccccttgtttgctcagaggaagggcttttgggcatctctggtgcctggtactctgggaggtgtttcttgtctattcagtgctatcaatttccctgtcacctgggctgcatcctgtgcctggactagtatagagtgtactagtacccagggcagagctgagtacaggcggttgtttggcgcatttgtctcctgtaggctccataggacaggatgggcaccgctgcttacatccaactggcaggggtgcatagaggcagtgtctgtgggggtccagcaggacatctgcagctggagctcccaccacacttgggtgacaagccgtggtggcttctagtgcctccattccctcatggtatcaggtggatagcaacgccacactggacaagtcacatttgggtagttatgccattcaaacacttgcagatacctaggacagtggaaggagatacctgtcccttgtctaggagagtcaggtcagccatggctctcctctggtcctacccatcagtgaagactgctatactcttgctaaaacccagatatattcctgaccttggcccagtggtagatttatcccattttccagttgaggacagagcctcggtgtcccctggctaggtggccagtgggagacaagtgcatcttagagtacgtgccacaagtcccgtctcaggtcagcccttgtctggatggggagggagggacttgtgaggtaaccacagggaggcaaggcgcttctaggcttcctgactgcttcccataaacagtaagtgttgaccatgtggcttgcatagatggtgggcaggtcagggcagtcctgggaggacgccgggctctctgatggcagtgacagcacacagggacaccaatataaagtcagctgtgttttttgtagtgcctttcctgtctggtggttcccagccagggctctggggctggactgtagagcctgagcctccactatcctagccacttggatccgtttcagtttgtgcatccctatcgtaggggcagttacacagcttagagcagacttaccccaacatcagctctcagtgaggatgctccgaaggaacggaggggagcaggttctgggagactctagccccagtggaagagaagacagtctcagataggaattctgatgcccctcaagtgagctaccacttccttccacccacccacatggctctgggtgcatccgtggaaggtgaggagagctgataggggtgtggagaacctggaccgtgttgctctcatggttgcatgtaaaatgtgccgttcctcagaagtgcagacgcagtgacctttggagctagcagctacagccctaggtccatacccaggaggatggaaggtgtgtgtcacacaaactggctcacaagtactaagggtcgttgtgcccaccatagccactctatggagacagccaaaatgtctcttggctgacccacatataaacagaagtgacctggtggaatgtcaactcagccgtaaaggtcacgcagtgctggtatctgtgacagtgcggatgtatgaatgtaccttggaaatctcccactgtgtgaaagatgccaaacagatatgggcgtctagcaggagttccacctgcatggaatggtctgaatggatgtacgtacaggggctgggaggagggcaggaggggtgactgccaaaggggaaggtttctttagggtgctaggcagatggtggtgatgggcatcaccctgactatagggaaaccgctagagtgctccttcgaggagagtcttgagatgagcattacagttcaattaaaaacaaggatgctgggtatgtagcacaggcctgaaatcccagtacacaggaggctgaagcagaaggaccactagttccaggacagcctgggctacataatgagacctatcacaaataccagaaaaacgaagtcctcggctgaagctgtcccctgccccttcccacaccctgggaccactgtctcccgtctttaggcctgctccagagtaggactggacttatgatcttgcctgcttttcttttctttctttttagatggggatgttgctggagagcccaggctggcctataggaactttaatcctcctgcctcaacctctgtagtactgactataacccagctcaccagctggcttccttcttgacatgctgtgggctaagaagcactaggcagagggtctgatgggaagaaaacggaggtagaaaacaggcgcaagtgagaaactggggcctgtggctagcagctgagaggggagaaggtgcaggggggacatggggagggaaggagggagggatggaaggagggagagaggagtagggcaggaggaaatgagggcaggaaggaggggtgaaggcaataggaggtgagggatgctggggagtaaggagtgtagggttgagaccccgacttctcaggctgagagccttgtgccctaatagtgccccagatggttggaatattgccttacgtggtcccatggccctggctgagcatgtcactttgtgtcttatggttttactgctgtgaagagaccccatggccaaggcgtatcataaagggaaacatttagttggagctagcttacagtttcagagattcagtccattaccctcagggcaggaaatgtggtggcacacaggcagacatggtgctggaggggtagctgcgagttctacatctggatcagcagacagcaggaagagaaagagtgagccacttggcttggtgtgtgcccactgagcaccccccccccaatctccagtgatgcacttcctccaacaagagcacactcacaataatgctattccctgtaagtctgtgggggccattttcattcagaccaccacactgggctttgtcctccccgttggagcagcctagctcagagtcctggctttcagtctagaaggcctggatttgaatcccagcttttccaactagtgacctttactcgctgctcagtctctgtatgttagctctcttaccgtgtgggttgtaacaggcattctctgggatggcttgtttccttctgcctagcccccagtgagacggtattcgaagatggagcctgaggagttgatgaggtttgatgaggtcccaaggataggtcccgtagtgtctctaggagagacaccagacaacccactttctgtgtccttgtttcacagacttagagcaagaaggcagcagctacaatctagaggaaggcctgtagcagaaacctgccaggctgtacctttatcttcaacttctcaacacccagatccagctaagcccgtatctgtggtgttttatgactcaggctgcactgggcaagcagggtgctcactacagcagtggaggcacctaggccagcatagcatatagaacaaagctggcatacctgctatattacatgtgtgtgtgtgtccatccatccgtctgtccgtccgtatgtccatccatgtctccgtggctgtgatggtcaccatgggccaatcacttctttctctcaattcatgtcctctgaaacctgggctacagtgctgcccacagaggctgtggccggtgtcagatgcaaggatccaggctgaggggctttgaacctgttagttgccatgtctgggaaccttgaggttttcatggggttggcatagagcagcgtcagtgtgtgggacagagtatgggaagtttagtgagaatcctgggcgagcagactggactggggtgccctggttgagacgcactccatgtggccttctaagaaggagcagtttgggctgcaggtccctggtacctccaccagggtggcagccgctgaatcagcatttccaggcttggtcctcagttcagatgtgagcagtgggttgtcccagtcatgctgagaggacaggactgacacctgtggttgcagcggtggctagctttgccccctgaaaaccttgggttaccgaggagggctcgtttgctcctgcccaggcagcacccaggattttccagatgggaaaatcccagactggaacacttgattcatcttctggacaggaggactctgctgtggcattgtccacggctgaggtcgtgggcccctcagctcagacagctcaccaacaccagtgcgtgggagagcaagcttgggagagcgcatgggctttgccgtgtgtcctgtttgtttgctgtctggtttggccggtatgtgggcagggcagcgaggccttggaatgttggcctgggtagcacagtggcttcgctggggcggagaagggcagacgtcagaaatgcctataaagcctcgagccaggaggcttccagacgtcagaggtttggtcactagaataactcagtcctggcccctccctgtaggcctgcacttaacagagggtgtgggctgtagtgattagagctacagctgagcccagttcatcgatgtaggccagttcttaacctctctgttcctctgctctctttcctgtgcgctgtgacagcagttggaggtgtctgagggtgtttctgggatgaactaagatggaatgtgcccctggatttgccacagtgtccggtagagtccgtcagtactcagtacacctttgctggacaggtgtgtggtgtggaagacagccaggcctgacagaaacctgctgccgcttccccgacttgatgctgtccagggcggctctcctgccatgccctctgctgagggtagaggagatcgcgagcctgcctcgggaatagtcacagacttgactcaggattcaggaacagacagcagctccctggggaaaggcctgctaggctcacgtggtaacactgtgaagcatggtcggggtcctgtgagaggcaggaattctggggagacttagagaagtccttggatgctacagaggaacacccagaggatcctgggtgaggtggcgtgcccaccactgccattgctggctcccctgcaggtattcctcatcatcctcagggttctgctctcctgttgctcgggtaacgtgcagaaggtccatgtcctgcttcctctcgctcccttccttatctggagagaaggtgatgtctaggtcaccgattgacctataaactcagcctgcgagtggagacataacccccaggaatgctgagcactgtgaggttaagtggaggggtatccacagcagagctatgggtgggatcagggtgctctggtcctggcgttgaggctggctccaaaggagagagagggaacctggatgcaggtgattgaagggagtggggccttggaccataggtgggatggggattccaggggtggagccagcatgagcataggcactgagacgggggactgtgaggggcagagctctggctggcagcacctaagggtgccaggacagagactgagacgggagtgcttgagtccccaggtcgtggtacgacaaggacgtttggggacacactttgtaaggttggtgttcacactctagtgccttggaagagcccgaaggacatttatcagcatccgggagtggggcggcagcaggttctgtgcttgcaggagggtaacccgctacgggaaaccatcgtagaactcctcaagcttctcctgccgtccgtcacacaggaggcggtagctctcccatcgatgcattaaaacgtgggtttcaagtggggagcagcggtgtgtaattcatttctcctggtgcttgatctgcgctcccaaggggagagaacaggaaggaattagtgagctctatattacaacttaattgcttgaaggaaaaatattattctttattgttaaccattagccgcttccagttttattaagctccaggctgcacggagcctcttggagctggccggctcctgagggccaggaagccctcagaactgtcccacctcataaacaggccttggatcttacatctcaggcacctgtacttgggtgcagcatccctgctgtgggttctgccccagtggtcccaggaaaggggctaggcagccagatcttctctcgaggtagaggatgctggttcatgttttatagtgtgtgtgtgtgtgtgtgtgtgtgtgtgtgtgtgtgtgtgtgtgtgtgtgtatgggggggtgggagggcagttttccttctagggtacttgatagtgctagctgaataccctggtatttagccacacgtttctctaggccacggcttctctttgtttccactctagacttaagctcttgtgttccagagtgctgtttggagggctagccctgccctgccatgccctgctatgccctgctgtgacctgttggaccctgccaccttccttggataagcaggccctgggcttctcagggtgtagggtgtagggtagaaactcaatgaaagtcctttcctctattcatcttctttcactgccggctccacaccaggtccctgtcccagtgaaggcccagctgaacccatgaagatggtgtgataataagcacagtgcagtggctgctgtgtgcatcttggtctgcctgagatgctgggacagtaacactgagtaacctgtgtgtggtaactcctgtcatccgaagtccagggtctgaggccaaggtgacacagccagaagcagagctggcctgtccgaatcccttgtctgcctgggctttgttcatggggtgctcctgcccctctcatgtgcccttaataggcactgttaggggctagtcccactggagctgtgtggctaggccagcagagctctaccgagctggggactgggtgggaccaatggcttacagaaggcagtgtggtgctctgagcctgagcagacgcagctctggtcactgactgttcgctgtcgtcattcgtgttcctccctctgccccatcttggtgacacttactgtgtgtggaagcaccagacgtacctgtctccctcacttgcccatgccatcccctgcctagagtgcccactcttctttgttggctaactcctgaggcccgtttcgtggcccccagaatgccctggccctcatgctttaaccccactgagcctctcaggctgctccgtgcctgtgctgttgagccgcctagccctctgcaggctaggtagcaggcaataagtgtgtttttcagaagaggaaactgaagctcaaggagatgcccgttccgctgagaccatgtccccagcctgtggctagcctgcagccagctgcctcctgtggggctcatttactgacgggatggaaagttgtatctgaggtgggggtgagaggctgccatggtcctgactgggacatgggtccaggatggggtaggagggctggaagaccatgctacacttggtttagctacttggtctggcattacagacttctcagaaccatcccagtggctggccagtctgccacatcaataaatcaggcaattgatggtgtcagatatttaatgggaaggatggagagtggggtgcacacaggcttgctgccactgcccccagctcaggggtgggtagcaccgctccctccaatgtgactttttggggggggggggttagtctatttctgctcattttagatagggaaggcagtgacagaaaatgaaaggattctatccaagcctagcgtggtgagctgatgagttcactgggtcctttcagaagtatgagtggctcagaggcagcctcaggacagagaagtccatcccagcatgtgtgatggctcaggagagctgcacacgcgagttcctgctctgcttaacccgagggaggggtttagagagtctgagagtttcttcctagtgaccctcctgcctccagaaggggacagttcagactaggtgtaacaactgtaccacatcaccacacaaccgcatatgataagccttggggggcgctgctgtggtgccccagaataacccaatttaggtaacttctgcaccttcagaggtgcaagcctgtgggggtggggttgagaacgggatctcggcatggggtggcgacagcagaggcctggaaatctggtgcttagccagtggttgatttggtgctagccaagaagccagtggagattctggaacctgaaagggacagggaacagaaagccaaagggagggggtaggttgggatggaagggagctggggagtgtgtgcagagcaagtctgtaggtggtacgtggtgagagatactgggcctgagcaacagacagccctgtacctaatgggtgcagctggtgttgagatgggagggaggctctaggctacgaggaggttcagggccctggcctccatgggaatgcgcatttggggcaggttgtgagtgacaggaaaggagggcaagcaaggatcctctcattagagccgtctggggtatgagacagccccagcagcgagaagaacgaagccgaagcctcggcagagcgttcatgagaatagtgtggatggtgagagcgccatggtacagccaggggtagagtgctgtgacaggtccccaaaccctcaggaattccctgtgggcccatgacgtggcctactatcagcggtgagctcctgagggctgagttaacttttctctcggttccttggcatccagtgctggctgcgcagggtgaagaacaggttagcaatccccagcaggcttagccaggcaggttagcctcacccacggctggaagcatcactcacaaggatcagccagggaggggtacctgctggcatcatgctggagaagggtgactggggggccctggtggggcttcagccgttcttatctgagacatggcagagtctactccagaaccagctgtctagtgatctccatatcctggtgacccccaaatcctgagaggaaggtggtacctgaacacagcaaacatctctagccttagcttctgtaccggaaaggatcatgatagaagagttaagagatgccgtgccagagacagtcaggcacattcaggtcttctgcgaggctgagactatgggccagaagtggtgcgtttgaattcctgagcaagatggtggaaggagacctgcccagatgggacagacattgggacttgtgctttccacagcagctggaaggaagactgtgtgcctctttaaccaggtcctgtctcttttgggtgttatgtcctcatgactgcctcttaacacagatatatttgtttccccttctgggcagggtactttccctaggctgtcaagttagcaggcaaaggagcaggtctgttacagacccaggctccaaggcctaggcctttgttcaccctagggcccacagtgcctctcacctacttcccagcatcccatgcagggctggcctgaggctgccgggagagccaagaagcagaaggcccttgagcagcgacgtccttgtctggaaaggtcctgtgtgtctctcaaagctcaggtcagacattccaaccttgaattcatcagcgtccccatccctctttcccaacctataatccagcccagtctagaactcacagccctctcgcctcccgagtgctggcttttgtcttatgcataatggcacaccacactttcttccatctcttttaccacaaagtcaccctgatcaggttcagacatttctactctgtcctctgttttctccatcccctgggttgcctcccccaaagctgagtttcccaagaagaggactgagtggttcactcctgtgcccagcactgcccacagaccagtgaggtccccttgctgggctcagaggagtttcttctctcccacctgcttgtgtcttgaaaacaaagccaccagtccacttccggggagaggcgtgggagctggggctggtgggaggggaaagaaactaagagaaaaagggcaggatttggcttggccgaggggtcttgcatgggctcacgctgccatttttacccatgtgaactgagctgtagggccccacctgaaggactcgcatgggacagtaggagctaagcagggtcagcctggtgtgacagtgacccggggacatgtgaatagagcagatgcaaaggccatttgagacagggtgtttctatacaagttgtgtttgaaagagtcatggctgggggtcagcagagaacagctcttgggccagcagtagctttgatgacacctggctgcatgtcgaagatgaacggaggggcaagataggggtcccaacaatggacactcatggacagggcatgactgggtcctgttggagcctggacatgatggatgggacatgcacggaggtgagggggtgacttgtcagacaggaggccatagcagccttctcagaaaggactcaccggaagagcaggcagcacacaggctctgtgggacctctccagggataatttgggcttgggtgggatagccaccgatcagattgtaagtcaggtgtggtatcgcagagagcgcagcagatcaaagccagggcgattgggcgctgattagaccctgcgaggaggccagactcatcaccgaggaatgcacacaggccggcccactgacggggataataacagatcgtgctggccgcagacaccgcggcgctcaggacgcagtcctctgtggcagggcagccacaaggaatggcatgtttgcaggctgccatgggacggggactcaagggtgggtggcagcatgcagggtcagctgcatggccagtgcccttggtggtctctgttggcctctttgcagcctgtgaagggggacttctctgccagctgcaccttggcagaggttggcccagtagcacttcatcccttgtcacttctatgtgcctgcctgctcacggccttcctctgcagtgctcctggcatgttctctcccaggactctgctcatctcctccaggaagtctcctatgacctcccaggcactgtttctcctgtgggtcccatgacctggtgctccccattgcggcttcttcagagacagtggacctttctgtgtgtcttctcggggcacagagcagatgtccaactgggttcacagatttggccagagccatgtccctggagccactatgccaatgaagaacaaacatgggctcagattggcttcccgttctcagagaaaccccaagacttctgtgcagagaggccacccaggtgaggaaagacaggtgactccccatcttgctggtatgcaggctgcatgcagaggctttgtgtagaggaagaaggacgaggtacctggtggatgagtctggacttgatcctgaggtcactagaggctcttgatctaggggaccatcagagaatggactgtctgaggggggccctgaaggttctggctggtatttctggctggcagaggaagaactgtcagccctgctgtgatgtgacttgtaccagttatgtcttgcagtgtgatgtgatgttcttccatagtggtccccatctgggtcccaatggctgcctctgatcttcagccttttcagctcagaggttaggagagagcaggctgtcccctggggtctgcagctctccataggttgggctagcctgaaatcaagccaaacctctgatttacaaacactgcaaacccatctgcctccatgtgttggcgctttgctgggtctgggatgctgtgcagctcagctctcttggaacaaggcaaaatgagcgacactgtgaggtctgggagtgactgccatgacccaggaatggtctgtgaaggggtggttgccactgctgtaggaccctaatttaggtctgacccccgacccctttctccaaacctgcagccctcagcccttcttgccttcttgttctgtcacagtcttgcaccagcagggggacaatcagagtgataggttttgtttttgttgtttttttttttaagatttatttattattatatgtaagtacaccgtagctgtcttcagacacaccagaagagggcgtcagatctcattacggatggttgtgagacatcatgtggttgctgggatttgaaatcaggaccttcgggagagcagtccgtgctcttaaccgccgagccatctctccagcccaagagtgataggttttaagaagcttttcaggtacttattgcagcccttcccctgcaaagggcgtccaaggaactggaagcgcagcagggaaaagctacggagacagagaagcagggtgtgcttctgagggagtggcgggaggtgtagacacgccccatgctggccaggacctcctggagctgcagtggagtcggccatcctggtattcagctctcctccaagcccatgagtgtccagtgtggggaaaggattccgctgcaagccctagcagaatagcactgtttaggaaacacgtcgtcataaacttggccacgacccagccggctggtgggtttcagacctgcatgcgactgtacaaagctcccattctgatctagatcccgcctgtgctcagaagtctctgtagcatcctcagcaactggccacccactgtgagctagagaggctctgggaactggtaccagcctccttcccaggtcactaatcaggattgtaacatgccctccccaggcctcagactcacactcccgggcctcagactcacactcctgtcctttgccaggttcctgctggggttggcggctgggactgagggtggctctcgatagattggagctggcggacttataggtataaccctttttccctataagaggactgccgtaggcatctccttcatccagtctagagaggagaatacagaagtctcctgtacagtatagaagtggggctggggactgaaccagggtcctttgatgctccagtctctgatgtgcagaggctctggtctggagaccacctgcctgacagagggcatccttgggtacctaatgagtataactcagtggtcatgggtgagcacccaggatgggatgggctgcacgccgcaatactgagtttccctgcctttcttctctgctaaggtactgttcccttctgcctcggtccttttcacttaacctggagtgttctcccctcccctggcttcagcaggctttgctcacttaatatgcacatgtccttgtgtcactatgaactgcaggacccggctattcccagctatcctggactgaaactgtgagccaagatgtatctttgccatcttcatttgtttctctcaagcattgggtcacagcagtgcaagggtgatagttaatagtctctcacacatgtaggtgctaagctcaaggcctgttgggcccatcatgaaaggggcagccctttcctggactcagtctccccatctgtaaaatggtaggtttgtggagggatcttttctttccagctgtggtagctcagccagctcttgttatgccactgagagagattccgtgtcagcgacagtgttctgcctcttcccccctcgcctggcctctgggtctgcccttattttctaggaaccaggtcagagggcaatggtatattccaatgagtgaggctagttaccctgtgcctatggggaccagtacttacgtcagttccagggcctttggcatctctagtgagcagtatcccctgtgaggacttgtcccgtgaggcagcccgcagggcagtcaaggatgcgcagtttgtctgggtcttgtattggaaaggaggtgactagcataaccatctagcagacgctgtgcttctcccgtcgggtagaaggacaggtcccgggtccgttcacaacgccatttggcccagggctgattctgctgatgctggctggctgactgaagaatgaatatgagttggggtgctgaggggctgtagaggaggcaggagatccccaccttcacacaggtgtccacctcggcctggggtgtcctgctgtccaggccagtttattcacgcagcacagcaatttcagatgccctttcttctcagagactggtggatcctggttaaattcgcgtgttgaacagccactgtgtgacttgaattagtgtgtataaactcaacacaaggtgggaacaaaggctgacagatacacgtgggtgccagaggctctgtgaaagagacaggaagacctgcccctgcgggacttggagcctccacgggggaggtaaactccacccgaagttccctgtccctgtagtgtttgtgtgtatgtagtatatgtgtgtgcaggtgtatgtgcaggtgtgtgcatgtgcttgtagaggccagagaacaacttttgctgtccaccaccactaccctctcctcccctttctctttccctcttccttctcctcctcctctttctcctcctctgaaaccgtgtctctcagtgggctagaactcaccaagcccattgatctgactgactctacttctccaaggctgggattataggcacacatgccaccttgcgtagcttgctggacctctcgctctctgtacatgtgtgtgtgtgtgttctcctccattgttctccaccttattatttgaggcagagtttctcactgagcttggagctcactgtttcctccagagtggctggccaggaagcccctgcgcaccgccagtctttgtccccaagtactgggcgggttgccatggtgtaggcttttctgtggatgctgggatctgaatgtagttgcttaggttgctccccagggagccattgttccgttcacacacctggctttattccataggttctgaggatcacacacaggtcctgtgcttgcagggcaggcactctgtatctccccagtccatatcttaaattctaaggtcacgtgtgttccacgcacttgaagcagtgcgtgtgtggaatgcccaagctcctccaacccttctctagtgccccccacccccgccccccacccccaccccctgggaagcaaccttgccctggaggctggatatttttcaagtccagcccgtcctgtcagtgcaagctatgcctgctctttcctggctgcttgtgctgggtgggacacagcttgcccagcttgcatccatgtcgcttctgcttacagctttgcatgaattcccccgagtccctgtcacaggtcctggcccttctcctggttcctgtcagtatagcatattcacctgctggcactgtccatgagcagccaggcactgagggtgttgcaacagtccagtcgaggaaggaggtcttgcccagtcttggctcaccgtggctgcactcctggctttatggttccaggggtgccttgtaatttcagcttagaagctctgtggagctggggctgggagcctctgcccaggtatcagtgtggtccatcccagacttggcaagcacagtgtgctatgtccttggtacagatgatctggccagtgtctgacataggtagatgtggtagagggcagtggatgtgtgcgccacctttgagaagcagaggaaatgttctcgtcatagtgaatcagcgtcctcaagtgggtctcatccagcctcctcagagagcagggcttctgactcctctctgctgctgtgttatggggagttagtcttcagagacattcgagtatcatctgcccatccattcatacccatgtgacatgtcctagaggtgtctcatgctggccctgaggtagggtttgtgtgcttgccgcgtgactgtcagagtcttgtctggtaccaagtgggtagcatttctgctactccacttggttcacagttcacagggggcttttcctttctgtaatgaggaggttttattacctctgagttggaggggaaggcaggggcttagcttcttgtccaaggtgacacaggttcctctgagcagaggtctggctgtcccatattgtcactgcagtgtggcattggtgtcagcaagtggcaggctgggtactcgtgggatggacagcacccacttcttgccagagaccctgtatgtgtggatagagcggtcgatgttttccagtctttctgggctccgtgtccttatctgtggcattgacataatgctctttgcttacaaggacatggctatttgatatttttcttttaaattagcagcagagacacattagacaacctctgaatgaagtggaagcctacgccccttcctctctccatccctccttccccgatccctccttccctctctttggtaagcttgcttcatactaagctatgggctggacactgagaacccagagaacccatgtgttcctgctcttggggcgttcagagacagaggtggttaaggaaacacatattttgcatccccatgttctgtaagtacacgaggcccttcctcaggcagggttgcttctggcaggagtgagggtagagattgtcatcaatgagggtcctctcccagaaaagatcaatgacttgcgcttgagagagccgtggaatgtgtgggctggtaccctctgctgagcagcatgtccagcagttcggacctctctgcagcctgctctgcagcaaaggtgtgctcagggctgccgtgaggcagctggccagtgtgcggtggccttccccgggtgcatgtctcagggttagactcagtgtgtgcttctgaagtgccggttcttcctgggatgccgtgcctctcgccgctcaggatgaatagggctgtgactcctggtgcccacataagagagttggggcatctgtggttcagggtgcagggattctgggtcctggggtcttcatgagtcgttctccaggtttccccacccccaggctgctgaggctcctgcccaagcagcagatgctgcagttctgacatttcccctcacatctgctttctctcctcctttcatctgaaacccaaccagactgtctcagtaaacaaacagtaaattcaacaattcggtctttttgaaatgccttgtagttattttaaggctatttggagatttattttaaaataaataattgtgggccctgcttccagggtcatccagctgtgggggcacctgtggctgctgcctcttgtccattctgtgtctcccaccagtgtcctgctggccttcgggggagataaggggaggtgggcgattactcctcctacgtccaaactcccgctgcatctgtgtctcgacagagacaactgcaaaggcaagaaaccccgtgccctgaggggaagacagactgggaggcagagtggtctagttttctttctgttgctgtgaaaaaacactgtgaccaaaagcaacctgggagaggaaagggtgtttggcatacagttgcatgtcacagttcattgttgagggagatctgggcagggactcaagtaggagcttggagcagaaaccatgggataacaccatgctggctcacgtgcaggttcatgctttgcagaaaccatgggataacaccgtgctggctcactcgaaggttcatgctttgtttgctttctcatacagccagaggtcatctgcccagggaccggtgctgcgcatgcacagtgagctgggccctcctacgtcatctcacagtcaagacaatcccttacagacatgcccacaggtcatcctgatgtgggacaacccctcaactgagactcctttctcacctaactctaagttatgtcacgttgatacttaaagtcagctaggatgcgccatccctttataactctatacacaaacacatcactttaaactatcaccgttcaactccttatcctcatcttaggttaacagatggctcagcaggtaagagcacccgactgctcttccaaaggtcctgagttcaaatcccatcaaccacatggtggctcacaaccatccgtaatgagatctgagtccctcttctggtgtgtctgaagacagctacagtgtacttacatataataaataaataaataaaataccaaaaacaaaacaaaacaaaaaaccaaaaaaaaaaaaaaaccccaaaaacaacaaaaaaaaaagccccacaaaagtttcacagtctttaagactttcaactctttgggtgtgagggtgagctggcttgtcattggtcacccttttgattgccaaaactgatttggctgttctgatgggttctttttcatatgaagctgcccattctgtaggaggacagccttccctgaatagaagactgccaagagtatgcaggcatctgtggtccctgggagaacctccaaaacaagttctcaagactttcaacatttttaaaatccaaggttttgcaaggtgaggtggtgctcacctttaatcccagcactcaggaggcagaggcaggcagatctctcaagagttcaaggccagcctccatagtgagttccaggccagctacagctacatggtgagatactagctcaaacaaacaaacaaacaaacaaacaaccccaagttacccactcagatatctttaaaaaccaaagtctcttaactgtgggctcctatgaaataagaaataggtcatatacttactcattctgaaagggaagaactgggcataagatcaatagattaaagaaaacaaactccaggtgtagatctacattcttaccttggcatagcatctggaactcatgatcttctgggctccaaagggcttgcacagtcacactttctcagctcctgtgttgatagcacacagctgatctcaggcataagcttcatacttggccccgtttttggcaaatgtcccagtctcagcatctccaatatcctggggtctctactccagttgaggctgtatctttaccaatggcctttcctggcatctcttcagggattctgagcctaccacagagtgccaagcctcatcttctctccctgaccctgtcaatcctgcggtttttatgtcttcaaaaaaaaaagtaccattagagagactcccacacactgccaggttcagctgccggcttgagatgtagcctcctcctcgcctgccatgacagtgtgctgaccacgtccctgtcccagtgaagcacagattttacctcaacgatgctaatctcttattaatcacacctgattattgatccctagttgatcagcacacacagattctcagtctaaaatatcacacatgttgtgcctgccccagttctgacaggagtcttttctgtgttctctcaacgttcttccaagcctccctaagagtggaccactagatagggctggagagatggctcagtggttaagagcaatgcctgctcttccagaggtcatgagttcaattcccagcaaccacacagtggctcacaaccatctgtaatgggatctggtgccctcttctggtgtctgaagacagcaacagtgtactcacatacattaaataaatcttttaaaggaaagaaaagaaaagtggcccacaaagctctgcccacagcactcagtggctttcccagcccaaagttctaaatatttcataaaaagtttacgtccgaggcataaaagccagatagtcatgttcgccatgatacagtccacttggctgcactcctttctgctgtacgtcccttgtgttgctgtgagaaaactccgtgtgcaaaagcaccgtaggggagaaaaggatttatgtggcttggacgtctaggctgtagtccgtagggcaggagctcacggaggacttgacgtggaccgtgatggagtaacaccgcagtgccggcccgcttgcggcctcacgcttagctggctttcttatcctatctgaccgtgggatcgtgccagccacagtgagctgggaccttctacaccagttaacagtcaagacaggctcccaccggcttgcatgtaggcctgtgtgattcggataattcatctggggctctcttctcaggtaagtctaggccgtgtcaatttgacagttagaactaagtaggacagatgagtttcctggctccagaaggctgggaaaggaacaacagggcttgttttagatttgctcccaggagcagggccacagacccaccccactgacagaaagaatcagaaagttggttccaaagaggccagatggagtcttctgactgggaggggtttctggagaccatcccgtccacagttttctctccacactccctctacagctgtcctagaccgaggggtttcctcccctcttgttctgcttctgactgacctgtacccatccactgaggtctcccctgagctccgtggcccaggaacccgaaatttctaagtttcaggagaattgcaccaaggaaagtctgggctggtggtcctaccccctcattacatgtgacagattctcctcacagacatggactagaaaatacccatgggctgaggcctggcttccttcccaaagcaggcaaattgaaattgaaatttctgggcctcggcttcgctgtgtggtgggatggacagcagtggcccgtctccaggtggtaattcctaggttctgagaactgggagctgctgtttctatctgaagacccctggccaactgtccactgacaggaccctctgtgctgtgggaatgcctgtgtttctccacctctgtgcataggcagaactgggcttctgaccctacacagtccactccatgtatttcccaaggagctagggccaggagcagtggaggagccccacaccatggggatgctttctaagtgccaggcacttcagagatgtttatttccccccacagctctggcacgaggagaccattgttcccatttcatacatgcgctgcctaaggctagcttggaaggaacatggggcaagcaccagaggccagctcctcccttgagcaaggggaagactgggaacctcgggatggacagttagcacggagagttgtgggaaggggggagcagggatggcgtgagagcaagcgagagacaagggtgctcctggcgggtgcctaggcctgggtccaaacagagtgaggcccaggccttgggagacgtctggtctgggtccttctatttatttgcacatcaccagatgtaaactaatttgtttagcaagaaaaaaaacatttttattgcctttaaactgggctgtttccctctggctgtccatcctgcactggtagactggccaggaagcagaacttttgggaacagtgtgaggtgaggttgaggctgcgatgtcttgtgtggggtgtttgtccgcagcggttccctagcagcgcctactctgccgcagcagcccgtgcatccgcagcccaggcatctctagcaagctgcaggctgcgctgtgttctcctgttctcttctggaggctcctgcttggactcccaagaggaagcctgtttggttcctcccgctctgcttattttgacacgtggcagtgtccattgttaactatcccaccatacctcatagacaaaccacagtcttaggcggactgtcaaccccttgtgtggagctcatttctgtctgggctcctgtttggtgcaggggaaagtgaaaggcgccttaaacacaggaggaaggagcactcaggtttcagggtacttactgtgtatgccagcactcaggttttagggtgcttactgtgtatgccagcactcaggtttcaggatacttactgtgtatgccagcactcaggtttcagggtacttactgtgtatgccagcactcaggtttcagggtacttactgtgtatgccagcactcaggtttcagggtacttactgtgtatgccagcactcaggtttcagggtgcttactgtgtatgccagcactcaggtttcaggatacttactgtgtatgccagcactcaggtttcagggtacttactgtgtatgccagcactcaggtttcagggtgcttactgtgtatgcctgctgctgggttaggtccttgctgtaggtcgcgtcttaatctttgggactgcagggtagggatcaccattctcgctctgcggacagtgctcacagagcccaagagtggagtgtgaaggaaggctcgtttgtgcagagcccactcctctcccctgcaagtgctccagagcgagcacctgcacttggaggggttctcagagctctgggtggagaaggtggaggcaggaagatgccttgcctgggccctagagggtgctagagggtgtgctttgagcttagagtgggggggttgaaggagagggagggttgcttctggccccaggacccaaacctggccaactggaagggcagttctgtgtctagaaaggggccaggttcctgcttgatccacccagttttcccctaagggattggtctgtgaccaggacagtgcgtgagcttctgctctgctgggtgtgggggcctctctcagtgagtgagtatcctgggaagtacagggtgagtaggtcacagtgggaccaatgcaggctctctagggatgctgttgaggctgccagctggggtgcagtgccgtgagtgacatgcgtggtgtagggaagaggggtgagagaggaagagaggcgcagagagatggggagcttcaaggaagctgtgaccagcgctcagcctacacagaggagacccagctggcacagagaagctcttctactcaggcttggacggctacttgccagagggtaactaggagtctggctccacccaagggtcttttcctatccctgtccctcaagtggagccacaaagcttgcaagtgatgcttgtggtgtggggtgtcccagattgcttcttcctctaggctcgatctaaacctcagaccccaaaccagagcaaatggtgctaatttgctgaatcccaaactgaacccagggattaattttttgtacctgaaccaagtctatgatttcttactggaaataaagagagaaggttctatttaatttaaagtctaaccgccttttctcctctacgattgcccggagttgcttggacaaggtcctgttggctgttggctctgggtgattttacaggctggggtggggatggagtggggagatacagtccccattgagagcttttgagggcaggtttggaggaaagggtggtagacagtgtcagctcaacagaagcagccagggggctggatggagtctgggtctttctgttccctgcttgtgtagtaagctctctggagatgggagagctggcctgttgcctggagtgaggggagtggggcgcaatgctctggaggtctgtgcatgaggtcagccacacaagctgatctgggtgatgctacctgccggcatggcttcgtttttcaatatcctcttaggtttccccgaagaccaaaggcaaattagggtgtccctgccggctgtggggcatcatggtccccttatttgaggctgaaccaggtacaaacccaagcctctgatatggggggggggtaatgggtgggatacaggcatctatgtccactcatcctgtctctctgtgtgctgcacgcaggtcagtaaacttctctaagcctcattgtaggcgtgggaaagatggcctagtgactaagggtgcactcatagtgtttctccaaaggacccaggtttggttcctagcacccacatcgtgcaactcacaaacactttaactccatgtccagggacttccagtgccttctggcatcagcaggcaccacatgtgtgtagtggacataaactcacacatcacacacacacatacataaataaaataaaaataaatgaattttttagaaatccagagaaataagaccctgaggtactgaatgtcttgtcctagctaggattaaggccaccgcatggagtaacatacagaaggtgctcagagagcaatggttgtgtgagtgtggggatgagagcagactcaggaaagaggccatttggggaattttagcttattgaagttatgtagaccccacttctgaccccagtccagcaaaagtgggggtgtttgaaaaccagctgttgcttggttacaggctttgtgcctcttcagcctgtgtgagctcctggcctgacgctagcgagcagtactgagttagcaatactaggtcatcagtactgggttggtagtaccaagtcagtgatactacatgctgacctattaccatggccacagtgaggaggggtctcaaagtctatctgcttcaactccccctggctcctggaagcttggctctccatattctgctgggatgacctcaggcctcagtcttccttctgtaaaatagagctgtcaggtcccttctagtgctgtagggttgtgagctggcatcctttaatggagacagcattgactcagtctcagagggctgtgctgaagccctaggttttgcttgtctgcctctacttgagccacttgagtcttcccttgcacacctcttggaagagtggttctcaaccttcctaatgatgcaaccctttaatacagttcctcatgccgtggggacccccaaccataaggttctttcatggctacttcataactgtaagtttaatgttatgagttgttatataaatatttgatatgcaggatatctgatgtgcagcccccacaaaggggctgagaactactgtcttggaggtcatgggcctttgtcctggacctgggctgggtatgccatgatgtcccctccgtgtttcacagggtcacaggactctgggtcagacaggtactatctgtgcatgccttgggagcacagttacctggtttctgtccccagagacagaacctgaggcaccaggaaaggaaatgactcagggctatggtagctgccaagggttctcacctcccagggctgcaggcttgctcacccagttcctgggccctgtctccattgagcctcaccatagagcaggcaggccctgagaattgttctttttggcggtttccaggggtgtggaagtgttggttgtgacacacacttcaggtagtgctggtccaagtcgccctgacccagggactgtgtagtttctcttcccagggctcctttcctacgtccctgctgagtagcaggccacaaaggcttcggtgtaagctttagagtgtcactgtcctgcgtggcagagggatgtgtagggtagaaggagctttctctttttttctgggaaaaattaggtccatgtagttggatttcatccttctaaataaatgtctgcttgtgcagtaaatgtttatgtgtgccactctatcccagcccctgagccaggagacaactgagaggaaacccagtgtgtgtgtgtgtgtgtgtgtgtgtgtgtgtgtgtgtgtgtgtggagtgtaggcttctgggagtggtgggtgtccacagggaggggccttggaaagctccctggaggtatgcccgagaggcagtcggggattgacgccaggttcccacctctctctcttggctgcttggctcactctctcctgaggcgtactcagaaggggccggctttcctgagacaggccattgtcttggaggtggccctgatgccactcaggcactttcagagccatagcatcccagccccattgaaaggttgtctaagccatgtcggtgatatcacaagaagtatggtgcgtcggccgtgggccgagttaaaagccgtagagcgacatggtcccaagtggagcgtgtgattagggagacagagctgatgcggatccaggcagaaccaggcttccacggcgttggcgccaatcgggctttataggcaggcagtggcagggaacacacagcacttgccagcttgggtgataaggctctggtgctggactggcacgctgggctgtgtgcctcagctgtgagtggggacaggggaggagaacagaactgctgcagatccaccccctcttgctgccctgggtgccccgacccacactgaatccaagctgcagggctgcctggagggtctgtaagttaaagactttcatgtgaccctaacacacctaccaggccaggagggtgtgatttgacttggcggagtgctgcctgctcagctagaaacctgcagctgagcacctggcattgcttctaggcgcacggctaagtaaatagtccaagaaggggcattctgggtatgaggggagcagggtggcttttctgcaagggaactacaagccagggaaagcttcccagagaaggtgactttcttgagagaggtatggatgtgtaggcattcacaggaatgtgagagagctctgtgtagcggagtggggccagacgggcgcagtggttccattctgtgcatggaggatggtggggcttgggcaggtggagcatcttagcgccaggtgatggaggccagctgttctaactgctccagctgtttcccaagacacccgcaccctcccgggaagctaaggcagcgccttcagcttcagtccacttagcaactggagcagtgaggatgcctggtaggtgcattagggtggccctcatgcctcctccctccaacccctcccatgttcctctccatctccctcatattcatggtctccttttcattataattacacacacacacgtataaatactaccttctgagcctgtttagtgttccttgtgtgtatatgatttcagggctgaccacttggtattggataaccagttctggggctcatccctgggaaagactagttctcccatttttagcagtcctgatttgcctgcagttgtctaagacggggccacatgagatcccccttccatgttagcgtgttcattggcattgccattattcgggccttgtttaggctgctacattgttgagatctaatgggtgtagcttcccgatcatttgaaggaggcaactattcacaacagacttcctcatccgctggctcttacagtctccactctttcttcttctgtgttctctgaggctcaggtgccagagttgtgttgcagatgtatgtattggggctgggcatccatgatcagtccatctctgtattttgccccgttgtggttctctctcattttctctctgctgcagagagaagcttctttgccggtggggtgtgtgtgtgtgtgtgtgtgtgtgagggctacacttctctgtgggtgtaatatttagaccatcgttaggaattatactggtctagtgaagaggcagccatgggtcctctttttagattccatgacgtcactagccccgatagtcggttaggtttctagtagcaggcatgattttccctcctgagcaggccgtgagtccacttagacggctgttggttaccaccaagctgtgaaggccactgttgcacctttaggggtgctgatcggtgttgtggttcatagacatcatggccaggtaggactgttcgttgcttccctcccttggcaacttgcatagcacttgctggttctctgaaagctagtccttggggaggaggctggtcattcttcctgttgatgcaaagggtcccctttccaggagcattagccaggccatcactgggtctgttctcattatgcaaggtacccaggtccctttccagatatggcttcatatgacccagccagctcagcggggctctgcaggataggttttgcgcctggacatttttcttctggttaaaagtattttgagagctcattaccgtgtcgactccacacgtagtgagcccgttgtccttcagagtcttctacttgttagaaatatctacagctgtaaagaaaggttcgtgtagttttaaacaattctccgctggcggctgtctagcaattctgcagtgactaactcagaatgagaagcattctctcacacgttcccctcacagagatgacagcagcttcccctgcgtctgctccctttggacactgcccacattgcgtgctttatagagagcttgagtcatttgtagaccttgctggccaggtgatgtgttgggtgccggagagcaaagatgatcacagcaccttggaaacaacacttggtaaaagagtatgtgaacatgactactagaccaagtcgggttagtgtactgggagccccatggagacaggagcttgctaggagggctcagaggccagcatagagtgtggtgcccagcatggatccagggatggatgtgattggggcagcaagggaatgcagaaaagacaaacatgtacatatgaacaggaaggctgggattagggaggactgggctttctgatagaggacctagagccccctgcctatcctgtttaatatgtacagtggaatagggatggatgtggggttgctgcgtgcagctgaatgaggtgggactagtatatacatatacatgtgatataaacaaggaggcatgcttgttatatacaactgaacaaggaagcaggtctagcttatctcattaggaggagcaatcttcagggcttcactgtctgggggagaaagctatggtggatattttctgcgtatactatctacattcacacatgggctcccccacccaagggagactttgccatccctctgatcctcaccctagggagaaagttttgccactcccttggggctgaggcatccgagtagtcggtgtggctgtacccatggcaacagcgcacagctcaggatttccttcactctcccttgctccccacggtggagaattcggacatcactgtggggcttgaaactttttcctgccagatttaatttagctgatggcctaggatgagctgaaaggtatcttccgtttagaaaattcagatacgggaccctataatcttttattaactctctactgggtgttgaaatctgggtgtcaggaagtgatgagggagaccctccctggctttaagtaaccaacagccctcctctcaagacagcatggatgtctgagatgattacatcaagaggttattgggcttacaagggggacattgggtgtaggtttgtggagttttactctgggaggtcactgtcccatctgcaaaccttatgcagaagaaactattgtctgactgactgcctcctcattgaagaggtgagttcagactgattcctgctggctgaacctttaaaaaaaaaagagatttacttattgttttgtgtgtatgtgtgtgtgcacccacactcatttatgtgcaccacatgtgtacactgaaccctttggtcagaactggtttatctccgaatctgaaggtctggtccagaaatgggattcctgtctgtgctgaggctgcttggccacttctatcttagggctgagtcattcggttaacaaatcagaccagtaatctccacatgttgtcatggagcctgtacaacatcaagtgtctgccacaaatcagaccaaatactgtcagctggccccactgggccgagagctgcctggtcattgagtctttggtggacagacggcttccttcccgaatccatgcatgtgaagtctattctactcacataacagatgacccacttggggctaagctggggctagttgctggctgctgcctgggatgtatccagcttgctttctgacagccttagccctcacacactgcctggcatggaggagatgctcaggtggtatttgtgagatgagggactaagaagaaactagttcatcctgcatacatgagcagaggggccaggctatggcctagccttatcaggtagaaaagatgccccccttccatccacctgcctgctgttgagcccctgctggaatcctgatcctgtgctgtgagatgggtccatagagggggctcaggtgatcacagaggggccatgaggctctgtttagactgccccttcttgagggtgctctgtctgtgcacacctctccacagccatgtttccaagatagagacaagttcagaactctagtgaacatagaagagacctggatggagggccccagcagaccaagggagatagactagtttagctaaaggccatctgaggggacatccaggcacaatccaggggcttcctagtttgctctgtgaccttcaacagtgtttctgaggaatatccatacagtaggaatattaggaaggtcaggtggttctgggtaccaacaccatgtgggagaaaatgagctataaccttcacaaatcacgaataagaaaatgttccataggccagtttaatgggaatattttcttgtttgtggttccctctttccaaatgcctctaggttccatcaggttgacataaaactagcttgcaccagcaaatgagcaagggtataagtgttaactacacagcttatacctccttgatatgcaacagcccaagaagaagcccagggccatcttggctcaaggttggtccccaggtttcagaagcacctggtgagaaggacagaagggaaggctacagtgtggggcatcaggagggaacagggtctaatcactggccctgaagctccctgcatggctggaaggcccccaggtggaaggagtttgggagatgccgctaagggcatggggtatgcagaactgtttccttggcccagggcctccctaggcttaggccctttttgagtttcagaagatttgggggaaatattccaggctggatccttcgagaggaacagagaggtgggaagacgaggttgcagaggagacaggctggaccctgaggggacctgcactatgtcctgtgcctaggatctttgtcaggcagagggcttggggatgtaagtcagcgtggctgtcattgatgcagaaatacagctttcatctcatataggagaataggttatttaaaaatgagtgaccatgtccctaaaacacagatccaagtctccctaaattctgtgttccaacatggtaactgagtgatcggattttatggtaagggaacgaagaaagtcatgatttaaggcacttttaaaatacattggtggtggcgctattaggcaggttaaggccaagtggggggaaaactccgtcataggcttccaatgtggctccctcagcctcttggttggtagaagctagtggtctttattcattccaaaaggctttatgtgttagtcacaaggacattaggtcagacacagagatgggcaaggagtgtctattaaagggtctggaggtagctcagggcagaactgcagcatcccaatgtctcccggccattgcagttataatcagtcttgcaaaaggtccagtgtccagttgcactgcttttgctcagactgacctgtctccgtatgagcaagtgcccagtttatggactcagtgttagcctgtctatgtgtctgaggcttcagacatggcttcaaacactgagtccatgtaagaagcatttggagtcttgtgggggaagagcaggcaagctgtagagatggagcagagctgctgtcaagagcggtgtgtgaagagtggctgctctgtgagtgccctggatggtgccggggtagaatggacaccagcaacctgaaagccagctttctagaaggtcagagcttagctttcacattccctggctgtgtaaccccaagctgtcttctgtgaaatacttaaccgagaagtagctgttgggggtgttggaaggaataactgttaaccccctcctctccctaagttgttctgccaggctcctgtcctgagcgtgtcaccagaatgaagggccctcatcgtacacgacctctcaccagcagaggctcagttgatcacacccaggctcttgacacactaatttggtttgtgttcaggacacatcgtttccctctccgtggtagcatctctttcccccactgcctactcttcagcctctttttcacagggtcacaggctgcacggctgccgtccttgtctacaaggcccctcctaaggatctttgtcatacactcatctttacatataatttgcttaatcgccaatttgttttttcctcctttggcctcacctctgaactacagacttctctagcaaatatctctagcctaactcattcctaatcaagctgccaggcgccctagccctgtctctctgttcctgctattccctcatggccttgtctttgctcttctatgccttacatctgactcaccctgaagatttgttggctctaccttgacaatgagggcaggggtatgacttggccgggccagcatcatgctcacttgggtaagttccctgtaatgtcttgtttagttttccctcctccatcttagcttctctggggccgagtctagtcactcacaaacccttgcagtgtgagccacactgcagtggtttgtaactacttgcttgcagtggtgacagtaaccatgcctttcattgtgccctttgacctcatcttgcagcatccaccctgtcctgccgacttgtcctcaaacaatgcttggctggttaataaagtgcttggcaggcatcttagttacttttctattgctgtggcaaaacatcatgaccacagccacacaaaagaaagcattgtatttgggaattcacagtacaagagggttagaaaccacgaccatccgtgtggcagggagtgtggcagcgggcatgagacaaccacgaggctgggagagctaactggtaatggcacggagttttaaaaaccacaaagcccaccccttggtgacacagttctaccaactgctgaccaagtctgggtctttgggggccattcttattcaagctaccccagtgggtgccgtgagtcactgcaggatggcaggccaggatcaagatgtctgtttgctgcttctctgcaaccctgcagtgccttggcctcctaggagggttgtgctcagattgggttcctgtcagcagctgctgatccctgcctgcctgggggctcagctgtgggctaggccttagaaactgaagaaaacaaggcaggcccaagacccaagagttccttactcagtggtcaagatagaccaaggagactagtgaggagccagtggtcactcaggctgagttggctctgctaaggccccgccttggttagtattgatggtcacagagactagaggagagatgcaaggacatttgggaggtagggaaagcaaagaatgggcttgtaccgcaaaagtgagcttcaagtttaaaccctggccgtgttgtttgtcagttgtcactgtgaccttgactgaaaggttatttagtttttctgtgccttaatgtcctcatctataaaatagagatgttgcagggtttgcatgagaggagttgggggtcgaaagagctgctttaaagagtaagaattatgtgagcatgtggtacagctcagtaaaccatggcctctgtcatgatcggtgacttactggattggggaatggaccaaaggaatgactaagatgatgctctaagtacttggagaggggacaggccaaattcagatgggagtgagaggaccagctcactcagttgggtaaggtgatcctgtagcgcatctggtgggaccactgaagctctctgcctcccacttcccttcccttcccatcaggaggcaggcagccccacgtaggatgagtttgggaacctggactgtttactgtccatcttgctcccctccccctgtggagacagcagagggatacagcagcggtcatcagtggtcatcagtgccgtcatcgttgtgataacgacaatgataatgacccccatttgtgccgagattcacatctcatacagagtgtcctcctctcaagtccattacctcacaggaacacatggttccaatagtggcgagggcacatttctcttgggccctaggaggccgctctttgggaagtctgtttcaaataaccgggtcgtacatctttacagcgctttgtgccttgcagagctgcgtcacgttcttcctctcttttggccctcccactcctttcgtggcccgtggtccaagaaagaactttgcatctgacaaatgtaaactggagggcttgggggtgccagccatttccaggctcaagagtcttcattttcagttagtgtttgtaagggcgggctgtccactgagtagcaaggcagccacttggcgacaaggaagctcggtgtccctgaggagcagaggctgctgtcccaacgcaaggcttcttatcggtcttcatgctgagcctggctctttgtttaaaggcatccaggctatctgggcactcaccactgtcctcctcagtgtgtagccctgcctgcctggtctcatgtgctgctagtgagcccttgagatcctagcctgtctgtagtaccttcctaagtgtcaaccaggcaatcctggtgcttcctgccagatgggccctatatggggtggtctggagagatgagctatctggaggcatttcactgttgggagattgtttccacttagagctaattgggcatgtgggagaattctgtttcaggatgaaacccaaatagtgtagcaggtgaataagccattgagatggctgcctttgccactccatatgactgccacctcagtctgccctacttcctccaggaagccttcccagctgtctttacttggggagaggtttttatggctcttacagtacagggcttctttgcttaccaccttcttgcccaccgtcacagtagcagtggcccaccatatcaggagaagccttagcaatctcgttttgtctcttctccagcttgttttccacgagccagctagtgctatcacatacatagatgtatatgtgtctgcatcttcctcagtcctgcagccgtgggggcgggggggggggagggggcagcattgcccaaggccatgcaggggaacagctcatagatgagtccactcagatctgtagtacagctgcctcctgcaaatccttcatgagaccagactgtagccactacacccagtagcagcttagctggggtcgcacttagattggcaacgactgttctcccattctcctgggagtctgtgctcccctaaagttctggggtccccacattcctgctcagctcgaggacaagatccttgtggtctggactgagtgcagggtctgaagctgtatgtggcctcttcttcctgccccatgtgaccagcacagggcttgtgaccactgggctggcagtcaggtcaggtgactctggctggagctcctcttcctctgcccttcctcggcttggggccggtgaagaagctcgagtggcaagaccaaatttctgacacgactgtgatcagtggctggctggcaatgtcctctctctgagcttgcttccttggacacacatggggcacacaattcacaatccctgaattgctatgacagttagaggagatcccataggtgaaggcctggtaacctgccaggcaggcagtcattaatgactgaaggacggatgtgtggggagggtagacatgaccagaaggaccacaaaggtcaggcagatggaagagactgtgaccttgctgtagtaaagctttggtgcctgaggggttagagaagatgaagcaagttcatcttgccaggtgctgatgagatgtctctccaagtcagagaagacctggaaaagcctatagctgtagaatgtgagtagccaggacttgtgtgatcttggacacattgcccgctgtagaggggctgggagtggcagaggcctctgtgtctctcagttgcttagctagggcaggtctggatccctccaccctaggcgcctctgaggctggcaaaagtcggctgctgaatctgtactgggccctcatcaagggctggctctgagttcttgctgtcttcaccttatgaatcctcacacaaaccctctgaggctggactaacgacttccactttacagaggagaaaactgatgcttggcgcagtcacagcttgtctcaggctggcacagatggtgaggggttggtgctataggacactggggctggggcgtgccctccgtggcaggtgggtaaagtgtctccttgggtctgctcagcaagggagctgtatggagtggtaccatggctgggaagtgagacccaggttccagtcttgctgtgtcactagtttatcacaggacctgacccaagtgctttggcaattctggctccagtttcccaagattatgtcaaaggagattaagtggcctgtgggctggtgtcctctactctgagaggccaccagtagctgactcctgtgacaggcagccttaccctgctgccagcctaccctcgcaccagacagctctcctccgaggggcttctgagtgccccagggattctcttctcctcccccaccaccctccttttggtctggactctggaaggctttcctcccgttcttggtccttttccttcttaagcccgtatgcagcattagaaacgatgctgggttcatccatgatatctccctgactagtgcaggccaggtattggtccacacctgccagttacaaatcacacccgtcctgtgatggcccctcttcacagaagaggcacctgcataggagagggctactcagccaagatggcggtaaagagtccaggcctgcgcagctccgtggttcactactctttggtctctcagacctttattcattcattctgctaactggcctcatcatgctggccctgtgtagagcttggtgcttttcctgtgcaagtgtgatactgtggcagatgcggaggtcagcttgactccagccattgttggcttagcactgtggacacaggaggagtgtctagctggagcaggcatgatgatgagtcagaggagggcagggttagccccatctcagagaacccatgctctagaggagactgacatggcctttggcatggcccttctcttccaagaagtcctgtctgtcttctccttcttgggtcttgagcccatggtggccactgggaagtctttgccagaatccagcttttcatattgtccttcttaaagaccacttggggccataacctgtgttgaccttcttattttatacatttgttattttactaactgtgagatacagttgcatcctgtatcaggtaccatgttagcacacaccagacaaaccccacgaacctctgtctcctcctccagcagcttcccattgtatggcggtaacgtggggtggtgaggggagaagaggggacagcaaacgtcctgggtatgaaggatgagtaagagtttacagagcttgggtaactgacagggaacagcaggcccagcagcgcacagccagagagcgccgtggagcaatggtcagcttagggcccaggagggcatggggctggagggtgttcaaggccacacaggcctttggatcccagtgaggaggtgtgggtgtgaccaggcccttagcttagcatggtaaaggaaagccaaaggaaccgcaagcagctaggaactcaggaggagagattaggttggtaggaagcataaggaatgaggacaggggcaaagatgggtagcctgtctaaccaaactgaccccccttggaaaaaccccaggggcctcaaattgctaaaaggttcatcaattttatgggtcagggatttggggccaggctcctcaggcaatcccttggctctgtgtgaggttcagctatcaggtgtctggctgagggtaccggatagactggtttatcatatgctggcgcatagtagggtcaggaaggctacccattaggctcttcctgttgtgtatgctgtattgtgtgatgtccagcagcgaagtcagggttctaggaggctcagaagggaaggggtgtcattcccattaaagctaggcccgcagccagggaacactgggttcttgtttgtttgtttgtttttgtgggttttcgttgttgtttttttgccttcagaggaagggtaccacatggatggctatcttactccacccctttgacctttctagaagagacttgtgctcctccaaggacaggagtaaggatgtatagagcacgttacagggactggagcctagctgtgtgggggagatacttaacctatcgcatagtttgggatatctgatctatcaacactggcttcaactacagatccacccccacacacctggaacactgtctgcctggtggacctcagggtcctgcagggttcaagcatatttgctaatgccgacagtgtgtcactggtcctccagctcccagcccaaggcacgaggttctacgtttagcctagggcagtgtgcctgtgtgctctaaactcagctttcactacctgcgagtgctgtctgtagttgaacttgaaaaggagccggagcacatctggccagagacggtatgatctgttactgatgaggtgtgggaaccaaccacatcactggcttttcttgcccatgggtccccaaacctcatggcatctctcaggccatggcattgttagggactgcccatagacacagtgtcctccagagtaccagcggaggtgcacatacctgcccctaaggaagtctgcctgtgtacccttcccctggtccctcgacacagagcgaggcctctgacaggatgtgcttgggtgtcagctgagctggactgccatgttcacccctgcggctgcagccagcctgtacctgagctgggccagcactggctctggagcgctgacaccatcccttatcagactcacaccacatgccggcacttcaccatctcccgctcgcccctccgaccccgtccctcaccacggaaggtttaattgttgcaaatgggatccgtgttctacagaagtccaataaatctgcagtctgtcttcctggtaatccatcacagattgcaaatcgcacgaggagaagtctataagatatgttcttcatgtctagcgtgttgggttattttcatgtcttgtggtttgcatttggccactgtcattttagaggaatttcctgccatctgcgttcaattgaattttctcacagatttgtgcttgcttgtaaatactcagagccggcagaaaagggagctttgttcttggtgacatttcaaaaagctgttcatgcagcttcatcggcgatctgcagtgcttatgagagaggggtgagaggtgtccctgggcctcaggatgagatgcgctcccctgcagggcggccagccgggtcagctctctacccctcctgcacccaacggctgcactccttcagtgctgtggccagttgtgttgggtgtatgcattccttggctctgtgcccttcctctgtaggaagacagttgggctgagacagttctggaccctagtgtccagagctcgtctgttcaagttcagctccttgtgatgagagacgacccggctctgtgtgactgtagagaagtcacttcactccccagagcctcagggtttgtcatgggccttggatacttaggacttgagtggcctgcagcatgcagctgggtctccatagctgtcatgtcttccttctagccgaagttgatcgtctgcctgctatgtgcccgaccttctgcttggacagaggaaataagaggatgcaaaagaaagagtgggccttgtctgtgaaggcttgggtgatcatgaccatcccgtggcagggcgagaagtgtcctcctgccctgctctagtgacagtgacaagactgttgccagatgccgttgcgtgctgcatgtgatcctaaattatttgctcttccgctcactttcagggacaggtgttttcttatccactgttgtatggatggggaagctggggtttggggatattaccgggccaccgaggttgcctgcctactcatgtgacacacagaggcatcagggaacaaagggacttgtctaaggtcacacagaggttctcatcaccaggggttgaacttgtaaatgagagaagttaagtctctcagggagtacactttgtcctgagggatctaaggggtaaaagtgtggggactgatatctggagtgatgaccgccacttttgtgtgcacaggctttggggcagactcccctctcctcatgaaaagactgaatgacagacaaagaagggaacttattggagactcatcttggagtttaggcagccagccatcttggtcttagttttctcaacagaataatgaggacaagcccctaaccagttggcttccaggtatgagtgtcttgttgggcctgtgcacaggacccctaacctggaggacagactttctgcagtcacctgctcctatatcatcagcatcattcttgtctctagagaagcaggctagactctaccttagtaaaagtcgccccctccccagagccttgctgtcctgccctctccttctaatcagaccgtctgggaagaatcagtgggtgcctttcttccatttcccagcccccctttcttcggagcccactcttggccttcaagtcctctggtatacccccgctgcttccccaaagccagcaaaagctaccatgtcactatggccaccacattagtgtctgatcttcactgcctttgagtgcatctacttcctcctcatggacgcagctttgtgatgttttgtagacatcataatcttctctagcagcccccttctgggatctgcctcacctcagtggctgttggtactcccctgatctggagtggctcaagcttatttaaggccgtccctgtgtcttcctgagaaaggggaggtagcattctcagatttaatgagttctgagcagagctgggactgtcctagctgcagcttagccgtcttccgcccactcgtaggtggagacttttttggtcactgctctgtctccagtacctaaagctgtgctgggcactgagaagcgcttagtacttgatggatggatgcataggtggagacttggagaccgtgaagcgccgtgcttcctgcgacaggagagcaacagtgttccctctctgagcttcttgatctcatggtcaagctggttggagtctgacctattcctcactgagccatctgtgtctgccccctagcagggcagaggcactttggctaggagccctttagtggaaacaaagacaggtggttccaagacccttatgtctgaggacctgactcaggcaggaacctctaacatgggatccctctatcctctttgccccctgccagcagccaggagggttgggcaggggtgagccagggatgagatggataacgcataggttggccgttggagaatggcactagaacaccagcctctacactctccgtgacctggtcgcagctggcttcaggccacacctccttgtcatcgaggatgtctgtctcaccttgatggtctcaccagttactgcagtatctccctgggcagaacatctgcatgcacacctgtgctggtggaagcgagacagtcacaaaggaagctgtcaggcagggggagtggggggggggagggagaggaaggaggagaggggatagagaaggatgacgggtatcaacagaacgagaccacgaaagccttcttactttctcccgtggaacgataccaaaagacacactggagccggacctcagggagaccagggtaggggtcttcaagctaccatctcattcctttgctgggcttctgctggggatggtcacaggaggaatgggtgtaggagtgagtccgttggggactaggaacagcatacttcctctgtaggaggagggagagccaaaaccttgaggtctgactccttatacactcatttgtcctcattcagagatcaagcttgcctgccttctctcagttgagcatccgctctactcccacccagggcaggcacgcctctcgtccatctttactccctatctgccatgactggccagagagaccctctgattctccatcacctgcagtctagcctctagcctgacttatttcccatgaccctagggcgttgccagagtaggtgtgccatcagcctgcgttgtgagacatagctgtggagagagagacctggtgagctgccataggctgcccttcctctccccgactgtctgtggtcctttaggaacctgggcgtgggagctgctattttatgcccactggaagcccagtgttgtcactcgatagcttgtatgtagctgcgtgaccagggtggagtggttttgggtggtccttttggtagccttaggtggtctttgtgtcatttctggtcacaaagtgcctctttgtcactttggaactcagttttaagagggtaacccaggaggtttgtgtcagaatcaaaccccaggacacatgtatcactgaagacacatcttcttgctcactgattgatctgtccttgtttggctgggccccgaggactcattagagtcagaccttgtccctgtacttagggggatgtgtggtcacagggcctggggattgaggcacatactccacccagcctaggatggtttggaccttctagagagggcctggccggtgtcctctctgttgtcatctctggtttgaatagctagtggttgagaatacattttcaggtaaggagatggggacttgatcccagcccacttcaggctcagccctcttcctcctgccctggacacacggtgtccagctcacatctgatatccccagcacagagctggcacaaatatgaccactgtagaattccaggcacttggcagctttactgtttgcagggctatggtggtctcatagaacaacccctcttcacagaaaccaagatcactgtgtccttaccgtgtgtattagtcagggttctctagagtcacagaacttatggatagtctctagatagtaaaggaatttattgatgacttacagttggcagcccaattcccaacaattgttcagtcgcagctgtgaatggaagtccaaggatctagcagttattcagtctcacgcagcaagcaggcgaaggagcaagagcaagagctagagctagactcccttcttccaatgtccttatattgtctccagcaaaaggtgtagcccagattaaaggtgtgttccaccacacctttaatcccagatgaaaggcatagcccagattaaaggtgtgttccttaactcggagattcaatcttctggaatccatagccactatggctcaagatcttcaaaccaagatccagataaggatctccaagcctccagataagggtcactggtgagccttccaattccggattgtagttcattccaaatattgtcaagttgacaaccaggaatagccactacaatccaccccttgtcaacttgacacaaataatatctcatgttcacatgaaacaataacaaggttgtaaatacgcctaacatgatataactatccctcgtacaatcgcaaacgcattagtaaatttacaatgggcattcatattactttataatcctcgtttctgcaactggttacgtggccttaattggtatttataactaccttcctctactacccattctgtatttccttcaccttcagccagcacctcagcaggtcttggctcttttcctggaggattgacccataccttcattcctgatgggtctgcgtcctttgtcatcctgcttggattaggctgttgtagtttcccattgactttaatcacaggacatggtagtactaagagacgccctaagggatctcctgcactccagacataatcttgcttaccaccattgtgaagaggtaatccaatttctccatggtaatctggatctatcacccctcctaacactgttattccttttttagcctgttggtttaagggcattagaagcccaaaatgaccagggggaagtctgagcttccagttcaatggaatgtttgttgtagctcctggtaggaggactcccctctctggagccaaaacttctaggccagcagaacctagagttatggggacaggaagcaaaaattttcctagagggtcactaggagtgatagtaagtggaactattcctttttccaccccttgattcctggacccatgaatcctggctatgggtgaaactgtaccatatatcgagcgctgattcaaagcatatactgccttctgaagaactctaccccagccttccaagctgttaccacctaattggcgctgtaactgcatcttcaaaaggccattccatctttctatcagaccagctgcttcaggatgatggggaatgtggtaagaccagtgaattccatgattgtaggcccactgtcgtacttctctggctgtgaaatgagttccttggtcagaagcaatactgtgtggaataccatgacgatagataaggcattctgtcagtccgtgaatggtggttttagcagaggcattacgtgcaggaaaggcaaatccataaccagaataagtatctactccagtaagaacaaaacgctgtcctttccacgaaggaagtggtccaatgtagtcaacctgccaccaggttgctggctggtcaccccgaggaatggtgccatatctggggctcagtgttggtttctgctgttggcaaatctggcaatcagcagcagctgtagccaggtcagccttggtgagtggaagcccatgttgctgagcccaagcataacctccatctcgaccaccatggccactttgttcatgtgcccattgagcaatgacagggatggctggggagagaggctgattgtccacagaacgggtcatcttatccacttgattattgaactcctcctcagctgaagtcaccttttggtgagcatttacatgggacacaaatatcttcacatcctttgcccatttggagagatctatccacatacttcttccccagatgtctttctcaccaattttccaattgtgatctttccaagtccctgaccatccagccaatccattggctacagcccatgagtcagtgtataatcgtacatctggccatttcttcttgcaaacaaactgtaataccatgtgtactgcccgaagttctgcccactgtgaagatttcccttcacctgtgtctttcaaggttgtcccagaaaggggttgtaatgctgcagctgtccacttctgggtggtgcctgcataacgtgcagagccatcagtaaaccaggctctagtcttctcctcttcggtcagtcgatcatagggaacaccccatgaggctataggcacatgtttggcagcagatggcattgtaacaggagtagaaaccataggcatttgagcaacttcttcatgtaacttgcttgtgccttcaggacctgctctggcccgatcacgtatataccacttccatttgataatagactgctgctgtgcacgtcccactttatgacttgcagggtctgatagtacccagctcatgatgggtagttcaggtcgcatagtgacttggtgtcctattgtcagacgttcagtttccactaaggcccaatagcaggccaagagctgtttttcaaagggagaatagttgtctgcagatgatggtagagctttgctccaaaatcccaaaggccttttctgtgattcacctacaggggcctgccagaggctccaaacagcatctctatcagccacagacacctcaagtaccatcggatctgctgggtcatatggtccaagtggtagagcagcctgcacagcagcctggacctgttgaagggccttctcctgttccaggccccacacaaagctagcagctttccgagtcacttggtaaataggcctaagtaacacacccaagtgagggatgtgttgtctccagaatccaaatagacccactaaacgttgtgcttctttcttggttgtaggaggggccaggtgcaataacttatctttcaccttagaaggaatatctctgcatgccccacaccactggactcctaagaatttcactgaggtagatggtccttgaattttggttggatttatttcccatcctctgatacgcatatgtgttaccaatgagtccaaagtggttgctacttcctgctcacttggtccaatcagcataatgtcatcaatatagtgcaccaatgtgatattttgtggaagatccaaacgatcaagatcccttctaactaaattatgacacagggcaggagagttaatatatccttgaggcaaaactgtgaaggtatactgttggccttgccaactgaaagcaaattgcttctggtggtccttatggacaggtactgagaagaaggcatttgccagatcaatagccgcataccaggtgccaggagatgtgttaatttgctcaagtaaggaaactacatctggtacagcagctgcaattggagttactacctgatttagttttcggtaatcaactgtcattctccatgatccatctgttttctgcactggccagataggagagttaaatggagatgtggtgggaaccaccacccctgcatctttcaagtccttgatagtggcagtaatttctgcaattcctccaggaatacgatactgtttttgattcactattttctttggcagaggcaactctaaaggcctccatttggcctttccaaccataatagccctcactctacagttcagggaaccaatatgagaattctgccaatttctgagtatatctatcccaattatacattctggaactggggaaatcaccacaggatgtgtccggggacctactggacctactgtgagtcggacatcagtcaaaactccattaattacctgccctccataagcccctactttaactggagggccacaatgtttcttgggatcccctgggatcagtgtcaactcagaaccagtatccagcagaccccgaaaagtctgattatttccttttccccagtgtacagttacccttgtaaaaggccgtaggtccctctggggaagaactggagaaagggtaacagcaaaacctttgagtgtcttatcaagatccttcctcagcggaacctggccaccccttcattcaaggggttctggatctgcaaactgtctcaagtctggaaattgattcactggccgagattgctgtttaccacgatctaatgtagcctttctttcatttggctgtttaccacgatctaatgtagcctttctttcatttgtttgagaatttttctgcttatacagatcaaacaaatatgcagtaggcttcctatgtatttcattcctggaaacaccatgattggttagccagtaccaaaggtccaaccgagtcatgccattataaatttcacctctcctgtgctgaccattactgggtatgttattataaacattcttttgtctacgctgtccattataataactagaatcaccttgtctcgggcgattcaatgctgccacctggcccttgttacctcggaatccaactaaacccagtgaatttaattcatctaattgagcagaagcatctccaatgctaagatctggcacaaggaaaagggaaagaacaaaacccttcaaatgtgctggtgcccctctcaccaatttgcgtcttatagagctggtgaaaggcatatcttctggaccttcccattgtggacaattatgctttacacaatatatccactctagcattgcaatttccctaagtcttaaaatcccttcatcaacactaagccatggaatatcaggcatctccaagtcatttccagtaggccatcttttgataaacacctcagccaaccattcaaacaaacttttgacaccttttttaactatgcgagcttccgtattaaacctagaatctctactcagaggacccatgtcaataaactcagcctgctctagttttatgttccttccacccttatcccacacccttaaaatccattcccacacatattcaccaggtttctgcttgaatgaattagcaaactcattaagctccttagtagtgtagcgaatttcctcatggactacactttctacctcccctctaggagcctgttttgctttgagtctggttacaggtctagaagaaactattggtgggccgtgagcagactctgcaaaattaatttcctcatgtggcgaaggcattatttcaaggggtggggctgagggtactacttcctcaggtggggcaaacccttgagaatctgaagattcaaaattctcagcttcaacatggtcttcccacacatccccgtcccatgttgtaggatcccattctttgccaattagagcccttactttagctgtcgacacactctgaggctgagacttgaattttcgctgtagttcagccaaccttacaatgagagtttctgtttgattttctgcaacttgagctctattgctacaagagagaagattctcctcaaggacacacttagcaacttttagatcgtttacttgtgtctggagcctttcgattttatcacacaactccttcctttcattcatcattctttccacagatactaagagcagccagccagtaaaatcattttccgattttttccccatcttgtagaaagctttgtgcactgaatcacctaattcattaagaaaatcaaggtcattagcttctttaagttcagaatatagtttcaaccatgggtcttcaaaattctctgagctcccaggagggagagaatctggagaggtttcaatagttgaaagtgctggtggatcaacaagccaattccagtattttaaaagattcatccttgtacttctgttactctagaaccactcctggtaccaacttctgtattagtcagggttctctagagtcacagaacttatggatagtctctagatagtaaaggaatttattgatgacttacagttggcagcccaattcccaacaattgttcagtcgcagctgtgaatggaagtccaaggatctagcagttattcagtctcacgcagcaagcaggcgaaggagcaagagcaagagctagagctagactcccttcttccaatgtccttatattgtctccagcaaaaggtgtagcccagattaaaggtgtgttccaccacacctttaatcccagatgaaaggcatagcccagattaaaggtgtgttccttaactcggagattcaatcttctggaatccatagccactatggctcaagatcttcaaaccaagatccagataaggatctccaagcctccagataagggtcactggtgagccttccaattccggattgtagttcattccaaatattgtcaagttgacaaccaggaatagccactacaccgtgtttgccctcaggtcgctccctagcctcagtactcaggtggagccaggacctgctgtccccaattgctgactttgaacagattgggtgacagcacccaattcactggtctttaggagtgagcctgtcctgcccgggcccacccggagacagtgctcaggaattcggaacgtgagatccactggatctgcatagacctcatgtttcatttaaggaagagtcctagcaattttccttggaaattatgtggttaattcgtgtttctgtttaggcagggatgggagtagggaccttattagtatcagtttgggaaaaaaacatcgaggccttcacggccaactgcttaatagttttcattgaattaattttaaaatgagaaccttgttttccttatttaatagtagaaaacgacttccacattccgcccaataacaagctcggctatgggctctccacaaacaatacacacattaaaggcatcgcgctgccctgtgtggaatgaagaattttgtgcaaactacttattataagcacacgatgcacttaactcgtttttttgttttgttttaatttttattttttactgtcgtggtgacagatccagcccagagctgaactgttaaatggcacagacgctgcccttttcagtctatggggaaaaaaacccggagtaacattttagaaacaccacagggttagaaggagattgttagttcattgggcccaacatcccgggctccctgggccagtgctcactgcctaggagagggtggccccagggatttcatggagggttggtagccaagcttacgctgccaaataaacataagccttgcggggaggggatgccagcaaagggcctggctctagggtcagggcacagcttgctccacccctctctttacccacctgtcccttcttctgctggggcagtcaggaagataatttttatcctagtgttgatgggattttatattgattggttgactcagggtcctgtcaggtgggtgatgctattctgtttccagcagtctcttctgggcatgtgggcctttatatatttatggtgtagtttaaagatttgtttttattttatgtgtatatgtagtgttttgtctgcatgcatgtatgtgtaacatgtgcgtgcctcttgctcacagaggccagaagagggtatcaaatcccttgggactggagttacagatgctaagaaccagacgttggctctcaggcagagcagccggtgctcttaaccgctgacccacctttacagcctttttatttgtgtttttaatagaaaatgtatagagtttgattgagcacaattgttagcttgtgtcgtggttttctttggctcattcgtgtgcccgattgtctgtctcccgggtctccacccctcccttcccatcacaacgtaatgaattaccccaaaccatgatgtcagacactaacaacctgcgcctgcctgccggctctcctctatctagtctccctgcctctatttggaggtgatcattttactaaattctgttcccatacatgtggaggtgtgcacaccagtgtatgctattgagtattacagtgattgaaaccacaatggactgttacagtgtccccaatgagccccattcatgctgccccactttttaaggaaccacaggcccgagtggatcagttctagagctacagatgaattcttctgaccttggtggccagggtcatacggggtttactctggcttctctcgcttagcgctgtctatgacatctctttgtgttgccctctgactctggcttctgccttctctactacaactgtttatcttttcccattgccagagaacgttgctttgtccccagttcttgacttgcggtaacgtaactgaaggtagctgtgtgttatggtgagcgttaacccatacctctcccattcaagtactgaccaggcctgaccctacttagcttcccaggagacaagatgggacagattcatggtcatagaaatatacttgtgcctctgttgggtggaaaaaatgggaatgcaattattgaaccaggggtgtgggtggctttagttttagttttctaaagtggcggtgccattttgtctcccgatagcagaatggcggagcgaggtagcatttgaacaaggagcactcacagtgttaggatactggatgtagcgggtgaccctggtcattcattttcaccgcagcgtgacagtcctttggctgtcctccaccctggttgctggatacttacttgggttgtgttccctgtctctctgtgctgtcatatgacactgctgtgtctctccagggcgcttgggcttgaagtttgaggactcttttataccgctgacagttcttgaggactgccagagacttctgcttgcttcttttgggtgtgtctcctggcctgggtaaaggcttagaaactgtctctttggggggtgtcgccgtatggtcttctggtttctttagtctgccaggccttctttcgtaacctgctgttggttgtggctgaagaggtgactcttcctgggggacatgagccccctgactgtccagggtgacacagtatgctgaaggttctcttgcctcaaaatttctcttacctgtggcacatccatttttatcagaccaggcggtcttgatggcgttttcctcagtaaacaagcacccttgagaacctagctgtgctggggtctgggagatggcactgaagcacacggaagaagtcccttgtagaatgctggaacagaggagaagtcaccaaccatggatggggccagggagcaaaatagatgtgtcagaatgcagaaagttctgtgggataaaacagagcaggttaaaaaacaaaatggccacacactgtgcgctttcacctgtgttactcagtggtccaagcgtccagcatggcaaagcttagaaggaactcccacaccctcactgtacaagtcctagctggaaggcccgctccacggtctgtgcacatgcgagccgctctgcaatgctcctctggcctccacttcaccccattcattaatgctttgattgaacatgacttagaaggagccaagtagactctggataaatcctagctcaattacccctttgttgaagtcagtttctctgagccctgacttctcctctgaggacagtggagatgacagcaatactccgtagggttgccgtgaagcagcttagcgcgatacaccctagtgtgaatggtgtggtccttccttccactctagcctggaacttggagctgcggtttgtatgcatctagattttagtccattgtcctgtaggacccgattgatctgtgggcgaagacagaagggtagacaagtatggtacgtgtcacttggtcacaatgcagtagaaagagaacagggacaggagacaggtagtacttaaaagggttcctggcatgtcccagcatgcctgaggacaccttggtatttgtgtgatgcatacaacacgggcatagagtaaaacccctgtggctagggcttttgttgctcagtctgtccctggcatttcctgggctcttatttgtgtccagcccattctgtgccctgaggagggctctgtgataaactaggtgttacctggatctcatgttcctgtgggagggacaaaccatggaccagcaaatgagctaggtagtttctgttagccctccagtgactttgtgtgggtctgatgtgttgttcctttgctgtttatacttgctattgcaaaatgcacctgagtctcttagaacaggagcctgtgtcttcagtgtcctgagtgtctctgaggcctggagccatcgtgtgctgcctcctcctccccctcttcttccccctcctccctctcccctcccccatttgactgctagttattattagtatttccctgctggtaggatgttctaggagagtagagatttctgtctgctcagtccagtgtcatcccccagccgtcgatggcagcttctggcatgtaacagaggctggagaatgctgatttactaacttcttcagcaagattgtgcctgctcgtatgataggtggtgatgaggagcgtcaggtaggatggttgggtgtggtgacacacacttgtgaccctgacgctgagactgaaagagccagtgagcttgaggttagcctgccctgcacagtgagcttaagctcagtgtggcctacatggtgagacctggtctcgaaacaggcagtcacaaagcgtagagcgggtgagaggatggggatgatcatgtcaaggggacactccttgctgggtcatcatgggcagaggctgagtgtgtggggggtgctctgtgggagggagccatgattgaatggcagtagagagaaagggagggaggtaagctaactcagaggatcggttatcctcttggtgagcggaaagccagcacgcacaaccaggagatggaagcgtggagagacattatcacctgcagccgagagagcagcttctttctgcaggaagagaacatgggtttcagtcatgacacctatacacatttataccagagaacacttctggggcaggcgtgtttctgagcatgctcaatgtaatgtcataggtcaagaaggaaagagttacacatttctgggcatgctcagcttaaagtaatgtcacacgtgcttaaaggggaagatggctgagaggggtgcttccgggcacgtcagtattataatgtgctaaatcgtttcaaagttgcctaaaagaacgagtcagagtggctaggtgcctctggttgctgcctcagaagctccgctaaagtcttagctgaaagaaaaataagaaataataataaaaataaaggataacacgggctgagagatggctcagcagttaagagcactgactgctcttccagaggtcaggagttcaattcccagcaaccacgtggtgcctcacaaccacctgtaatgggatccgatcttctggtgtgtgtctgaagacagcttcagtgtactcacatacataaaaataaataaattttttaaaaaagagaaaaaaaagaggataacacagaacatgtggtgatgctagcatttacctccatgtgcctgctgtgctcagtggctaacaggatctgacaaaggcacctttctgggtttgtcagttagccctagggaggatcctggctgtctccctttgactagctggcttgggatcatgcaggaatgcctggctagcatctggacccccaaggctgggctcccagatcattgagttagaggcaggtgagccttccttttcaaacctctcagtcttcatatgtgatggggggacagacagctttgaggtcctacttgcttgtgtgcgtgtgcgggtgtgcaggtgtgtgtgtgtgtgtgtgtgtgtgtgtgtgtgtgtgtgtgtgtgttagctgtttcattcatctggaagctgaggtggacagacccagtagctcactgtctgctgtctccttcttccctgtggcagccatagactcaaggagtctgaggcctagacagaattgtcaaatgcatactacctgtgtgtgatagagtctgaactagtaccctgtcccagtaggtgccccctgctgtcctaacttcaggttgagctcaggtcaccccaccagggttacaagggcatttgggacaagcccccaccctatttctaatgggctgtttcctattattccgaaggtttgcccctaagacttgctggggtaaggttgaaacttcacagaactgggaagaagaaacccaagtgtaaatcaggtttatggccggggataatgaagtctgagttcaccggccttcttgatttcttccatatgaagccctctgcttttggtcacagtagaacatagtggtttcatgtgtccagtccccggagcccggaaggcaggacattcagatgtatctgtgagcaccgattggcagccccgtggctattacatgataacagtctttacaccctctctccctgctcgggacttggcttgtgcccaacgcaggtaacagtgcacttccagggcggggtaggtcacaggtgtgccaaaaggatagcggtaagacctgcagccaggcagactggcggacaattggggagggtttcccaggagaactgatccaagagagacaaggaagtcggaggagggtgagctgcagaagggtgtgccaggcagaggaacaggcatatgagcactgagagtagatagaggctggagaggcaagtcctctaatccggaggaaggagggcattcctgtgcggcatacttggccttcttggatgacatgttgcttaagcaggatgggaacagcgtggatgggagttcatcagctcccacaggagatctgccttgacaaagacctgggcaagcgacaggagaggcggtgatggtgatggacggcctgtgagcttaactccatccagtgatcctaacaggtactggagaggtgatacatacatgcagccggccatgctgcttgccctccgtccaagcacggcagttgagttctgggcaactgtgttgctggtgtttgtaattatggtggcaatgggatcctgctcatcagactgccttaggctgattgagcctggtaatggggaacagaagcagggtgccatattgcttggggggggtgtcttaagtgagtacagggtctctggccctgttgggtcactgaggtgccctggctctgactagctggctttttctccctgtacctgacagtgcctcacactgtgggcccttctcagtgctggagttgagggaggggctgacggcagtttcccattgagagcacttacactgttcactccctggcacctttctaagactaactcttccttcccagacgctcaagagttagcattgtgtcagagaggtgtgccaagcataccgctagccctctgctaccaaggtcccacgtctctggggctttatcatcaccttggctctcatcttcatgaatcactatgacaacgccacccccagggtagaattgtccatctatgtcttttcagatgaggagcccaaggactaaagagggttcatatagttgggtacccagcccccctccaactctcctgctttctgaacattgcaggacctcagagctcttacagggggcagtgagatgctgcattgaagaaaccattcctctactgaggcagatcatgcataagagccatcatccacagggtagaagggagagaggccaggccagggatggggaaacacgctggaaagaagtccaccgctgcccaggtgtgggatctgaaaagaggaggacttaccattcagagacagccatgcagagtgaggagccagcttctgctgagagcaataccttgcctacttggggagggacagcaagcgtttccccggggctgctttttaacatggtggatgggtggggcctgggaagggacccctgaatagtcaagtggctttgcctcattcctcggctctctgggctgagggttgaacattgattgacatgagacagcctgaactgcccatgccaaatccttggattgtcccagcactgggttgctgggagctgaggacctttggatttaggcttctctacgtggagagggaacagggatatttgcttctaagctgctacatagaaaagctgttacaaaatggtgcatgctaggtaaggaaggattagcagtgcctgagctggagaggggtatggggggacggtacttttgacttggtagggaggagaggtggaggagatgctgccagtatgctacaacatggggacagagtctggggtacctgtccctgtaagttgctaacggaaactatcctgcagtctcgtgttgaatcactactgtcctggaaagccacagacaccctgaaggacagcagccaagggcagagcaaacatgggtgtgtgtgtgtgtgtgggtgtgggtgtgggtgtgggtgcggggggggggggggcagcctagatgtgatgactcctgatctggccttcccatgtggttccaagtgtgccttttgagagtccttcacccctgccctgtctctccatctgtccaacggcctgtgtcactcagtactcacatagcaagggcgaccctctggagccaggctgctgctggttgcggaggcccagctgggctgctgattgttctgaagagttgagggctctttcaagggaaactgcagtcaattagtggagagtggaatttgaaaggcccaaatccctttttaccccaaacttcaaatctggttttcgtcagtggctttgaaaacaaataccaaaagaaccctttttttttcaaaaattattttttccttctcctcttttcccatccctcccttgcattctcttgtttcctggccttttatctctttgaacagtcagctttggtagcctggcctggctctactgtgatggctgagcaggtgggtgtgtggctgcgacagaccccaccctcccagtgcatgcctgagccttgtctcctcttggtggggaagccatgtggacacggtctgcttctctgtgccctgggctcagcccagaatgtgtgttagagcaggtggcctgcaataggaacaggaagggacgtggagggcagggtgctcatggagaacaaagaggacttaactgtaggcaggcacagagcttggcctcagagagtcacctacgcctatgtcacccattgttgagctgttcacccattcatgctgtgaatgtcccctgaatacctgccccaggccagacctgattccgagggtcccaaggcaaactgtcctaacatcagctccaagtagcctggagtctagtcgggagagggcaacaactgtcgggagaaccaggcatgagaggaacaaagaaggtcctggctatttgtagtagtcagcagaaactgtgacaggtggcagctgaccattgcactggcttctgatgggcaagaaagagtttgccaaggaaagggggtagaaaagggcattccagcagagtgagtgagagagctgaggccaaggtggtcagaggctagcccaaggatggatgtggtgtaaggtggccagcccaagatgtatgttcttcatatctcactacaacacatataacacatagtgttttgatatcccatgagatcctgggacaggcattgtgtcctgtggacaactctgaggtcgatggttgatgctccatgccactgatgaaccaacaggctcacaaagagggaagcctatcccaagggccacaagtgctcagctgggggaaaggcatttgggggaggggcacagagagccaagtaactggtacctagaggcttccagcagacaactagcatctagggacaacctctaaggacaaaggggtagagcttgtgtgtagaggtttcaatggatactcagcagaaatgtcagacttgggatttgaacctaggacaaaagcagtgaagccacagcgttcacaagcacagcctctcgctttctcagtgggccctgaagctctgctgagctcatcctctgctctgcctttgtcgcctctcctcacgccgcagtcttgatgcccttagtcaactgagcttggtctctgggagctgcctgagccaggcaggaccatgtgtgtgtgtgtgtgtgtgtgtgtgtgtgtttgtgtgtaagacagggacaaccaagtagcgccaacctggtttcagatcctggttctaccttgacaactgtgccacttaattctcttcttcatataacagcacagtgacaaagctccttctttctggggctgtgctgaggagtaaggagactgtctaagtcaaatgctcggcgttgtttatttatacatagcacgtgctcagtgaatggctgtgtcattgtaggattccagacactcacgtggtagtaggtttagacccagacgctgtccagcagaaagatggggacctcggaaagcctgccactcacccctgggtctgtgatgacaagcagagacctaagctggctgtggtggaatatgttcccaaagccagtgctcagaggagaaatgggagggtttgtcacgtgtttgaagccatcctgagctgcagactaaaattctgtctcaaaaacccgaaggaggaaacaaaccaaccaacctgagacccagggaggggcattgtgggaccatccatcaaagagatgtagaggacccgcctctctgaaccagccttcctggcttatccactcctcatttagcactaccaaaagctgacatcactgccctgtcgtcataccccatacttgaacgtcctcatccttactgaagcatcttaaggaagggctggtgccttctgactctttgccttcctgagggctgatacttggctctgatctccattctgacctcttgcttctccaggcatctgacttctcatgctttggggcattggctagctgtgtccctcctctaagcactcataacagtaccttgatatcaataatttaccagatgattaaagatgtgctgggtcctaaacgtctcatagatattagtttaacccacccccaccccgttttgcagaaatatcacctgaggtttggcaaagtatcaggcttgagatttgaacccagatccaaaggtaggtaagtcacagtcttcagcgatgtgtatagtctcttgcctcctccatggtctggctgtgagggcagtggggttctgggaagagtgctgtgtactgtagggtcccccaacctctgaaaaaaaaccctctgaagtccttggtactagcttcctggtcctggggtctataaatattcactgagtaaacctgcatttgctttggccctgtttagagggctggaaaaattcccgccctgtctccaaggtaagacaggaaagaaaatattatcgtcaccctctggtggaaccaactccaggcctctgtcccactgaatcacctgagggtcgggtcgcaccacagccacagagaggagctgggtttccaaccaaaacaacgcgtgcatctgcctggaatttagtgattgatttatggggtgcctcctgccagcactgaggagagggacaggccttctccgagcatggctgagcacggcctcccagggaagaggaaagcccttggggacggaggcctggtatgatgcagtcagactcacaactaagagaggtcataccaggctttcgagagcttggcgctgtgtcctctctgaacaccctctcttgtcttctcagggctcctcatcagtgtgaccagtgactttcagcttctctgtgtcctctgatgcagtttaatctccagattaatcctgtggagcaggcaatgagaccccatgttagctggggagcccagggttcagagaaacgggatctggtcacagccgtttgttcccagcggcagatcctgcctttcagcctttcccttggccacctctgcagagctggaaggtcttaggagccctgcctgcctctgccttcactccctgccctggtgtggatgcagccacgcctgccaggggcctgagagtttggagtgactgtaatgtccccagcagtgtgcgtcctcatgcagcctgccattctgtctgctcacacctctcccttggcctttcctgcctgctccctcctgggcgcactccctgatccttgtccattgattcacttgggctaccattttgtctaccattactcctttgggtttggatcctagaggatcccaggattgtgtaaggactcctggtgaggtgagttaggcaggaccccatggtggtgtggaattgggacatggacaggaggctgcctgctccagcttctctgctcggtgccattgctccatcgtgtcacagtggctctgctagggctgggtggcaggcaggttggggagacaaagaggatgtatctgttcatatctagctgaccaggtcggcattcttgattctgcagtggctcctggttaaccctgtctgtggaggaggacatgaccatctgaccatagaggactgaacccacctcgaggaagccagtccctttggatagcagtctgttttgttcagcgtctcctgtagctgccccattatgcagctctagtatccttaccttattcaaagtgcaaatgtttcagacttatcaggcttggaaacacctacataggcaaaaccacctgagcacctttaatcataaataaaaaaatgtccccagaattaaaaataaataatcaacacccaagaaatgctggattttactgcactgagggtttatgtccgtgcatgcgtgcgtgcgtgtctctctctctctctgtgtctgtgttcatccagaagatgacactggagcccctagagttactggtggttgtgggactcctgatgtgggtgttgggaaccaaacctgggtcctttggaagagcaggacctactcttaactactgagccatctctctaaccccaaggcttctgattgggtattacagatgctcaacctatagttgcttcaaagagccaactggggggggggggtgtctccctggcccttttatgatccatcaccaccgaccagttgagctgtggtcagtacctgtcaaaataaaaagggaactcccttattccagagagaggccattcctgctttcaccctgcactgtccccagcacagtccctggtacagatcatcttctcacctagtagacagaccccccttccttccctccctcccccttccccccttccctcctccctccctcccagagtccagtgtctagcattagatcttccagttctggaaacaccaccagcttctttcatgttcctgttcccctgcacatgtgggtctgttccgtgactattctcttcttcctgcctaattgactcttgattgccagtcaccaacagcaagcctcttggtaactgtggagtgcttacatagagatagtaatattccttccaaagatggggtcattctgtatctgctattggatgtgctgtgcccagtgacaccagccagggcctatgggaacttcacacatcacatacatagcatttgactcattcatgagattagcttaaatacaggctgaggtccaggaaagagtagacagcaaatgagcctctctgagaccagagagctgcttctcttgattcatatggatcctactccagagacccactcgaggtctcctgccctggcacctaacatggcttcagcaccaattctgctcttcttgaagaatcggaggctccccccccgcccccccgccccccgaaccctcccttgcccttgcttgcgggtttagggatcttgctcctagggcccccctatcagtccagcagagagcagcttggtctcagtggccaccagtgagggccagcagagtagctgcttgtagcatgcccgggtagtgggtaagccttttaggggggtcctgctgcctcactcccaggccattcatcccttcttcctcacttagtagacactatctgggacctgctgtgctgaggggccaggaccagaagggaatctgacccggtccttcccacaaggccccgatgagctgatttgggggtgctgtgcacacccaaatgctgccggctgggcaggaatgctgggaattcctcacagtggagggccatgagaggggaagatgccaggatactgagcaggctcccagaaggccccgcaggtagctcctgagtggctgagtaaggaggcggggtgtggggagctggagacgggtgggcatgggcgagcagtcagagcccctgtgtgctggtgtcggaactcctgtgtgctgcggtcaggaaagcctggccgtggagtcagcctgtgttggttggttttctgttgctatgacagcatacccaaggcagagtaacctaaaaagaaaaaagtgtattttggctcaggctcctgatggacagaaagttcaagaccaggcgtccacacctgggaagactcttgggctacttcagctcagggcaggaagtaggtgagtggacatgtgcagaagtggggggtcgggggctggcgtggagctcagggtggaccgcgtgctcagtgtgtgcgactctgggttagattcccagtatcatatataaaacactagaactgcagcgttcccagagctgggagccggaagagcaggagtcccaggccattctccgttgcatagtgagtttgaggtctgttcaggctataggagaacctgcctcacataaacgaagagaggaagagaaagaggcagcctgaactcccaacagttgacgcatttcctggagaaaggcatgactgttctgggaacctcgtgcctccctctccccaccccaccactgtcaagctgagaaattacgattttaacacaaacctgtccttagtcttgacatttatcttggggctgtccaccctggctggtccctcgagggtagtgtcaggcgtgatcatggtgagtacccatactttaccgtcacccttccagttacagaggacgctttcgctaagacagagacgtttcctgttggccccctgtaaacatcttttgccacattagtttctcttctctccaagctgactgtttccttttaaaattgagtggattttgaattctttatttttttaaacttgcttgctttattaattattaatttatttatttattgtttatctattttattttgtattttaagcgtgatctatctgcatgtatatctgtgtgccagaagagggcattagatcccattacagatggttgtgagccaccatgtggttgctgggaattgaactccggtcctctggaagagcagccagtgctctccacctctgagccatctctccagcccgtgggaacatttcttttttttgttgtttttgtttttttctgtttgtttggttttttgttttgtttttttaattttttttttaggtattttcctcatttacatttccaatgctatcccaaaagtcccccataccctccccccccactcccctacccacccactcccacttctagccctggcattcccccgtattgaggcatataaagtttgcaggaccaatgggcctctctttccactgatggccgactaggccatcttctgatacatatgcagctagagacacaagctccgggggggcggaggggtactggttagttcatattgttgttccacctatagggttgcagacccctttagctccttgggtactttctctagctcctccattgggggccctgtgatccatccaatagctgactgtgagcatccactcctgtatttgcttggccccggcatagtctcacaagagacagctatatcagggtcctttcagcaaaatcttgctagtgtatgcaatggtgtcagcatttggaggctgattatgggatgaatccccgggtatgggagtctctagatggtccatcctttcatctcagctccagactttctctctgtaactccttccatgggtgttttgttcccaattctaagaaggggcaatgtgtccacactttggtcttcgttcttcttcagtttcatgtgtttcgcaaattgtatcttatatcttgggtattctaagtttctgggctaatattcacttatcagtgagcacatatcatgtgagttcttttgtgattgggttacctcactcaggatgatgccctccaggtccatccatttgcctaggaatttcataaattcattctttttattagttgagtagtactccattgtgtaaatgtaccacattttctgtatccattcctctgttgaggggcatctgggttctttccagcttttgactattatatataaggctgctatgaacatagtggagcatgtgtctttcttaccagttggaacatcttctggatatatgcccaggagaggtattgcgggatcctcaattttctgaggaaccaccagactgctttccagagtggttgtacaagcttgcaatcccaccaacaatggaagagtgttcctctttctccacatcctcaccagcatctgctgtcacctgaatttttgatcttaaccattctgactggtgtgaggtggaatctcagggttgttttgatttgcatttccctgatgattaaggatgctgaacattttttcaggtgtttctcagccattcggtattcctcaggtgagaattctttgtttagctctgagccccatttttaatggggttatttgattttctggagtccaccttcttgagttctttatatatattggatattagtctcctatctgatttaggataggtaaagatcctttcccaatctgttggtggtctttttgtcttattgacactatcttttgccttacagaagctttgcaattttatgaggtcccatttgtcaattctcgatcttacagcacgagccattgctgttctattcaggaatttttcccctgtgcccgtatcttcgaggcttttccccaccttctcttctataagtttcactgtctctggttttatgtggaattccttgatccacttagatttgaccttagtacaaggagataggaatgaatcgatttgcattcttctacatgataacaaccagttgtgccagcaccatttgttgaaaatgctgtcttttttccactggatggttttagctcccttgtcaaagatcaagtgaccataggtgtgtgggttcatttctgggtcttcaattctattccattggtctacttgtctgtcgctatactagtaccatgcaggtttttatcacaattgctttgtagtacagctttaggtcaggcatggtgattccaccagaggttcttttatccttgagaagagtttttgctatcctaggtttctcgttattccagatgaatttgcagattgctctttctaatatgttgaagaattgagttggaattttgatggggattgcattgaatctgtagattgcttttggcaagatagccatttttactatattgatcctgccaatccatgagcatgggagatctttccatcttctgagatcttctttaatttctttcttcagagacttgaagttcttatcatacagatctttcacttccttagttagagtcacgccaaggtattttctattatttgtgactattgagaagggtgttgtttccctaatttctttctcagcctgtttattctttgtgtagagaaaggccattgacttgtttgagttaattttatatcaagctactgcaccgaagctgtttatcaggtttaggagttctctggtggaatttttagggtcacttatatatactatcatatcatctgcaaaaagtgatattttgacttcttcccttccaatttgtatccccttgatctccttttgttgtcaaattgctctgactaggactttaagtacaatgttgaataggtagggagaaagtgggcagccttgtctagtccctgattttagtgggattgcttccagcttctcaccatttactttgatgttggctactggttttttgtagattgcttttatcatgtttaggtatgggccttgaatttctgatctttccaagacttttatcatgaatgggtggattttgtcaaatgctttctctgcgtctaacgagatgatcatgtggtttttgtctttgagtttgtttgttgttatataatggattatgttgatggatttccatatattaaaccatccctgcatccctggaataaaacctacttggtcaggatggatgattgttttgatgtgttcttggattcggttagcgagaattttattgaatatttttgcatcgatattcataaaggaaattggtctgaagctctctatctttgttggatctttctgttgtttaggtatcagagtaattgtggcttcatagaatgaattgggtagagtaccttctgcttctattttgtggaagtttgtgaagaactggatttagattttttttgaaggtctactaaacccatctggtcctgggctttttttggttgggagacaattaatgaatgattctatttctttaggggatatagggctgtttagatcgttaacttgatcctgatttaactttggtacctggtatctatctagaaatttgtccatttcatccaggttttccagttttgttgagtatagccttttgtagaaggatctcatggtgttttgtatttcttcaggatctgttgctatgtcttccttttcatttctgattttgttaattaggatgctctccctgtgccctctagtgagtctggctaagggtttatctatcttgttgattttctcaaagaaccagctcctggtttggttgattctttgaatagttcttcttgtttccatttggttgatttcgcccctgagtttgattatttcctgctgtctactcctcttgggtgaatttgcttccttttgttctagagcttttaggtgtgttgtcaagctgctagtgtgtgctctctctagtttatttttggaggcactcagagctatgagtttccctcttagaaatgctttcattgtgtcccataagtttgggtatgttgtggcttcattttcattaaactctaaaaagtctttaatttctttctttattctttccttgaccaaggtatcattgagaagagtgttgttcagtttccacgtgaatgttggctttctattatttatgttgttattgaagatcagccttagtccatggtgatctgataggatgcatgggacaatttcaatatttttgtatctgttgaggcctgttttgtgaccaattatatggtcaattttggagaaggtcccatgaggtgctgagaagaaggtatattcttttgttttaggataaaatgttctgtagatatctgttaaaaccatttgtttcgtaacttctgttagtttcactgtgtccctgtttagtttctgtttccacaatctgtccattgatgaaagtggtgtgttgaagtctcccactattattgtgtgaggtgcaatgtgtgctttaagctttactaaagtttctttaatgaatgtggctgtccttgcatttggagcatagatattcagaattgagagttcctcttggaagattttacctttgatgagtatgaagtgtccctccttgtcttttttgataactttgggttggaggtcaattttattagatattataatggttactccagcttgtttcttcagaccatttgcttggaaaattgttttccagcctttcattctaaggtagtgtctgtctttttccctgagatgggtttcatgtaagcagcaaaatgttgggtcctgtttgtgtagccagtctgttagtctatgtctttttattggggaattgagtccattgatattaagaaatattaaggaaaagtaattgttgctccctattatttttgttgttagagttggcattttgttcttgtggctgtcttctttttggtttgttgaaggattactttcttgctttttctagggtgtggtttctgtccttgtattggtttttttctgttattatcctttgaagggctggattcgtggaaagataatgtgtgaatttggttttgtcgtggaatactttggtttctccatctatggaattgagagtttagctgggtatagtagcctgggctggcatttgtgttctcttagtgtctgtataacatctgtccaggatcttctggctttcatagtctctggtgaaaagtctggtgtaattctgataggcctgcctttatatattacttgacctttttcccttactgcttttaatattttatctttatttagtgcatttgttgttctgattattatgtgtcgggaggaatttcttttctggtccagtctatttggagttctgtaggcttcttgtatgttcatgggcatctctttctttaggtttgggaagttttcttctataattttgttgaagatatttgctggccctttaagttgaaaatcttcattctcatctactcctattatccgtaggtttggtcttctcattgtgtcctggatttcctgggtgttttgagttaggatctttttgcattttacattttctttgattgttgtgccgatgtcccatgaacagcagtgacactggggaccaaatcattctctgagcctccagagtttccctctcttttaagtttctggatgctgctccacttctgccttgactgctttccccaacaccacccctgagcatcatgtgcccactttcttttccctgggggatcacagtggctttattttttccttttgacttgtaagggtcccactatacagcccaggcctcagactcatcgacctttgtattcctccaacctcgtcctcccaaggtctgggaccacaggcatatggcaccatggccaccttcaccatggccacttaactcgcctttggcacattggcatgggcgccagggtgattctgtcacatcttcttctgttggttgacagttggagattttgtcctgtagcgtgtggggacatgctcactatgaaggtactgctagccatcttgcagtgcatgagccagctccctggaacagagaactctctacccccagggttcaggggcgctgagcttgacaaatctcataaggagatatagcagtatgactcttcttttgaaaagaatttttatgtttatgggtgcttttctaaatctgtatatcacatctgtgcctggtacccaaggccaaaggccagaagaggtcatcagatcccctaggcatggatttactgatcgttgtgaactgccatgtgggtgctaggcactcaagtcctctggaagagcagccagtgtcttagactctttgtgttaaatcatccaaactgcatttcatcctgatgtggaggtaaagcaagatctgtacaaagccttcagtgctttgtctctcagtctcagtctcagtctctgtctctctgtctgtctgtctgcctgcctgcctgtctctatcttggtctccccccccacacacacacatacacacacacacacacacacacacacacacacacacacactcacacacacactcacacacacacacgccatctcctttgtgccctctggcttactccacttcagtcttgttggcttctagctgctctagaaacattgctttgacctctactgcttcagggtctttgcactgtccggtttcatctactcagctgtccccacccccacctagacatctcctgcttcagatgattgcctaaatgggcttccacctagaggccctccctgacacctgtgtaaagtgtgcctctccttcagtcacctcctccttatttctttcctgtttggttttcctatttatcactaacattctgttttgtgagtgtatgtgtgagtatgtgagtgtgtgtacgcgcgcgcgcacacacacacgtgaatgcgtgttcacatacatcacatgatactgaagaccattcgaggcaagctcttcacttttgagttctagctccaacctgtaatttacctattttatcttttgctccccccccctccagaacatgagcccccatgagagcagaaattattacctatttcattcccagctacatctccagctccgagagcaagccctggcccgtagcaggtgctcaggaaagaatgtgctgagcagatgatgaatggacagtgccgtgccgagcccaggcacgtacatgagatgtgtatgatcgtcgttattactctgcacgctgcccggagagatgctggaagccctgccgcagggtctgggcgagtgtaacacagatggggacaattggttaactatttggggaaaaaaaaaccataaagttaaatcctcaccaaatcatctcacaaaatccattccagatggattaaggggtgaagtgtgaaaaatgaagacacacaaaaaattaggagagattataggggagtactgagctgacatctagctggaagaggaataggtttacataatggctgacgaagggattgtaactaaaagactgctgaagtgcttcaggttgtaaacgtttttatatgtaaaacttggtctttcgctcggtatgctgtcccgtgcctataatctcagcattgagaagctgagacaggaggattgaaagcttgagcctagctgggatacacatctcaaaaagtgagacctcatctcaaaaataataaatgaatgaatgaatgaatgaatgaatgaatgaatgaataaataaataaataaataaataaataaataaaatgggtatttgcttcaaagaccaaaggtgatagtgtttatatagactaaaagatccccagaccaaaaacagagacagacaggatgctcgggtatttgttcccagtcgtaacatccaggaagaggaagttgtgccaccctttgtcaccaacgggctcacagcctctgctgggctacagcagggatgctctcacgatggcaacttgacagcgatgttccaggagcctggcaagcatccatccactttggtccaggaattcctctccttggaagtaatcctggggaagcatttggagctgtggacagtgatttatgtgcagggggtgctcatccgggtgttatttaataaaagccaaaaatcgaaagtgtccgaagtgcttgacaacgggagagccaagtgtgcattgctgacgtgaccagacgtgtactattcagaggaggcctttgggagaatccctgggagtaaaaggaaaggctcagcatttgataggtttgtagtttataacaaatactgacaaatgtgactctgtagtaagaccccaggtgtttgtgcacatgtatgtatacgcataaccacatacacggttgtaaggaaaatggatatatcatattttcatcagtctgagagatggaatcttttttaaaggtttccccaagttcactagagataagcctgtgcagcagtcagctctgtgtgtctcagggaaaggaaagccttgagtgcaggcacggagtcagctctgtgtgtctcagggaaaggaaagccttgagtgcaggcacggagtggtcctaaggcttctatgtgtgcaggaacgctctgctctcagctttgtagaattccccaagttagcaactatttatagtccagaggctccagccttcatggaccctgtgttcgagggcacagcctaacagtgagcaaatgtaacagcgtgtgccgggaaggatgacagcaggcaaggcccctcactggatgtgtgatcccagggacaagagacagcccagtgtgcctagtgcactgtcctgcacagataaggcatcgagtaagctggaagggaagtgggcttgctgtgcttgagctgtgggaggaggctggtgtgggtgaagcaggaaggggtgaacaaggtacctattgatgcgccccaccccatctttggatccaggcctgttcacatcgaagctggtctcacgcttcacgcatcctcgggcatgcatgcaccctgtcaacaggtagtaagccaggcagcaaggctgagaggctgaataaaggactctgaggagttaacagtctaggagcgggacagaagagccatcagtggcacatggtccgactgctctcttggcttatttatgggactgtgaactgcagatgtctggtggggtctggtggtgggagggcatctgatctgggctatgtaggagtgaatttgccaagctgaagaaggcagagtggtagttgaggccaccacgaaacaggaggcatgggcggggttgcacggccagagtatgatcaagaatagcacatacctttagctgtctgactggtgtgtgtgcatgatggtacagagtcaagttagagagggtctgaggtgcctagctgaacagttgagacttttttttaaaatatggtagacacatgcaaggtagagatggtgccaggctagccactatggcatcccagagccaccaagattttgtctgaaatctggggcaggacctggaactgtgtctcaggagctgccttagaggttgtgatgatatgcaaggggactgagatacccatgcaaggtgggaggcagagccagggttcagtcctcagagccttccccaggatctgccccttctgtttctgggagctgggttttgttttgttttttttgtcccagtacttgagccttgctcattaagtgccccctgtctctatgctgtatgctgtatcctgctttgagtgagatcctcagcacacagtggactgaactggccttggatctgccagatgataagaaatatgggaataggaagtcttgacagtaaagggggtcctgtggtagtgcacagccctcttgattcaggccacgttagttcatctgcagaccctcaggttcctggtctgttccctagagctgatagttccgtcggtgtctgcgtgcctggtactttcagttctgtcttcagagtcactacagggcaggcacggaggtgtgtggagtgaattagtgaggtgctctgtacatgggagctttcaccctagcatcttttgcctgccccggtttatttgaggagggccccctggggatgaggcaaatgaagtgttatctttgtcttgggcaaagagacccaggttaggtactggcaggagcataaacgtaggaaatggccgtgggaggtgccctggaaacatcacctatgaggctaaaggcctacacgtggaagaacaccaatctcttaggctcctaccacaagctggggagcttgctactactgagtccctagctctcccaagtcctctaaggcccccctttcactacactctgttagaggactttggagtcagttgaccaggcgatgtagcctggaacagcacaccttaaagtcctcatccataaaatgggcacagttctgcctcccattcttggtacctggtctatacaaattgtatcaagctcacagccatgttatcagtgttcattctatgtccctgctccttccctggtccatctcttatccaggggatgctggcagagggttcctagagggtcgccagccaggccccattggccttaaattctgctatctcttcgagataataaataggaacaaaacccaaatccattattagcttcttctagactgcatgatttatggcttcaaagtgtctctagttaatctgtcgaaaggaattttaaatttcatccgtcttccccgctggcagccccttctaccttgaattcagctgtttgacatgtctagtaccggagtcccattttaaaaacatttacagtgggccggtggaaaaaaaatcggagctgaacggcaagcatgaggctcaatttggcacatctgctttgaattccacgcatctccacaagtgcaaataacttgaaagttcgccctgggaagaggtctgctgtgcccacgagggctgccttaggtgactttaaaggagagagggataattactgaaaatgaattgtcactgaaatagaaacctcccgaccggcctgctgaccatctgctgctcacatgtgcaggctgcccgcacctcccccgccctgctctggaacgctgtgctcgctggcttttaaatctctgcttcccagggcactcaggtgatgttctggtcaagacgggatggaactggggtagagctgctccgttccagtctatggctgtgggacccagaatttgggtataggtggagattgcctagaatttgatcctgtgctccaatgggaaaggctctgacagtaggccctagggacatggggttgggtctgggtcaggcaagcacactagagaacgcatttacatgggatatggtatgtatgtaggttagatgacaacttgcaagagtcatttctttctttccaccatgtggaaagttctaggtattgcactcaggctgttgggctttaagcctttacccactgagccatcttgccagatgtggcatgcttttaaataaggagatgtgctctcagggtggcagtgtgggatgattcagtcagaagattttccagagtggttgaggaaggcctccctggcgagctcagaatcctttgggagtgtcagcctcagggtatggaggccacaagaagaggctttgcagcagagcgcagctggaagaacactatccatgggtgaggctgctggctgtccaggacagcgtggagggagctcccagccagggctgtggtggcagccccttgggaagttaggggttgtctgctatttatttgacttgtctctagctggattaacagccccactagctacagcggtgggaacttcagtatttggccaggctctagtcacaccaatgagaggtgtgggcactgaacagaggaggctggacctttggctctgagtcctgtctgtctcttgctgtccccagaacagccctcttccagcctgccatcccaccgagaggcagagacagtgcaatcattgtcacaagcctttcatgacaccggtcagtggtcactgggaccccctcagcccttccaagatcacctggctatggcatctagggatgacccagacccctctgagggaaggcagttcatctgactgcaggctgctatgagcttctctgagatactaccatggcacccatgacctgtggtagtctcaggaccaggacttagcctgaacatgaatactgtttttgcacctctctgagtgagaattttaagttaaaaccaagatttttagtttttattcagcccaattgttcagcttgttgttgtgttcatccatgtctgtccacacacaggtatgtgtgaccctgtgttctcttgtggatagaggaccttagctagtaaccctctcaccctgactccccagtgctgggattataggtgtgtactgccaagcccggcttttaatggcttctggggttcaaacacttgtacattgagcattttacccatggggccatcttctcagaccccagtttagcaactttttaagtgtttcctattggttttttgttttgttttgttttgttctgttttaagaacactttgcctgtcacaagtgtgaaagtttggggatcagatctgaatcagtttctcttctgagctgcccctgggctcctttggaacgtattgagaaacatgaagattaaataaagttatcatgtagcaatagacccatctccaggtacacagtaggtgcctgctacaaagctgtaggcctaggaattgggatgctgtctagggtagggagataggaccacatatgtacaggcattgtttattgaatgactgactgagtagatgaagaatgagaaaatgaatgagtgatccacataaaaccagagacagtgaaacctatagaagagaaagtggggaaaagcttcgaagatatgggtacaggggggaaattcctgaatagaacagcaatggcttgtgctgtaagattgagaatcgacaaatgggacctcataatattacaaagcttctgtaaggcaaaagacaccgtcaataagacaaaaaggccaccaaagattgagaaaggatctttaccaatcctaaatcagataggagactaatatccaatatatataaagaactcaagaaggtggactccagaaaatcaaataaccccattaaaaaatggggctcagagctaaacaaagaattctcacctgaggaataccgaatggctgagaaacacctgaaaaaatgttcagcatccttaatcatcagggaaatgcaaatcaaaacaaccctgagattccatctcacaccagtcagaatggctaagatcaaaaattcaggtgacagcagatgctggtgaggatgtggagaaagaggaacactcctccattgttggtgggattgcaagcttgtacaaccactctggaaacagtctggcagttcctcagaaaattggacatagtactaccggaggatcccgcaatacctctcctgggcatatatccagaagatgttccaactggtaagaaggacatatgctccactatgttcatagcagccttatttataatagccagaagctggaaagaacccagatgtccctcacaatggagtactattcagctattaaaaagaatgaattcatgaaattcctaggcaaatggatggacctggagggcatcattctgaatgaggtaactcaatcacaaaagaactcacatgatatgtactcactgataagtggatattagcccagaaacctagaatacccaagatacaagatacaatttgcaaaacacatgaaactcaagaagaacgaagaccaaagtatggacactttgccccttcttagaattgggaacaaaacacccatggaaggagttacagagacaaagtttggagctgagatgaaaggatggaccacctagagactgccatacccggggatccatcccataatcagcctccaaacgctgacaccattgcatataccattaagattttgctgaaaggaccctgatatagctgtctcttgtgagactatgccggggcctggcaaacacagaagtggatgctcacagtcagctattggatggaacacagggcaccccaatggaggagctagagaaagtacccaaggaggtaaaggggtctgcaaacctataggtggaacaacaatctgaaataaccagtatccccagagctggtgtctctagctgcatatgtatcagaagatggcctagtcggccatcattgggaagagaggccccttggtcttgcaaactttatatgcctcaggacaggggaacaccagggccaagaagtaggagtgtgtaggtagggggttgggggggggcggagagaatgggggacttttaggatagcatttaaaatgtaaataaagaaaatacctaattaaaaaaaaagaaaatgaatgagtgaatgagtgagtgagtgaattaatgaatgaatggtttgcttattagcatttggtctacttaggttagaagggtaagaagggtaagaaggactctatgactccatcatttgaggctgtaatgtgagcttattgatgtagcctgccaggccccacccacactggcagcccctcctcaacttcagtgcttcctggaccccatctgaagtacttaagagaccacaatatatctgtgatagccagagactggccttgcccttgggggctgttcttcttcttcttgcttatcttttggcaaacagcctttctggatttagcttgagtcatctcttggagggatttgtctctgaccccctcccgctcataggcaagaaaagtcagtctgtgaagccgcagcctgccagccgggcctgggactgccagcagctggactgagggatttgttccctctcagaatggacgtgagggtgcactcttcagccctttagtgaatgttggtcatgagccttcaggcaagctgggtctgccactgttcttgatttccctgaagactgagtataaagcacccgatgactaagctcaaagcacttcccacttgcttcggtgcatttccttatgtgcatgggctcctctttgggaaaagtgactgcacaggtgctgggttattttatactgagttgcttgccctttaattcttaacgtgcacgagttccttgggtatccttggcacaggtctgattgattggatgtatgtattggaatttctctcatttttggccatgccttttccctttacagagaaaggggggttttacattttgattcagtcaaatctattgggttttctttttttggtttgtttgtttgtttgttttacatgcttcctgccttttgtgttctaagaaaactttgcccatctcaaggctgttgtgttctgttctgttacagttttagcttttaggctagatcagttgttcatcgtgagttaagttctatatgtggtgtgaggtagagtcaggttcatcttttccattaggctatcaggttttctaacactgtttgttgaaaactctcttctcctcattggtgtaccttggcacctcattaaaaaacgtcatcggaagggccggggctatggctcagtcagtaaaatgcccgcagtgctgcaggcctgagcttgatccccaagccatgttaaagccacacacaaccgcctgcatctgtaagcccagggctggctgtggtgtgaagttagaaacagacggatccctggtgctccttggccagacagccaagctgagtcaatgagttctaggtgattaaaagcgcttgatgctctttcagaggacccaggtttgaatcctagtaccctctggtggctctcaactgtctgagactccagttcccagggatctgatgccttcttctgacctccacaggcaccacacacacctgcagtaagcagatatatatgcaggcaaaacacccatacacataaaagtaaataaacaaaactttaaacagatagaagataattaagatttaaaaagaaagtggagagcagttaaggaacctacccatttcggtctttgtccttcatgtgtatgcatgtacacacgtgtactcgtgcaccctcagacacacttgcacacatcttcatgaaggcatacatagcacatacatgccccaaaagcaggggcgagaacaatttgtgccgaccacactcctgcaggtttatttgtagacttcttcgacttcacttcagcttttgtgaaaggtcttacacccctaccacattgtcttaatccctgcggttttacagacaatcctgaagccagatagtgcgagtgctctcagttccaatgttattttggttatttaggtccttcatgttttcaaagacacatctgaatcaacctgttatattctcttagcaaaaacgttggtcactaggattacatttctaggccagtttggatagatctggcatcttttttttttttaattcattttgtttttaatcatgagcacgtgcacatgagtgtgggtgccctcagggagaagagcgtcagagcccctggaacacagggctgccgggaagacttgggttctctagagatcagcacacacttctaaccactgagccctctctccagcctctagacttgacattttaacgatatcgatccttctgcctatgcatacagtgcctttctccatttattaggtcttctttactttctctttggtaatattatttattttccagtgtaacaaaaggtcacatacaacttttgttaattttattcataagcatttcatgggctctttttgctgacatagatagtattcttttgaaatgatttttaaaaataatttcttctattgcctaaaatggcagttacatcgtacatgttgacattatatccgggggcatggcggagctaatgtactcactcctcttttgattgattagggagtatgttagagctttcatggaaactgccatgtcctctaaggttgatgagttaaacttcttttcccttctttgtgacttttcttttcttgctcagtagcatcagccccggccactagtggaatgtgaagtagacagtacatgtggccgtctgtgctttgttgctggtgcagggcacagcatcctgtccttcacattgagcctcaggcttgctgccatttgtcattgccatgctttgtggattgaggacattctctcgtgtttgtagcattatgctgagaatgtccatcatggatgggttgtggtcctgcatcttctgagctgatcaggggggcttttcaccttattcttctggcttcacaagttatctgttgttgtagaatctggtctcttttcagctgacctcattagatcgtgatgtattatattttcgtatattattgaattatttttttttagcatttctaaagagtagtcagcagaatgtatataaatatatgtatatataagtatatgtgtgtgtgtatgctattcatatgaaattgtaaagaaaaaatgttaatagttgactgtaaatcataaacagccctcgggcggatggagtaattaaactaagaagcagtgaagtatctgtgtcacagccgtgtgctaactgctagaagaaaggttatgacgtttctcgagaagttagaagaatcccctgaaagagggcgggttgtcaacacagaggacagcatacagtgctagctaagaccaacgctgggtattagtgttgtgaggctgctgtgtcacagcagcctagacttggtaacttaaaatgctggcgtttgtaaacagcacatgtttattctcagatgactctggaggccaggagtccaaagtcaagacaaccaatccaggcttcccccaggctccagcagagaacttgttgtctgcctctttcttcttcagcctcctaatcctgctgcctttctccttcctgtgtttgtctccttaatggggtgtaaggccaattgagataacctgggatgttcttaccttaacattctttagtttaagggctggagaggtggctcagccgtgaagagcactagctactttggcagaggacccaagttcagttaccagcctctatgtcaggctgcgtgaagttgcctggagctcagtgccaggggagctgacagcctctttgggacttcatgggcaccttctttaaggtgcacacacacacacatacacacacacacacaattaaaaaataaaatccttaacttaattatagctaccaagctattttcccagatcctgagatgtggtatgaggtctttccaaggaaccttccgtccttccttcagcctaacacacggtagagctttaaaagtttctgtatgacacgttggcatttgattactgaatcagatgccataaagcccgatagtaaatactaagagactaaagttagccatccaggaccgttgattttgaccagcgtagtattttttttttcagtactgcttgaattcgaagtgaaagcatattaactgaaactaaaccatcccctgcaacataaattgttgtcagcttttataaatggttaaaataaacaagtttggtctaactgtttatttggaaaatttgggaaagaagccacttggcagtgtaatttgtagctaattggcttttgatgaattggtcatttgacacattgaattttggccagctggctcttggagaatcagtgttcagcctggggcttggtgcctggggctgtatgaggctcttgggggcaggctgtatgctgggcctaagatctaaaggtcaccttagaccagggaaaggacccagaagacctacgcatccctcagtcctggccagggccctgtttccacataggtgatatgagactttggggactcaactaataccaacacttggttgattttagtgcctgagctgagtgccttgtggtggctattatctcatttaaccactgcctgtatagcacatgtgatctttgctttacagataaatggagcacaagaaggagatagcttacctggctaagagccgggtaagtagcactcacattacgggaggctgtctgtctgtctgtctctctctctctctctctctctctctctctctctctctctctctctctctctctctctttctctctctcttgatcgtagccacaggggccaaatgaacctgctttccttcctcgcccttgcccttagctctgatccgccagttcattcgtaaacgtttgctatggactctatcgcagtggcacctgctactgattctccacctgagggaaccgaggcaggatttgatgtgagactcagccgtgttctagcatcacagctgaacagaagggaggctgttcctcttcttcccacagtccttcagcctccagccagggctgggggcgaccacgacttggcttctctggcagtgggactaacccccaggccccgccttctacccccagccctccttgtttatatccccgttaatgggcccttgcaggccagcccagctgccatgggagagccagggagccacctggagctgtgtctgctgcagaatgtttacggtgcatggagccaaaacgaaacaacatccatcttgctgactggtcacaaacactgtgcccattcatggccaggcggtggatggtcctttgcttggcagtgagctgacacagcttggaggcttgcaggacagccagaaagtgaggcgaaggcaggtgggctcgggtgaaatcacacccaaacccaggcagccctcttgccattttccgtttcccccatccctctccccaggtgtgtcgtaagatggagcattaaaacttgtctggagagagttctcagtccccacggtggctttagaagtacaggtcagcctcagccacagaccttaggaaggcctgagtgaagtcatctgtccatgtgaggtgcctcagcatgggacagctatctgacagcctacacgagtcttgcctctgatagactcagcagttatacactagaggaccagtctacacattgaccgtaaagaatttccatcctggtatgaatctatcattgcactgcctcttgtttaagggtcctgtgacttaactggcctctgagggcagcagtaatatcaggcacacccttgtattggcgcgcagagttcagcaacaggtgcgtaacactatctttggcccagattagggaaaggttttaggccaggcacccaagccagagcaggaagccccctgttagtggtgaagatccctggtgttatatctaggagcaggaggaggaagcagggtcaggctcctgaaggccctcctttgtgtttttatagtgcagggatcagaagacaccaacactgggcaacacctgcagtctctgtgatttacagaagagtacataaagtcccaaagcttgggaggtcacaggctggaacggcctaagactcttggccttgctctctgcccagagtttccctggactcagccttacatgtgagggggctgggttctatatgcagagttatggaaacagagcagagaactcaacatctttcccatagaggcaaagaaaggcaaagctttgtcctcaaccaaccacccctcagcggcccattgctttcttgcagcctgtggagcaagggcagggtcttatttgcagcctgtgcacctgagcacagcttccagtcgtctacgttcgagcccacttctgcttgcagtgaaaatgtgctctgctcacgtgtactcagtttttgtcttgtctcttggtgtttcagtggctctcattagagcaaaggagttgccacgtgccctgagcatcccatggcagaaacagagatgaatgtgtgtgtgtcttttcacaatgctggcatttattgaataacaataaattcagaaggtgcagtggttttaataacaacactttgccaggtaatcaatcccacataccttaaaaatctgaccaaagtgaatacaggttcttttatcccaatattagtaatatcaactcccacagcacacaggtgaatatctgtatctatgtatctatatcagtatttatatgtatatctgtatctgtataagttacagagtgaatacaggttaaaaaggctacagcaggcgtccagaggtttcagaagcaggctgacagagaaaatgcatgcagagtttattgatgagctgacaagccaagccagctgcagggaaggagctgctgctgctacagagatgcagccacaggagagtcaggcttgagagtgcagatgtggttgctgtagagacaagggtggatcaacaggcccaagtccagatgtgcaaggcgtgcaggaggtgaatccagtgggccatgtctgcagcaggagagcacactgagctgaatgcccaaggagtcctcctgtccatcctcgacacagtggtgtcaggtgatggcttggcagggttgtgccatcacaaagatgtatggagtccagatgagggagtgatacccttggtgtgtatgtatgctacctggtctggcataacaaatatgttcttgagtctggcttcactggtatgattttaatacgcccacatgcccttaaggtctcagtggatcacaatacatctatgtggcagcaccttttctattctcagaactaagtcatttgccagtctgtgctgcgtggatagtgcccggcagagagtggtgtttgtgggtctcccaggcatcatcgaacaccctcaaaataaagtcaaaagctacttagagaatggagcctctcaggataagggccagctccagacactgttttatacaatacagagtgactgaacagcagccatcagggcttccgtgggccctaccccgtcacctgaaaagcttctgagctgtctttcctaatcgtcttctctagtctccgaggctccatctcccttgagacctagcccagccatctctagacattgctccgttcagcattcattccttcattcagtggttctcccaatggacgtttgtacccttacctgccagctagctgccccagtgcaaggggaactctgggaggaagggcacactcagtgggggaagaacaagaggaagcagagcagagcaggcctggggaagctatgaacagtcaggggccatgtaactctggcgtgtggtgggaattatgggggatgggcttccgcaccgaggccagactgaaggtaggtcttccttggttggctcagaagttatgtcctcggcagtggaaggatcaggggttagacatggccacatctgggccagatttttgttatttagagagattgcccttgccatgatagagtggtggtcacatttagagggcctggtccagatggaagatagtggcttaaaagcaaggcagagctggcaaggataagggaaagaagggatggaaagagggaagagctgagctgtggtcagagcatgcattgaagtcgtccccagagctcagccagtgccagtatctgccaaggctcccataggtgcgtgggcatagcctgcacgcctcagcacagacccagactgccggtttcccttgctgggcagcatgtttggaaccatgtgccgaccttcctgtaggtgctgtcaagttcaagacctggccacggaatccaagaacagaccttggggctgttagacccagtcttgttctcagaggacctctgtggggtggggtgagatacacttgaaccaaatcctgaccccgccatgcttgctcttgtgaccttggatgaagagtaggcctgggtgagaggtggcctgcagggcacctttctcctaacaggatccttttctgtactgtctcccatcactcctgacatgggatccatctatgatgtcccctgcccaagctgctgattagaaaagttccgtatagagttgtaattgcactcggaagaagagacgaaagggaggcgcccggaagcacagcgttctcttatgtgcatgcccaggcaccattggctgggggagggggttgttgttcacacctgtgtgtgccacctcgcagctagatggctgcagatctactaagttgcaggcagagtggccgaggtgacatgattctctgaattggtgaacaacccttggacggcttccaaaccaaacacagtacagcctttccctaatttacatggtagttccaggcctggaaattttagtgtatgttacagccatgaaaaaggcgctttgttttctgtgtaaaatggacctatgttctggcttccacaactgggtcgcttgtttacagatattttgaatacctggcatgatactaaacagttagattaggactgacccacccttggcagtaagttgggcctaccccactaaatgacagtggcctccagatactatggccatcagaaatccccccccccccgcagcccctgtgatttccattgtgtcccctggagtggccctctcccaaccctgttgtaatgcctgggctaagggtgtagcctggaggacatggagggagagcgagggagcaccccctctccaatcacagggcttcctgggggagggccctcagagctgggtggattcggatctggggtggagctagggggatttttcagatgccagaactgtgccttgccctgcaggctttcccagcctttggaccaatctgtggaacaagtaaacttttccacagggcatagagtcctgagcctctctgaaggccctggaacccttcccacatcttcctaccaggccacatctctgcatggtggtgcttcctgggggtggggatggcctccttcactgggtggtggtaggggctcctctctccttggataaagatgctataatggaaagcatagcatgtgtttactgttcctaaggaatgacgtggtggaagaggggtggctcaatgacactggaccttatcaagggagtgtcttaaaaacaaaacaaaaaacaaaacaaaaaaaaaacagttcaagtgtggtttctgccaaatgtgcttgctttccctcttccctgccatcctgatttcagattctgccagggactgggatgtttttacagttactgatccttctaagatgggaaggaaccaccatcttcatccctactttcgagttgtctggagctgggtaccctgaacctcccctgctgccagcccatcttccttcttgggttttatgtattttctagcagttggaagaaacattacagcgttgagagaggaaacatgagcataggagttgactaatgagcttgtttccaaggaagctgaattgtgaggttgtaaatggatacttgtggctcgggacttgtgtccatcctcaagttgtctagaacccaacagccgttccctctgggtgtctggccaccccattactgtccaacagccgttccctctgggtgtctgaccaccccattactgtccaacagccatttcctctgggtgtctgaccaccccattactgtccgacagctgttccctctgggtgtctgaccaccccattactgtctgtgctttctgagcttccataaaaacaagtgacttttggtgaccgttactgtcctccctgtgtgatgtgggccaggcaggacgcctgtggagttccagagcctccgagaaagtgcagggtgggaaaggagagcttggagcctgggctctaagagttatcatgggctctcaggcccatcttaatggaggcactgagctctgtggacctgaggcatcacggggtgagtggaacagaagtggtagccagacacatagagagctcagctttcagttttggaaccgtggaagccgagagtgttaagaaatgtcaggatttattcactgggctacctttcccgacatgttttctttccttgggtccctgacactccagagcctccactgccaccgaggggcacttagcatcttttctgactgagctggtcagtgcccctgtaacagggacagaggggttccctgaggaagaagagtctctggccctgggaaaccctagcagtgaatctagacaaatgtttccatctccctttaggctgtgctctggtattcttgtcttttggttgcttgtggaggcccagagcagcagggaggtctgcacacgagaagcctggaggatggccattgagaagcctgacggcaggaatttagctcaagcccatttttgatgtagcctcgaagattgtttattcatttcaaaagcagtatgcagtgttttaatatgtgccatgcagagattgtaacctgttagaatttatacactgtaaaatttacctatgcttgtggacagttttatgagatggggcagtggttagcgccatggttctcaacctgtgggtcgtgaccaactggggtcaaacaatcctttcacaggggtcacatatcagatatcctgcatagtagatatttacactacagtttatcacagtagcaaacttacagttatgaagtagcaatgaaataattttatggctggggtcaccatgacatgagcaactgtattaaagggtcacagcaccgggaaggttgagaaccactggtctagagtcacattgcctgtttcctctgtcctgaaagggagcagagaaagatagaagccctcagtgctaaaaccaaacctcatctgaaatcttccccatgcggagccagactgaaccccgtggcccatcctatctgccattgcctactcaggatccatggcttcctgccaggacacactgtcatcaccacccacggagtcctgcaggacagtcccagcctcttctgtgaccttaccacaaccttctccctgttgcttgctgggcttttcttcctgagccttctgaggtcactggtctcagggcctttgcttactgtcagtgcttgtcctgaggtggcacgtggcttctgccttccttctcctcaggcccctgctcagtcagcccttccctgactctgtctggcctgcttctcactctctagcttcttgcctttgtttgaactcataacctgtagtatctacctgatatcacagggttaatctgtttctttatctttgatattatcttctcttctaccagaacgcaagcaccaggagagcagagatttttactttttgttcagcacatctgtggtgcccagggcagagggaaactgtgtttctggtgtctcaccctggcacacagttaggtattcaatgcatgtgtgttaaaaacacattagttgttagcatttgatatagcaactccttttccattttttaaaccatcaagccttttatttttattttttaaatacgtggaatttcaaatcctgcccttttctggtgtttgtttttgtggcattatggattgagcttggagtcctgagtgttctaagctctaccaccaagctgtattcctgttggccttctcattggatgtaattcttaaaaatgttttcttttgtgatgaagatttttttcataaatccagaaaaaaatatgctctaacgccctgtgatgaggcggactcttctactttgtgtttgcgactggcagttgcttatcctgtgtccagctcctccaagtagatggtgaggatcccctctctcacagagactgggctggcagaggggatgggaaccaactccctcatgacctgcctgatggtcttagttccttgtcagatacatactttgtggcttacagaccattcagtaagtgactcacatgagctgggaagagaaacttgcagaatattattcacagactacatttttgagcaactgaaacttttgagtcatgcttgctgcgtcatggtggctgtgttgtgctggtgtctcactgtgccacgtctgatgcctgtcaaatttctgtttatgttctgtagttggcttactcattggacgcacaaaaagcctcctgttcttttctgttatcggtgtctctcaactcttaagctaacagatttttattttggggtggagaggaagttgggagggaggtgagtgtgggcacgtatgtggcactgtgtgtgtacagaggtcacagagtctcaggagtatatcctctcctgccgtgatgttagggatcgaatgcagattgtcaggcgtacacagcaaacacttggcccaccaagcctcttctcttctcttctcttctcttctcttctcttctcttctcttctcttctcttctcttctcttctcttctcttctcttctcttctcttcttttctttcttcttcttcttcttcttttttttttttttggtttttcgagacagggtttctctgtgtagccctggctgtcctggaactcactctgtagaccaggctggcctcaaactcagaaattcacctgcctctgcctctgcctcccaagtgctgggattaaaggcgtgcgccaccacgcccggcttctccttttatttctaaatgggaacaccaagagtcctctgcagagaaaacagaggtgttaccgatggcataaagtgatagagcctttttggagggcagttcggatggtaagtaggaccatgcttaaaactgtgcttatctttggactcggcaattccatatccagaaaattatcctaaggctttaaatatgaataaatataagataggtgatcatagcctggttgtttcagttttctcttttctaaacaacttaaaatacaactataaggaatgaagtctggtgcagccaggaaataggatactgcacggttgttacgattacttattggaaaagtgttttcacatctggactaaaacaaatttggcttctatcagactaaagcgtctgccttggtctgttttctgttggaccattcattacctgaggatgggtactgatcctgagcgagatccgccgactcatgggttggaggctgggagctgatactaaagtgccagcctctggcagaggtcttttttactgcttcacggcatggtagaaggcagggcatagctggaaggtcagtagagggcaagagagaggaagagggaggcagactcccaccacgacaaacctactcctcgggagaaggcattgtctgttcataagatacggcgtgaagacaaactcctgtgaaataagtaccagctaggaacagaaacattggctcatggtgtcacacaacagaagttaaatttcaacatggtggggtttggtaggtagggtacatttaatgcttcaagtcattgaagagctgacaagccagccaggactccacatacactgaggtgactgtgcctgtggctgtcataacttcctttcaggcatttaccacttgcctaactcaagggagctttttgcagccttactgtgcgtgtagactggattggagcttagggctaccaggataaggctgagttcccagggagacagatgtgctgggattgtaccctgtcatttcacgttgtctttcctgcccgctcacatttgctggtgcagaaatggcacaggccaaggaagaagagtgggagcagaggctggcagtcagaggctgagaagcgaggcgaacgttcagtggcttcaagttctaggaagacagatattgaaattcagagtcaagaagaaggagcctgacatcccctgccttcaacaggagccccgaagggtgctgcaaaggatgagttgcaaacacagccacagatatgcaggggtacaggatttgggaccaggagtggggaaacccaacacagattcagaagtggtccagctgctcccgttgttagacacaggaacatggggtagaatagtggagttaaagtaagggagggggaataggaatgagatgtgccgtgttgggggcggggctggatggagttgagaatcctgtgctcttgccctgccaaagaagtaggaaaagaactaacctgttagacagtaatatgtcaaggtaacttaaaaagagaggaagttgtgtatgataagctaacaggcagaaagtagaataataaaaaaagtctaatatgaacaaagttgaggatgaagaaggaaaaatacagaataggtgagacaaataaatagtaagattatagatttaattccaaacatatggaaattacattaaatataagtggtgtaaatggactaaattttcacattagaaaggttaccagagtgcataaaaagctgatgtcatagactgttgtattaacaaggtcttttttgttgtgacggctgtgaaatgctatgggatgttatctacccacgcgtgctgaacgtcgctcagctgttctgcttggggagcctggtttactgatgtttgtcctcacactggctctcccctgagacacgatggcttcttgttaaacaagatgacttcatctagttcccagagctacccaccgcatgcaggagagcaggggacgctccctcctggtcaggaactcgagtctctccaggcttctgacctaaaccaacttgtttattgtccgtgctaaccccaggctgagagggccaggtcctgctgattggcactggcgtcaagagaacagcaagggcaggctgggtgcacagccagagcctactccctgccgggcacctcgtatattgtggtggtggtttcagcttcataagccgggccatggagaagagaaagcatgtgttctacacaggtcacagggctcctaaataaagtcaagatggaaacctggccagaatggccacagcgtccgcacagcatcctccagactctcctatctcatttttaagcttgataaatagggtccatcttctttcctgtctctatcagcatggggaagggacggattggatactggggtcgtggactgggtgtgttctcttctttaggttcacatgctgaagtcctaacctgcagtgtgggaagcaattgaggttagacgggcttatgagtgcctgagtagcagaactactgcgttcgtaagaagacaccagagtgttccctgctctctttctgcatgtgcgcatgtaccaaggccagaccatgagagcccgtgctgagaaagtagtgtctgcaagctgcaaaaaccagccttcacaggaacccaaactgcctaggagtgcagaacctaagccttctggtctctagacttgagagaaaataaagtccagttgttcaactactctgactatggtgcttgtaacagccacaggagactcacccggggagagtggtgggcgcctctcccctaaggcatggagcgtatctccagggatgatggagcgtgccttgtatgctcacagctgtttagaggtgtgaccgtagacacttcccatactctgcttctggaaaacacacgttagcatatgtcacagacagagccaagggcacaggacacgcacgtgacaggcatgccatctcttttggtattcgtcattagaaatgagtagaaaaaggcagggcaagagactgttaattattttatcatcccacttttgacaagtaaggctgtatgcagatacacgcagggaagaccaaccaagccctttcactctctccatgtccactgcggttctctggcaagtgaccactgactacttcattattttcaatttttttttaatgttttgcaattagtatgtaacaatgtgaggaaagaattggctcttctgagtgtctggatgccagcatgtttagagccacatgtgtaaccatcttggactgttcgtggccttcactcagccctgtaaatctagcttagctttagggggcggggagtggtttatgcggctcttgtggtctgggataaaaggcagtgggcagctctaagcctggtacaagagttcagcgtcaggagctgctgctgcggaggcctagtccaggttacaggcccccaggtagctatgactacaacagcactgcagcaggtaaatggctcgccacctctcacttcctctcccctagtggtcccagctacacatcaggcctcgccgttcccaatgctaggaaaagagtatacagactagcctggtaaggcaggagtggcctgagaagttctttgtcttcttctctgtgtccccagtgagctatggactgggcctgggaatagaaggcgtggagccctcggtgccctgtgctctctgacaccccactgctaggacctggcacatgaaattcagttcccatgttcccattgctcagaggggaaagttgaggctcaacatagcctatgctcatactgtcagggaagggctactggcaaggcactcctgacctgaggctgtctttctgtggtcactggcactcactgcccaagaaagtgggctctctccttatgcctgtcgaaaacaaaacatagggccccttttcagaatgccagccactccagaaaccattccaggggctctgcatgcgctgtttcctttgatgtgcacgtccacccctgcagctgagattctaaaccccatttttcagatgaagaaactgaggcttagaagggaagatcactcagtttggagtcaagcatccccaaaaggcaatgctggacttggatgttatcctaggtgtgtgtgtttagttcccctgaattacaccgtctgacagagagcatacaagagtgtgaacttggcacagttcacagacacactctctcagcatttccagccatctgttgcgaacctaactatgagctctgaagagggacatatccttggggaggagggagggaggcagaggtgtctagagaacctacctgctgggtaagatggctaagccccttcctcagccgggcctctgttttctgtaaggagaaggttgggccagttcacatctggagctaggtctgctaactagagagactaagatctctgtcctggggctatgatgggcctttttgggggaggggggagcagagctttaactgttcccacaagccagtgtggcagttcagtgggggtggagggtggggagcgggtggcaacgatccaactctggtgataaggatgagacatctgggcctgggctgtgtgcatgggacacccaagtagcaattgctcccagggctgctgtgagctgggtggtggggtagctttagaggctgagaccagggaatgttctaggggagaggagcttgcataggcctctagattagtgaggatgtgcctcagcaaaacattgtgagaataggctcactcagacagatgggtgagcccagcctagggggccctagagccaggctagcaccccttccatctgtatgcccacccgggctgacgttccattgtcagtgccacctgtaaatgtctccccactttgcagatgtaggcgagcattaacagttccgaactctgaatccctggccccttcagtgattgtcaggccgacagccgtggctgcctttattgtgtgataaaatacctgtccaaggtatcttaaaggaagaaagactaatttcagcttaggtccgtgaccaagaaggtatagacgcaggaacatcatgacagcagagagcttgggctgaaagccaggtctcagtgtagccctcagaagcctgtcagccgtgcctcctgtcccaagtgcttcacagcctccaaaaaccgcaccaccagctggggaccaagtgttcaaatacaggggcctgtgagggatgtcccattccagctaccacatcccaagagctgggacctgggatctgatttccaatcaagccctattggcagcacagccccacctgctcctgtgttggttttggttccgctgtggcagagctaaggagtagctgtggctcctctggttgttggctggtttgccttttgaagaaatacttcgctgactggcggtggagttgggtatttctgagctctacacttgctgtgtggacttgggcaacttacataccactctgagcctgcctagccagctcctgggggctcggggggttggtggtgttataatgaagctcacagttgaggactatgaagtttttataaactgtgaagtgttgtagcctgtccctggcctggtacctggttggttctgaaaacctgcttgttggaaaacagaaatcagttaccccgttggctttcctggtccccacaggtggctgcaaatggctgcaggaggtcccctagccttggatgctaaatggcagcaagtttcagcaccaaaccacattccctgtgggagtcaccttggacatggttctgttttatagtcaaggcattatagtaacttgcaaagcttctgtgtgtataatatgagcctcttgcttcaggctagctcactgggacttcgttcaggtggctgagccccgcatttggtgaaccctaagcctggagtctgactttggaccctgtagacccagtctgacaggagatggtctgcttctccaacagccttgcctgagccccaggaagcacaggctgagtaatagcatccttcttactcttttgagtgttccgtttaagctaagtccccgccatatgcactctattatctcccttgtcaccaccaccctatgaggtagttgttagcaccattatacagacaagaaaactgaggccagaccaggaaatgagaccagtttcttacttggcttcagaagtgtcagtccaggctctgctgtgaatatgctatgtagtgacacaagtcctttcccacttagagcctgttttcttcatatactacggaatatatggccagttccagccctggatttccaagatttcttgccttttgttagggggcttcatgtgctaggagaaaaccaggcagtagggtccgtttttggtttccgtggctccaagggagacacgtcgtctaggtaccccaccttatcaggatatccctatatcaaggcagcaggacaccatagtatacctttcatcctcccctctccagagacgatccatccttcctggagatgagaatgaacatgagaaagactgctctgagtctcatggtgcccttggggatggagacaaagactccaccaaagaggagacaagacctatatttgtcccagcagacttgctgcgtgtggcaggcaggagaggctgtgtctctggggcctttggtttgatggccagccttccaggaagtgggagatgtggggatgcctggcatggactagtgccccagctgtgcctgccagtggttgctttgtgaccttgggtgaccaactgtcagctctggtgctcagctctgccaagttcagaggtcagactttcccacagggcccagtggagaatgagaggcaaggagttgggtaggctcgccacaacagccagtgccaactttgacctcagagcctgtcccctcctttccaacatcagttgtttgtttattgcaaggaaagatcttgctgtctccaacagagaagcagctaaaaggcccaagcattgatctactacttggtgctgatgactctaatcccctcaggctgtctttcttgtgtgaccttggcaaagcagttaaccccactctgtctgctttccctggagtcctggttgggattgcttgtgtaggggttgggggctgcaggacaacactcaagcctcaaagtccaagtggtttggattgtgtccctgagcctgggccaagtccctccatctcttcagcccatctggaaaaatgctatcatgagcctgccctcaggtgagtaccaggaagcctgaggaggaagggctgaataagtacaagacctgtcactactgctccctaagtcagtcattggtgctccccaagcccatcaacagtcctccctaggtcagtcactggtgctccccaggcccatcaccagtgctccctgggtcagtcactggtgctccccaggcccatcatcagtgctccctaggtcagtcactggtgctccctggatctaccttccttctctgtaccctagtgcttgcagggggaggtacatgacccagagatctcaagaggtcctgggagggagcttagaaccacccttaccttcagggtctgggagaggtcctggcagagcagccccctgtgatgctctctgctccaaatgccattaatggtcataaaattatccacagtattttagaggctggtcccatggtttgcctcaatgggacaataaaactgcacgcagaaatgatctggaaaaggcgaggacggccaagcagagccggagttaaatgaactgcagcctgctctcattaactcaggccattcacatttgttaccagggatttctttttaattaaaaaacaaccaagttattaaagcataaccctaagaagggctggctgggcagccgtcgaagccagcagagcgagcggtccgcacagcaggatgggtcagtgccgccagggaaatggcgtgaaacagatgttattttgagctctcacccaacccagggccttgaaagctgtgtttctcatctgtacactgtaaaatgtgattcagagctgggggagttagtattgtccgtcaccggcacaaggatagcctgtgctaaagagagtgtcagctgagctaggactggggatgctgtcttaaaagaccacatcttatcacactcagtacaggggcccaacacagatttctagctgtgtaatttgcatgagcctcagtttccccgtgttttaaatagaaacaagcaggtagtggggaacccatggcttgttcatagtgtgtcccatgtggcctcattgctgtccttgtccttagcttagagatttcccatcctcttgctcagaattgacagtacctggactacttgggcttggtaacgttgtattcggtctccaaggaattttaaggtttctaaatgctgagacacctaggagtgctcaccatccaggggctcctgtgggtgctagcactgtggatcccacagtgcccagaggagcacatggtcaggctctcatggagatgtgcctgagacatggaatgggttctgggaagtgaggaagttcatgtcgtgacccctaggccccaaagcagtatccctaaaagtcctaggacagacagccaaaggataccttccagattaactgcactttcccaaagatgagcagctgtgctcagggaagatgggagaggtttacagcaagcgtgtctgacaattttgttcactttatgggatttgtagagctgttccatgtatagagcctcatctgaggcattcacggagcctatagaagcaggcaggagtgtggagtatagggttggtgggtcatcagagaaccacaggccacagatggataaccagactgtctatggtgtgatttgggaagcaggatagagggggctttggtaccagttggcagcagatgcagttggactgttacctccctcctgcccttggcacatgtacccaaaacatctccaggtccagaacccactggtggaatccaaccactgaggcagacctagggctgtcccccaaccccaagtccagcacattagaacccaaacccctgcctagggtctgtgctcagctcccaccctgaggcggtgttttcgagcttcaaggaaccatcaggttggctcaccagtgtttgagctcctggaacaggtgtgtcgacaccacagacacccagtgtctgctaacagtcattcgcgcattggcatacctgtgcgtgcacgcctgatcacaatcaagtggtcctgccaggtggttttgggagttaaatacggtgtccctgagcctcaaatgtggggtgctcagatagctgcaggggttccctactcatttggcaccttacccacctcctctttccttttgtttccattcccccagtgaacgggagctacgggcacagctcagagaagagcctgctggacctggacctggccgagggtcccagcccctcctgccaccagggtctgtttcttcctgcagggaccccaccaccccggggtcacccccctgtctgtgagaagctgctgcacttcccccacccaaacaggtatgtgcccgttcctgtccacctcacctggtctgcttggacacaagagggccagggacttgtccgagtctgtttgttacacagaggtgcctagtgtagcctctccttgttggaccaggggcttctaagggacacaaagatggcttaagtgaaatgcatgtccctgtttcacaagctgaggtggcttctctgagagcatcaggagcttccacagagcaaggaggctgtgggcaggctaggaaaggaaatcctggaaggattcacagttcttagtgtggaaaccattgctgggacccaccctctccccttgcatgtctccgtggaagcatactgctcacccctggggcagcatgcctggagcctgcctcacaggcgagaggctctggatactagcccgggtactactttgtacccggccacttcaccggctgcgttgcgcctaccccgggccacgtgctgggctgctgctggagtctttgggaatggaggcaaggatctccacttcaggaatgatgaacagtgggagacacagataggaagacattgggaacttcaactccccgaggggcgggctttttagatatcacttctattttgaaagactttgaaagatttctattttgaaagaatatagtcatttggtcatactgtcataataaatgtcatagactgggatggcttaaatacaagaaatttatttctcataattttggcaggccaggaagcccaagatcaaggggttaacgggtttggtttctcctggcctgtctcctccaggcgctgtgaggcagtcaaatggctatccctttatctgtgtggtctgtacacccatcttataagggaatcagacatattggggccctctgaaccccacttagctttaattgcatccttaaaggccctgtctccaaacacaggcttcccacatatgaattgggagagggcagtccattctataacaggaataaatatcttttagtcttcaagctgactactagttaatgacaatcacatgtgtctattgcctggattagtcactgagactctggcaggacactgtttgtagagggagggcaggggcaggggggtgcattttgtgttgggctgtgccagcccaagatgtgattgcactgaataaagagtcttcaacccaggttataggctttaggccacagaagcatggatcctgctccccccccccaggtgtaagaccctcttccccccacacctctactgcaaaagctgattcctcaagtgcagcagctttgcaaagagtccaaaacaatggtcaaaaggaggtttacatctttgacttagaactttgatgccctaaagtaatgacatgttctcagaaaggcacttggcatcgaggatgctggcttcccggtgatggcattgtgagagtgccaggattctgactcataaagactcaggttgtctgctgtacttggtccatcactgtggctttgagatatgaacctggtaaagcaggtattctttaaaggtttggtccaggaccaagtagaacatagtgcctgttattccactgtcaagaaagaaggagtcagctctccgggggtggggggatacctggtgcatctgcatcttagtcactgtgcactcagggctctgggctcacaaattctagagagaattgtagagctcagccccaaagcagccagaaagccatttgtcaggcctcccagggcagggaggaaaagccaggccctgccacgcccatgagacctcaggctgggacctcccacaattcccactgaggccattcaggtgactgtcccttatgttcctaactcagtcagctactgaaccattcattcccctttgctgatgtcagttcgctgacctggagacatttgcaaaagctctaagctggtgcttcccacagctccctctgccatcctgcctctgccagcgtggtgccccgctaagccgatactttcacatggtctccttcagtagaacctgctgctggctgtggcaggcgaagtgtccctgcctctgcggataggatgtataattgttgctcggcagagatgctgggagggaaagaggctcagtggacagttgcttagtgtctaaaaggcagacagttctttgtgcttccactcaaggagagcttgcagtcagttctgcacaggagatggaaacttggctcctggtccaggtctgatgctgacttgctactgcatgatggcctggtggtcctcaagcctcaggctgactccctcctctgactctgcagggatacagggtcagaggcaaagctctgagtgcctttgggtgccggctctccttcctgcccctttcctcacttccattctcttcacctgtcgtctggcaccgagggggcaggggctagctctccaggccaccttacagagctcagttctggctacaagagccatgggacagtggtcctgaagagcaggagtaatggcaccgtgcctagccctctccttcccagggagctgacccttgaccccagccgttaccctcatctacatttagaggtgtggcctcttgtctcacagataaggaagcagatccagacaaggcgtgctcatcaggctctgatcgaatagccagagctccagttcctgtagctactggcaaggtatttctgccagcatcaccctccatatctctcccattttcagtcttctggtagctctggtattcctacctccatcttaccttgtcaccgtccacagtggtgcacaggtctgtgcaggagttccggtctcctgttggacagttgaagggttctcttccgtggtaaagatggcttctgtgccagcccaccctgccattctgccactttcaccattgatggggatagagaccccaaacttgcccagaatgaagcgatttgccccagtttgcccatctagaacagagctgaccccgacctcagagctggtctcccagctctcagacggttctcccaagaactgtagcttcccacacacctgagctcagaattctgggccatcctgccaagaatcttgtccccttgctgtgaccgtgcctcaggctcctacccagtcacaccaactcttggaggacaagctgaggagggccagatgtcactaaatgaaggaaagagtgtagacattaaaaagctaaggggcgtgtaagaagaatttaaactccactggacctgaagacgcctgtggccttttgcccacagaaatcacttgagggtcagtggagagggtcaagactgactccagggaagctagagaggccagcaagatggtgacaggagcaagggtgactggggtctggccaagggctgtgtccttcccgggttcctcatccatgcctcctgcctcctctgcctcctgcctcctcggctggctacatcccctgcctcctgcatccctctccaggccacgcccttctcgcatctgcttcctttctcttccctgcccttggcacagccggtcctggtcttccctgtgttctgcttctccaccacttctccccagacccctccatatctctgccctctgcttcccaggagccctgcctgacctctccagtgccagcctttcataatcactctggccactttcttccaggtcacccagacctcaggctacgtttgtgaacggcagcctcccagccgctcagcacatcaagcaagaagccctaccggactaccaggccatggtcagcgcccacacacccctgcccacccactgccgagccccatcgtccatgggtctgccctcagacctggactttccagaccgaggcctcaccaaccctgcaccttcctgctaccttctgggcaatgaacccatctcagacctgggtccccaacccgaggcccacctccccgagggcagcctgaaacgctgctgcctcctgggcctgccccccacctcttcagcctcctcctcaccctgtgcctcctcagatatcaatcctgtcatccactcctcccagacagctctagttagctgtgtaaatggactccgaagcccacctctgccgggagacctggggggccctcccaagcggtcacggcccgggcctgcatccagtgacggccaggagggcagcttgcagcttgaagcatgccggaagtcaggcttcctgaagcaggagcccatggacgagttttcagagctttttgctccacaccaccagggtttgccacccccttaccccttgcctcagttgccaactggccccggcctcggaggcctagggctgggcctggcaggtaggatggttgccggtcggcaggcatgccgctgggtggactgctgcgcagcctacgagcagcaggaggagctggtgcggcacatcgagaagagccacatcgaccagcgcaagggcgaagacttcacctgcttctgggccgggtgtgtgcggcgctacaagcccttcaatgcccgctacaagctgctcatccacatgagggtacactcaggcgagaagcccaacaagtgcatggtgagtcccgggctgccaggcccgcttagtctcccctcagagctggaccgtccactgcatttgagagactctcttgggagcaaggcagagctcccacaaaatccagggctgtgggtgtggccagggtataagccgtgtgtgtgtttctgtcactgagaaccttggcagggctcccttatccttgacaggtgccccgttccccacactgatgggttagatggatataagatgtcatttagcatgccatgtggccttctgcacctaaaagaagcctggcgagacttgccttttccatgtcaccggatggtggtggcaggtgaggagtgtgccctcctccggcactggcctttacacagtgtacttccaagacaggttcctgggctttctgagcctcccagtgttggcttctgtgagaggtaccgggtccctagggcaggcttgaagctgaggtcaagacttagcataaggctcgcacagcaccagcagtgtctggctagggttgagacaaaggtttggggagaaagaagcatgagcatgcatgagcatacacgcagaataacacgatttagatgcaggttccctgacctcagtcccttctggctggccacgtccatgttctgtgatcgctgtagtggaaagtcctatcagaggctaggagagggatcttagtccctgtacccaccctcccagtccagaagcagcagcctgggaacaaagggctggggcttgagtgtgctattcaacacaaacaatacagaccacccacaccacacatgcctccctccctagctaccaggcatagactgagtgtgttctccagtcttgaagataaggaatctatcagttcatagacaggctacaggtgggcagctttgaggagagaaggaggtagaggctggggcttgggagcgattgtgctgtgcacaggtgtgggggaagggcactgagctgctggagaatataacttgatgtctttgggctgagggtatacaggcaggagggctgagggctgagctagtccctgaagaagacgcatggcctaatgaaagatgtgcttctccgagatggggaaggtcaacaagtcgataggtcagggcggcaggagacagcctgtccttccccaagacagagaccatagttatcaatcacatattcttcctgtcgggaatccatggtttctgtatggtcctagggagtagcctgagatacgcagcagccttcttgtgctagggccttgcatacagactcgcagtgtccaggagtggcttgggagacgacctgggccatccttgagcccagtgggggcgctggttgctcaaagcagcctcaggatcttcctggtcaaggttctcataagattccagcctctgtatgacactagacttggttctgtcacgaaccttgccctgaatggccacggccttagggatgtgtcctcaagccattagggaccagactgtgacatccccggatggagtttgaagaggcctggggagggagcccagagggcttcatgggactggtgtttgatgcctattggagatacatttagtctttatcaggactttcatatacagagaatcaaaggtcagaggtcagatgagtctggtccgctacccacattcttggaacagtggtgacttcacagccccatgtggccctcaccacagctctgtagataggaactgttggcccccgttttacagatgagaaaacagagggtcagagaggttgcacagctgtctctaggtcacacagctagtaagagaaaataatagaagcacaagatgacagaggctcagaatcagaatggtagtaatgattacaaaagcaagtttgcaaaattgtaccactagtaaccactggcgtgggattgaacctgcatccctgtccactgcagtgcccttgttaatttagatccttctctggggcctgccggctgacactgtccggtaaggctgtgtgcagtgggagcctctatgaagccccaccccaggctgctcccgcccaccgtgggtgagcatgcactccctatggtagagtaggtaaccagggcagcgggccaaccccggcccacacccaggtgaattgaacagctcccgtccctgtaaaattattaatttctcatgacaggcctgtgctactatgcctgtaacagacaaaggaatcttggacctgaggcctctggaggacaggaaatggatgagacattttgattgaggagtaaatggcccactaaaatagcagttaatccttttagtatctccccggcagcgttgccacggctttcatgaggtaagcttgcctcgccccatcgaactgcagcacctccgcgctggcagctggtttaaatttagcccgtggagcctctgccctactgggtaccaggcatcctgggtgtggagagaggagcctgctccccaccattgcctcttagtcctgagccctgctggcctccaggtaaccccgccctcccttgacccttcctgcccggcggcaggtcctgccctgttcagtagcagtcagcctgaggaagcttctagatctccctgcaatatcctgttgtggttcatgttgctcccattgtctcagtcttattaaatccggggcttcatctgcgttaccataacaagcctatgtccaacccaaatactcacaaggcgcctcgaagacgtggactttgaaaagattagtcccctgctttcttaggttggttccaccagacagtcagaatcctcattgtctgagctgccgaagccagcagccagccgtggggcagagggggaaagagtggcttctcgggatagctagctcttccctgccgtgggctctgtaaaacaacagcaatgcccagaaaggtccttgctactgtgaagaatgtttaacagctctgtgactgctgcatggggggcatgagcagagccacaaagcctgcctagagtcccacgtacctgtctcccacttcgactctgccatttgccagctctgtggctcaggcaggtgacttaccccctctgaaccgtagaatacttatttataaaatgaggcattgacagtatttaacttgtagggttgttgttagcaggatgcaggaagagtgcatgattaatcctgttccttcccccccgaccccccccccccccactggtgcatcgatgtctctgctgatgggagcaaccttcttggtgttgctttgagccagacctaatctgcacaggtgtgtcctgtgctggttacagcagaaatcagatggttcccatcctgcccagcccaggtcggccacagggctacacctctccagcttctacacctctcctgctggccagttgggtccaggtgcgtttgaatggcagccttggcacagttgagcgttagagccagagtgagccaaggaaggcaaaactaaaccccaaggcttctgggtggacgcatgttctctctctctctctctctctctctctctctctctctctctctctctctctctctctctcgtacacacaccagaattacatatgcctgctcctagttgccttgcctcaccggtgcccctcccccacagctgatctctggtgcttggggagggggggtaaaagtgggcaggatattctgagagtctccttcttttgcaaggactcagtgtgagcctagcttagaacaaagctgggcaaactacaggacccagaggtcagcctctaagggtcacagtacatgggaagtctcgggggaaaggtgaactggacagccagccggctctcagtcacctagcctaggtgcgccctaccccttaccttgcatagcctcttccttcagcagtgcttgtccagtcttagcaggtagactgcctgagtaaatgcccagctcctggcttttgagcgcagctttgaggctaacgctccaactcaccacagcatcatagcctcatcccagggattccctggggcccggagatgtctagatggacaggtctgttggtccttccccataaggtcacgctgggagccatgtgaggtgacttgggaattctgccattgtgaaaagcatcctctcacgtgcttataagtattaagtcttgccgtattctgctaggtgatgggggatgagaggaagccaggagctaaggatggagaaccaagatcagtggtccccaaccttcctactgctgtcaccctaatgctgtgacagcccctcatgttgagggtgaccccaaccataaaattattcttattgctacttcataactataattttgctgctgttacaaattacaatgtaaagatctgtgttttccagtggttgtctgattctgaaaggggtagtggcccacaggttgagaactactggcccagaagtgtagcctaggtcaagctgatgaagagggtagcacactatgaatatccctgggtattgctgaggtggtgaatcagtgggcaaaagtatttactccgtgtggacctgagttcagtgctccagcaacaccaaagcccaacagggctacgcacatctgtgagtccagtactggggaccaggacgtggtactgtggcagacctcagcatcttcctggccagacagttgagctaaaatggtgaacctccaggtttagtggagaagcaattgaggaagatattctgatgcaaacccctggcctccacatgcagccacacacagacacacctcccacatacacacacctgcatacacacacacacacacacacacacacacacacacacacacaccagccctatccttggagcatccaaggcacagggatgctgtcccactgtccagatggcaagggtgttctaggacacagaggtcagcatccccatttacagttggggagggcagtgagaacagcagtgttcacactcagactgggataccaggagtaggatttgaacccaggtctcagcctgactccagacacgtcctaccatagctctggatggggaaatatccaaacttgaaacttattctgaaagctttcaggggcccttgcctgggtcctgggatcacagaggagaatcaagtaggttcagaagccctcagccagatagggagacaggcaactaagtcgccaagggagccagtgggctgtgacttggtgggttctctgttctctctgaagggacaaaaaaattacctgagaacaagcccctggcccggatgtcagagttcctagccaagtttgaaggctatgtaatggagtgtgctttgacacctctgcctggggaaatcgacagtaagaagtccagagtagccactctggcccttcagctcaggtcaccaagtcccaccgtgctgtgcaaatgggtcctgagtatcacttcattctaggggcgggccgtgcccgaggagggagacggtgttgaccaaagacagctcgtgagttggcatccagaagcgccccgagtgctaacttcttctggggtcagtcagttccttgtccacctcattaggagacaagggaggcagggccagagccagagccagagcctgagccactagagcgttccagagtcttgtggacttgcctccttcctgtccctcagggctttctcccttcctgggctcccatttcttaaagcctcatctaggccttggcttctggtttctgtgtctgcactgtcttcccaacttggctgactcctgcttcccctcacgtccttgctcagtgcttcctgcccagctccttccctgagcctgctgtcctcccgggagccccctggtgcggccggttgtctcagagactgtgttactttacccgttgctgcgacaaaatgcctgacaaaaataaccttaggggtgagcatccagtttgagacgtatacagtaataataaggtatacagtaatattcttaggggtgagcttccagtttgagacgtatacagttgttcgccatggcgggaagacacggtggcagccctcacagcgtgttgaggtcaggaaactgagagataaacacgctggtacccactttgctcagaccttttgaattcggtctaaggcctcagccccatgtgttacccacattcaaggtgagtcttcctccacatcactaggggtgtgtttccatggtgattctaaatccagttgggttgacaatgaagatttaccctcagccttggtagcagaggagcagtcatgggcctttttgccccagggctgggccaagttccctatgaagccatggccccaacaagaccctagtactgtcctgtctgcccccttggctggtacttgagagtttttcttcaggaacacctgtacctggaagctgccgtcagcttgtgcaagcctgatcaaacctctcagcctagattgtcttgcctgtgaagggaggtgactgagctgttatgtgacttcagacaacagcccataagaagctgtgacaaacgattctgaatgttttgggcaatttgccctctgaatggtgtgtgttggtatgtaggtgtgtgggtgggtgtgtatgggtatgttggggtgggagtgggagttccctaaaggaacaggaaagactgttgtacaaaggattctactgtgggctggctttgtcaggcttctgcctgcaacccaatgtgcctgcttctccatacctgcacctatgcctgcctgtcaccactaggtccagccccaacctcccatacctgcacctatgcctgcctgtcaccactaggtccagccccaacctcccatacctgcccctatgcctgcctgtcaccactaggtccagccccaacctcccatacctgcacctatgcctgcctgtcaccactaggtctagccccaacctcccatacctgcacctatgcctgcctgtcaccactaggtccagccccaacctcccatacctgcccctatgcctgcctgtcaccactaggtccagaccctcccaacacattataattcagagctcatgtagaagctggatggtagccttctgtaatctcagcactctggaggtggagacagatgatcttaggggcaaactggttagctagactagtgactcagcaggctctgcttagtgagagaccctgactcagtaaataaggtggggtgtgatggagaaagacactgactttaatccctggccccactatacacatgaacacacggacagacagacacactctctctctctcctctctctctctctctctctctctctctctctctctctctctcacacacacacacacacatcgctctgagccaggggctggtgagccaggctgggcaatggccaatgcccatgttaaccccaggttcagatctgttactctgctctttggtcaactgtaagaccttgcaagtcacacaatctctctgagcctggatgaagggaggcatgtcgtgagggtgaggtggagtgggggctgtgtcctgccactatgagaagctggagtacaggagccctagtgctccccaacccaactgcacgtgttgatgagagtccctgggagcggcaggctcaagggaactggaacaccagccaagtgctgtgagctcagcgctgctctgtggaatatgaaaaacattaaggatttaaaaaaaaaaaaaacacacacacacaaccttctgaatcgaatttaaaataacaaaagaaaaacagcttttgaatttaatgtaaacggaattgaaatgattcccttgttggaatgagtatcagataaaatcaacaaggactctgagaaagtgtccagactgccttttccaagggatgtcacggagatggcgagctcctcagtggagcattaaatggtcctgtctctgttgtgtggctgatgtctggccagagctggcattgccctcgcagggacggtgtgggacagacctcaccagggtactatcctgccaagtgggttctcccagtggaaggtactccagagaaaggatggctccccctgaagagacgccttcccttccccaacctctcagcacagctttactgctactctgggccaggtttaaccagagcagtaaggggatagataggatgggatgcagtatgggacgcagccttccccagggtaccatgaaagctgggctctcacagattcgtaggaacttctcagctacagaccaagaggaaaggcattgtagggaaagggatgataatgtttacccttgatgggtgtgtggtagtgctgccttgggtcttgatgcatgtacaatgtctagcagcagagtggagattctaattgagcctcagcaatcatcagagtgcagtgagcaccttgcactcgaccccagagtcagcggagagtccctgaagggtcctaagttggggagtgacatgaactgacatgaagagaagacatcaaagcctttgaaggggactgtcctaatccagagtcccttttctcaagatccttcagagcagcacagctgggactgtgtcccagaagcagggcttggctgtcagccagcactgggcatcctctggctgcgcccagttggctcagcatctgcacacaattgggcattagagcccagtccccattagaaacgatgcccagcacatttaccagtggggaatggagagacgtttcccatcacgtggctaggtaaagtttgcacaactttcctttgttttccactggcccccacaataaatcttcctcctctgccaggatggggccacggagatctgtgagtgattagggcacggctccacctccttgccctgcattcttcctgcatgggtagtggtaggatgtgctgatggatctgcgactatgtggtcttgcaagggtctgggaggtgagctatggggagtcagtctcctgccctctctaaccatggcagctcccttggcggacctgctgtgttacagacataagctccagggaacaggaagtgtggtcaggctggccagagggctctacaggactgtgcgtctgggtaagacctgagatggatatgaacaaggctagctgagtgatatttggtgggaactgcctaccagccctttgctttgtaggatccataagacaccagggcctagaagtgacatttgcacctcctctggctgggtttttggtaggcagtatctgcttgggaaatgtgcaggccatgggtagtggaaacactaaaatctacaaggttacagccatggccaagtttcagttccccatgtcctttcccaactctctacgctgtgaccctcccacgacctgtagctactgtcacatcagcactgatccatccagctatgcatcggagcatctgttgacccacgtggtttgggggtccatttggtctgcagcatcattgctgacagacttcgacgctaggaaacatggttagaactccaggagtctttagcgtgcctgggactttcttaggctctacactcccatctctatcttctcacaaccatgtcccctctggcatccagaacgggggaagggattcacccagaccacagctctctctcactgcctgggtttattcctttgctatttgcagatttcagaaactgatgtccatagagagggttcggctccctgcaggtcaaggaaccatgtcccaacatgcctcctagacagggttgtctgtggtgcctactatgtgctgggccccatgctaagggctcacatgcctcactgtcaatcctgaagcacactggccatgtcatatgcactgagaaagaagcttccaggttttggcgtgtcactaagggttccttccagccaccacgtcgccccagtagctgatctgaaggtgcaggacatgtaaagatgacctgctgagttcaaggcaggaatggctaacatatgttcaatagaatctcctttgttctggcaatctggtactcaaggcaccgcctggctgcctcccgggagacctgttgagcagtgggttcatgcctcactcctcctgtaccctggagcagtgttgtgactgatggccatgggtgtcaggcccagaggaagctaggtgggtgcaaagcccacacaagcgagctgtgcttctgctgtgggctctaggagctacctaactgttgggacctcagtgtctctatgtggggcaaggactttgattagatgaagtttatagctattctgctcctgactcatccatccatccatctcgaatagcacagagcaggggcacggggcacgttctctccccactttcgcaggcaaacttatgtggctggagctagcaactctacccctttggagcctcattttccctcatttgtaaagtggaagacctggactctagcattttgagattttctggaactcttcagagactcatagttacatccccaatgtcctgacttgacctgctggaggaaagagtgaaccatagtggggggagcaggcagggtcctcctggaccccaaccctagttctcttccagccatgctggcctcttggctgcttctcaaacatgctgagctcattagaaccacatgctttctgcatagctattccttctgcccaaatgcccctaccccagtctgacctttatgcattcaaaccccagctcctatgttccctcctccaggaggccttccctgacctgtagcccactctctggtatctgtatgagttcctacttcgcagccattaagctccggggaagttcaagactttttaagatttttgtttcttgctgtgttttcagtgttagcaccagttcccagcccattgtaatgtgacccacaggtcacaacgaatgtcaccaaattaggggagagtggtccagaggtgttgagggaaggtcttgggaggggcccaggtagggtattagaaggcttataacatatgtgtgtgtaagggactcatcagtgccagatgagcctgagccgggccatccaggaagcccagcaccagcagcatggtagatgctgagcacttcccagcctggttccttccttgctccatctgctgcactagtccagaattcctggagtatcagaccccaacaagaccccatcgatggccccttaaccagctaggaaactcatcctggttcccttgatggtgctacagactggggagacgtttgcagtgaggtccttatccagtagatgtcccgtgtgcagtcacagctgcatctggtcactgaaccacagagcctggcttcatggccaaggccatagctggggtccaattcagcattaattcacaacacaaagattcgtgcagccttgggatgtagcaggggacacagtgacactacctacagctgccattgtgaagagtgggaaggagccagctgtgcactagtccagaattttcttcagagtgtgccatcttctgagagcagaagcctgccgccgcctgtgattccgtggcaaatcgtttgaccttggcggcagtccactttacggatgggaaaaactaaggctggagcgcagtccctggccttaccctgctctggttacctgtgtcaggaggttctctgtagagtgtgagatggaagtctcctttctcttgtgtttaccctggaggcctaggaggcagccattcctcaatgacagtcacctgggactactggtggccttggagctgatctcagcacttaatctgcacccaagaggtttggctgaaatgagaattggcaccataagcaagccctaggcttcttccttgttgcctcctaccttcaggagggtggccttctcacaggaaaggaaggggaccagaggtgggggtatgggatctggggggtgaaggtgtactttggtctgagaaaggacaagaatctaggctgcatataggagcagacagactgtaaggagccctgtccctgtgggcaggagttggcatggcccagagtctaggtaagtcctaggccagaggttctcaacctgtggctcgtggccccttgaggtcacctatcagttgtcctgtataacataactgcatgtcagatatttatgcatatcatattactgcatatcataccagtagcaaaattacagttatgaagtggcaatgaagtaatttgatggttgaggggccaccacaacacgaggaactgtactaaagggccacacacagcattaggaaggttgacagccactgccctagacacttacagtggcgttggctgggctaccatcttgttcctggctcagtttacctgtctgcgctgtagaaatgataaagccagcctgtctgcctggggttgtttacctaccctcccgaagattcatcctgagacgattgttcacctctccatcaggacacttggatagtggagcaggaagtggctgaagttgcaggcatcacacagctaccagtggagaggcccagggcaggccaggtttctgccacatgtctgctggggtgaagagatgattccccagccagctctgccctcaccactgcctctcagtgagtactgaggaaggcaggtgtggtccccagagcagggggctccgggctccaatcagctccaatcggtaggaacagtagagctgccgggcgcccgcagtcaacacctccgtaggaagcctgctgcttgctcccggaaagcaccagccctttaattgtctccacattgagaaccaagagtgtctgccctgatgagtagctggtgaaatcaaccagggaccccctcccacagcccacggggcaggcggtggttcggccttgcagagctgagctgttggaggtggttgctaggatccaaattagatgcacctggctgggggcctcacttcctccataaattcatgatcagtggagagagaagcatgggtgcagccttgggcctcaggtgagccgggaaggtgggacttggaaatgtgcatttcctattggtgaaatgggaaaactgagccagacctcgggtctttaaaaaagttggcatagagatgaatggaaaggctgggtgtgtgggagtcccatcctcactggcctcccagggaggcagggggagcaccacagagtccactctgatggtctagcctgttctactctagatcattctctttcttagcagtgagtttttttcttgtctttgtagcttttgcaagagaggatctacatgcagatacattgtcacctgattagagaatgtatgtggacaatctaagcttgtttagttaactggagtgaaccctagggttttttttgtctatttgggggttttgtttgtttgttttttattttgaaggtttcttgtagccaaagcagagagggcaccttgttgacttgcagagtcccttttgtctaggaaaggcgaccagcagcttgtgtgttaaggctgtcccccgccctccccgattctgccccactgtgagggcttgtgggagctgatgtggtttcctaagaagtcactcgaccttgtgttccataagttactctgcttggcctttgagagaagccaagcgagtgtgagttgtggaggtgaggaggaggcacagtgctaggagatttccgcagcaaacagcctctctgcaggctccaaggctcaggctgtgacttggggctgacagagccacttttcaaggagtgtggagcctggtgcactccgtggatcttaagaggagacactacgattggcctggctgagctaggtcatcagaaagcaccaaagaaccggtgagaggcacccaggaagaggcaagtgcgtggtccaagtctaaagggtagttgcttcagccaggcctggcaaaggaggggagccactgcctgctggggaaagcctcctctggctgtatggggcccatgtacccatgggtgttcttggcgactcttacagtctgcgggccctgggcctacaagcgggaaacctacattaggttctccggagctaattatcgcatgcccatagccacctgtagtctgtgatgctgaggtcttggtgccctaggggtcaccctggcagggtgttccatctaccgggctcctcggatccaggctcctggaggaggcatcactctaagcaacagtctggagtatttggtgtggggcacacttgccattcaaggatgcacggggatctgagagggagcacggagaggtagggacctggaggcgctgagttccgcctgagttccttgagcaggtggatggatgcccacgcagtgaccagactgcttgggtggaagaagacgggcacaagctggagcccttggcccaatgctggcccagactgtgtgtagtcctgtgtgtatgtgcacatgtgtgcagcagaccacttgccctccgcagcaagcatccacaggaacgggcagaggtatgtggggatttgctcagagaggaggggttacaacgggtcagcagctgggagctggaagacagaccataatactcatctagctgggtaaggatggaggcaaggccagagaggccccgtgaatggagaggtggcctgcctaccgacttcctcagtatatgcatgtggcagagactgccaggacatttgacgtgggtcttctacctcatcggctaggaacaatttccttttgtggatgagaaagatggaggcacagagaagttaagtcacccaggaatacacggctggcactacagctcagatccaatctcgaaggaaagtgtgaatctgggcggggctgtgtgagggaagtggagtggagatggcttagggcaagaggctttactaggaggtctcgcagccagctggcccgagatggggaggaagaccagactggagaacagacccttgctgagggtagcatgggcgtgagggctcagggcagagctccagagactagcgaggagcactgaggggcttctgcaggggtgggggtgtctgaggtgggacgccaggcacatcgggctctcctgtgagaatgccatggcgtcccaggtgcagggcgagcagtgtgatgcttgtgctcttttgggctgtgtatgtgtgctgcatgaggctgcacctctgtcttcactaaggtgattcactaccaatgagttgagcacctccccttaggatagactgtccccacttccgctcacgtggagccctgcttcccagtggtcttactggtcctagggattctgggtgtcttctgccagttcgtgacgtacagttcgacttccctgattgcatttgtcctccagatgagctcttcgctttaaagaattatcatccttcctcttgtggtgtgctttgtgactgttgcaagaactctttacctgaatctgtcaacaaattgtctcgggttgttagggattcatttttaaattttgctcccactttttgagtttcagataggtagaaatccagtttccacctggtcatccagtcatttgagggtttgctcaatgatttatccatcccccacatcctggtgacagtcagtttcaccccggcacatacataccaggcttctctgtatttatggggctgtttctaaggctttggttgcaccagttgggttttctaccctacacaagctaccacatcacctttttgtctcttggcattgactttacagagttttaaattttagtgaactccaccttgtgaaactatttcctgtccatcaccaaacctaaagcctaaagctggtctcctctgctgtcgtctaggggttgcatagttttctgctttgtatttagacccatgatccatttctagtttttcaagctaatttttgtggctaatgtaaggtctattctagatatttatttatctattttattttgtatatggatgtccagttgtagcacatttgttgagattactgtctacactgtgtttcttttgcttctctagctatatttgtgtgaattcgtttctggactctttagtctgttccactgatccacttgtctgttgttttgcccgtaccaaactgctttgcttaatgtagcgtcataatgacattatagtaagagttaaagtgtagtagtgtcagtgtgctgaggttgctgtgttccttcagtattgtgctagctaccctccctgtgtctttacctctcctgataaactgcagaatcaattcatcaacatccacagagtagggccgagcagtagctcggtgatagagcctgtgtgtgtgtgtgtgtgtgtgtgcgtgcgtgcatgcgcgcatgcattcgtgcacaccagtgggggagcgcattgtgtgtggggggtgctccgtggtattttgactagaaggacattgaatctaaagtcagattgggaagaactgccatctttttgttttgttttgtttttgttttttgagacagggtttctctgtatggccctggctgtcctggaactcactctgtagaccaggctggcctcaaactcagaaatccgcctgcctctgcctcccaagtgctgggattaaaggcgtgcgccaccacacccaactaagaactgccatcttgatagcagtcttatccatgaacatgggatgtttccccctttcctcttcgtcagttgcttttatttgttgttttcctcccgtagatcgcacgcacgggaatgctccttcccttacgacagtgtcactggctgataaacttttgttaaagttgagaaattataaattgaatcttactagtttggggactttctctaggcttatgcccaactgcttcatttttaaggagtgtcgggttttaaatttcagaattcattgttcattcctagtgtctaggaacatatagctttttgtacattaaccttgtatcttacaattgctgtgatttgcttatgccagcagttgtgttttgtttctagtttttacacacacttccgtgtcagctgtgaatggcctcacttcttccccagtctgggtacattttctttcctttccttgtcttgttatccaaggcttcagtacagtgttaagagggatgtctagagcctcaatccaggacattctttttattccttatttttcttaagaacattgagtggcatcaactcttagactgattagtacacatccagaatatttacatatatagtatgtgtattgcttaattttcaaatgtggaattaaagttgcagactttgctgttaaccctaagagcaaagccactgggttataatgtgctgtcccttctacagtcaccactagtgatgtcaccacgagtgatgtctgctgtgttctgatttctcacacatggctcctctgttctgatatcagggttttaagtggtcttgcagaatgagttgaggggtatatcttttatattactggaagcatttatgtaagactagaatgacactgtctttggacatttgttaaaacttgcctataaaactgtcttcatttgatgttatctttgtgatattttaagctactgcttcaattacgttaattggcatggaaccattaagacctctatttcctttgaagtcatttcccgccttcaggaatttgttgattttgtgtatgttttcaaattcatggccatgaatcagtcacatcttgctttttcgtcttgggttttgttttgtggtactgagtactgaacctagggctttgcacgtgctggacaagtagtttcccattgagctataaccccagccctttcttgccttctattttgaaacaagttctctctaggttgcccaagctggctcaatttagcacccacctgactcagcctcctgagtagctgggatgacaggcattgccaggtttcaatcagtgttctcctcctcctcctcctcctcctcctcctcctcctcctcctcctgctcctcctcctccttcgtgtgtgtgtgtgtgtgtgtgtgtgtgtgtgtgtgtgtgtgtgtcccatatgtgtgtgtactatgtggccagaaaagggcattggatcccctagagttggagtcacagacggttgtgagccacctctccagctccggctcttaccgttgaaacctctgctggatttgtaatgacagtgctctccttgctagcattgttcatgtgcgtgccttctccctgtcttcttgctcctttcccagaggtttctctcacttcctggtcttttcaagaacgagttcctgacttctttgtctctgttgtctctgtttagaccacatgagcttttgcatttatctttattatctccttcctttcctgtctacagtgagtctcctaggacttcagttccagcttccttcttctcctggtggaacagtcctgtacagcgcacccctcccctctggcacccccgtccctgtcctcagaaccctggtgcggctctggcctcactgctcagccccagatgaatatcactgtactcgttctgatactttggctgaggcaggaagtctttcattttccaaacatagggacatttccccccttttattgccttctaattaaattccactataattgaagagcattatttacacggtctgacccttagacctaatatatggcaggtttttgtaaatgttgctgtgtgcttgagggaaaatgtgcacacttggtttttattccatagaaagtcttctcgtagtgtccaaattggcaactttattcacttctttttgcttaacatattagaaatggaaagaagcgtgctctcttcccccgtggtggtaacctgcgtttccatccgtgtggctggctttcattgctgcaccgtttggcctctagaagtataaagctttttatcattagaaaacggatctagtaaagtttttttccccctgcaagctctacagggtcagatactatcgtggctgcaccagcctgtggatggtgtgtgcctgggacatctttgctaccctttcactttatccgttttacattcttggcttttaggtacgatcagtggaattggcaggtggggattatttcattgtgttgttctttagtcaggtctctttcaattagcgtgtggccttttcagtgctgtggtgaccaatgcatttgggcatgtttccctttttattgttggtagttgttgtttaaggctgggcaagaagtcagcaatcctctgcctcaaactcccaaatgctgagattacaggactgcaccaccacagctgggtccatgtttctgatttttttttaagattttatttatttattatatataagtacactgtagctgtcttcagacgcgccagaagagggcgtcagatctcattacgggtggttgtgagccaccgtgtggttgctgggatttgaactcaggaccttcggaagaacagtcagtgctcttacccgctgagccacctcaccagcccctgtttttttgttttttgttttttaaagatttatttttgtatatgagtaccctgtcactctcttctgacacaccagaagaaggcaccagatcatgttacaaattattgtgagctacctgtatgtggttgctgggaattgaactcaggatctctagaagaacagacttacatttttgccttctgctttttcggtgcccccatttttttccctgagtttctgttaattggtttatttttctgaattaagaacaactattcgccccgcctcccgcctggagtcttggaagttatatactcaagttcttttgttggtgattagcttcagattttatcagacattcttgacctaaaagagcagacagtgagtgagcgctctgcctgtgtctgtagctgcatgcaagcttcaggccactgctggccacccggtccctaccgtccctgtgtgccagctattgcgggggctgaagggctctgaccctggcctttgcaactgccacatgttccgagcgctgttgtttcatggcagaggcgccgagctctgcccagcaagtgtagattcagctgcacacccccacatcttacgcagctcacaccttccttccgggatcggtttccagcttcctggtacctttgcttgattggcagttctttttctcagtactctgactacattattcccttaaacctggcttctgtcttggcgatgagcatggtctcgaggttgtactgtgcagcctttctggcctttgctcttatgtgcttcaacattttggtcctctcaagtctctgtacgttcgcagctctggttggtacacactctctgtggccctcagcttttcttccacaccatcttccctcctgaggctctgactagaggcgtgccctctcagcctggcgtctagccctctggttggaattcagctcctcctcctgggtcagtgctgtctaagctactgtttacctaatttaagtcacgaagacacttttaattaaaatttttatttttaattctgaacttttctaatactgctggttggttttgccagcccctcggtataaggtcatgtttaaacttttacttgtggcagagagtgcacagctgattcttggctgtggatgtcttggctgaatggtgtgtcagtcacttcccaccccgcccccttttcgagacagggtctctcagtgaccctggagcttgctgactcagcagactgactggtcagcaacctcagaagccgtcttgtctctgcctccccaatactgggattgcaatgtgaccaccgtacccactttttctgtagagagccgaggatggatatcaggtccttgcttctgtggcaagcactatgcagactaagctttctcctcaggctcttcccacgtcttgtggttcagtctttaaacagttccacacactgaaacccacgctaggcgacgtctgaccttgaggctcatttcccaccggggttctcccatctgcctgccttcttgtgtttatcatcttcagtcgtgagatggtatagcatgcctttgtcttgtaaaggggcacagtggttcttccttatccacacggtgttggttccaggacctcccacagatcacaaaatctgcagatgctcaagtcccttatgtggaatgctgcagtgtttgcttgtaatgtgtgcccagccttcagctactttaaatcatctctagattcctccttacatctcatctatcccgcaccgtaactgctgtgtgcaaatggctgttttgctgtctgcatggagaataatgaccagaaaaaacagaacatgttcagtacggatggagtttgggacttaggggacacttttcatccttggatgcttgaccgtacgcaggaagcatttgagggtagcagaggccaaccatagtgttaaatgtttctgcaaaacactgagaacccggtagtcagccacttcagagcccgactgtgtctcctgtgcaggccgaggtaaagatggagctgaaccccagaaccacctgcgacctgctgccaactcctgtcatcaggcaattcacagccaggctttaggctgcaactttctggttggtttcttcccagtctctcagaacgtcctgtagccagcatagtaaaacagtccacagttctgcacggtttggttgtgttacggcagaatggcccaaaacccataccccaggccctggggtaaagtgatccgcagtgactgcagctaccccagacgctgtagaatggatctcagaagttagtaggaggcagagcatgaagaagcgccttttaaaaaacgaccgcgtgcgttgctgttctgagtgtttgccatgcattatcaccccaagccccctcgcagatccaggcctgtcagcacccgcatctcaccgtaagaacagcagagaaggcccaccttcagcacagtgtcacggtaggttgctgttggaagaggaggaggagttgccaccatatcatctggccagggagcaaggctgcgtccttgttgagggacagtatggtgcagtatggagtgctggctgtgactcagataggcaagggatgcggctctcacagacctttttcagggtgaccctgagtcagtacagcttgtggcctggcctgcaggtacctcatcccctggcctcttaggagcaagcttggggtgggtgcagggatgtttttgctcacgtaggagtccctatgaaggtgtgacccctgttgtttcatttatgtgattcccctgctctggtatgtccccctgtccttttgtgtcccatcttcatgcagaccctttgacacctgcttcctgtcacctctgaggaccgtctctgacctttctcgctggttctctgtcactcactagggcctgtgccccagcaagtgtttaccatgtcaccatttggtccgcagcacagcctgtgcgtctttgtggcatggggtcaccccgcttctgcctgctcacctgcgctctgctcacacaaggctgaccactggccactcacatgctctcgtgttgcatgaatcatctttcttccgtgggcagattcttggttagttacaaaggtctctgtaactcttgtctgcttcccgctcttgtttttgtgtgacagggaaagggccttgagacttgactcccccccctccccggggtttaaaccctgccctcaccacttgctattgtgtggacatgacttctgtgagctttaccactgcagtaggaagaggtggaccatcctcctaggggtgtagagagaagtgcatggagaggggatgccctccagcctccctgcccccctccactgctggcatcttccccccaacccaaacaggctggctctccaggcttaagctgtactggcgtcacatcgtatgatgccactctgtggggaactgctgagccctctgactcccttcagcctcagccaagcctggcgtgttctgagacttccccgagtgtgccccttgccgggcccattgctgtggtgttcgcctgacgaggaggcagctgccagcccactgtttaccttgggcgcaggggctcctctctggcaatgtttccagccacagagcgagggcagatttatggactgggttacacagaaattatttaaggccctggtcggccaacaataatgtaaagacacacagtgcctgtttggagacagattgcagtgccggtgcccacactgcggtctgatttacagcataggctgacgatccaattagcaggcacagcaggctttgagctgagaccatcgccttccaggattatgtagactttccaggtcccagctatgatgaaattatagctggaggcttgtaaagtcatgcatggcttgtaaatgcagtgtagtcttaactgccctcctgctcagcttacctgcaaggcccaggtctagatggttgttcagaaatcctgggagattattgcagatagacctggctccctctgacttactgtgtgaccttgtataagtgcctaacctctctgagcccaattttcttttcttttcttttcttttcttttcttttcttttcttttctttctcttctcttctcttctcttctcttcttcttttcttttcttttcttttcttttcttttgtttgtttggtttttcgagacagggtttctctgtatagccctggctgtcctggaactcactctgtagaccaggctggcctcgaactcagaaatctgcctgcctctgcatcccaagtgctgggattaaaggtgtgccccaccactgcctggcccaatcttctcatctacaacttgactaatagggccaagcgagacttaaaaagagtgacagaaacttgtgaaaccaaagagaccagatgaagattgtagagaactcagctgcatccctagcgtgcataccataatggagtgtgtgttcaagatcttaaaaagaacaaatatcccatggcgtcctctctggttatctgcctaggggcaagacagaggaacatgggggtactgaagactaaaatactgcccacagttcttcagatagcagggcctgtggtcctgcatggtctatgttagtccaggactcatcagctactttcatctacttacttctgaggagccctctaagcagatgtagtcagggtcttcctaaagcagctgcttcatgcacgaccacccctgtgcctggagcaccctggacacatgttgtttgcagattcatgctgaaggcctggtagagctgttgtgttgcctgtgccctaagggaatccaagtgtcaacctcaccagcagcctgccccagcctgtcacatatgccccatcccccacaccccctccctacccagaatccctctcactgagtcacaaggactgagacctaatgaccttgggagggagacatatgtccattgaatgtatgtatgggtttccctggtcaaacctgcctacagaagacttttatctttgtaagctgggagtgggttcatacctgcagccctcgatgtagaaagcctagaaacttagctgggaccccagggacaagtcagcacccacccaggttctaggcacagctggcttttctttgctctgctttgctgtgtgctgcaaacccctggctcctccgtagattcttctcgagtctgaattatggaggtggtccctgcccgcagtgaagccctgcgattcagaggttctgtggtgctgtgtgcaaactgagttgcttgggatatttttaggagagtccagaaggcctctctgcttgagcagtcagggaagtctccccagaggagaaagcacttgttctccaccttgaaggatgaggggttatgggctggaggaggaggagcaggaagtcttttccaggggagagggcactgtgaccaaaggggtaggtgtgtgcatgtgtttgtgcatgtcaatgaatgcagttgtgcatataaaatatctacagtgtagattgtgtgggtgggtgtgagtgtgtgtaaatgtacacacatgtcagtacatgtgtgtacagtcatgtcagaaatgtgtatgatgggatggatgcatggctgtagatggtgagacaggcggatctggggaagagtaagagccaaaagcacggatctttgccccagtctacactacaggtgtcataagagccaaaagcatggatctctgccccagtctacactacaggtgtcataagagccaaaagcatggatctctgccccagtctacactacaggtgtcataagagccaaaagcatggatctctgccccagtctacactacaggtgtcataagagccaaaagcatggatctctgccccagtctacactacaggtgtcataagagccaaaagcatggatctctgccccagtctacactacggtgtcatgctttgacccaaaggacagaggaagccagactctggtggcccggctggagcaaggcaaggccagcagtgggcagtcacgggggaccaggggcttaactagcagctgaggggagtgagagcccttctgactcagagtggaaaaactcaggccagcgctaaggctgaacaaatgccaaggaaggattgtggcctgaggacttttggattgacagtctctctccatgtgtgacaagccagcagttcccagtgcctacccattctgtgtcaggtcagcctctgagaaagactggacacagccaataaaggcatgaccccagccctcaagagagtgtgaacccaaaacgatgggggtctatacaaaatgttggaggccagcaaaacctcctacactttattatttctgagaaaccccgaggttctatggaacattctaagcccttctagctctggtctgattcttaacttcttacccctatcaattggggatttatatctttccttcctcatttcagtttctaggacaggcctgggactgctgtgtgctgtccagatatcccaaccctcaggctgtagggcactgtgaagccccagaactggctggagcctggacacatgtggcaggttaaaaagcactggccatatcttagaaggctatacatattctgagacagaagagtaaagaaacacagtctgatggtgccccacccccatgcctacacccatgagaacatatgactcaactcatgggggaagatgggcacccattcatttattcactcactcactcattcattcattcacacaccctttgatgatcgccctttgagtcaagcccctgtgggctctatgacacaagtggaatgttcgccttgatcttgacagtgtagtgggaaagccagacaggagaacaaggcacatgggcaagtgaggaactagttgtaatctgttaggtgtggatacagtatgacccaggagtattattgtcttcaggaatggcaaggatgagattagaaaggtcaggagggtccaggctgagttcatacacacggaagacctagtgtttagtgtggaaatcatgatgggttacaaatagaggcctctccaagctaagctcacttcgactgtactctgcccctctctgaccaggatctttgactgtcccctgtccttcccaggctaagatcactttgctgtcgtctcccttccgtgtcctttgactctcacctatgcatccagggcataagctgtgacttggtccagccattctcttgctcatcacacacatttgacagtgagttctctaagcccagcagacatggcctgcagcagcctatggcagccaccagcccctccagctcatccctcatcctccagggcctggtcctgagctcctgagtagctctgtccctctttgattaggggctgtcctaatgacatttgattggcagtaactggagtccacgtcagctcaggcggaggctatttctgtgttctctttgttctgctaacaatgacttcagaagggcatatggaatggataaggggctatgtagaagacagaacccttcaagacgctagagataattagacaggaactatttagctattttaacgtgcttcatttataaaaaagacatatttacctaatctgtcagattttgcaggaagtgaattagaaaaaatagtctgaaaagtaattggagtgaaaagatattttatggtagctgcttcatgctctaagtactatgtgactgtttcaaggcccccgtgatgggccccgagagccagatccacacttcccaagcgaccctgaacatcatgctggtgcctagaacctcagaggcatctaagcttcatcttgtcgtctctcttgcctgtcccagtgttgaaagccatctctgcccctctctctggattcagcttccaatccaagtggcacagaagagttgcacaatgctgtgcctcggtttctcaatctgtaaagtgcagatgataagcacactggagtgattggatcctagggactgggtgatatagcatccatgctgtctgggaccttcaagcccatagagactgagtagatacactttgttaccagacccagtccataagaaccttgtggggatggagggctctcctggtgctgttcacgtctcccgtgcagccctagccaagcatgaggctgctgacagaaggtctcggtggcactgtccgtgagaggaggctgcccgtggtcgatgcttcccttcttcccaacctggagggaaaccttttctgctcaatgccatttctatgtgctggcttcccactggtcactcttggcacgttccacagagactctttccaggcctctccagaggccaaaggacaagaccccagctccagagtatggtcagggctgtgtcacatgctctcacacacacagcagagagcctttggttcataggtgttctatactccgtgtttagaaagctccatgacctgtgtccactgcaggctttccaaagctctgcccaaggggacagcagcctcatggcagaaacagagcctggttagagattgtaggagctgagcaagccagagaccccccctcagctagcacctcgctctagagagagagagagagagagggagggagggaaggaaggagggagggagagagagagagagagagagagagagagagagagagagagagagagagaaagagacttggtcaccagtccttagatgtgaagtagatagaaatctttctggaacattctggcctcaggatccctgagcttagaagagctggtagggagcctggaggtggtggcacacgcctttaatcccagcactcgggaggcagaagcgggtggatttctgagtttgaggctagtctggtctacagaatgagttccaggatagccagcgctattcagagaaaccctgtctcgaaacacgcctccccccaccccaccccaccccccaaaaagaaaagaaaagcaggtagagtcacccaagtgtgctcagagatagttttagacatccagagagtggctaggccactccctgcttttgtggccttcaatgctatactgagaaagcatcatcgccatatctctgaggctatgataggacctgggaaggcagtgtgttctccttcaaggtggcatccccaggtccagaggacctacgggcaggttggtgggcctgtgttgctgcctctgcaatataaccccaacccatccctacccacacccttcccctagagtccctgatacccacagactcaaacccagacccttcattgatactaacaacttagttagccctgtggtaacagatttgcattgaatccaagctcttcctcatagttccaatgttactttagccatgtcagttgtcatctctgaagtccaatctctttttgtaagctaatgtgtatgtatgtgtgtaagtagccaggaacctgcctaacctagtgttcactcctccggtatccacacattgccctgcagggctgaggtgggatcttctggacaccttgaagaaagaacttggttccaagtcttgttcatctcacagttcatcatttgacccagatggctgacagctcgcccagactagtagtggttagtctggctttctctgtacctggaggcgcccctgacagggaagggggagggaggctcgctggagccagctgggactcttccttgtccactctgacccagcactcactcaccttctgccacatttgggctgggacctttgaagtgactgttcccctaattagatagagttctagaactctggagcctaccatggtgggggtggggcggggcggcaaggtgtctcttttcactcccagccacctccctgacaaaggtcatcttagtttgtcacatcacaaggccagcggttttcctaatcatattaccctgtctgaccttgtctgctcgctgaattggtgtctgtgtggcctcactagttagtgtgtgacctgtagagggcagagggcacatgtgtgccatttcccatgctgtccatcagctggggaaatgccctgtaccttaagacactttacaggtgtatagtgaatgtgtgtgggcacatgagggcacagcatccaagaactatgtgaactgcagctggtgtgtgtgtgtgtgtgtgtgtgtgtgtgtgtgtgtgtgtccgtccctggagcagattcccgtctcctgggaaatctgagggaagtagcccagaccgagagacggtgccttctaagtacacctgcccaggtacagggtggtgctatcctgctcttccaccagggccacccagagctgtggggtgggtcatggggtttgaggaaaggccaggaaggctggcaaccccacagctagctagtaattgcttgcagcttagactccgtaggccaaggttgttgttctgtgtttttaaaagccaggtaaaagaaatctgtgtcggtagcctcttgcctgctcaactcccatccaaggttgaatgataaaaatagaactaggaagcctcacaaggggccgcctcctgtgtggctgcggctgggtagagggccggaccgagcagggtgggggtgggaggctcttaggccatgggaatgtttgctttgcttcccccaagaactggctgtggccatgtaaatcacccggaggagatctagagggtcccaaatgcatctgttggttttcataaagaaagaaagacaaagacaacaatctttgtggtctgcataaagtattttaatcattaatgaacaagaaagagcaaacccctggagcaggcaggaatttgagggggaactggtgagggctgcccatccccctgccacctgggccccctctgtgcagagcaggggaaggcctagaagcagcaggagtgggggaggggccgacaaaggatttgaagggacctgcgggggttctacccactgtggccataccgtccatccttcatagcaagctgctgagcatgtataaggcaggagaatgccacagcagagagtccgggggtggggtttcaggctcagtgacagtgtacatgcatgtatctgtatgtgtggtaacctttcagctgcttaggaaatgtcatttgtctacatgattacatgtctactgattgtgtgtctacatgactatggggcaacataatctgaataagtgtggggcattccaagtgtgtcggatgggtatagttacgtttgtggttaggaaggctcgaagatgtaaaagtgtgggagtgtaagtgtgagctttagtgtggcgttggagagtttgtattcatgtacatctgttgggtgtaggaggttggtcccttatgtgctcagtgtgctgtccataatgactgacctggcagcttcctggcagcctaggaatggtcaggacagagacagtcatcccactgacaagagggcgtgagtctggaaggaatgtcccttctgtggacttaaggtccactggttaggtatgtgcatattggagatgtggtagaaacaggtaccacatattcacaagaaccattacccatctgcatccaaggtactcacgtcccaaacactaccccagacacacttcttaaccagtgatgccccgcgaacctggatgtgtataatacgtgcattacatatatgcaatatatactgcgtagactggtaggatatagatagagagatggtagagatagagatattacacagactacaggatgtatgtgttttgagtgtgtttaggtataagtttcaggaggttggtatggttctaacatatgccaatgtatagggagtgtcacagggctgtacagtatgagtatgtgcccactagatatacatatgtacttgggcatgtgtgtgttagaggaagtgggggaggatattttcagtgggtggagtgcatatgtagtgagtggagaaaatgagcaggtttgactcagcctgtgacagggtagtgggatggttagctttgccttgaacaacatgttaggtgtatatcatgtgccatatgtgttaggtccaaggggttggtccatgtgtgctgggtgtgcactgggtgtatttgaagtacatgtgtattgtgagtacatgttgggtgtttgtaaggagttggtgtgtactgggtgtgctttgtgcacctttggagtataaaaagttggcgtgtgttccttgtttgtatgtttgttgtagaggttggtgtgtgcatgctgggtgcttattgtgtatgtgttgggtgtgggaagatactgtgtgtgctaagcattcatgtacatctgttgggtgtaggaggttggtcccttatgtgctcagtgtgctgtccataatgactgacctggcagcttcctggcagcctagaaatggtcaggacagagacagtcatcccactgacaagagggcgtgagtctggaaggaatgtcccttctgtggacttaaggtccactggatccagtggtggctggggtactcttcttcagcaagaggctccatctgggcataggttgtggggtgtgggtgggccaccacaggttgcaagatgatggctcctcacttccccagggggctcagtaactacaagaagtagattactctgtttctttatataaatagcattgtcatgagaaatgtttcagagcaatagaagaggactcccagtcaggtggaggagactgccagatgggcccatttctgccttacaagagggagtgctgagttcagtgtctagactgagcgaaaccttgccaggctatcagcaggacactccacagagaagagctagagagagcttaaggcccagcttgccctcgtcttggatgaggttgtacatctccctggttctcaggttcccactggggttgttgaaaagattatgaataattaggattgttaagtgtagtgttataaaaataaaatctcccaggattgcacagtacaccatgccatcctttttcgcagcagtgttatagtcccttatgtgagttgagataaaatgtacctagtcaactattggactagtcttctgctatttgtattttataaccagaacatccattaactatcttattacataaaactttgatctccaggcccacagtggagaatttgaacctgtccctatgagtctgtgttcatccatgcccagttagaactctggagtcagagttcccctggctgctgcaaggccagttcccaggatgtacgtgcctgagccccctgtgatgtcattagcctggagcatcattgtccatggtttgcagggatggatcctgagccacagcctttactctctgtaagagaatagtccttttcttacccattagagcatgcttgcctgtaaggaatctaactccttttgggataaaatcaaatccagatccctcaggattggccagaggggagaggaaatccccaaactgggctcagtctaatgaatctaacacctgctgggagagccacacctctttacggggtcatctgctactcgaaaccatgtcaaccccaagaggctctccttcctagaaggagttttggcagggtgggctgtttgtcctttgtgccccaagatctgctctgccactgactagatggctgaaatgagcaggttacagctttgtggctgcttgtctgcagtgtggggacaatgacagcagtgacctgtggagccatgggaggaagatgcttctgcctggaaaggcttcagcatggccccacgtgtggtaagaactgcacatgtggcttttgttcagttcaaaagcagtcacctctgcagtcacagatccgggaaggggctcatacctgggccatattaataatctcatagctctgggtgggggagggtataggggactttcgggatagcatttgaaatgtaaatgaagaaaatatctaatttttgaaaaaaaagaatctcatagctcaatgacaaagagacaaacagtgggctttttaatggacaaaggcttgagtggtgtttctctgagcagtcatagagaaaaagatgtttgatgtcattaaccaggaaaacagaaatggcaatgagaagaggcttggcgctacttaatggcagtaaccaaaaccatggttctcacacgagggcttgggaggtggggagacccaaggaccatcccacggtgatggtgccctggccttagaaaactagctgcaatccttcaagctaacatacggttggcatatgtgtggactttcattccactctggaagatactgaagggaggatgaatatccacaccatacgtgaatactcatggcctccctgatcatagccaaagtctaaagagcagcctaggtgtccgccagcagatggacagatggacgccacacaaccgaatacttctgtgttgttgttattgatgtatgaatttatttgtgtgtgacgtcgtgagagcatgactgtcacagagcacttggggacgtccgagaacggcttttggtagtcggtcctctcctccacttacgcatgggttccaggaattgaactcaagttgcgtggcttgcacgcaagcagctttacccactgaccccttgcaggtccccagagaacagtgtgaagccctaaaaatgactcaggtgctgtatacaccacaacacagcacagaggggtcttagaaacacgaagctaagctaaagaagctgggcataaaacctacatgcggtgtgattcaacttacatgtcaacaaagaaaataaagggccaatctacaggaacagagagtctggctgctgcagtttctggggactcgagagagagagagaaacaaggagtgagtgacagccagtggcacagggtttctttgggtgaaaatgttctacaaaccattgtggtgatggttgcccaggcgtgaataggcttgacacccaccgaattatatgctttaagtggtgggttttatggttgtgtgtgtttcttctctgaagagctattgaaacgcgtcactgaagcctctgggtgaccgcagggaaacacaccctgtgtatctggcctcaagcttgacctttgtggcttggcttcacagggcaggggccaaggcagctcagcagcttctgagatggtgcaagggctgccctcaagcccaggcccagggctgtggccacaacacaagatagaacgtcagagtggcataagtttgggttctgaaagatcatttatgacagttttatgtggggaccggggtgagagtggcctcagtgccctgagctcttcctgggtgtgatgagccctatttctgatgagaaatggcagcctcgtgccctggtggtctctgatgactggcgtgagcagagaggcacaagcaatactcagtcctggcctacacctgcgtcatcaggtttgtgttccgtgactcagtcctcccggcggcagtgatcgctgccgtggcaattagccagtgtgtcctaagatcgtggcctcaccattctgcaggtccccgctgaaagccttgctggttttaggggtgctgagtgtgttgtaggagctttgccaagccagaccggatagtgggagggaagcaggctcacagacgtgactaactactgtgtcctgactcagaagaacccgtgcagtagaggagcggagtccaaggccagagcgccatctagcactgtgaccatgacagaagggacaaccctgacagcatccactactgcctctactacctgtcacccttatccagctggacgctgttgttggaggagagagaacggggccggcagtctgagcacagggctggaagatgaggtcaggcagccataaccaatctgatgctcagcctagggcctgtctttgttgtttgtgtctgtggtggtaaagcaaagagccttctccttccccagcagtgggatgggacaagaaggctcaccctgacggactggtttgttgccatcagcaggccgtccgggaacacctgcagtgcatctggtccattgccaggtcaaggccaaggctgccccgtgtctgaatctcttcctgaagcagcaagaatgagcacagagtctggaagttactgtgctgagtgtgaggctgtgatggagggaggagcacagctaggaagcctaaggaatcgaggagcatctgaaaggggtggcgatgagctggagtagcccggtgacggggacggggcattccgagtggatgatggtgggagggggcacatgggtcctggctgcctggagcagaccaccctttactgctcctgccgccctgatcttgttttagtccttacactaaatctactgcagggagggccatgtcacctctgctgcaggacaggggtcaggtgacatgctcaaaacctgagccagtaaacatcagagctgggacttgaacttagcctagcccaggccccacccgggtgcctcgggaatgcgctccagtgtttgaggttgatgggtgttgagggtgcgtttgccccttcccagggctacggggctacggtatagccactgccctcctcgtctgtcatcaccgtttaatgaaacctgccactctgtctccctgatgcctctgtcatcactggccgccatgtgtgacacaggatcccagtgagctcatccttgcgagccaggcatccatgaccttcattcattcagttcatccagggagtaacttacgggtccccagtataggcctgcggctgccagcaccaggagctccgtcccaagccgtttgcccatcgtttccctaagcgtcagctcctcctggaagtcaccacgctgtcctcgtgtttttatgaaaaactttagtggaagaccccgtgcccctgtcacataggagtgagtgtcccatccagacatgactcagaccttcctaccgacacaaggagaataggcacgtcctgtaacccagggttacgtgctgaactaagagacaggtctagaattgcaacttcaaacaatccataaagggcttcactgaccactagctttttacagcagagcgtagtatgaaatgggagacagtgagaagaaagtttgaagaaagtagagcatggcgagggaaaggtcacccctaaggataccactcacacgtcacccctaaggacaccactcacacttgaccactgtgatcattttggaggggtttccaggcctttctctgtcatgtgataaatgctctaaagagagccatgctgagcttgcttttcacagtgtattttggacatctaaccttgaacagttggctagggtaggggctgggggcatgaaagagtgaatgaattccagttggccctactatgtcaacagctagccagacctgggaatcacctagcttgcctttcccatgtctccaggcacccagttcccttcaaatctctgcctccctcggccttggacctcaggttctgcatctgtgctaaggactgtgggtagcatagtacttcagctgtggtactgtctcttcacaaagcgctgaggtatctcatccctgccactctcacaacggagcaggcatgctggaggccacagttagatggtttgtaccaatcatagccttagaggggcaccagaatctactgacacctgagttagacagcgggatatcagaatcagaatccagacacgggtgactcagagatcagtatttcgtccaccaggatgaacatgtgggaggaggaagaccctgcctgggaccgcaagggtgcatatactgccagatctaggggacagagaaagccttggtaagcctttgggctgctggggtgtccccacctatcacatgagcgcctctggaggcccaactaaacttgacagatttcctgggctgagataggcttaggtggctcaggactttatccttaaaggtacctgctgtgacatccctattgctgctgagctctgacacttaagggtattccaggatgagggaacagtgttgtgaaagtgaagttaggttgcccccgtaggctaaggagctggactagcaagcctgtgggggatcggggtgggggcaggctatgcttctattctaagcctaaggtgggtttttaacagtgacaacagcagcaagtctcagaatccatgaccatggggtagatgctgggcatgaaaggtgatgggtagagcagaattcagtagatcagaacagcaacagggaagccagcaggagagtggggcggggtatatggaggagaggaggcctccctagtttggatcctggacaatagtttcctcctggatcaagctgcagacgccactttctatcctcagccctgttggcagtctcggggtcctgcctgggcccaagtgctaagcaccagtttcttttctttgaactctgggtacccaggtcacagctgagcggctcagtgcagaggcatctttctctttttgtcctcctctgtgtttgaagccaaagcttaagttttaatttcacattattcctgacataatgctaatgagttccagccgtcccctgagttggggtagtgaggctctgttgagccaggcagctgggcagctggcttctcagaaagtacttcatgtctccggagcgcagaggagcgtggtgcagcgggcgcttcctctcaggggcttgcttatcacatagacacacttctttggttttcattgctggtgctccggcgtggtgacagccttgtgtatctctctggcccgagagaggaagcacccatgagatagtggaaaacccaggcctttatagtgccccagcctgggctggactatactgctagagaattgtggttttaggagtgctcgatatggccgtgaccacattttctggtgagttagtaactgttcttactctagtcatttctttactgtggtcggtgtttcctcaatccctgtaacactttgtttttgattcagtacagagtaagcaccctagtgttgggcgtccgtggatgtctcatctaagaagagtagtactgtggcccctccatctaggggagtgtgagatcagccaagcaggcatagacagggctgctgagcctgattgtgggtgctgttggtgcctgtgatgtcttccttcttgtctgtgtttctccgtgttacttatatcagagctcattctgaagcccacagctagaatgtgaggagaatacgtttaacttttaagtttgccgcagcatgggcgtctgtgccaggagcccacctccagaaactcacagaacagcacctagccgtctccacatcctcccacagaacagcacctagccgtctcctcatcctcccagttctgggacatgcagtcttatatgtagaattgaaggaccctcagtctcttaacactgacaccggctaagccatgctgtcccatttctggtcgatgtctacttgtcccctgtctgtctgatcaagtctgtcctggttgcaaaaatctgtcttcccaactggtagggaacagacttgcttgagacatgcagaaactaccaaaaggccagttgggctagatagagtcctatcagcacccctagggtttggcctccccactaaggagatcctccctttgtcctggccagagcactgtttgggagctgggagttttgggggactttgctctttccccctcatgctcgctcctggactaacttgggaaactgtcactggacaaactacctccccactctgccctggaatctggctatagatgaggaaggggaagggcttcctcgggtcatatttgctccttgtttttatttagtctttaacagtatttacacacacagggaaccaccctggacactcttgtgtgggttaatggcagaagcatcatttgaatattaactacatgactcatacatacaggctgcagagttctttccacttggtcctgagcttgctcagaagtcatctccactctgtcgcctttaagtacttacagttctctctgttccacacacacccactctacagatgagaactgagtgagacctagtggggaaaggcacttcccaggggacctggcctagtgacgggcttctgtatggtaacagggtgtcactgtggacactggagaatgggccatatgcagaaccattgacagcagatccctcttgactactgtgagcctccctaagaagaggctaccccttagagatacctggggtctaaagaaaggatacacttgggagtacaggaatatatatagccttgggacccagtgggtgtctggatgctggtgtgtcatcggggaagcattgtctctaggaggcatggctttcttccagagcacatgtactggctattctgaggctacccttattccacacgtgtgtcagtttctgcgtgtgttgtgttgcacacaggctacatgtcagtgagtgtctctagagctggtagaattggcgtgtttgtgtaggtggtagtatctgaggcttgtgtatctttgggtatatgtacaatcgtgtgtgtgctgtactgaatgtcatgagttgtagggtgtctctgtgtgtgtgggggggtgtagaatatgtatgcagggcttatgtataagagagcatatctaggtcttggtgtgtatgtaggagtgtgctggtgtcctgggtctgtctttgacatactgctgggttgagaggaaaagagtcccttttagggtacatatgtatgtgtgtctgtgattctaggtctctgtctggtggcctggtgtgcatgtgtcagagaaaaagctattcccctgcctcccctctctcgaaaccctggccccagccaagctgctctgtatacccctgaaaggcagggcatcttagtccttgctggctgacaaacaagtctctaggcttctctcaactactaggaatagaaacctgtttcgtttgtggacaaggctttgggcccaaggtgagcctgccccagagtggtcagtgtggtcctggtaaggaaaggttcaggcaccacatcctcagaaccgtcttcccgggtcaggcatgactcctttgtcccttctcccctgctgtgctctgtgcctgccactaccacagtctgtcagcttccctgggtggtatcaagggctcctggtacctgggttagcaagaggagattgccacactggcctgccgggcatgagaatgtgcctcgcggtgcaaagcttggcacaggggcgaacctcaacctcacccttattgagctcaaggactctcacctgctctgggccccagtctctacctttaatggagtcaggcgctctgtctacctaatgggattgaggagatggtatttagggagtttggtctggtcgcaggtacgtagcaaatgtgacagctgccgcagggcagagttccctactgcatacagctgcctacttattctttgtgccctaagttggactagaccttcctccagaggcctcatagtctagaggcagagtcctgaccagtgtccttcagccccacttctccaggccttctttgtctcttcccatcatgccttggccatcccccagaggactggcaaagcctggggctctggagaaccctgtgaaagccacaaggggattataggctggtaagctcactcggggccagcctgagtctctcccaggatgaggggctgaggagtggccctcaattcccggatggctggcctttcaggcaggccaagcacagcattgtttgacatcttgggaaagaatctggcaccgcacacttctcccatctaaatcattgtaaagtaacttaaaagtttgtacaaaaatgagaagttgatctctgaatggcctgccaggctattgtaacccaatatattttacccttaaaccagaaatgtgtttgtttagaagcaagtcactggcaaatgccaaatctggaggcatttcctcccctgccctgcctcacgcctcattccccaaacctcttctttattctctgcatccagcttcttcattagggcatgagaaaccgggccttccttcctcttgggccctgtgagccgcagggaggaaacttgacattcattaggcaccaactgtgtgccaggtcctactctgggtctatccatgcatttacttctcagagctgttcacagtagttccgtttagagagaagaaaaatgaggacggggatgggatatgatttgccccaattgatgcttctatgtgagtttctgcatggtgtcatcctacaccttacgtgtgaaacagagcccttgattcttctccaggggccagagggaaagacatcgtcaccagagccaactgggtccgccacccagaaggttctcctgcatgatctcagcccttaccgtgagcaaaaggctcagaaggcagcttgctgagaacctaaccagtcctgcggatgttaagcctaagccaggcctaagaggtcctgggctttgactaaccccccttcccacccagaactggaagggtctctgggctgagcctgcaggggaagatgctaccccctccctggctccagcaatggctcccctgtctttcatcttctgccctccgtactgtgagctgcacgcagcagtaatggattgcagtggacttttttcaattaggctgattttcccctgcctgttactataaaactccagatggtccggggcttcacggttcctggggtattttcccccattattttaatcttctttcgtaacgcgccccacatgtgggaacagggcctttcaaggctctgcactccactctgtggatcagccctgctctcctctccactaggctgcccgggtttccctttctgtgtggaggcaccacagagagcagttcccaacgtcagttgagaatcacaacctatgttgggcccactttgaaatcaacgggagttatggcctggtttaggtgttctgtgagtaacccaaggcactggcctaagagttcaggggtgtgtagccacgtcagtcagagactccaggccagctctgattggctatgtgagcgtgttgacatcacccttctgatcctcacttctgtcatctgatgcagggtgaggctgggaagtcacttccccagcagggtgtattgccttgattcggcgaaacacagcacagcccgtgagttcacaccactcactctaattatgtatttcagtgttgatagacacctatcttgacatgttttgggactattagacacccaaggtagctttcccccaaaagtcaccttgtctattcccatgtgtgtgagcctttgcttcagggtagatgaaccaggagtatatctggttagttgaacaatgtatttaactttaccccgagcaagaagtattcatggaactgtctgaaggccactgatggcatcgagaccaggcccacaggatgctacacgcacgctttggttcacaaacagcacccttcccatgtgcccaggagatctctgggctctggacaggtaaaatgaattcacctgagagcgttccctcagggtgcatagaggatggactggtaccacactgtacaagatgctggagcacagcggtgtgcgagctgacgagtctgtcaaggatcaccagggccagctcatactggccagaacgcaggggaaagtgtgtaataaagtgtgagagagccgagcccagttgtagcaaggaaggataaggacatccttatctcgatgccaagtcctcattctgggtgccacatgggaatgggctgggaagaggcaggcagctctgtggtacccaggcgagtcatgctacaaagggtagagaaagtatgaagaggtattggcgagatgggtacctccaagggctggtggcagattggatgctgaggttaaggagaaggaagcaggttcctagcttggcatctgggtagaaggtggtcccatcagcagaagccatgctggagcaccccaggtgagcagcctgctgtcatgttagctatggcagtaatagtacaagagtgttgtgtaaagttttccaggatcctaatgccagagctcactgctcctccgtgtgtgtgatcagaggcagtccccagctccggtttctgccttccctctgctttccatgatgctggctcagtggtcctctagaacccccgtgcccctgcagcccgaaccgccagtgcttgcgcctgctctccctgagatgagtgtcttagcacctcatttgtcaaaacccaggcatcagggctcagcccttaggaagggtttggcaatccccagccctgcaactgaaaggccccccgcattctgccccttagagctctcagcagctgctgctctatgtgggacagtaaggcattttgggcctttgttgttggaagatggagtatggtgagggagttagccaggtgtgggatacaggggttgaggagtaagaagaggaaagggtaggagaaagtagggggacaccaacagacagacggacggatgcacgtgttcatgtgcacactcagctctattagagatgtcgcaggccagagactgcctcaggacaaccgccacttagcctgctctgggaccttaagcagcagcgacagtgacaaggtcagaacttgctgtgtgttatttcacgtaggactcctgccgggtaaagttagtgccattcctctggtttgcagataaactgagacccaaggaacttgcctgggatcactcaggaagtgagcagccaacagttgccaggatagtctgtagcagaggccatatctctaatcagtgtgctttatgccactgagcctccacccctcatctagctaggcagttatacatacccagaaggctgtggggacagcatgctgtggcagcaccaggcccatttgaacaagacacaagtgttgacccgacggtcactctcagcatcttctctatctctgacctcatctgacaggccactcccaggacacaaacccttccaccactgttgcaagtggagttgggaaggctcctcctaggaggtgttgcctaagcaacagagacgtgtatttgctaaggggaaaggaaggaaagggccttcagggccttgtgaacattaagaaaaggccttccggatggagactcctggcaagttctgagtcatctctccacagctgatgtgtgggcagtatggggtgggagagacgtttggggccatcctggaagacatagatcctgaatttctgaaaaatgtatacattaaattgtcagagtgggaagggaagacttcccaggagaggtgtggactgaacaaaggcacagatggggaaacaggtgttctgaagcccagacaaggatccatagattcctggcgggagtgatccccagagtctgacagactctcgaagtcagagaaatcacgcagctgggagttactgatctccaacagccttccccctaaaccagtcagctagcaactgagggccaaagtggatggtgagaaaagccctgtttctgcccagggtccttcatcacatagtggtggacttggcacaaggtcattgctggtgagcagggagacactgaccacttaatattccacattcaggatttcctttgttctgataaagcaacagaccccataactagagacccaggttgacccggcagcggtacgcactgtgacccacgtggtatctataaagggccatcgtctctgacccttgtcattgtggctgtacaggaagttctgccatggcctctgctcttgacaggcctcagtggtcctctgtaactgaagcaatggcctcctctctgcctcttctccaagtctgacttgccactagcagctaaaggtggcttcttatatgtatatatacctggcactgtccccattcctctaggtcccttctgattctcataattcacatatgactctgtccccctagttcccagaggatgtgtctggccctgcccccacctcccatttccagagtatacatctgatcagcccttctgtgatccccatggttcctagtgtgaacagcttctatctgagcttcagggcagcctaactctgactccctaacacaatcctcccacagcattccccagggcacaacatacacaagtgcagaggggctccatgggggacctgtgccctttgcatgtgctagagcctcagactacccattgttgcccactgtcctgtcagccccctgtagactgagctctgctgtcacggtaataggttctcagaagcatggtgggatgagtgaagacgtaggtgctgctcgtatgaccagatactgtgtcttccacacccctttccatctctccatgctctctcttcatcctcagtagtcactggaaatcagcaagcatagtcaccctacctaggctttgacatcacatctggcctgtaccttgtcttcgctgtgtcatgtgaccttggttgggcaggtcacttcactttaccgatgttttggagacaaggacattgatgtcacgtgcttcagaggtttgtaactaaacaccagcaccatgctggagcagcaggttgcagccactgtggacccccaccataccctgaagtacacatagccagaaggctagctggctggcatgggctggcagtaggtgacaccacactaaaggctggagtctgtggcttcacccagccagccagccagcccctgagttggactttggaggctgagcacgaggttgttggatggtcaggagagatgtccttccttgctgagggacatgtgtgaacagaagctgaaaactgcctgctgctagaggaggccaggcagagaggcaggcaggtgggagaatgccacaggaataggatgctaggtggcaggcctgtgaggagggggaccgctggattctattggtgtgttggggctccagggcagctgtctggagagcagggcaggaggcatggaggacacacacacataagccaaaggtatggaaaggaggtccaggctcagcagaggctgcagggacacacagttgaggccatggagggtgggcaggatgggcagaggccacaaactcttcctgtcctttctcttaactcatttcttcacacactcactgccaggaacctaacagccgtagtgtgtctactacatgtccagtacctagcaagcagcccactagatggtgggcagctaagtgacatgtgaccccgtgacacacacatgtggctactgaccagtctcacaacagttatcaaggatggcacccattgccattcattgccctccttgtacgcccctggtggactgatggatagccatagcctcacctttaaagaggtacagatacaccaagggctcagggcaccccagagcccctcagggatggagcccttccagtccaggctgggctcccactttctggggttctatgctccgacacggtttgccttggagttgctaatggggtgcacaactggctcatgtgtgggtcccaaggtcaaaagcagaaaggtcatcttgtccccctagcagtttctaagtccagccactttcttatgcctgaacacttctcaggggcagtgttcccagaggcttaggaaagcagcaggtcgttggtctgtgccaggtatgcatagggggtgctgatgagcgatgggtgggccctcggggctgagggcacccctcatcagcctctcctgcagttcgaaggctgcagtaaagccttttcccgtctggagaacctgaagatccatctgcggagccacacaggcgagaaaccatacctgtgccagcacccaggctgccagaaggccttcagcaactccagcgaccgtgccaagcaccaacgcacccacctcgacacggtaggtaggcctgaatgtggaggccagggtggaacagggctgcacgcagaccccactctcccagcagagtctctgcctggacccacaggctctgccacacagggcaggagtgggtggggcatgctctttgtattgtaggataattagtagactccctttccactaagtagatagattttttttttcccgtcatcctccagttgtagcaaacacaaataagttgggtatggccatatgtcctcagtgagaatttaagatgggcagacaaaacttaacaagcacctccagttgagattatcctccttggcccacggggaccagcccaggccccagccagggcctttatggggaattgggtgctctcactcaatcccacatgctttctttggaaggccagctacatctacatcctgagcaggccacttcctgctcttcctactcctcacttgtggacaatacatatgctctctgaggctatctttctccctaaaggagctgtagttggtgactcgtgagatccagcctgacggtttagtgttgtctcttcctacctccctgcccttgcttatatgtcaggtagcagagacccacagctctgtcccaggtcgctccactctcttcccctgtgcgagtaaggacatcagcagttgacatagcactttcaggaagtctctgacccatgtgacacaggagggaagtgggcttaagggtcctgtgcgtgctcacgatggtatggccgactctggcagaggtgacagactcagtgttcatgccagtctcacagggaggatcccaaccctgtgtgcggtacagggctggagtctgctggaagaacccatgtttggacttcagagcagttctctgccgtgctccgtatgtttgggtggctgggatggggcttggctctacactatagataagcctatcccatccagtacacagatgagggaatgggggcctgaggggctaactgacctattcttgcaacttggaagacataaaacagtagaagttaaacctgagaagactcagccctccaatcccagccctgtacctcacagctgggaacccagattgagaaaagtgagaggaaaactcacaggaaactttgaagccaggccagaccctggactgtgccccctctctcttccagccacatagtgtcacacacctgccccagttcaggcaaagctgagactcaagtcagattgtttctgtgtccatggcaggcaagatgcagttggccgggcccaaggttctttattggaaaagcttgaagggctatgtctaagcagaagggacagagtaacaaggaagagtctttgtccccaactcaaactcagcttccctcctcttagagagagatgtcagtggctgagacctgaagtgcagtggctgagacttgaagagatctgtggctcttccaagcctctctccttggtcacctcagccttggggtccttggcacttgcccttgagagatgtagcttatactgcatcacccctagatttgtcttgaagccacagtgagcctctgcccaggcctgggacatgtccccatgacctctctagtttccggttccttcctcatccctgcccttacagtaattcccagctctgcttgatgagtgaacatagcccctgagtgctaggctacgtcagtttcacacaagcccacgggattaccccaggccttcatctatctaagaagtctctgacaggtctgtggggacaccaaactccactcatatctgcccagacagcacaggcagctccttgttgctagaccatgtcctagcccacacccctcaagtcagcagtgggttaggctcacgcataggacctgtcacagagacccagcctcagttccagctctccctggcatcagagagcacagggcccatggcaggaatgaagttctctggacagactggagaaggcaggctgcagctctgattttggtactgccacttgtcagaaaggtcagatgagaaccccctcttacctcctgtaccccagttttctcagcggtaagaggtctgcctctcgagaatagagggtatagctgagggaaatgataagaggtctgcgtgcaaaccaggcagccctttctttcctagtctgttatgtcagccaaattctgccacaccagtatgggattggccctggcagccattgttccagtgtgggccccagtttccccatcagcaggatgggcctctgtttatttgattggtctgtttagaggccctgagagaaggtagtcccctaacagagaggcacacacacctgctggctatgacatcttctgtaaaactggggcaattctcatggatgggtatgggactcagagggaaccacaagtggcccatgcgtagcagggctgtgcccaggaagccttcaaagacagaatgtcttccatgtcaagctcacagagctcacacaagcccagtcttggagcttatttggggattccgggcctagggtttagctggtagcttgggatagccacagggtttcctgagggctcaagggagctgggcccaaggaatgttcaatacacgtcaagccctggaaaaacaaacatgaagatgggccttgctgccctgtttccaagcgagtgtgcctgccacagagagctgtttgtttgctttttgaccaaagcgtgtggatttgtatcagcattcccaagggacaaggcctggggccagccagctcaggggcagggcagggctcatgggagagtgtcctggggttgaatagggctcctcagagagagagagagagagagagagagagagtctgtgtgtgtgtgtgtgtgtgtgtgtgtgtgtgtgtgtgtgtgttagtggaggctgggtctcatgtgaggagctgtgcgagtgtgtgggactcaatgctgaagcaggtggtggggcagtcccaggcagacctccctgtccctcccctgccttcctgttggcctagtcagcttctcaccactgtctctctctttttgttgttctccattctttaacctggcaatctccgtggcagctccaaggtatggttatggtcatctctttctgcttctccccacctggacagtgggcacctctcccagctcccctgcctctggctggggcggcattgccttgaagcttctgggtagctccacttggtgtgtctgactggacatgacagtccctggggcttactctgagaggtaccattttctaaacacacatgcctgctcacccgcacaccagcgtgcccacacacgcatgctcacacagacttccatcctctgcagttccaggaggcaggcctgagggctatgaaggcactcagtgtgcttcaactataggaagccctgtgaacttcctgcctcagtagtatctgggagccccaagtactggctgcccctcggctatgagatgcatgtgggctgtcagttgcccttcgctggtcttctggaacctgagcagaattagataggtccctcactggtgccacggagggacagtggaaagatgggcagcacaggcagacagcatggcctgtgggaatctcaggtcaatccagaagacaggatctgagcgagggtaaagaggaggtgatgtgggttgggtggggtaggagcagcattgtgggaaacgagggaggacaatggtagaaggggaccctagtcagtgccaagagctgacccagatctatcagaggtccatggaggggtgcacagaggcaggaccatggctacactgtgacaggagctctaggacttagcatgggaggagattagggtctgggagatgggagaacctgaggcaggaatctaaacagaagcaaggggctggcttgggtcccctgttgatcttccagggcagtgggatggtcataggaagtgagtttggcccatgcccgtgcagacagcacagcctttgggcctcccggcttgtgctttttgtggtcagatcccccaaccctagttactggaaggaggacctcgcttgctgtgacccatagtaccacttggacctgggcctgctgcctaacagcagtcatccaagctggccctgggatatcccctcttcctttcttcattctcctgcccaggacaacttctcgtgtggccctgctccacatactgcttctatgcagctatgctatgacagcattttcgtcacagccagtgtgagctagacagacccatggggttcagtcagtccccctttgcagactggcctgtagtcctaacatggtggcagaatggggacctgtcactctcccccccccagaccaggtgacctccccttatctgtagccttcagactgcagcctgccccttacctacccccccttcccgtggcaggccacataagggaccgtcatcttcaacagtggcctcttacctatccctcatccccctctgatgttacagaagccatatgcttgtcagatccctggctgctccaagcgctacacggaccccagctccctccgcaagcacgtgaaggcccactcagccaaagagcagcaggtgcgtaagaaggtaggtggccacaagcccagtcccatctctgcagacatctctaatcccagccacttgtccagcatcagaccaagtctcccatgactgcatgcacagtactctctacacaggtatacgtgcccacttctcttccatctgcccatgtctttctcccaaccagtcagtataccagcccacaacccttcccctatcaccccaagttgataagacattcacccaccaacagactggtacccttagctgtggcactgatacccagccctgggctaggcatcacaggtataaaataaaagatatacccattttcagtgatgtgaggcagacaagaggaggctccttgccatatgataatgacagcagggaggtcacctctggcacagataaggggccttggcccatcttcatgagaacagaaaggtcccaggtaaaagcaccaggaaccatgaagaataggggtgtgactctggtcccacctgcagagtctgccccggccaccttagtggcctacgcttctgtgtttcctgttcctcggtggctggctgggccataggtgccggtgtggactgagtgacagggcacagattcacaaacacagaagggataggcagctgagtcaggtaagaagtgtgtgcctggcatgaccagggcacatgcagagccagtgtaactctatacataggtgggcagtcccaaatgctggatgctctgcctaaggtttgagctgtgtcccaaggatattcaatgccatcttgaggggaaaacaaaacaaaacaccatggcctcgagatgcctctggggctgtgtggaggagcggcgtgttagagtaaaatggaaggctagagctctgcactgggagcaagggaacaagtggaaatgccctgggataaaacgatggagacctgggctggcatgggggtaggggtgggggtggggacacatcgagggtggtgcattccaccaggaccaaaaacctaggagaatgttttacccgagaaagtctgcaatacgctgctggggtctccaagttataaagtaaggaacaggccagccaccttggagcatcagcaaagcatcctggcctacagcactcaatctccaagggccatgggagtcgggtttactcaaacatcagaggctgtgagagtgaagggacgggtggaagccgactgaagaacaccagcaaagtgaggagggcgagcccctttactggcttaagcagttgtgagcagtgagcactgtggtccacgcacatgaccttgaacacgcagaaaataaaacagcaaagctgtatgcagactgggggagcaggcggcttccgtcccatgaacaggcagatagagacggcactaggaacacagcccccccgtgatggaagtggataggatcgcagggccaggggtctccatcctagaggcactggggacccatttgaaaggtagagcagaggaaggtggagtgagtgcatacagcttaagtgtcattacatccttagctcactgagacccgggctggggagaggggggcgctcaggaccccaggggatacaccatggtggacaagccacaaacccagtggctcttcaactcagtgtatagctgtcatcctgcaagtgggcctttgcttatgtccatttcagagacatgaaaactgagtctcagggtgctaatgaagtagcccagtcacgcagcccaggacagacagctgggctgccagatcctgacactgcccttggcagatgggcctcgggttagcaagggatggttcaggcttccgtaggctggaactaggtgccaaagaggctgctgtcccagcacactggtgcctaaggacctaagaatcctgccctggttttcccaggcccatgcttggctcacattgagcccccagggacctgggctgcagatctgcaaacttcccaacaataagcatccattagagccatcagggatgatggatctctctgggtaaggcgacccttgtattatgacaagcccttggcagccctgctgcattttggagagaattagaaatgcttttcttccttaattctcctctcctgctgttcctaatccaatcgacattgcggtctgtaagaacatgccttggccctcttttgccagaaacttcttttacatggaaaacagctgccgggtcaggcgctgagagcccagtgtgagactggcacgcctttcccacctggtcagccctccctccacctgggaaggtggcaagcccctcgggcctgactctgggctgcccaaagacacaggcaaggctgtgaccttctcagcacctgttttgacctggtaaagtgagcatttggacagagccttggctcttggctgctaagctttgggattcccctggagaagggtcacctcccattcaggtgcctgtccccttgtcaccccatgtcttgaaagccccaccctctgtctgtcttagctccacttggagacgtatagatgctccaaggtgtcccaggtgtcatggaccagatcatccccagccaagctttgctgctagccagacagctctggtccacacagcaatccactgggccactactgtgaccctgtggaagtcatgcagctggagcccagccaaccctacccatgatgctctacaaatgatcttatggctcagtcatcacctcactactgcaatggtagaggcagtcagtgctccagaacacaggcaagatgtgaggcgcagagcagttattaatccactgacacacaggcaataagaggtggcatggagctggaggccaaccctggattctttccatcgtccctcatagtcacagggaggatggatggagcgaggggtgaaggaacggttgctaagcaacgtcatgcctaccagcacaggttgccctgatgggagctgcaaaggcccattagtcacacttaaccacccctcccaggttatgctgaggccactcctctaaagctgatccctcggggaccacaaggagaccctccccaagagacacaggggggttggggatttagctcagtggtagagcacttgcccagcaagctcaaggccctgggttcagtcctcaactctgaagagagagaagagcgagagagagagagagagagagagagagagagagagccccaactggagcctgggaggctcccctccctcctaacctggctcactgggctaaaggtaaacccagcctttctggtataacttcttcctaggaacagcctccagtaaaatccttgcccatcaaatcctgtgctagcgtctcttctcagaagcccttatactgtccactagcccactttcctgatgaggaaaccaggatagcgaagaaggtcctacttcaggctcctgactttcctgccttgcccccctgaacagggggtgcctgtcgccaggtggcctgacactcatgcttgctcacctcctgcttcccagctgcacacaggtgccgacccagaggctgatgttctgtccgagtgtctgtccctgcagcagctccaagcatccacactgttgccggccagcagagggaagggcagccaaaccctgagccaggagctcctcccaggtatgttcacacgaggcagagccagggcacaggggcacagccagtggatcctttctctgccaggcatccctcagagggactccggtgtactgtcagcccaagagcacatgggcaagacttgttgcagggtgcgttgtctcttggacttcatcgcttctgtaccccacacttccaggatgtcttccacaactctgctaagtaggtcaatgccataagatggcgcccactcccccttggtctgatggaggcctcttggattcgctctgggcacctacctgttctcactgcagtaagccttctccccctcttccttcaccaagatggctcttagcctgctcccagaacctgcctccttgtcccctcctcttggaagtctttcttggggctgtcagtttcccttctgtatccccacaagcctgtggcagggaaattcctctggagaaagcctgagtctgcctcatcccagagtcagtagcatctagcttaggacacaagcagaaccagccttgagggacatgtggccctgcctgaaatactgtgaagagagtctacccctctgagtgtaagccaattcttccttgtggcacatctgcctaagactcatccacagtggggccctccaagaagtaccagaccagccccagtctggacttgagaagtcttgctcagtgcccagactccttcagaagagaaagctgaggctttgagaggcttgggcaacatagccagaagcacaacctactccctgctgagctctgtgacccagcatagggctctctgggcctgactctgaaggcccatttgggcaacacttatagccagtggtccagcaagaacagctgctgctggctgcaggcaccctcgggccgtggcaggaatcctggctctgtggcctgggaacactgcccagctgcgggcagcatggcctttagattcagtgggaggttctcgggctgcgtcttccacccctgaggaaaccacagcctcagagcagagcgctcagctgcccactgggggttcttgaattactccaaccatttaaaagagcatttttttttctaattatattctttcccccaacactgaagtaatatcagattttaaaacatgtcagggcccttaccagacagccttggaagaccaggttttcacccatcttcaggcctactgtgtgaggatgccctgaggtgcatacagggaggagggatacgcataccccagctaggtaccccaaattccaaaagatcagaactagaagggacaggaagggtcttattctccattgctgggaccccaagctgaggactccagaaaggggctcccactgatgggggcacagctgcctgggccctggggcctgcagcctctgtgcatccaccttgagaggactcaccaggcaagaagagctaacccatgtagatcagcagcagctggtcagccctggagcctctttgtcctcactgagggacaaggacctcagtagttgtctctcttaacagatggcatctcatctcgcccctagctacaagctacattattgagaaacaaatccgacaggacacaggcacaggtggaaggcctctggctgatacgcctgcctgctgcctagtgtggatggaatcaagtcttcctcagtgccccttctgggcctgctgtgaggacaaagccgaaaatgcaccccagtggctgttcagggccagtatctgcttcccacttggggattcacttggggagtgcctgcttgagcccagactgtcccagaggctttgctcttcaccgaatgccacctcacacagttcttcatacctgctatgctctggctggtactgacctcagctctccaacccagaaagcagcaggaaggatagcatgccaggcgctccctggggctgggctgcacacagacgcctgaggcacagttgagaacagatgttagggccagaggttaagtgctcatggctgatgattgatgttgctgctttacagcccaaagtgatatggtctggggatgctacaggcccagtccccacctccatggcccttacaacctggtgattcttctgtcccatcctctgtccgtgtacacgctctgtcactgccatgcatctctctggggctgtaaagacaagggcatcagccaggacagcatcaaaggcaggggacagattctagaagaatcagaagagtccaaaactgaggggatgaggcagtgatgatataagcttgtcactgcctgttctgggaggggacacgtacctggcaggcatgtgccaagacaggtagtgaggatgtgagaagtaggtgtcagcgtggcagagggacccatctacattccagccatcccagctgtcaggacctcagaattgattcaagatggcaggcaagggggtagtgaggagagtgtgaagcagagcgggctaagcagcatgacgccatgtgctgctcgcacgaccttcccctccaggtacctcctgaccaaggcaggaggatgcatgcacagccaggacaggcagcaatgggcggcagcactgctgaccctgcaggacacagcactgctgaccctggcagtagggttctcaaggccctgaagcagcactggggggggggggacattcacagggtgcttggtggctgcctgggggtctgggtatcatggtcagctgaccacagtgcctgtagcagagccaaggtcctccttggacactttagaccccagctcagctgaagcctctgcctcaaataggcagtctctccgtttggaacatggtctgagtttctaaactgtcagattaggcatgtggagaaccttgcaaggcacataaatatagttaccagccatgatgacctaataatgggacttttcttagtgctacctagctacagtcacctgtaccacacaagcagctcgtctggtcttcaaggcagagtgctgctatcaatagtggcagcttaaaggcactggggccacattatcatagctccttgatgctgccccgggcctgtactgtaccccagccccattcagtgccctgataatagcctcctataggttctggtcagaatgtcccgtctaggatgagacagccttagaatgctaagcagctgagctcaccctgaaagaaacctgagtgttcccaggctagaccttaagtggtcttaaaaagctatagtgtgagctcctgtacccgtacatccttacaactagaacaacacttaagaagtaacagccggcaggggaagactggccagccagtgaacatcataccagcactgctgagagcataggccttggggaaagtcctccctcatgtcaccttcatgcaagaatccatgttcttgatttcaagaacattaatatttctattcagataagcatattaggttaacaagagggcaaaactggtcccattctttttcaggtaaagttgggtgcatctgagaccttctactcgtgagctggtgatgtgactcaggtcttgcctagcatgaatggagacctagatatcatctccagcaccacaagataaagtgcaggccttgtgccttgtacctggaggagggtccatcctctcctgacagaggcttcttggcagtgttgccctctgtggtccccagaccctcagctggaactctggaccaacatgtgactgacctagaactgttggtctgttacccacctgtgtgactcccatccttgagtggggctctcacgtaggatatacagcctggagaactctctgtagtaggtcttcttggggcctccagagtcaacctgactaggtcagctattcagccattcctttacagccggtcacgggggagtctcaggctctgcctgcctgtgtctgcagaggcaaggtttagtttatgcaaaggaagccccactgtcaggttctaccaagccctctccattttaaggtcccacctgcttctggggatacagagccatccttgcttttccacgatagctccatggagatttgaagcagctcactccaagtttcccacaccatgtctctgacagtcccctctgcctctcattaactctgtcattagagtaccctcacctctgtcagccacaggctgaaccagagtgataccatcaccactcctggtggagtgtccctcttgtcattctctccataagcagagagtcatgactcctgagatgttctaccctagcgagtagccaaagcccagtaacctatgtttggttctttgcaggtgtgtatcctggctccgtcaccccacaaaacgggcttgcttcaggcatcctgtccccctcccacgatgtcccttccaggcaccacccactggaggtccccactggttcccaccaccacctgtcccctctgcccacagctgagagcaccagggatgggtatgtatgacaagccagtcctcgggggccacacctaagcactcgcgtgggaggtggacaggcaggtgagctgcacacaatctgttgtgactggcgacagcaggctacatttatgcatcactgactggggcctgtgctctgtaccaccttctgtgtaagatgttggtgcctcccagggatgcccctaccaccggaggcaaatgtattcgtactcagtcttccagctacccacatcctccctccctgcagaagcctctagaaacatctgctgccaccaggctctcaggagtacccctcagcatctccctggttctctggacatttgagaccagactttccatagagaggctaacctgggtcccgttggaaccctcagagccttccacggcgccactgtattcagtgtgttccttgccctgttgctggatctgggaacatgctgccatcctcttacatgatcagaggggtcccagtccccaccccacccccacttgtgtttatcaccaatagtgacttgcttgaatttgttgcccatcaacttcctccttccttgccctccctctcgaaggctgcatagccttgaccgctgtacacttaggaccagtccggctttattaaccacatctgtggcttgctactcctcccagggtcctctggccattcattcatctgttctttcctgatccctgtgagcaactccctggccccagtgctgggccactgatggctttgtgaggagatgctggacaggcggcacagttcagccatatgggggaggagcaggaagggctgcggagacccaggaaggaagccagtgggccagatgggctgcctacaggcgtagggctaagccaggttccagggtctgtccggtgggcaggtatggggaagagtggcagggacaaaggcaggcatggtgagagctgggatatgctaggatgaagaggaactgtgatgtggaatcctgagctggagagaggcccccccagaaccagacataaggctagtaatagaggtatcctaaaggttaggccaggccaacggcatgctgaggggtggtgaagggctggggataagagatggcagcctgagctaaggctgggcagatggggagagaacccagatagcagtcatggcacagacttgatcctggctaggtgagggaacccgactgcttgagctctccctgaacctcttcctccgtaaaaggcattaaaaggcctttctccaggaaggtggtgaggaccacccaccctgcctggcattgcttgtgtcacttgggtcccctccctagtctcccctgccctttctgtccccatctcaggccacctgcttagccagtggccagcctcaactaagacattaaagccaaggccgttgcagggcctgggccggttgcttggcctcattcatcacccgcctgccgtcggcataagtacccatcaggaatgcctacagggagaggcagagtgatgacaagacaggaccggagcctacccacccgcccctggcctgcagagtggcgagagctgtccccagccgccaggcgcccgggttccggatgcctgctggagaggagggcctctcggctgacacagcctggggctcccctgaatgggtttctttttcagccccagaggagaaatgggtgggaaggccttcggctttcccggcttgccaaaatcaacagggactggccacttggggctccccagcggtttgctgggacgtttaaatagcttgacagaattatctgattcactcaaaaatatgctaagagggcctgacttcatgaccaaatgtatttaccctggctcagaggagacaccaaatttggtgggtttctttcccagccttgcacaaggaaggcatggagccctggggctaccatccaggtccagggtaacttatataggcttcaactgaagctcaagtgcatgtttctggagttacttgtctggcttcagagaggtacctggctccaggtgactttcttgccattggagtgaggcagaccagcctgtgccggctgcgtgcagttgcgttctgtttaattttactgtctgtgaaggtggcagaggagaatttatggcaggtctggacagagaaaataaaccttgaagttgcacacgcttcccccagacccgtgcttgcttctgcattgtacttcatccgtggggtttagaaagctactttgtggcagggcatgttggccatatacctgaaaataaaaactgggagtactgaggcaggaggatggtgagttaagggcagcctgggctaaacagtaagactctgtctcaaaaagaacctgtttgtggacgttggccattgtttgccacatgccaggacccttttgtgggctctggttatatagaaggccatggatggccctccctgcaaggcacagctgggttttggagactccaatgagaagggggcaagtcagacaccccacagttctgaaggaaagaacttcgtatctactgtgtctttgcctgactgacgccggctaggagaatgtggcccagccttcacagttggcagtggacagagggagaccctttcccccatggtatagaaacattaacagttgggaaggttaaatccagagagacaatgaggacttggctcctgaccttcagacagtggcatctcaagtgggtggaagatggcagtgtattggcaagcctgtagggacaggctgccagggctgaaggaagaggaggcacctccagtgtgtccccgagatctcctgtctcgccactcagtcctgcttccttcagagtggacactgacctgcctgccccgtgaatctagcttctagtagaaggatgaggataatgtgccttgcctaacaaggtggcctagagccatggtcctgattttatagtctactatatagaccagcggttctcaaccttcctagtgctgcgaccctttaatactgttcttcatgttgtggtaaccccccccatcatcataaaattatcttgttgctacctccaaactgtaattttgcttctgctataaattataatgtaaatatctgatatgcaaccccttggagtcgtgatccacatgttgagaactgttaatgtagatggttcaggaaatgccccaggccaacctgaagtcttggggacctgtctcctgctctcaccctctctgcggtacatacatgcactgtcaggctacaagcctgagcgtgggttcagagtccctgaggtagaagtcaggttaggatggagatctgagttgggagcccggcttgtaggatctgcagtaaagcacaccttcaacatggctacattcaaggcaccctgcgtctctcttaaaactgaagttttagctcacaagagtttccaaagagcacaggcttgagcctggatcccttaggttaccttgaatgtaggctgtagcccttccatcacaggagctatgccataggagacaccaggcaggaagacaggagaggctctctgaagtacactgcacagcagagtgagatgccagcccgcgtgtcaggtgtgccagcttcttctccttccgcattgcaccattgatcaatgaggacttgtgcacactgtgctgggtcattcgcttacatggctttgattatactctcatcaaaacctgtgaggccaatgccttgcgaccatgcaggcagaaagcctgggcagcctctctcaactttggtttcttggtctaaaaagggacctaaggaatcaggcttcagatggtctgtccccccccctcacccagaatgccagagtggcacacagtggagggctggttgccatcagcggtggatggtatttaacccacgatagatagtggcggctgcagcatgtcaggtgaacacagcaccctgcaggaaaactgagcctcccaacatcccacaaggtctgtgtctgatggaagttgccaaaagtttaaactctggtctgcagatttccaaggccatagacatctctgttccttgtttattggcagacctgaatgctgagttctcagctaaactatagctatagcaggaggctcacagacacggtcatggcattgtaaattagtgcaggtgctgaggcacctgttgtacctctgtcctgaagtcatatggtactctgttctttccagcctggggcccagtctcctttcacccatggtcagcccactgaaggggcttggtcccccaccgctaccaccagcctcccagagtcagtctccagggggacagtcattctctacagtccccagcaagcctacctacccatccttccaaagcccaccacctctgcccagcccccaaggtaagctgcattcaaccactcgtgacccagaggactgacacagggtctgggggaggctggttgggttccaaggaccatattgggctcctgccttaatgacaggtaagagaggcctgctggttagaggaagactgggtgagtgtggtagatttgggagaaggccaaccgttcccccttgtgggtctccagtatcggccacagactttagcaggccttcacctttcctgtgctttctcattcacaccctcaatgcctggaccggacaacaagggggctgatcccctgatgacccaggtgcttctgtctgtcaccaccatccctgcctcctcttgcaggctaccaaggcagtttccattccatccagaactgcttcccctacgctgactgctaccgggccactgagccagcagcctccagggatggactggtgggtgatgcccacggtttcaaccccttgcgacccagcacatactccagcctcagcacacctttatccgcaccaggtaagcttcccaagacctcccttccctgaacactaccaaggctggccctgtctaatggccccatatagagatagaagcagaagcttgaagaagcccagtctacctgagctagggtacacccttgctagaatgactgtgttcctgtcatacctttgcccagctgtatctaaatactgtcagtccctagattctctgaatgagagaatggtccaaatggtgtttcatactgagtgttaatcaagtacatgctgagttctggctcagcacagccttggagcctgcagataccgctgatccctgctgtaggaagagaagcaggaaggggtagggcctggggcatgcctgtttcgtgcaggaccagggaactgagagtgcagggattggaaatctgatgctctaaccctgctgggtgcagaggtgctgtggacagggggggggctgcatgctgcccagagctggaatcaggctctatccagcaggcagagcagaactcaggacagcctattgggtagttcctccttctgaattacctaagtcaacccctactcaagtcagtcccagggtccagatctagtgaagatcctaaggctcagaccaaagagaagctgaggccctatggagggctcttgagtcgcctgtgagcagcacagtgggggacagaggaccacggacactggacatggggccagtcagggaggccaccagtaaacacagacctgtggctccatcatccctgggggctttgctgtgtgtgcatacacacgtcctctgtgtctgtgttctcccttgccattttgtggagcacatctgtatgcatgtagggctggcccaggtggttccaggcattttgaggattacccatggcatatgcgtgtattcttatgttgtactgacgttcacatacctctctcacatcgttacctaccctgagactcacatctaactccatagacatacatgaacatacatgcacacgcacatgtgagactcacatctaactccacagacatacatacatgaacatacatgtacatgtggagctgcttcaggttgctgagcttgtatgtacatggacctgtgtttgagtacaatgaacatacatctgtggctatatatgagccacacatatacatgacaaggctgtgtgcaggccacctctgtgattagttacccactctgtgcctatactattcattgccaccatcctgtgttcccagccccaacccaggggggatttattaacttagcagtctgacattagaatggagaggggcctcttcctgtctgtacccagcacctattccaactgtatttgactgcctcacggctagctagccactgcccattaaaatgttaataatcccaggatgaacaaatatgtgggccaaaaggcttcaggacagttcaccccatacccatttcatagctgtaggagactgagacccacttaccaaagccacataaaatcatgacagcagctcagggactagacattagctgggatgatcctctcaggtctctgcttcaaggcaaaaaccctgagcggtgacctcactctggaagcagcatgtcttccagaaaagccaggtgtccctagggccaagtggacttgcatttaagtgccagccccctacttactagcagtgtgaccttagacatctgcccctctctgagcccaccttttcaccataaagatagtaagccccatgtcactgaactgctgtgaagttaacttaattggtcttaatgatcacttactaggtttcaggaatgctcccttccctcttgagtctgggggcgggggggggggggctggaagaataggcagctgggttccttctcccaaaccctctaagagtagaaactacggtgttccccccttctccatccttttcccccacccctgtctctccttattccctctctctttctgtctataccagtcaaatactggggaaggatatgccaagccagtctgcctgtcttcccagcttccctggcccctttggagtgcaagacccccatgtctcctctccacccagatcatcagcacccccacctctaagccccataaaaggaacataacaggagaggccccagatgggccacatgccagcctctggcaccgccccctctgaccccctgctttctgtgcacaggctacgagaccctggcagaaacgccgtgtcccccagcgctgcagccacagccagctgaagacctggtacctagtggtcctgaggactgtggcttcttccccaatggggcctttgaccactgtctgagtcacatcccgtccatctacactgacacctgaaggaaggggcgctgctctgcctgcctgcctggctcctgagctacttcacctacctgccatctgctggtgcttcccacacggggcagcaaggccacaccacagggtacttccctacctggagggctgtctggtccagagctgcctgccaggagctatggccctctgacagccccatggctgtgtcttcctctctctccataaggttctcaaatcacagacctcgtgtatatacaatgtacaggacctcttttccgccgccctgcaagttttatatttttggttttacaagaaaaacattaaaaactggaaacta

SEQ ID NO: 14 - protein, 789 aa, mus musculus
MHCEVAEALSDKRPKEAPGAPGQGRGPVSLGAHMAFRIAVSGGGCGDGNPLDLLPRLPVPPPRAHDLLRPRSPRDYGVSKTGSGKVNGSYGHSSEKSLLDLDLAEGPSPSCHQGLFLPAGTPPPRGHPPVCEKLLHFPHPNRSPRPQATFVNGSLPAAQHIKQEALPDYQAMVSAHTPLPTHCRAPSSMGLPSDLDFPDRGLTNPAPSCYLLGNEPISDLGPQPEAHLPEGSLKRCCLLGLPPTSSASSSPCASSDINPVIHSSQTALVSCVNGLRSPPLPGDLGGPPKRSRPGPASSDGQEGSLQLEACRKSGFLKQEPMDEFSELFAPHHQGLPPPYPLPQLPTGPGLGGLGLGLAGRMVAGRQACRWVDCCAAYEQQEELVRHIEKSHIDQRKGEDFTCFWAGCVRRYKPFNARYKLLIHMRVHSGEKPNKCMFEGCSKAFSRLENLKIHLRSHTGEKPYLCQHPGCQKAFSNSSDRAKHQRTHLDTKPYACQIPGCSKRYTDPSSLRKHVKAHSAKEQQVRKKLHTGADPEADVLSECLSLQQLQASTLLPASRGKGSQTLSQELLPGVYPGSVTPQNGLASGILSPSHDVPSRHHPLEVPTGSHHHLSPLPTAESTRDGLGPSLLSPMVSPLKGLGPPPLPPASQSQSPGGQSFSTVPSKPTYPSFQSPPPLPSPQGYQGSFHSIQNCFPYADCYRATEPAASRDGLVGDAHGFNPLRPSTYSSLSTPLSAPGYETLAETPCPPALQPQPAEDLVPSGPEDCGFFPNGAFDHCLSHIPSIYTDT

SEQ ID NO: 15 - DNA, 8495 bp, artificial
  plasmid
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttaaaaagcaggctccaccttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacggggatctcaaaattgtcgctcctgtcaaacaaactcttaactttgatttactcaaactggctggggatgtagaaagcaatccaggtccactcatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccagcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgcgacagggccttttccaggtcggaccaccttgccttacacatgaagaggcactttggggatctcaaaattgtcgctcctgtcaaacaaactcttaactttgatttactcaaactggctggggatgtagaaagcaatccaggtccactcatgaacaattcgcccttcaccatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggaagggcgaattgcctgcaggaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 16 - DNA, 6131 bp, artificial
  Plasmid
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttcaccatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataaaagggcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 17 - DNA, 14545 bp, artificial
  Plasmid
ttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatacggcagacctcagcgctagattattgaagcatttatcagggttattgtctcatgagcggatacatatttgaatgtatttagaaaaataaacaaataggggttccgcgcacatttccccgaaaagtgccacctgacgttaactataacggtcctaaggtagcgaaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcccgctgatcttcagacctggaggaggagatatgagggacattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgaaagcttgggattcgaatttaaaagaaaaggggggattggggggtacagtgcaggggaaagaatagtagacataatagcaacagacatacaaactaaagaactacaaaaacaaattacaaaaattcaaaattttcgggtttttcgaacctagggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaatttcgactgctagggctagcttagccaccatgcaattgtctagaatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgtctagaggttctggtgccacgaagcaagcaggagatgttgaagaaaaccccgggcctactagaactagtatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgactagtgaattcttaggttctggtgagggcagaggaagtcttctaacatgcggtgacgtggaggagaatcccggccctggtaccatagtcgacatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcactttggtaccgtcgacggttctggtcagtgtactaattatgctctcttgaaattggctggagatgttgagagcaacccaggtcccggatccgatatcatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcaggatccgatatcacgcgtggttctggtgtgaagcagaccctcaattttgacctgctgaagctcgccggagatgtggagtccaaccccggaccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagacgcgttaagcggccgctggatcctctagactgcagctcgagtacccatacgacgtcccagactacgcttgagtttaaacacgcgtggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccatgaccgagtacaagcccacggtgcgcctcgccacccgcgacgacgtccctcgggccgtacgcaccctcgccgccgcgttcgccgactaccccgccacgcgccacaccgtggacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaagggctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttccggaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtctcgccttcgccctcagacgagtcggatctccctttgggccgcctccccgcctgtccggatggaagggctaattcactcccaacgaatacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggcatctatgtcgggtgcggagaaagaggtaatgaaatggcattatgggtattatgggtctgcattaatgaatcggccaacgatcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaacatcatcaataatatacctt

SEQ ID NO: 18 - RNA, 82 nt, artificial
  miRNA
ccuauguagcggccaucaaaguggaggcccucucuugagccugaaugagaaagugcuuccacuuugugugccacugcauggg

SEQ ID NO: 19 - RNA, 79 nt, Artificial
  miRNA
acauacagugucgaucaaaguggaggcccucuccgcggcuuggcgggaaagugcauccauuuuguuugucucugugugu

SEQ ID NO: 20 - RNA, 84 nt, artificial
  miRNA
uuccauauagccauacucaaaauggaggcccuaucuaagcuuuuaaguggaaagugcuucccuuuuguguguugccauguggag

SEQ ID NO: 21 - RNA, 69 nt, artificial
  miRNA
ggugagacucaaauguggggcacacuucuggacuguacauagaaagugcuacuacuuuugagucucucc

SEQ ID NO: 22 - RNA, 69 nt, artificial
  miRNA
ccaccacuuaaacgugguuguacuugcuuuagaccuaagaaaguaagugcuuccauguuuuggugaugg

SEQ ID NO: 23 - RNA, 74 nt, artificial
  miRNA
guucccuucaacuuuaacaugggaaugcuuucugucucaucgaagaguaagugcuuccauguuuuaguagaagu

SEQ ID NO: 24 - RNA, 68 nt, artificial
  miRNA
ccucugcuuuaacaugggguuaccugcuguguuaaacaaaagcaagugcuuccauguuucaguggggg

SEQ ID NO: 25 - RNA, 66 nt, artificial
  miRNA
ccuuuacuuuaacauggaggcacuugcugugcauuuaaaaauaagugcuuccauguuugagugugg

SEQ ID NO: 26 - RNA, 75 nt, artificial
  miRNA
aggccgucacuguugcuaacaugcaacucuguugaauagaaauuggaauugcacuuuagcaauggugauggaccu

SEQ ID NO: 27 - RNA, 79 nt, artificial
  miRNA
gguacuugaagggagaucgaccguguuauauucgcuuggcugacuucgaauaauacaugguugaucuuuucucaguauc

SEQ ID NO: 28 - RNA, 69 nt, artificial
  miRNA
ccaccacuuaaacguggauguacuugcuuugaaacuaaagaaguaagugcuuccauguuuuggugaugg

SEQ ID NO: 29 - RNA, 73 nt, artificial
  miRNA
gcucccuucaacuuuaacauggaagugcuuucugugacuuuaaaaguaagugcuuccauguuuuaguaggagu

SEQ ID NO: 30 - RNA, 68 nt, artificial
  miRNA
ccuuugcuuuaacauggggguaccugcugugugaaacaaaaguaagugcuuccauguuucaguggagg

SEQ ID NO: 31 - RNA, 68 nt, artificial
  miRNA
ccucuacuuuaacauggaggcacuugcugugacaugacaaaaauaagugcuuccauguuugagugugg

SEQ ID NO: 32 - RNA, 72 nt, artificial
  miRNA
uuggguaagugcuuccaugcuucaguuuccuuacugguaagauggauguaguaauagcaccuaccuuauaga

SEQ ID NO: 33 - RNA, 51 nt, artificial
  miRNA
ucuguguaaaccuggcaauuuucacuuaauugcuuccauguuuauaaaaga

SEQ ID NO: 34 - RNA, 67 nt, artificial
  miRNA
gugggccucaaauguggagcacuauucugauguccaaguggaaagugcugcgacauuugagcgucac

SEQ ID NO: 35 - RNA, 69 nt, artificial
  miRNA
gggauacucaaaaugggggcgcuuuccuuuuugucuguacugggaagugcuucgauuuugggguguccc

(19) United States
(12) Patent Application Publication
(10) Pub. No.: US 2014/0323411 A1
(43) Pub. Date: Oct. 30, 2014

(54) Method for De-Differentiating A Cell

(71) Applicant: UCL Business PLC, London (GB)

(72) Inventor: Konstantinos Kostarelos, London (GB)

(21) Appl. No.: 14/356,348

(22) PCT Filed: Nov. 5, 2012
(86) PCT No.: PCT/GB2012/052749

§ 371 Date: May 5, 2014

(30) Foreign Application Priority Data
Nov. 3, 2011 (GB) 1118964.4

(51) Int. Cl. C07K 14/47; C12N 15/113

(52) U.S. Cl. CPC C07K 14/47; C12N 15/113
USPC 514/19.3; 514/44 R; 435/377; 514/44 A; 514/1.1

(57) ABSTRACT

The invention relates to a method for de-differentiating a cell, i.e. the induction of a pluripotent phenotype. In vivo de-differentiation is carried out using defined factors such as transcription factors, miRNA, DNA, or proteins. This leads to the formation of pluripotent cells, without teratoma formation. Defined factors may be administered to cells such as liver or muscle cells and are useful in therapy.

### FIELD OF THE INVENTION

The present invention relates to a method for de-differentiating a cell, i.e. the induction of a pluripotent phenotype.

### BACKGROUND OF THE INVENTION

Forced reprogramming of somatic cells into a pluripotent, stem cell-like state by the ectopic expression of specific transcription factors results in the generation of induced pluripotent stem (iPS) cells. Such transcription factor cell reprogramming has been achieved in vitro today by either viral and non-viral gene transfer, protein translocation, and more recently miRNA and is changing the landscape in developmental biology, can potentially resolve all ethical concerns about the use of embryonic stem cells and open further opportunities for regenerative medicine. The original discovery by Yamanaka and colleagues that the in vitro expression of four transcription factors, Oct3/4, Klf4, Sox2, c-Myc (OKSM) was capable of reverting isolated adult, fully differentiated mouse and human skin fibroblasts into iPS cells^{(5, 8)} constitutes the fundamental and most widely used reprogramming technology today.

Due to the paradigm-shifting nature of the phenomenon there is still limited understanding of the exact mechanisms and pathways implicated in induced reprogramming. Moreover, the entire body of experimental evidence today is based on the concept of extraction and in vitro manipulation of the somatic cells to be reprogrammed, leading to an array of caveats. Namely, low levels of reprogramming efficiency, primarily use of viral vectors for effective transcription factor expression, long, cumbersome, difficult to reproduce and complex cell culturing conditions that make clinical translation of the iPS technologies seem very distant.

The initial reports of in vitro somatic cell reprogramming involved the use of retroviruses to stably transduce skin fibroblasts with the cocktail of reprogramming factors⁽³⁻⁵⁾. This methodology of gene transfer is still today the most popular way to reprogram animal and human somatic cells despite the risks from insertional mutagenesis, stable transduction and long-term gene expression of known proto-oncogenes. There are continuous developments for the discovery of improved methodologies to create iPS cells from isolated somatic cells in the most efficient and safest manner. Despite these efforts, the vast majority involves use of viral vectors and long-term culturing and treatment of cells with multiple rounds of gene transfer vectors, growth factors, antibiotics and other cell media cocktails to promote reprogramming and select for pluripotency. All of these are considered major culprits for the potential risks associated with the ensuing cells as recent studies investigating the genomic integrity of iPS have alluded to. In terms of in vitro iPS generation using non-viral gene transfer vectors, plasmid DNA⁽⁸⁾ or RNA⁽⁶⁾ delivery using liposomes or electroporation are the most commonly used approaches. Compared to viruses, episomal vectors are safer however transduction and reprogramming efficiencies are much lower⁽⁹⁾. Alternatively, Warren et al. reported somatic cells reprogramming in vitro by direct delivery of synthetic mRNAs⁽⁶⁾. Although this methodology offers significantly higher reprogramming efficiency, high RNA dosages, multiple rounds of transfection and complex cell culturing protocols are still needed⁽⁹⁾.

### SUMMARY OF THE INVENTION

In vivo cell reprogramming (cell de-differentiation) using defined factors has never before been attempted, due to the belief that it would lead to the spontaneous occurrence of teratomas within the tissues where pluripotent cells are generated. It was therefore surprising to find that in vivo de-differentiation using defined factors, led to the formation of pluripotent cells, without teratoma formation (the animals tested were kept for a period of 120 days and monitored for teratoma formation at frequent intervals).

Another surprising finding was that the rate of induction of the pluripotent phenotype by the method of the invention is at least 10-fold more efficient than the known in vitro method using the same defined factors. Not only are about 10 times more cells induced to a pluripotent state, but the reprogramming process occurs much more rapidly than the in vitro processes of the prior art. Using a method of the invention, cells can be reprogrammed within 24 hours. This is compared to the in vitro methods, which take 3-6 weeks and are very laborious and require careful selection of culturing media, for example.

The present invention is based at least in part on a study showing that following a single hydrodynamic tail-vein injection of two plasmids containing genes Oct3/4, Sox2 and Klf4 and c-Myc respectively, these genes are highly expressed in the liver tissue of Balb/C adult mice, leading to direct hepatocyte cell reprogramming in vivo. The reprogramming process occurred very rapidly and within 24 h after injection both liver tissue sections and the total population of extracted primary hepatocytes exhibited significantly higher levels of various pluripotency markers followed by down-regulation of all major hepatocellular markers. The primary hepatocytes from the reprogrammed liver tissues were isolated and cultured in vitro and found to exhibit significantly higher cell proliferation, staining for alkaline phosphatase (ALP) and all major pluripotent markers. Importantly, the animals exhibited no sign of physiological (liver function) or structural (histological) abnormality or teratoma formation within the 120 days period studied.

Further, direct in vivo reprogramming was able to silence adenoviral transgene expression and significantly alleviate hepatotoxic resposes characteristic of high-dose adenoviral transfection. These findings indicate that direct in vivo cell reprogramming of mammalian tissue can be safely achieved by non-viral gene transfer and leads to rapid generation of pluripotent cells within tissues in the absence of long-term tissue damage or teratoma formation. In vivo induced pluripotent stem cells can be extracted, isolated and cultured as stem cell colonies within 48h and in vivo reprogramming can be used to silence genes in tissues that may be implicated in pathological phenotypes. Therefore, the in vivo reprogramming of the invention is useful in therapy.

A similar study has additionally found that pluripotent cells can be generated in muscle cells in accordance with this invention.

According to a first aspect, an in vivo method of de-differentiating a cell, comprises transfecting the cell with a defined factor.

According to a second aspect, a pluripotent stem cell is obtainable by the method described above.

According to a third aspect, a pluripotent stem cell as described above, is useful in therapy.

According to a fourth aspect, a defined factor induces a pluripotent phenotype in vivo, and is therefore useful in therapy.

According to a fifth aspect, a pharmaceutical composition comprises a defined factor and a pharmaceutically acceptable excipient.

### DESCRIPTION OF THE DRAWINGS

FIG. 1 shows direct in vivo gene transfer with reprogramming factors. Balb/C mice were HTV injected with either 75 μg of pCX-OKS-2A and 75 pCX-cMyc in 0.9% saline or 0.9% saline only. At days 2, 4, 8, 12, 24, qRT-PCR analysis of hepatocytes was performed to determine the expression levels of (a) reprogramming factors and (b) pluripotency genes. In (c) flow cytometry analysis was performed to determine OCT3/4 and NANOG positive cells in the liver extracts. In (d) expression of hepatocyte markers was also determined by qRT-PCR. The expression levels are in relation to HTV-injected saline group throughout.

FIG. 2 reports a dose-response experiment showing expression levels of reprogramming factors and pluripotency genes. Balb/C mice were HTV injected with either 75 μg of pCX-OKS-2A and 75 pCX-cMyc in 0.9% saline or 150 μg of pCAG-GFP in 0.9% saline or 0.9% saline only. At day 4, qRT-PCR analysis of hepatocytes was performed to determine the expression levels of (a) reprogramming factors and (b) pluripotency genes. The expression levels are in relation to HTV-injected saline group throughout.

FIG. 3 shows flow cytometry analysis of in vivo-reprogrammed hepatocyte extracts. Balb/C mice were HTV injected with either 75 μg of pCX-OKS-2A and 75 pCX-cMyc in 0.9% saline or 0.9% saline only. At day 2, hepatocytes were isolated and stained for OCT3/4, SOX2 and NANOG. The expression levels are in relation to HTV-injected saline group throughout.

FIG. 4 shows expression levels in whole liver extracts vs. hepatocyte population. Balb/C mice were HTV injected with either 75 μg of pCX-OKS-2A and 75 pCX-cMyc in 0.9% saline or 0.9% saline only. At day 2, whole liver extracts and hepatocyte population were obtained and expression of (a) reprogramming and (b) pluripotency factors was analyzed by qRT-PCR. The expression levels are in relation to HTV-injected saline group throughout.

FIG. 5 shows the teratoma formation capacity of in vivo IPS colonies. Balb/C mice HTV were injected with either 75 μg of pCX-OKS-2A and 75 μg of pCX-cMyc in 0.9% saline or 150 μg of pCAG-GFP in 0.9% saline or 0.9% saline alone. At day 2 extracted hepatocytes were isolated and cultured on either Matrigel-coated plates or MEF feeder layers. (a) Phase contrast microscopy images were taken with light microscope (100×); (b) RNA was isolated 1 or 8 days after culturing. Real-time PCR analysis was performed to determine the expression levels of reprogramming and pluripotency genes. Expression levels were relative to saline group; (c) Cultured cells on Matrigel were stained with ALP; (d) Staining with ALP or CD1Y was also carried out on in vivo induced pluripotent stem cell colonies cultured on MEF layers; (e) In vivo induced pluripotent stem cell colonies were stained with Oct3/4, Sox2, Nanog and SSEA-1 and imaged with a fluorescent microscope (100×); (f) Hepatocyte extracts on day 2 after OKSM-HTV injections were isolated and subcutaneously implanted in CD1 nude mice. After 5 weeks, mice developed teratomas, as shown. Teratomas were dissected and stained with H&E. The development of different tissue types was observed with a light microscope (100×). M, G and A stands for muscle, gland and adipocyte tissue, respectively.

FIG. 6 shows cultured mouse embryonic stem cells (E14) on gelatin and MEF feeder layer. Phase contrast microscopy is shown of mES-E14-TG2A (left), MEF (middle), and mES-E14-TG2A plated on MEF feeder layer (right) (100×).

FIG. 7 shows the cultures of FIG. 6, stained with pluripotency markers. Immunofluorescence staining is shown of mES-E14-TG2A on MEF feeder layer for (reprogramming) Oct3/4, Sox2 and (pluripotency) Nanog and SSEA1 markers (100×).

FIG. 8 shows tissue liver section staining with pluripotency markers. Balb/C mice were HTV injected with either 75 μg of pCX-OKS-2A and 75 μg pCX-cMyc in 0.9% saline or 0.9% saline alone. (a) At day 4, livers were collected and frozen tissue sections were stained with anti-Oct4 antibody, anti-Sox2 antibody, anti-Nanog antibody or BCIP/NBT to determine ALP activity (400×); (b) H&E stained liver sections at days 2, 4, 8, 12, 50 and 120 after HTV injection of OKSM plasmids or 0.9% saline. Images were captured with light microscopy (100×).

FIG. 9 shows immunfluorescence staining with Nanog of different liver sections after HTV injection of OKSM plasmids. Balb/C mice were HTV injected with 75 μg of pCX-OKS-2A and 75 μg pCX-cMyc in 0.9% saline. At day 4, liver tissue was collected and frozen tissue sections were stained with anti-Nanog antibody (magnification 400×).

FIG. 10 shows the effect of HTV injection on hepatotoxicity and liver damage. Balb/C mice were HTV injected with either 75 μg of pCX-OKS-2A and 75 μg pCX-cMyc in 0.9% saline or 0.9% saline only. On days 4, 8, 12 and 120 sera were isolated. (a) Levels of liver enzymes and (b) albumin were analyzed; (c) Liver sections were PAS stained to determine glycogen storage levels. Representative images were captured with light microscopy (100×).

FIG. 11 shows that in vivo-reprogramming according to the invention silences Ad.luc transgene expression and hepatotoxicity. (a) Schematic representation of in vivo Ad.luc and Ad.luc followed by OKSM plasmid injection protocol. Balb/C mice were injected with Ad-luc (3×10¹¹pu) via intravenously (i.v.) in 100 μl of HEPES buffer. 4h post-Ad injection, pCX-OKS-2A and pCX-cMyc plasmids were HTV-injected; (b) Whole-body imaging using IVIS Lumina and ROI analysis of the abdominal area; (c) Hepatocytes were isolated at day 2 and luciferase gene expression was determined by qRT-PCR; (d) From the same hepatocyte extracts, DNA was isolated to determine methylation status of the CMV promoter. Methylation ratio was obtained by qRT-PCR following BsaHl digestion; (e) Whole liver tissues were homogenised and the chemiluminescence luciferase assay was performed at days 5 and 9; (d) Blood sera were obtained from naive, Ad and Ad+OKSM groups on day 5 & 9, and levels of liver enzymes were analyzed; (e) liver tissue sections were stained with H&E and images were captured with light microscopy (200×).

FIG. 12 shows protein levels in liver tissue after Ad.luc and Ad.luc followed by OKSM plasmid HTV-injections. Balb/C mice were injected with Ad.luc (3×10¹¹pu) intravenously in 100 μl HEPES buffer. 4h post-Ad injection, pCX-OKS-2A and pCX-cMyc plasmids were HTV-injected. Whole liver tissue was homogenised and total protein levels for each group were analyzed by BCA assay on day 5 and 9.

FIG. 13 shows induction of pluripotency in Nanog-GFP mice. Nanog-GFP mice were HTV injected with 0.9% saline alone, 75 μg of pCX-OKS-2A and 75 μg pCX-cMyc in 0.9% saline. At day 4, liver samples were frozen and sectioned to image GFP positive cells under fluorescent microscope (10×).

FIG. 14 shows induction of pluripotency in muscle. (a) Balb/C mice were intramuscularly injected with 0.9% saline alone or 50 μg of pLenti-II1-EF1a-mYamanaka in 0.9% saline. At day 2, muscle samples were collected and stained with H&E to observe the muscle histology, BCIP/NBT to determine ALP activity in the tissue or anti-NANOG antibodies to assess immunoreactivity. (b) Nanog-GFP mice were intramuscularly injected with 0.9% saline alone, 50 μg of pCX-OKS-2A and 50 μg pCX-cMyc in 0.9% saline and frozen muscle sections from day 2 were imaged under fluorescent microscope for GFP positive cells (20×).

### DESCRIPTION OF THE SEQUENCE LISTING

SEQ ID NO: 1 is the DNA sequence of the defined factor Oct3/4.

SEQ ID NO: 2 is the protein sequence of the defined factor Oct3/4.

SEQ ID NO: 3 is the DNA sequence of the defined factor Sox2.

SEQ ID NO: 4 is the protein sequence of the defined factor Sox2.

SEQ ID NO: 5 is the DNA sequence of the defined factor Klf 4.

SEQ ID NO: 6 is the protein sequence of the defined factor Klf 4.

SEQ ID NO: 7 is the DNA sequence of the defined factor CMyc.

SEQ ID NO: 8 is the protein sequence of the defined factor CMyc.

SEQ ID NO: 9 is the DNA sequence of the defined factor Nanog.

SEQ ID NO: 10 is the protein sequence of the defined factor Nanog.

SEQ ID NO: 11 is the DNA sequence of the defined factor Lin 28.

SEQ ID NO: 12 is the protein sequence of the defined factor Lin 28.

SEQ ID NO: 13 is the DNA sequence of the defined factor Glis1.

SEQ ID NO: 14 is the protein sequence of the defined factor Glis1.

SEQ ID NO: 15 is the DNA sequence for the plasmid pCX-OKS-2A.

SEQ ID NO: 16 is the DNA sequence for the plasmid pCX-cMyc.

SEQ ID NO: 17 is the DNA sequence for the plasmid pLenti-II1-EF1a-mYamanaka.

SEQ ID NO: 18 is the sequence for the mouse miRNA mmu-miR291a.

SEQ ID NO: 19 is the sequence for the mouse miRNA mmu-miR291a.

SEQ ID NO: 20 is the sequence for the mouse miRNA mmu-miR294.

SEQ ID NO: 21 is the sequence for the mouse miRNA mmu-miR295.

SEQ ID NO: 22 is the sequence for the mouse miRNA mmu-miR302a.

SEQ ID NO: 23 is the sequence for the mouse miRNA mmu-miR302b.

SEQ ID NO: 24 is the sequence for the mouse miRNA mmu-miR302c.

SEQ ID NO: 25 is the sequence for the mouse miRNA mmu-miR302d.

SEQ ID NO: 26 is the sequence for the mouse miRNA mmu-miR367.

SEQ ID NO: 27 is the sequence for the mouse miRNA mmu-miR369.

SEQ ID NO: 28 is the sequence for the human miRNA hsa-miR302a.

SEQ ID NO: 29 is the sequence for the human miRNA hsa-miR302b.

SEQ ID NO: 30 is the sequence for the human miRNA hsa-miR302c.

SEQ ID NO: 31 is the sequence for the human miRNA hsa-miR302d.

SEQ ID NO: 32 is the sequence for the human miRNA hsa-miR302e.

SEQ ID NO: 33 is the sequence for the human miRNA hsa-miR302f.

SEQ ID NO: 34 is the sequence for the human miRNA hsa-miR372.

SEQ ID NO: 35 is the sequence for the human miRNA hsa-miR373.

### DESCRIPTION OF THE PREFERRED EMBODIMENTS

As used herein, “de-differentiating” or “de-differentiation” means returning the cell at the least to a pluripotent state. It also includes retuning the cell to a totipotent state. Preferably, it means returning the cell to a pluripotent state.

As used herein, “defined factor” means an agent, which can be a transcription factor or miRNA, for example, which can de-differentiate a cell to a pluripotent state. The term is widely used in the art and includes transcription factors, such as Oct3/4, Sox2, Klf4 and c-Myc Nanog, Lin 28 or Glis 1. A defined factor may also be an miRNA sequence, such as miR-291, miR-295, mi302, mi372, mi369, mi302 or mi367. A defined factor may also be a DNA sequence or a protein.

In a preferred embodiment, the defined factor is one or more of Oct3/4, Sox2, Klf4 or c-Myc. Preferably, two or more, more preferably three or more, more preferably four. Preferably, the defined factors are Oct3/4, Sox2 and Klf4. Alternatively, the defined factors are Oct3/4, Sox2, Klf4 and c-Myc.

In vitro somatic cell reprogramming has been achieved by co-transfection with different transcription factors or by ectopic transcription of miRNAs. Until now, Oct3/4, Sox2, Klf4, c-Myc, Nanog, Lin28, Glis1 have been used in different combinations as transcription factor-mediated cellular reprogramming²³⁻²⁶. Also, defined miRNA molecules (miR-291-3p, miR-294 and miR-295) have been shown to increase the efficiency of in vitro reprogramming by Oct4, Sox2 and Klf4²⁷, whereas other miRNAs (mi302, mi372, mi369 or mi302/367 clusters) were also able to reprogramme cells to a pluripotent stage²⁸⁻³¹.

In a preferred embodiment, the cell is transfected by one or more vectors comprising those transcription factors. Suitable vectors will be known to the person skilled in the art and include viral vectors, such as those derived form an adenovirus or a lentivirus.

In a preferred embodiment, the vector is a plasmid. A suitable plasmid for use in the invention is pCX-OKS-2A. It may be combined with pCX-cMyc. A further suitable plasmid is pLenti-III-EF1a-mYamanaka. These plasmids are known to the person skilled in the art.

A defined vector according to the invention may transfect any suitable tissue, such as liver (e.g. a hepactocyte) or muscle. In a preferred embodiment, the cell that is transfected is a hepatocyte or a muscle cell, preferably a skeletal muscle cell.

Any suitable method can be used to transfect a cell with a defined factor, and these will be known to those skilled in the art. For example, the vector may be delivered directly into the relevant tissue. A preferred method of administration is by hydrodynamic injection, or by using occlusion-assisted infusion, for example by using a balloon catheter. If the liver is being used as a source for cells, then the catheter may be placed in a hepatic vein. Alternatively, the vector may be delivered by intramuscular injection into muscle tissue.

A hydrodynamic injection is a method of administration known in the art. It can result in a very high transfection rate. A hydrodynamic injection is characterised by the rapid delivery of an agent in solution, for example a solution of naked plasmid DNA encoding a transcription factor. The volume of the solution containing the agent is preferably equivalent to about 8-12% the body weight of the animal to which the agent is being administered. The solution is preferable administered at a rate of about 1 to 3 seconds per mL.

In Study 1 described below, the animals tested were Balb/C adult mice, and the hydrodynamic injection was into the tail vein. It is proposed therefore that a similar method would be effective in humans. Therefore, in an embodiment of the invention, the defined factor may be delivered in solution via a balloon catheter inserted into a vein for example a hepatic vein, using occlusion-assisted infusion. This results in a large volume of agent moving rapidly into the tissue to be transfected and results in high transduction efficiency. The details of these methods are known in the field.

Due to the ability of the defined factors to return a cell to the pluripotent state in vivo, the defined factors of the invention are useful in therapy. There are a number of therapies that can be effected by the invention as the defined factors can reprogram a variety of diseased states. In the example herein, hepatotoxicity is reversed. However, the defined factors may also be used in the therapy of cancer, for example. The method of the invention is particularly beneficial as a tumour may be targeted with great specificity.

Other diseases which can be treated by agents of the invention are those conditions associated with degenerative cells or tissue damage, and pathologies associated with aged cells with accumulated mutations.

Further examples of diseases which can be treated by a method of the invention are those diseases that are associated with loss of tissue. Examples of diseases are liver cirrhosis, Parkinson's Disease, diseases involving damage to the heart, such as cardiomyocyte damage following myocardial infarction, or stroke.

### Study 1

In this study we hypothesized that direct in vivo somatic cell reprogramming by transient overexpression of the OKSM reprogramming transcription factors in living tissue can take place. In order to test this hypothesis we chose the most naive, non-viral gene transfer technology available today: the large-volume, rapid, intravenous administration, termed hydrodynamic tail vein (HTV) injection, of plasmid DNA^{(10, 11)} encoding the OKSM reprogramming factors. This gene transfer methodology circumvents most complications or risks associated with viral gene transfer vectors as has been previously shown in numerous preclinical^{(12, 13)} and clinical^{(14, 15)} studies allowing unprecedented levels of exogenous gene expression in hepatocytes. Balb/C animals were injected by HTV with an equimolar mix of two plasmids, pCX-OKS-2A and pCX-cMyc, encoding for the OKS and M reprogramming factors respectively. HTV injection of plasmid DNA results in high levels of gene expression in hepatocytes^{(10, 11)}, therefore primary hepatocytes from the injected animals were extracted at different time points. qRT-PCR was used to analyze the gene expression levels of various reprogramming (Oct3/4, Klf,4, Sox2, c-Myc), pluripotency (Nanog, Ecatl, Rexl, Cripto, Gdf3 and endogenous Oct3/4, Klf,4 or Sox2) and hepatocyte markers (Alb, Trf, AAT) in the hepatocyte population directly on extraction at different time points after HTV injection.

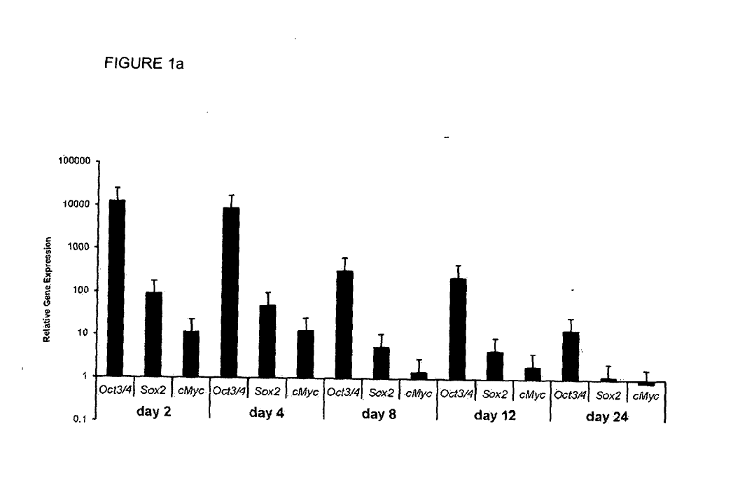

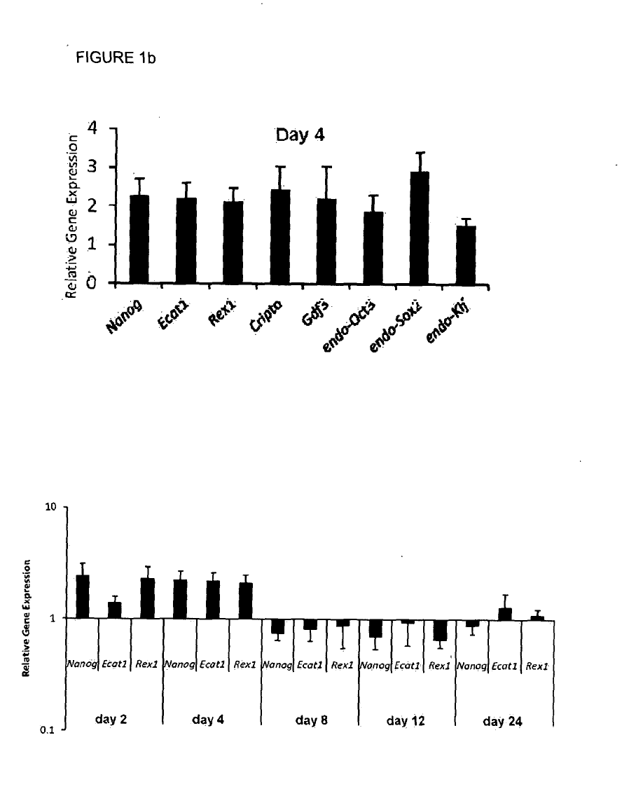

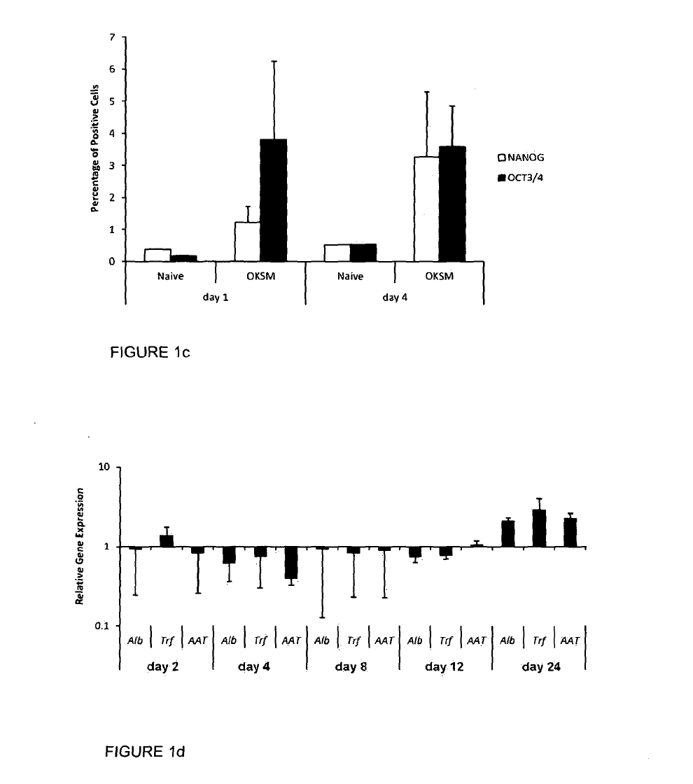

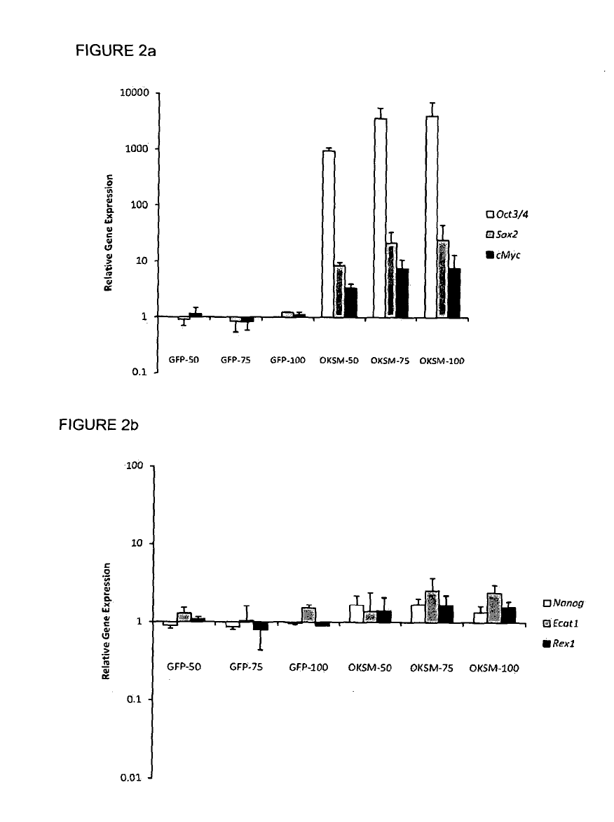

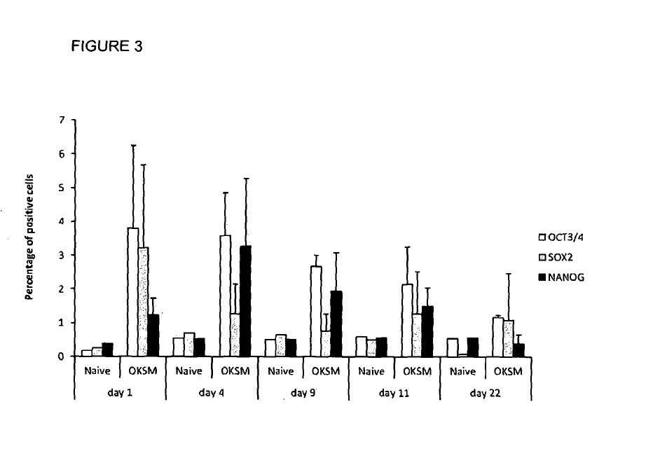

A significant increase in the gene expression of all transfected reprogramming factors was observed on day 2 post-HTV injection that decreased over time (FIG. 1a). At the same time endogenous pluripotency markers were upregulated, reached peak values on day 4 and decreased to background levels from day 8 onward (FIG. 1b). Protein expression in the hepatocyte extracts from the transduced liver tissue using flow cytometry indicated that on day 1 only OCT3/4 was expressed, whereas by day 4 both OCT3/4 and NANOG positive cells (3-4%) were detected (FIG. 1c). On day 8 onward, pluripotency factors began to be downregulated reaching saline levels by day 22 (FIG. 2). The effect of plasmid dose was studied next by HTV injections of OKSM against control plasmid DNA (expressing GFP controlled by the same CAG promoter) at an escalated dose regime. A sharp increase in the levels of gene expression profile of the transduced reprogramming (FIG. 3a) and other pluripotent (FIG. 3b) factors was observed only in the case of OKSM plasmid DNA injections, and the levels of upregulation reached a plateau at 75 μg/animal.

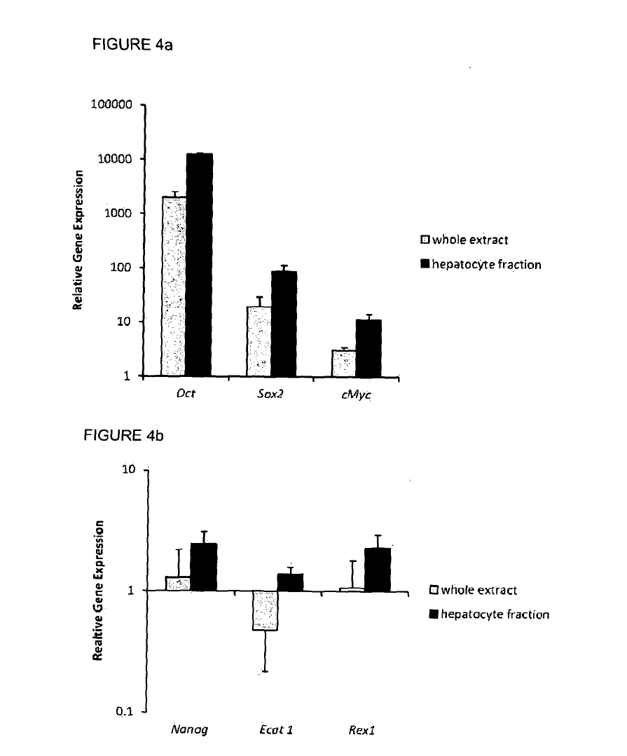

It is well-known that hepatocytes are the main target cells from gene transfer by HTV, and this was also confirmed here as the hepatocyte fraction contained cells that expressed significantly higher levels of reprogramming (FIG. 4a) and pluripotency (FIG. 4b) genes compared to whole liver extracts. Most interestingly, examination of the expression levels of hepatocyte markers by qRT-PCR indicated that on day 2 they were similar to those of animals injected with HTV saline, while at day 4 significant downregulation in hepatocyte marker expression was considered as another indication of the generation of induced pluripotent cells manifested by the de-differentiation of hepatocytes (FIG. 1d). Later than day 8, hepatocyte markers returned back to control levels, while on day 24 upregulated expression levels of these markers was thought to indicate the presence of proliferated and re-differentiated hepatocytes.

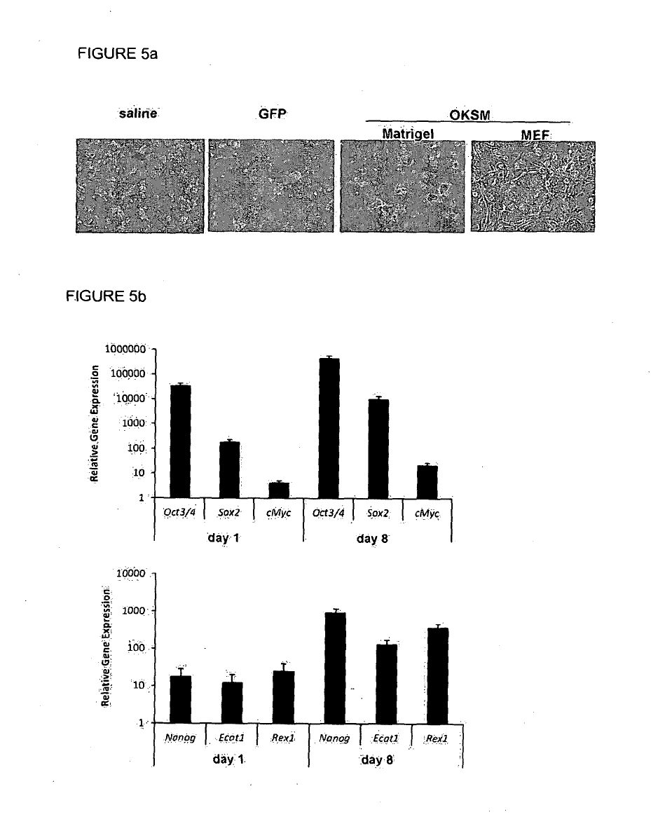

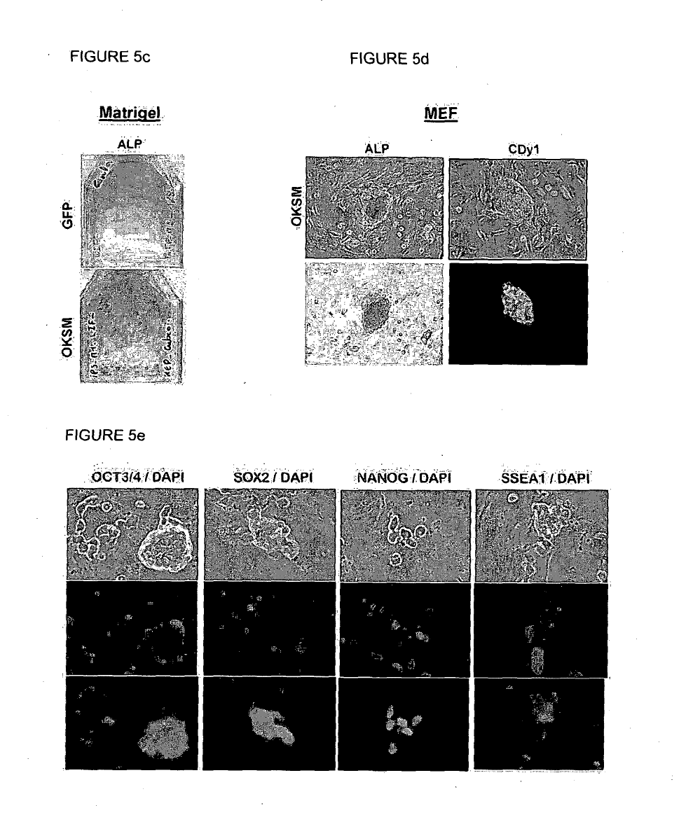

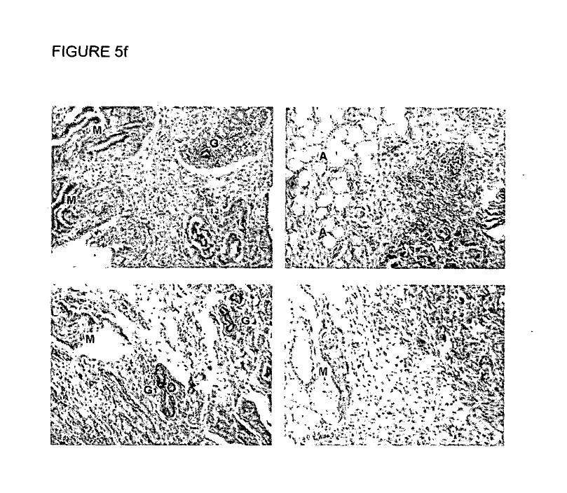

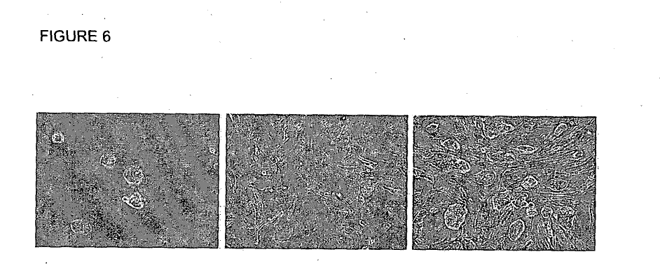

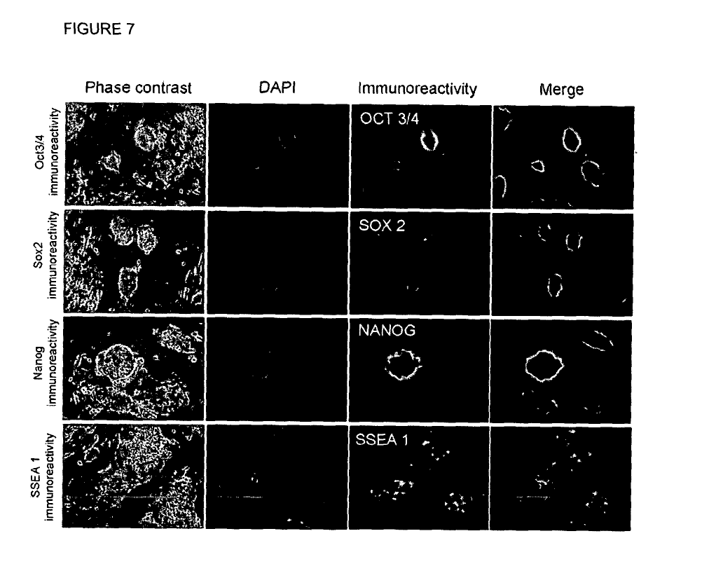

Isolation, culturing and characterisation of the in vivo induced pluripotent cells from the liver tissue by extracting the hepatocyte population from animals HTV-injected with the OKSM plasmids was carried out next. Primary hepatocyte fractions from liver tissues 2 days after HTV injection were extracted and cultured on both Matrigel-coated plates or on a mouse embryonic feeder (MEF) layer (FIG. 5a). After 8 days in culture, distinct colonies were formed only from hepatocyte extracts of the OKSM plasmid-injected animals under both culture conditions, however, the quality of the colonies on MEF was consistently higher. These liver-extracted colonies were similar in shape and morphology to those obtained from embryonic mouse stem cells (mES-E14-TG2A) grown on MEF (FIG. 6). Gene expression analysis by qRT-PCR of the extracted cells revealed significantly enhanced pluripotency genes after 1 day on MEF that were even further upregulated between 10 and 100 times after 8 days in culture (FIG. 5b). To further characterise the liver-extracted cells, a series of pluripotent markers were used to stain the colonies. ALP stained positively only hepatocyte extracts from OKSM (but not GFP) plasmid-injected animals cultured on Matrigel (FIG. 5c). Colonies extracted from the OKSM plasmid-injected animals cultured on MEF, stained positively for a variety of pluripotency markers detected colorimetrically (ALP and CDy1) (FIG. 5d) and immunohistochemically (Oct3/4, Sox2, Nanog, SSEA-1) (FIG. 5e), in an identical pattern to that obtained for mES-E14-TG2A cultured on MEF (FIG. 7). In a separate experiment and in order to investigate the pluripotent nature of these cells, primary hepatocyte fractions were extracted 2 days after OKSM plasmid HTV injection and 5×10⁶ cells were subcutaneously implanted into CD1 nude mice bifocally. After 5 weeks, all nude animals developed teratomas and the presence of different germ layers was histologically verified (FIG. 5f).

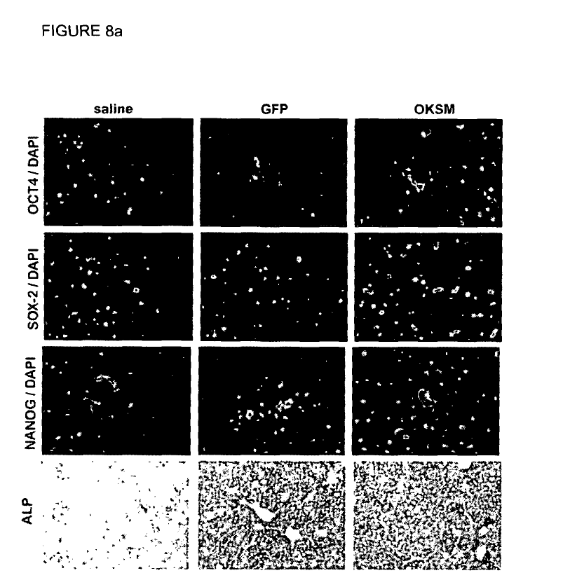

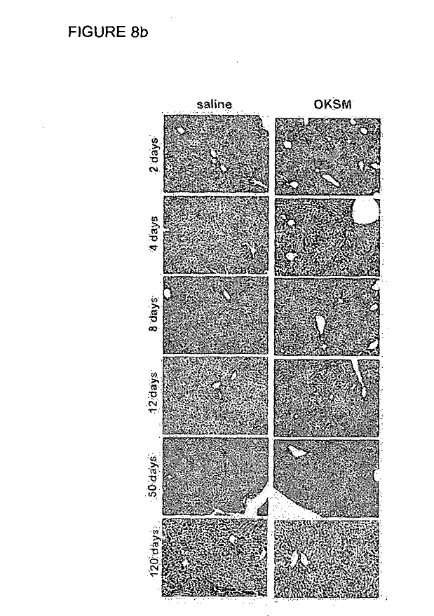

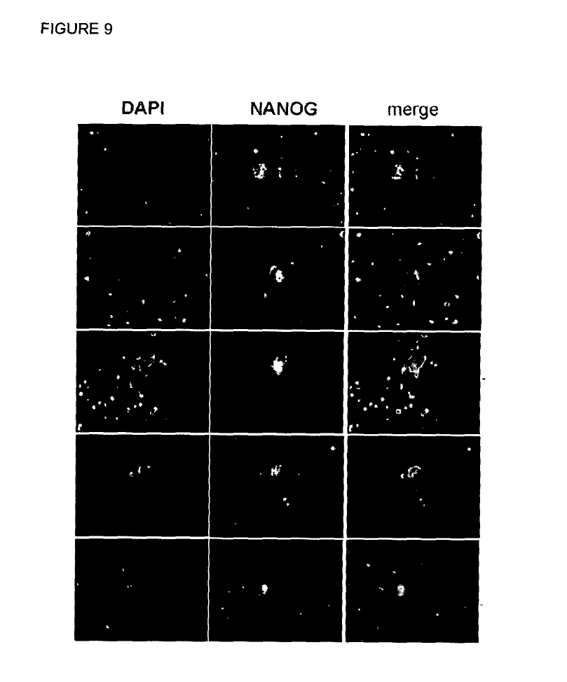

In order to further interrogate the occurrence of in vivo cell reprogramming in liver by the forced expression of the OKSM transcription factors, tissue sections from transfected mice were immunohistochemically (IHC) stained at day 4 (post-HTV) for hallmark reprogramming and pluripotency markers (Oct3/4, Sox2, Nanog, ALP). FIG. 8a shows that distinctive ICH-positive cells were observed for all four markers tested, only in the case of animals transfected with the OKSM plasmids compared to saline or GFP plasmid HTV-injected groups. Positive staining for Nanog was obtained reproducibly in all liver sections in all OKSM-transfected animals indicating the presence of pluripotent cells throughout the liver tissue (FIG. 9).

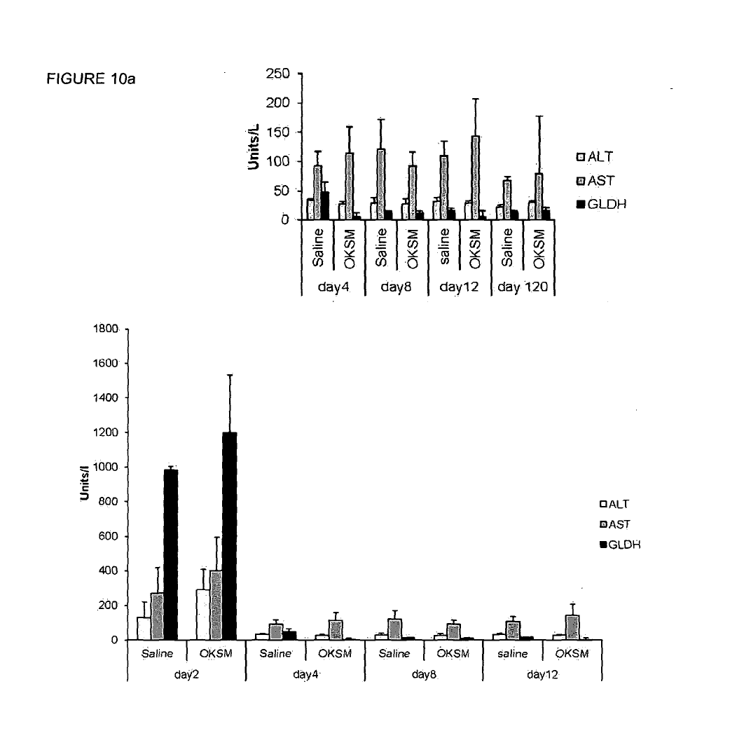

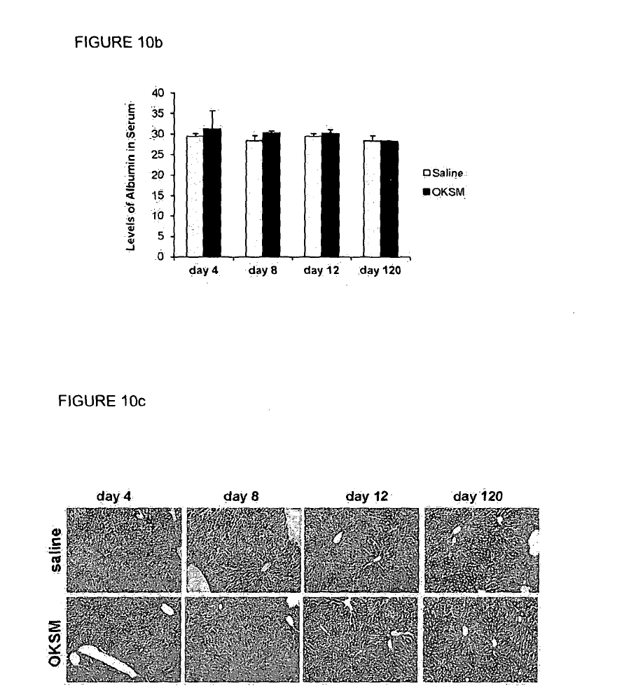

One of the key concerns from in vivo cell reprogramming may be the spontaneous occurrence of teratomas within the tissues where pluripotent cells are generated^{(1, 7)}. To address this, animals HTV-injected with the reprogramming factors were kept for a period of 120 days and at frequent intervals (days 2, 4, 8, 12, 50 and 120) different groups were analysed haematologically and histologically (FIG. 8 and FIG. 10). HTV injection of plasmid DNA can result in moderate tissue damage manifested by increased serum levels of liver enzymes at a very early time point that was also observed here for all HTV-injected groups (FIG. 10a). H&E staining of liver tissues indicated that HTV injection of OKSM plasmids did not lead to any tissue damage or development of teratomas, with all liver sections and animals exhibiting healthy structural morphology and behaviour with no signs of dysplasia (FIG. 8b). Serum levels were also analysed for liver enzymes over the same period, with no aberrant changes in the levels of ALT, AST and GLDH between the saline-injected and OKSM-injected groups (FIG. 10a). Albumin levels and glycogen staining of liver sections (FIGS. 9b and c) further confirmed no hepatic structural or functional abnormality throughout the course of the study for any of the animals. These findings in conjunction with the gene expression analysis in FIG. 1 suggested that in vivo reprogramming in the liver was occurring rapidly after HTV-injection of the OKSM factors and transiently.

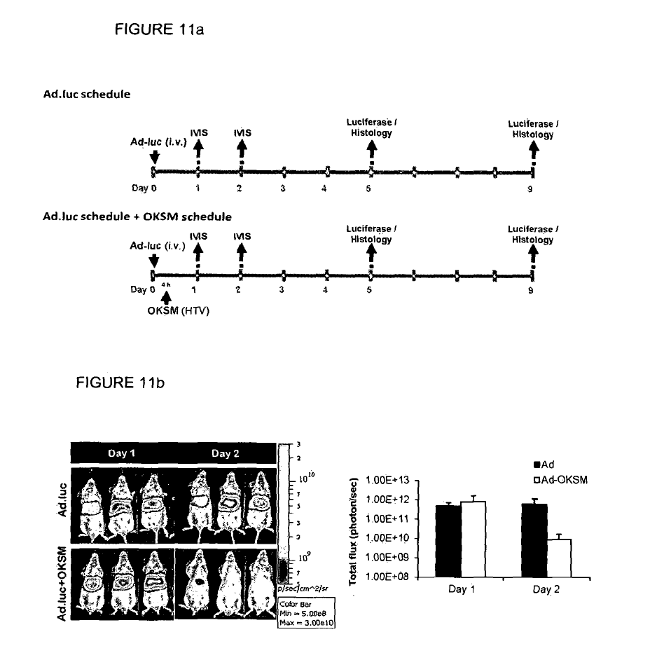

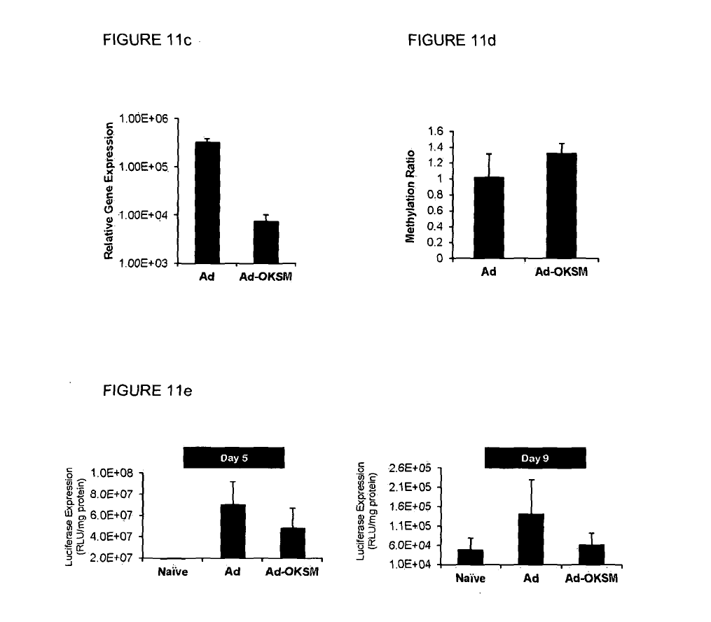

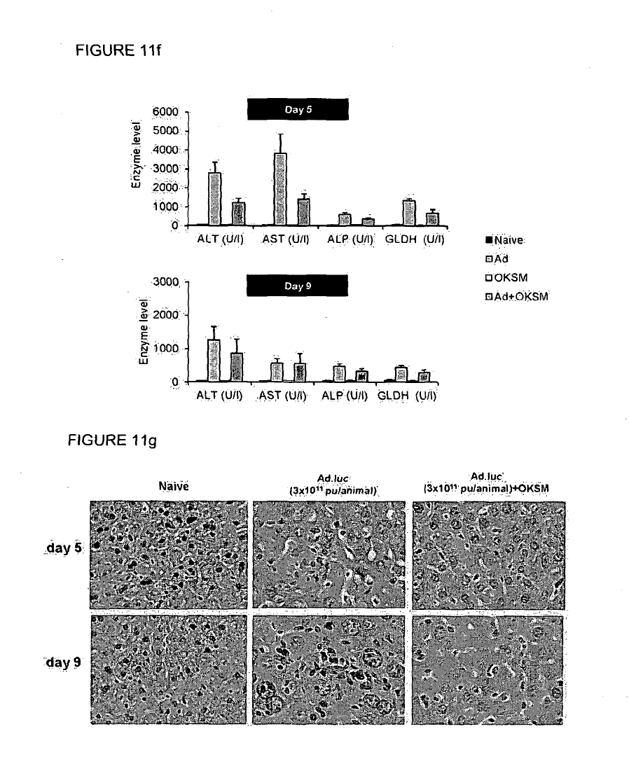

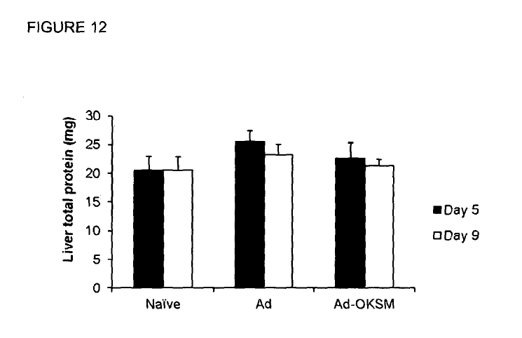

Based on previous evidence that silencing of viral promoters is a reliable indication of true somatic cell reprogramming^{(8, 16-19)}, we hypothesized that direct in vivo reprogramming should also be able to silence viral gene expression in vivo. In order to test this, Balb/C mice were intravenously administered with adenovirus (Ad5) encoding the luciferase transgene under the control of the CMV promoter (Ad.CMV.luc), and after 4h the same animals were HTV-injected with the OKSM plasmids (FIG. 11a). Adenoviral luciferase transgene expression was studied by whole body bioluminescence imaging (IVIS Lumina) on day 1 and 2 after Ad5 administration under aneasthesia, and at day 5 and 9 using the much more sensitive chemiluminescence assay and histological examination of the excised liver tissue. Significant luciferase gene silencing was observed at day 2 by bioluminescence imaging (FIG. 11 b), also confirmed by qRT-PCR analysis of the tissue (FIG. 11c). A PCR-based CpG-island methylation study of the liver tissue extracts further indicated that transcriptional gene silencing was taking place (FIG. 11d). Adenoviral luciferase expression at longer time points by chemiluminescence indicated that luciferase expression in the liver of OKSM-injected animals only remained downregulated on both day 5 and 9 (FIG. 11e and FIG. 12).

It is well-established that Ad5 infection can lead to severe hepato-physiological abnormalities, tissue damage and toxicity^{(20, 21)}. We therefore investigated whether in vivo cell reprogramming and the generation of induced pluripotent stem cells in the liver can offer any physiological improvements against Ad5 infection. Significant elevation of liver enzymes in serum (FIG. 11f) and tissue damage (FIG. 11g) were observed both at day 5 and 9 after Ad5 (10¹¹ particles/animal) intravenous administration in Balb/C mice. Strikingly, HTV-injection of the OKSM plasmids 4h after Ad5 administration led to significant decrease in serum enzymes (FIG. 11f) and prevention of liver tissue damage (FIG. 11g) at both time points. This is considered proof-of-principle evidence that in vivo cell reprogramming and generation of induced pluripotent stem cells by the over-expression of certain genes (such as transcription factors) can lead to ‘phenotypic resetting’ of epigenetic mutations and genomic aberrations that are associated with pathological conditions in host tissues and can lead to therapeutic outcomes. In this context we propose direct in vivo cell reprogramming as a novel therapeutic strategy.

The following table shows the primer sequences used for qRT-PCR in the invention.

SUPPLEMENTARY TABLE 1
List of primer sequences used for qRT-PCR

| | Forward Primer | Reverse Primer |
|---|---|---|
| Oct3/4 | TGAGAACCTTCAGGAGATATGCAA | CTCAATGCTAGTTCGCTTTCTCTTC |
| Sox2 | GGTTACCTCTTCCTCCCACTCCAG | TCACATGTGCGACAGGGGCAG |
| C-myc | CAGAGGAGAAACGAGCTGAAGCGC | TTATGCACCAGAGTTTCGAAGCTGTTCG |
| Nanog | CAGAAAAACCAGTGGTTGAAGACTAG | GCAATGGATGCTGGGATACTC |
| Ecat1 | TGTGGGGCCCTGAAAGGCGAGCTGAGAT | ATGGGCCGCCATACGACGACGCTCAACT |
| Rex1 | ACGAGTGGCAGTTTCTTCTTGGGA | TATGACTCACTTCCAGGGGGCACT |
| Endo-Oct3/4 | TCT TTC CAC CAG GCC CCC GGC TC | TGC GGG CGG ACA TGG GGA GAT CC |
| Endo-Sox2 | TAG AGC TAG ACT CCG GGC GAT GA | TTG CCT TAA ACA AGA CCA CGA AA |
| Endo-Klf | GCG AAC TCA CAC AGG CGA GAA ACC | TCG CTT CCT CTT CCT CCG ACA CA |
| E-Ras | ACT GCC CCT CAT CAG ACT GCT TTC | CAC TGC CTT GTA CTC GGG TAG CTG |
| Cripto | ATG GAC GCA ACT GTG AAC ATG ATG | CTT TGA GGT CCT GGT CCA TCA CGT |
| | TTC GCA | GAC CAT |
| Gdf3 | GTT CCA ACC TGT GCC TCG CGT CTT | AGC GAG GCA TGG AGA GAG CGG AGC AG |
| Alb | GTTCGCTACACCCAGAAAGC | CCACACAAGGCAGTCTCTGA |
| Trf | ACCATGTTGTGGTCTCACGA | ACAGAAGGTCCTTGGTGGTG |
| Aat | CAGAGGAGGCCAAGAAAGTG | ATGGACAGTCTGGGGAAGTG |
| B-Actin | GACCTCTATGCCAACACAGT | AGTACTTGCGCTCAGGAGGA |
| Cbr-Luc | GTCGATGGTCGGCGATGAATCTTTGAGC | TAGCTCTCGTTGACTGGAGCCAGGATCATA |

### Study 2

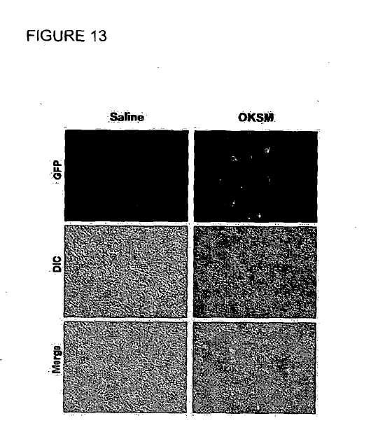

Considering the critical role of Nanog in the control of pluripotency, we performed a separate experiment using the transgenic strain Nanog-GFP (TNG-A) that carries the eGFP reporter inserted into the Nanog locus. Confirmation of the enhanced generation of pluripotent cells in the liver was offered by the presence of eGFP-positive cells in frozen tissue sections imaged by fluorescence microscopy (FIG. 13).

In addition to the liver, muscle tissue was tested for the induction of pluripotent phenotype in vivo. Intramuscular (i.m.) injection of plasmid DNA has been previously shown to result in efficient gene expression in muscle fibers. Balb/C skeletal muscle was intramuscularly injected with pDNA encoding for Oct3/4, Sox2, Klf4 and c-Myc (pLenti-III-EF1a-mYamanaka) to provide further proof-of-principle evidence of in vivo cell reprogramming. Reprogramming plasmid pLenti-III-EF1a-mYamanaka, which encodes for OCT3/4, KLF4, SOX2, cMYC and eGFP under the control of EF1a promoter, was purchased from NBS Biologicals, UK.

Balb/C mice (4 animals/group) were anesthetized with isofluorane and skeletal muscle (TA) were intramuscularly injected with 25 μl of 0.9% saline including 50 μg of pLenti-III-EF1a-mYamanaka or no plasmid. Nanog-GFP mice (3 animals/group) were anesthetized with isofluorane and skeletal muscle (TA) were intramuscularly injected with 40 μl of 0.9% saline including 50 μg of pCX-OKS-2A and 50 μg of pCX-cMyc or no plasmid. Mice were culled at day2 after i.m. injections and muscle tissues were collected.

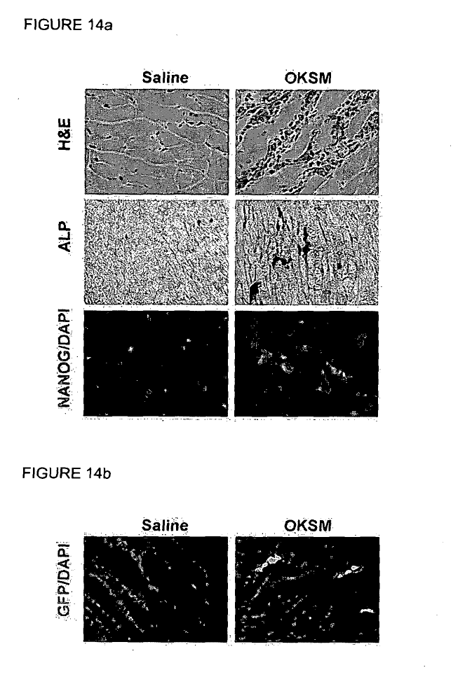

Two days after i.m. injection, muscle tissues were collected and histological analyses were performed (FIG. 14a). H&E staining showed formation of proliferating cell clusters between the skeletal muscle fibers. Treatment of muscle sections with BCIP/NBT substrate resulted in dark brown colour development, indicating ALP activity in the tissue. Moreover, positive staining for Nanog was obtained reproducibly in all muscle sections in all OKSM-transfected animals. These findings indicate the presence of pluripotent cells in the muscle tissue. To further confirm enhancement of pluripotency, Nanog-GFP mice were intramuscularly injected with pCX-OKS-2A and pCX-cMyc (FIG. 14b). Cell clusters with eGFP positive nuclei were observed in muscle sections by fluorescence microscopy. These results suggest that in vivo enhancement of pluripotency in muscle by overexpression of OKSM transcription factors is rapid and efficient enough to be detected in situ, similar to the liver tissue.

### REFERENCES

1. Wu, S. M., and Hochedlinger, K. (2011) Harnessing the potential of induced pluripotent stem cells for regenerative medicine. Nat Cell Biol 13, 497-505
2. Yamanaka, S. (2009) A Fresh Look at iPS Cells. Cell 137, 13-17
3. Takahashi, K., and Yamanaka, S. (2006) Induction of Pluripotent Stem Cells from Mouse Embryonic and Adult Fibroblast Cultures by Defined Factors. Cell 126, 663-676
4. Takahashi, K., Tanabe, K., Ohnuki, M., Narita, M., Ichisaka, T., Tomoda, K., and Yamanaka, S. (2007) Induction of Pluripotent Stem Cells from Adult Human Fibroblasts by Defined Factors. Cell 131, 861-872
5. Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., Slukvin, I. I., and Thomson, J. A. (2007) Induced Pluripotent Stem Cell Lines Derived from Human Somatic Cells. Science 318, 1917-1920
6. Warren, L., Manos, P. D., Ahfeldt, T., Loh, Y.-H., Li, H., Lau, F., Ebina, W., Mandal, P. K., Smith, Z. D., Meissner, A., Daley, G. Q., Brack, A. S., Collins, J. J., Cowan, C., Schlaeger, T. M., and Rossi, D. J. (2010) Highly Efficient Reprogramming to Pluripotency and Directed Differentiation of Human Cells with Synthetic Modified mRNA. Cell stem cell 7, 618-630
7. Miura, K., Okada, Y., Aoi, T., Okada, A., Takahashi, K., Okita, K., Nakagawa, M., Koyanagi, M., Tanabe, K., Ohnuki, M., Ogawa, D., Ikeda, E., Okano, H., and Yamanaka, S. (2009) Variation in the safety of induced pluripotent stem cell lines. Nat Biotech 27, 743-745
8. Jia, F., Wilson, K. D., Sun, N., Gupta, D. M., Huang, M., Li, Z., Panetta, N. J., Chen, Z. Y., Robbins, R. C., Kay, M. A., Longaker, M. T., and Wu, J. C. (2010) A nonviral minicircle vector for deriving human iPS cells. Nat Meth 7, 197-199
9. Stadtfeld, M., and Hochedlinger, K. (2011) Induced pluripotency: history, mechanisms, and applications. Gene Dev 24, 2239-2263
10. Liu, F., Song, Y. K., and Liu, D. (1999) Hydrodynamics-based transfection in animals by systemic administration of plasmid DNA. Gene Ther 6, 1258-1266
11. Zhang, G., Budker, V., and Wolff, J. A. (1999) High Levels of Foreign Gene Expression in Hepatocytes after Tail Vein Injections of Naked Plasmid DNA. Hum Gene Thar 10, 1735-1737
12. Andrianaivo, F., Lecocq, M., Wattiaux-De Coninck, S., Wattiaux, R., and Jadot, M. (2004) Hydrodynamics-based transfection of the liver: entrance into hepatocytes of DNA that causes expression takes place very early after injection. The Journal of Gene Medicine 6, 877-883
13. Herweijer, H., and Wolff, J. A. (2006) Gene therapy progress and prospects: Hydrodynamic gene delivery. Gene Ther 14, 99-107
14. Toumi, H., Hegge, J., Subbotin, V., Noble, M., Herweijer, H., Best, T. M., and Hagstrom, J. E. (2006) Rapid Intravascular Injection into Limb Skeletal Muscle: A Damage Assessment Study. Mol Ther 13, 229-236
15. Wells, D. J. (2004) Opening the Floodgates: Clinically Applicable Hydrodynamic Delivery of Plasmid DNA to Skeletal Muscle. Mol Ther 10, 207-208
16. Brambrink, T., Foreman, R., Welstead, G. G., Lengner, C. J., Wernig, M., Suh, H., and Jaenisch, R. (2008) Sequential Expression of Pluripotency Markers during Direct Reprogramming of Mouse Somatic Cells. Cell stem cell 2, 151-159
17. Chan, E. M., Ratanasirintrawoot, S., Park, I.-H., Manos, P. D., Loh, Y.-H., Huo, H., Miller, J. D., Hartung, 0., Rho, J., Ince, T. A., Daley, G. Q., and Schlaeger, T. M. (2009) Live cell imaging distinguishes bona fide human iPS cells from partially reprogrammed cells. Nat Biotech 27, 1033-1037
18. Chung, S., Andersson, T., Sonntag, K.-C., Björklund, L., Isacson, 0., and Kim, K.-S. (2002) Analysis of Different Promoter Systems for Efficient Transgene Expression in Mouse Embryonic Stem Cell Lines. STEM CELLS 20, 139-145
19. Shao, L., Feng, W., Sun, Y., Bai, H., Liu, J., Currie, C., Kim, J., Gama, R., Wang, Z., Qian, Z., Liaw, L., and Wu, W.-S. (2009) Generation of iPS cells using defined factors linked via the self-cleaving 2A sequences in a single open reading frame. Cell Res 19, 296-306
20. Shayakhmetov, D. M., Gaggar, A., Ni, S., Li, Z.-Y., and Lieber, A. (2005) Adenovirus Binding to Blood Factors Results in Liver Cell Infection and Hepatotoxicity. J. Virol. 79, 7478-7491
21. Shayakhmetov, D. M., Li, Z.-Y., Ni, S., and Lieber, A. (2004) Analysis of Adenovirus Sequestration in the Liver, Transduction of Hepatic Cells, and Innate Toxicity after Injection of Fiber-Modified Vectors. J Virol 78, 5368-5381
22. Khorsandi, S. E. et al., Minimally invasive and selective hydrodynamic gene therapy of liver segments in the pig and human. Cancer Gene Ther 15 (4), 225-230 (2008).
23. Maekawa, M. & Yamanaka, S. Glis1, a unique pro-reprogramming factor, may facilitate clinical applications of iPSC technology. Cell Cycle 474, 225-229 (2011).
24. Takahashi, K. et al. Induction of Pluripotent Stem Cells from Adult Human Fibroblasts by Defined Factors. Cell 131, 861-872 (2007).
25. Yu, J. et al. Induced Pluripotent Stem Cell Lines Derived from Human Somatic Cells. Science 318, 1917-1920, doi:10.1126/science.1151526 (2007).
26. Gonzalez, F., Boue, S. & Belmonte, J. C. I. Methods for making induced pluripotent stem cells: reprogramming Ã la carte. Nat. Rev. Genet. 12, 231-242 (2011).
27. Judson, R. L., Babiarz, J. E., Venere, M. & Blelloch, R. Embryonic stem cell-specific microRNAs promote induced pluripotency. Nat. Biotech. 27, 459-461 (2009).
28. Anokye-Danso, F. et al. Highly Efficient miRNA-Mediated Reprogramming of Mouse and Human Somatic Cells to Pluripotency. Cell stem cell 8, 376-388 (2011).
29. Lin, S.-L. et al. Regulation of somatic cell reprogramming through inducible mir-302 expression. Nucleic Acid Res., doi:10.1093/nar/gkq850 (2011).
30. Miyoshi, N. et al. Reprogramming of Mouse and Human Cells to Pluripotency Using Mature MicroRNAs. Cell stem cell 8, 633-638 (2011).
31. Subramanyam, D. et al. Multiple targets of miR-302 and miR-372 promote reprogramming of human fibroblasts to induced pluripotent stem cells. Nat. Biotech. 29, 443-448 (2011).

1. An in vivo method of de-differentiating a cell, comprising transfecting the cell with a defined factor.

2. A method according to claim 1, wherein the defined factor is a transcription factor, miRNA, DNA, or protein.

3. A method according to claim 2, wherein the transcription factor is selected from one or more of Oct3/4, Sox2, Klf4 and c-Myc Nanog, Lin 28 or Glist

4. A method according to claim 2, wherein the miRNA is selected from one or more of miR-291, miR-295, mi302, mi372, mi369, mi302 or mi367.

5. A method according claim 2, wherein the cell is transfected by one or more vectors comprising the transcription factor.

6. A method according to claim 5, wherein the vector is a viral vector.

7. A method according to claim 5, wherein the vector is a plasmid.

8. A method according to claim 7, wherein the one or more vectors are selected from the plasmids pCX-OKS-2A, pCX-cMyc, and pLenti-III-EF1a-mYamanaka.

9. A method according claim 1, wherein the cell is a hepatic cell or a muscle cell.

10. A method according claim 1, wherein the defined factor is administered to the body by hydrodynamic injection.

11. A pluripotent stem cell obtained by the method of claim 1.

12. (canceled)

13. (canceled)

14. A method according to claim 1, which comprises inducing a pluripotent phenotype in vivo in a subject in need of therapy.

15. (canceled)

16. A method according to claim 14, wherein the therapy is of a disease associated with loss of tissue.

17. A method according to claim 14, wherein the therapy is of cancer.

18-21. (canceled)

22. A pharmaceutical composition comprising a defined factor and a pharmaceutically acceptable excipient.

23. A composition according to claim 22, wherein the defined factor is a transcription factor, miRNA, DNA, or protein.

24. A composition according to claim 23, suitable for delivery via a hydrodynamic injection.

25. (canceled)

26. An in vitro method of culturing pluripotent stem cells, comprising obtaining a stem cell by the method of claim 1, and culturing the stem cell in vitro under conditions whereby the stem cell proliferates.
